(19) United States
(12) Patent Application Publication
(10) Pub. No.: US 2025/0163182 A1
(43) Pub. Date: May 22, 2025

(54) ENPP3 AND CD3 BINDING AGENTS AND METHODS OF USE THEREOF

(71) Applicant: Janssen Biotech, Inc., Horsham, PA (US)

(72) Inventors: Smruthi Vijayaraghavan, Lansdale, PA (US); Eilyn R. Lacy, Wayne, PA (US); Yoshitomo Hamuro, Lawrenceville, NJ (US)

(21) Appl. No.: 18/795,687

(22) Filed: Aug. 6, 2024

Related U.S. Application Data
(60) Provisional application No. 63/531,157, filed on Aug. 7, 2023.

(51) Int. Cl. C07K 16/40; A61K 39/00; A61P 35/00; C07K 16/28

(52) U.S. Cl. CPC C07K 16/40; A61P 35/00; C07K 16/2809; A61K 2039/505; C07K 2317/31; C07K 2317/33; C07K 2317/524; C07K 2317/526; C07K 2317/55; C07K 2317/565; C07K 2317/622; C07K 2317/92

(57) ABSTRACT

The present disclosure provides ENPP3 binding agents, including bispecific ENPP3×CD3 binding proteins, and related compositions and methods of use.

### CROSS-REFERENCE TO RELATED APPLICATIONS

This application claims priority to U.S. Provisional Application No. 63/531,157, filed on Aug. 7, 2023 the disclosure of which is herein incorporated by reference in its entirety.

### SEQUENCE LISTING

The instant application contains a Sequence Listing which has been submitted electronically in XML format and is hereby incorporated by reference in its entirety. The XML copy, created on Aug. 6, 2024, is named JBI6827USNP1_SL.xml and is 290 kilobytes in size.

### 1. FIELD

The present disclosure generally relates to a T cell engager that binds anti-ectonucleotide pyrophosphatase/phosphodiesterase family member 3 (ENPP3) and cluster of differentiation 3 (CD3), compositions comprising same and methods of using same.

### 2. BACKGROUND

Solid tumors have been a challenge for antibody-mediated T cell redirection therapy, with very limited, if any, drugs yet approved by the US Food and Drug Administration (FDA). Limitation of the approach may be related to the lack of cancer specificity of targets leading to toxicity occurring at subefficacious doses. Unmet need remains across solid tumors to prolong and improve outcomes and overall treatment duration.

There exists a need for innovative approaches for effective targeting of solid tumors and minimizing the treatment toxicities. The present disclosure meets this and other needs.

### 3. SUMMARY

In one aspect, provided herein is a binding agent comprising an antigen binding region that binds to an epitope of ENPP3. In some embodiments, the epitope of ENPP3 is selected from the group consisting of SEQ ID NO:295-297, DVP, and SEQ ID NO:299-305.

In some embodiments, the antigen binding region comprises a VH and VL domain selected from the group consisting of: (a) a VH domain comprising a HCDR1, HCDR2, and HCDR3 of a VH as set forth in SEQ ID NO:22, and a VL domain comprising a LCDR1, LCDR2, and LCDR3 of a VL as set forth in SEQ ID NO:23; (b) a VH domain comprising a HCDR1, HCDR2, and HCDR3 of a VH as set forth in SEQ ID NO:45, and a VL domain comprising a LCDR1, LCDR2, and LCDR3 of a VL as set forth in SEQ ID NO:46; (c) a VH domain comprising a HCDR1, HCDR2, and HCDR3 of a VH as set forth in SEQ ID NO:68, and a VL domain comprising a LCDR1, LCDR2, and LCDR3 of a VL as set forth in SEQ ID NO:69 or SEQ ID NO:70; (d) a VH domain comprising a HCDR1, HCDR2, and HCDR3 of a VH as set forth in SEQ ID NO:91, and a VL domain comprising a LCDR1, LCDR2, and LCDR3 of a VL as set forth in SEQ ID NO:92; (e) a VH domain comprising a HCDR1, HCDR2, and HCDR3 of a VH as set forth in SEQ ID NO:113, and a VL domain comprising a LCDR1, LCDR2, and LCDR3 of a VL as set forth in SEQ ID NO: 114 or SEQ ID NO: 115; (f) a VH domain comprising a HCDR1, HCDR2, and HCDR3 of a VH as set forth in SEQ ID NO: 134, and a VL domain comprising a LCDR1, LCDR2, and LCDR3 of a VL as set forth in SEQ ID NO: 135 or SEQ ID NO:136; (g) a VH domain comprising a HCDR1, HCDR2, and HCDR3 of a VH as set forth in SEQ ID NO: 158, and a VL domain comprising a LCDR1, LCDR2, and LCDR3 of a VL as set forth in SEQ ID NO:159; and (h) a VH domain comprising a HCDR1, HCDR2, and HCDR3 of a VH as set forth in SEQ ID NO: 177, and a VL domain comprising a LCDR1, LCDR2, and LCDR3 of a VL as set forth in SEQ ID NO:178.

In some embodiments, the CDR sequences are selected from the group consisting of: (a1) the HCDR1 comprises the amino acid sequence of SEQ ID NO: 1, the HCDR2 comprises the amino acid sequence of SEQ ID NO:2, the HCDR3 comprises the amino acid sequence of SEQ ID NO: 3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6; (a2) the HCDR1 comprises the amino acid sequence of SEQ ID NO: 7, the HCDR2 comprises the amino acid sequence of SEQ ID NO:8, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6; (a3) the HCDR1 comprises the amino acid sequence of SEQ ID NO:9, the HCDR2 comprises the amino acid sequence of SEQ ID NO:10, the HCDR3 comprises the amino acid sequence of SEQ ID NO:3, the LCDR1 comprises the amino acid sequence of SEQ ID NO:4, the LCDR2 comprises the amino acid sequence of SEQ ID NO: 5, and the LCDR3 comprises the amino acid sequence of SEQ ID NO:6; (a4) the HCDR1 comprises the amino acid sequence of SEQ ID NO:11, the HCDR2 comprises the amino acid sequence of SEQ ID NO:12, the HCDR3 comprises the amino acid sequence of SEQ ID NO:13, the LCDR1 comprises the amino acid sequence of SEQ ID NO:14, the LCDR2 comprises the amino acid sequence of SEQ ID NO: 15, and the LCDR3 comprises the amino acid sequence of SEQ ID NO: 16; (a5) the HCDR1 comprises the amino acid sequence of SEQ ID NO:17, the HCDR2 comprises the amino acid sequence of SEQ ID NO:18, the HCDR3 comprises the amino acid sequence of SEQ ID NO: 19, the LCDR1 comprises the amino acid sequence of SEQ ID NO: 20, the LCDR2 comprises the amino acid sequence of GAS, and the LCDR3 comprises the amino acid sequence of SEQ ID NO:6; (b1) the HCDR1 comprises the amino acid sequence of SEQ ID NO:24, the HCDR2 comprises the amino acid sequence of SEQ ID NO:25, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO:29; (b2) the HCDR1 comprises the amino acid sequence of SEQ ID NO:30, the HCDR2 comprises the amino acid sequence of SEQ ID NO:31, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO:29; (b3) the HCDR1 comprises the amino acid sequence of SEQ ID NO:32, the HCDR2 comprises the amino acid sequence of SEQ ID NO:33, the HCDR3 comprises the amino acid sequence of SEQ ID NO:26, the LCDR1 comprises the amino acid sequence of SEQ ID NO: 27, the LCDR2 comprises the amino acid sequence of SEQ ID NO:28, and the LCDR3 comprises the amino acid sequence of SEQ ID NO:29; (b4) the HCDR1 comprises the amino acid sequence of SEQ ID NO:34, the HCDR2 comprises the amino acid sequence of SEQ ID NO: 35, the HCDR3 comprises the amino acid sequence of SEQ ID NO:36, the LCDR1 comprises the amino acid sequence of SEQ ID NO:37, the LCDR2 comprises the amino acid sequence of SEQ ID NO:38, and the LCDR3 comprises the amino acid sequence of SEQ ID NO: 39; (b5) the HCDR1 comprises the amino acid sequence of SEQ ID NO:40, the HCDR2 comprises the amino acid sequence of SEQ ID NO:41, the HCDR3 comprises the amino acid sequence of SEQ ID NO:42, the LCDR1 comprises the amino acid sequence of SEQ ID NO:43, the LCDR2 comprises the amino acid sequence of DAS, and the LCDR3 comprises the amino acid sequence of SEQ ID NO:29; (c1) the HCDR1 comprises the amino acid sequence of SEQ ID NO: 47, the HCDR2 comprises the amino acid sequence of SEQ ID NO:48, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO:52; (c2) the HCDR1 comprises the amino acid sequence of SEQ ID NO:53, the HCDR2 comprises the amino acid sequence of SEQ ID NO:54, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO:52; (c3) the HCDR1 comprises the amino acid sequence of SEQ ID NO:55, the HCDR2 comprises the amino acid sequence of SEQ ID NO:56, the HCDR3 comprises the amino acid sequence of SEQ ID NO:49, the LCDR1 comprises the amino acid sequence of SEQ ID NO: 50, the LCDR2 comprises the amino acid sequence of SEQ ID NO:51, and the LCDR3 comprises the amino acid sequence of SEQ ID NO:52; (c4) the HCDR1 comprises the amino acid sequence of SEQ ID NO:57, the HCDR2 comprises the amino acid sequence of SEQ ID NO: 58, the HCDR3 comprises the amino acid sequence of SEQ ID NO:59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO: 62; (c5) the HCDR1 comprises the amino acid sequence of SEQ ID NO:63, the HCDR2 comprises the amino acid sequence of SEQ ID NO:64, the HCDR3 comprises the amino acid sequence of SEQ ID NO:65, the LCDR1 comprises the amino acid sequence of SEQ ID NO:66, the LCDR2 comprises the amino acid sequence of QIS, and the LCDR3 comprises the amino acid sequence of SEQ ID NO:52; (d1) the HCDR1 comprises the amino acid sequence of SEQ ID NO: 71, the HCDR2 comprises the amino acid sequence of SEQ ID NO:72, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO:76; (d2) the HCDR1 comprises the amino acid sequence of SEQ ID NO:77, the HCDR2 comprises the amino acid sequence of SEQ ID NO:78, the HCDR3 comprises the amino acid sequence of SEQ ID NO: 73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO:76; (d3) the HCDR1 comprises the amino acid sequence of SEQ ID NO:79, the HCDR2 comprises the amino acid sequence of SEQ ID NO:80, the HCDR3 comprises the amino acid sequence of SEQ ID NO:73, the LCDR1 comprises the amino acid sequence of SEQ ID NO: 74, the LCDR2 comprises the amino acid sequence of SEQ ID NO:75, and the LCDR3 comprises the amino acid sequence of SEQ ID NO:76; (d4) the HCDR1 comprises the amino acid sequence of SEQ ID NO:81, the HCDR2 comprises the amino acid sequence of SEQ ID NO: 82, the HCDR3 comprises the amino acid sequence of SEQ ID NO:83, the LCDR1 comprises the amino acid sequence of SEQ ID NO:84, the LCDR2 comprises the amino acid sequence of SEQ ID NO:85, and the LCDR3 comprises the amino acid sequence of SEQ ID NO: 86; (d5) the HCDR1 comprises the amino acid sequence of SEQ ID NO:87, the HCDR2 comprises the amino acid sequence of SEQ ID NO:88, the HCDR3 comprises the amino acid sequence of SEQ ID NO:89, the LCDR1 comprises the amino acid sequence of SEQ ID NO:90, the LCDR2 comprises the amino acid sequence of GAS, and the LCDR3 comprises the amino acid sequence of SEQ ID NO:76; (e1) the HCDR1 comprises the amino acid sequence of SEQ ID NO: 93, the HCDR2 comprises the amino acid sequence of SEQ ID NO:94, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97 and the LCDR3 comprises the amino acid sequence of SEQ ID NO:98; (e2) the HCDR1 comprises the amino acid sequence of SEQ ID NO:99, the HCDR2 comprises the amino acid sequence of SEQ ID NO: 100, the HCDR3 comprises the amino acid sequence of SEQ ID NO: 95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO:98; (e3) the HCDR1 comprises the amino acid sequence of SEQ ID NO: 101, the HCDR2 comprises the amino acid sequence of SEQ ID NO: 102, the HCDR3 comprises the amino acid sequence of SEQ ID NO:95, the LCDR1 comprises the amino acid sequence of SEQ ID NO:96 the LCDR2 comprises the amino acid sequence of SEQ ID NO:97, and the LCDR3 comprises the amino acid sequence of SEQ ID NO:98; (e4) the HCDR1 comprises the amino acid sequence of SEQ ID NO: 103, the HCDR2 comprises the amino acid sequence of SEQ ID NO: 104, the HCDR3 comprises the amino acid sequence of SEQ ID NO: 105, the LCDR1 comprises the amino acid sequence of SEQ ID NO: 106, the LCDR2 comprises the amino acid sequence of SEQ ID NO: 107, and the LCDR3 comprises the amino acid sequence of SEQ ID NO: 108; (e5) the HCDR1 comprises the amino acid sequence of SEQ ID NO: 109, the HCDR2 comprises the amino acid sequence of SEQ ID NO: 110, the HCDR3 comprises the amino acid sequence of SEQ ID NO:111, the LCDR1 comprises the amino acid sequence of SEQ ID NO: 112, the LCDR2 comprises the amino acid sequence of GAS, and the LCDR3 comprises the amino acid sequence of SEQ ID NO:98; (f1) the HCDR1 comprises the amino acid sequence of SEQ ID NO: 116, the HCDR2 comprises the amino acid sequence of SEQ ID NO: 117, the HCDR3 comprises the amino acid sequence of SEQ ID NO:118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO:120; (f2) the HCDR1 comprises the amino acid sequence of SEQ ID NO:121, the HCDR2 comprises the amino acid sequence of SEQ ID NO:122, the HCDR3 comprises the amino acid sequence of SEQ ID NO:118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO: 120; (f3) the HCDR1 comprises the amino acid sequence of SEQ ID NO: 123, the HCDR2 comprises the amino acid sequence of SEQ ID NO: 124, the HCDR3 comprises the amino acid sequence of SEQ ID NO:118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO:120; (f4) the HCDR1 comprises the amino acid sequence of SEQ ID NO:125, the HCDR2 comprises the amino acid sequence of SEQ ID NO: 126, the HCDR3 comprises the amino acid sequence of SEQ ID NO: 127, the LCDR1 comprises the amino acid sequence of SEQ ID NO: 128, the LCDR2 comprises the amino acid sequence of SEQ ID NO: 15, and the LCDR3 comprises the amino acid sequence of SEQ ID NO: 129; (f5) the HCDR1 comprises the amino acid sequence of SEQ ID NO: 130, the HCDR2 comprises the amino acid sequence of SEQ ID NO: 131, the HCDR3 comprises the amino acid sequence of SEQ ID NO:132, the LCDR1 comprises the amino acid sequence of SEQ ID NO:133, the LCDR2 comprises the amino acid sequence of GAS, and the LCDR3 comprises the amino acid sequence of SEQ ID NO: 120; (g1) the HCDR1 comprises the amino acid sequence of SEQ ID NO: 137, the HCDR2 comprises the amino acid sequence of SEQ ID NO: 138, the HCDR3 comprises the amino acid sequence of SEQ ID NO:139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO: 142; (g2) the HCDR1 comprises the amino acid sequence of SEQ ID NO: 143, the HCDR2 comprises the amino acid sequence of SEQ ID NO: 144, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO: 142; (g3) the HCDR1 comprises the amino acid sequence of SEQ ID NO: 145, the HCDR2 comprises the amino acid sequence of SEQ ID NO: 146, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO:142; (g4) the HCDR1 comprises the amino acid sequence of SEQ ID NO: 147, the HCDR2 comprises the amino acid sequence of SEQ ID NO: 148, the HCDR3 comprises the amino acid sequence of SEQ ID NO: 149, the LCDR1 comprises the amino acid sequence of SEQ ID NO:150, the LCDR2 comprises the amino acid sequence of SEQ ID NO:151, and the LCDR3 comprises the amino acid sequence of SEQ ID NO: 152; (g5) the HCDR1 comprises the amino acid sequence of SEQ ID NO: 153, the HCDR2 comprises the amino acid sequence of SEQ ID NO: 154, the HCDR3 comprises the amino acid sequence of SEQ ID NO:155, the LCDR1 comprises the amino acid sequence of SEQ ID NO: 156, the LCDR2 comprises the amino acid sequence of AAS, and the LCDR3 comprises the amino acid sequence of SEQ ID NO:142; (h1) the HCDR1 comprises the amino acid sequence of SEQ ID NO: 1, the HCDR2 comprises the amino acid sequence of SEQ ID NO: 160, the HCDR3 comprises the amino acid sequence of SEQ ID NO: 161, the LCDR1 comprises the amino acid sequence of SEQ ID NO:162, the LCDR2 comprises the amino acid sequence of SEQ ID NO: 163, and the LCDR3 comprises the amino acid sequence of SEQ ID NO:76; (h2) the HCDR1 comprises the amino acid sequence of SEQ ID NO: 164, the HCDR2 comprises the amino acid sequence of SEQ ID NO: 165, the HCDR3 comprises the amino acid sequence of SEQ ID NO: 161, the LCDR1 comprises the amino acid sequence of SEQ ID NO: 162 the LCDR2 comprises the amino acid sequence of SEQ ID NO: 163, and the LCDR3 comprises the amino acid sequence of SEQ ID NO:76; (h3) the HCDR1 comprises the amino acid sequence of SEQ ID NO: 166, the HCDR2 comprises the amino acid sequence of SEQ ID NO:167, the HCDR3 comprises the amino acid sequence of SEQ ID NO:161, the LCDR1 comprises the amino acid sequence of SEQ ID NO:162, the LCDR2 comprises the amino acid sequence of SEQ ID NO: 163, and the LCDR3 comprises the amino acid sequence of SEQ ID NO:76; (h4) the HCDR1 comprises the amino acid sequence of SEQ ID NO: 11, the HCDR2 comprises the amino acid sequence of SEQ ID NO: 168, the HCDR3 comprises the amino acid sequence of SEQ ID NO: 169, the LCDR1 comprises the amino acid sequence of SEQ ID NO:170, the LCDR2 comprises the amino acid sequence of SEQ ID NO:171, and the LCDR3 comprises the amino acid sequence of SEQ ID NO:86; and (h5) the HCDR1 comprises the amino acid sequence of SEQ ID NO: 172, the HCDR2 comprises the amino acid sequence of SEQ ID NO: 173, the HCDR3 comprises the amino acid sequence of SEQ ID NO: 174, the LCDR1 comprises the amino acid sequence of SEQ ID NO: 175, the LCDR2 comprises the amino acid sequence of VAS and the LCDR3 comprises the amino acid sequence of SEQ ID NO:76.

In some embodiments, the antigen binding region comprises a VH domain and a VL domain selected from the group consisting of: (a) a VH domain comprising the amino acid sequence of SEQ ID NO:22, and a VL domain comprising the amino acid sequence of SEQ ID NO: 23; (b) a VH domain comprising the amino acid sequence of SEQ ID NO:45, and a VL domain comprising the amino acid sequence of SEQ ID NO:46; (c) a VH domain comprising the amino acid sequence of SEQ ID NO:68, and a VL domain comprising the amino acid sequence of SEQ ID NO:69; (c′) a VH domain comprising the amino acid sequence of SEQ ID NO:68, and a VL domain comprising the amino acid sequence of SEQ ID NO:70; (d) a VH domain comprising the amino acid sequence of SEQ ID NO:91, and a VL domain comprising the amino acid sequence of SEQ ID NO:92; (e) a VH domain comprising the amino acid sequence of SEQ ID NO: 113, and a VL domain comprising the amino acid sequence of SEQ ID NO: 114; (e′) a VH domain comprising the amino acid sequence of SEQ ID NO: 113, and a VL domain comprising the amino acid sequence of SEQ ID NO:115; (f) a VH domain comprising the amino acid sequence of SEQ ID NO:134, and a VL domain comprising the amino acid sequence of SEQ ID NO:135; (f′) a VH domain comprising the amino acid sequence of SEQ ID NO: 134, and a VL domain comprising the amino acid sequence of SEQ ID NO:136; (g) a VH domain comprising the amino acid sequence of SEQ ID NO: 158, and a VL domain comprising the amino acid sequence of SEQ ID NO: 159; and (h) a VH domain comprising the amino acid sequence of SEQ ID NO:177, and a VL domain comprising the amino acid sequence of SEQ ID NO:178.

In some embodiments, the binding agent is a bispecific protein or a multi-specific protein.

In some embodiments, the binding agent further comprises an immunoglobulin (Ig) constant region, or a fragment of the Ig constant region, wherein optionally the fragment of the Ig constant region is an Fc region or an CH3 domain. In some embodiments, the Ig constant region, the fragment of the Ig constant region, the Fc region, or the CH3 domain comprises at least one mutation. In some embodiments, the at least one mutation is selected from the group consisting of L234A/L235A/D265S, F234A/L235A, L234A/L235A, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index. In some embodiments, the at least one mutation is selected from the group consisting of T366S/L368A/Y407V, T366W, T350V, L351Y, F405A, Y407V, T366Y, T366L, F405W, T394W, K392L, T394S, Y407T, Y407A, L351Y/F405A/Y407V, T366I/K392M/T394W, F405A/Y407V, T366L/K392M/T394W, T366L/K392L/T394W, L351Y/Y407A, L351Y/Y407V, T366A/K409F, T366V/K409F, T366A/K409F, T350V/L351Y/F405A/Y407V and T350V/T366L/K392L/T394W, wherein residue numbering is according to the EU index. In some embodiments, the binding agent comprises knob-in-hole mutations, wherein the knob mutations comprise T366S/L368A/Y407V, and the hole mutation comprises T366W.

In some embodiments, the agent comprises a bispecific protein comprising an antigen binding region that binds a second antigen other than ENPP3. In some embodiments, the second antigen is cluster of differentiation 3ε (CD3ε).

In some embodiments, the binding agent comprises a first antigen binding region that binds to ENPP3 and a second antigen binding region that binds to CD3ε, wherein the first antigen binding region that binds to ENPP3 comprises a VH and VL selected from the group consisting of: (a) a VH domain comprising a HCDR1, HCDR2, and HCDR3 of a VH as set forth in SEQ ID NO:22, and a VL domain comprising a LCDR1, LCDR2, and LCDR3 of a VL as set forth in SEQ ID NO:23; (b) a VH domain comprising a HCDR1, HCDR2, and HCDR3 of a VH as set forth in SEQ ID NO:45, and a VL domain comprising a LCDR1, LCDR2, and LCDR3 of a VL as set forth in SEQ ID NO:46; (c) a VH domain comprising a HCDR1, HCDR2, and HCDR3 of a VH as set forth in SEQ ID NO:68, and a VL domain comprising a LCDR1, LCDR2, and LCDR3 of a VL as set forth in SEQ ID NO:69 or SEQ ID NO:70; (d) a VH domain comprising a HCDR1, HCDR2, and HCDR3 of a VH as set forth in SEQ ID NO:91, and a VL domain comprising a LCDR1, LCDR2, and LCDR3 of a VL as set forth in SEQ ID NO:92; (e) a VH domain comprising a HCDR1, HCDR2, and HCDR3 of a VH as set forth in SEQ ID NO:113, and a VL domain comprising a LCDR1, LCDR2, and LCDR3 of a VL as set forth in SEQ ID NO: 114 or SEQ ID NO:115; (f) a VH domain comprising a HCDR1, HCDR2, and HCDR3 of a VH as set forth in SEQ ID NO: 134, and a VL domain comprising a LCDR1, LCDR2, and LCDR3 of a VL as set forth in SEQ ID NO: 135 or SEQ ID NO: 136; (g) a VH domain comprising a HCDR1, HCDR2, and HCDR3 of a VH as set forth in SEQ ID NO: 158, and a VL domain comprising a LCDR1, LCDR2, and LCDR3 of a VL as set forth in SEQ ID NO:159; and (h) a VH domain comprising a HCDR1, HCDR2, and HCDR3 of a VH as set forth in SEQ ID NO: 177, and a VL domain comprising a LCDR1, LCDR2, and LCDR3 of a VL as set forth in SEQ ID NO:178.

In some embodiments, the second antigen binding region that binds to CD3E comprises a VH domain comprising a HCDR1, HCDR2, and HCDR3 of a VH as set forth in SEQ ID NO:200, and a VL domain comprising a LCDR1, LCDR2, and LCDR3 of a VL as set forth in SEQ ID NO:201.

In some embodiments, the first antigen binding region that binds to ENPP3 comprises CDR sequences selected from the group consisting of: (a1) the HCDR1 comprises the amino acid sequence of SEQ ID NO: 1, the HCDR2 comprises the amino acid sequence of SEQ ID NO: 2, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6; (a2) the HCDR1 comprises the amino acid sequence of SEQ ID NO:7, the HCDR2 comprises the amino acid sequence of SEQ ID NO:8, the HCDR3 comprises the amino acid sequence of SEQ ID NO: 3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6; (a3) the HCDR1 comprises the amino acid sequence of SEQ ID NO:9, the HCDR2 comprises the amino acid sequence of SEQ ID NO: 10, the HCDR3 comprises the amino acid sequence of SEQ ID NO:3, the LCDR1 comprises the amino acid sequence of SEQ ID NO: 4, the LCDR2 comprises the amino acid sequence of SEQ ID NO:5, and the LCDR3 comprises the amino acid sequence of SEQ ID NO:6; (a4) the HCDR1 comprises the amino acid sequence of SEQ ID NO:11, the HCDR2 comprises the amino acid sequence of SEQ ID NO: 12, the HCDR3 comprises the amino acid sequence of SEQ ID NO: 13, the LCDR1 comprises the amino acid sequence of SEQ ID NO:14, the LCDR2 comprises the amino acid sequence of SEQ ID NO: 15, and the LCDR3 comprises the amino acid sequence of SEQ ID NO:16; (a5) the HCDR1 comprises the amino acid sequence of SEQ ID NO:17, the HCDR2 comprises the amino acid sequence of SEQ ID NO: 18, the HCDR3 comprises the amino acid sequence of SEQ ID NO: 19, the LCDR1 comprises the amino acid sequence of SEQ ID NO:20, the LCDR2 comprises the amino acid sequence of GAS, and the LCDR3 comprises the amino acid sequence of SEQ ID NO:6; (b1) the HCDR1 comprises the amino acid sequence of SEQ ID NO:24, the HCDR2 comprises the amino acid sequence of SEQ ID NO:25, the HCDR3 comprises the amino acid sequence of SEQ ID NO:26, the LCDR1 comprises the amino acid sequence of SEQ ID NO: 27, the LCDR2 comprises the amino acid sequence of SEQ ID NO:28, and the LCDR3 comprises the amino acid sequence of SEQ ID NO:29; (b2) the HCDR1 comprises the amino acid sequence of SEQ ID NO:30, the HCDR2 comprises the amino acid sequence of SEQ ID NO: 31, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29; (b3) the HCDR1 comprises the amino acid sequence of SEQ ID NO:32, the HCDR2 comprises the amino acid sequence of SEQ ID NO:33, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO:29; (b4) the HCDR1 comprises the amino acid sequence of SEQ ID NO:34, the HCDR2 comprises the amino acid sequence of SEQ ID NO:35, the HCDR3 comprises the amino acid sequence of SEQ ID NO:36, the LCDR1 comprises the amino acid sequence of SEQ ID NO:37, the LCDR2 comprises the amino acid sequence of SEQ ID NO: 38, and the LCDR3 comprises the amino acid sequence of SEQ ID NO:39; (b5) the HCDR1 comprises the amino acid sequence of SEQ ID NO:40, the HCDR2 comprises the amino acid sequence of SEQ ID NO:41, the HCDR3 comprises the amino acid sequence of SEQ ID NO:42, the LCDR1 comprises the amino acid sequence of SEQ ID NO:43, the LCDR2 comprises the amino acid sequence of DAS, and the LCDR3 comprises the amino acid sequence of SEQ ID NO: 29; (c1) the HCDR1 comprises the amino acid sequence of SEQ ID NO:47, the HCDR2 comprises the amino acid sequence of SEQ ID NO:48, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO:52; (c2) the HCDR1 comprises the amino acid sequence of SEQ ID NO:53, the HCDR2 comprises the amino acid sequence of SEQ ID NO:54, the HCDR3 comprises the amino acid sequence of SEQ ID NO:49, the LCDR1 comprises the amino acid sequence of SEQ ID NO:50, the LCDR2 comprises the amino acid sequence of SEQ ID NO: 51, and the LCDR3 comprises the amino acid sequence of SEQ ID NO:52; (c3) the HCDR1 comprises the amino acid sequence of SEQ ID NO:55, the HCDR2 comprises the amino acid sequence of SEQ ID NO:56, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO:52; (c4) the HCDR1 comprises the amino acid sequence of SEQ ID NO:57, the HCDR2 comprises the amino acid sequence of SEQ ID NO:58, the HCDR3 comprises the amino acid sequence of SEQ ID NO:59, the LCDR1 comprises the amino acid sequence of SEQ ID NO: 60, the LCDR2 comprises the amino acid sequence of SEQ ID NO:61, and the LCDR3 comprises the amino acid sequence of SEQ ID NO:62; (c5) the HCDR1 comprises the amino acid sequence of SEQ ID NO:63, the HCDR2 comprises the amino acid sequence of SEQ ID NO: 64, the HCDR3 comprises the amino acid sequence of SEQ ID NO:65, the LCDR1 comprises the amino acid sequence of SEQ ID NO:66, the LCDR2 comprises the amino acid sequence of QIS, and the LCDR3 comprises the amino acid sequence of SEQ ID NO:52; (d1) the HCDR1 comprises the amino acid sequence of SEQ ID NO:71, the HCDR2 comprises the amino acid sequence of SEQ ID NO:72, the HCDR3 comprises the amino acid sequence of SEQ ID NO: 73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO:76; (d2) the HCDR1 comprises the amino acid sequence of SEQ ID NO: 77, the HCDR2 comprises the amino acid sequence of SEQ ID NO:78, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO:76; (d3) the HCDR1 comprises the amino acid sequence of SEQ ID NO:79, the HCDR2 comprises the amino acid sequence of SEQ ID NO:80, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO:76; (d4) the HCDR1 comprises the amino acid sequence of SEQ ID NO:81, the HCDR2 comprises the amino acid sequence of SEQ ID NO:82, the HCDR3 comprises the amino acid sequence of SEQ ID NO:83, the LCDR1 comprises the amino acid sequence of SEQ ID NO: 84, the LCDR2 comprises the amino acid sequence of SEQ ID NO:85, and the LCDR3 comprises the amino acid sequence of SEQ ID NO:86; (d5) the HCDR1 comprises the amino acid sequence of SEQ ID NO:87, the HCDR2 comprises the amino acid sequence of SEQ ID NO: 88, the HCDR3 comprises the amino acid sequence of SEQ ID NO:89, the LCDR1 comprises the amino acid sequence of SEQ ID NO:90, the LCDR2 comprises the amino acid sequence of GAS, and the LCDR3 comprises the amino acid sequence of SEQ ID NO:76; (e1) the HCDR1 comprises the amino acid sequence of SEQ ID NO:93, the HCDR2 comprises the amino acid sequence of SEQ ID NO:94, the HCDR3 comprises the amino acid sequence of SEQ ID NO: 95, the LCDR1 comprises the amino acid sequence of SEQ ID NO:96, the LCDR2 comprises the amino acid sequence of SEQ ID NO:97 and the LCDR3 comprises the amino acid sequence of SEQ ID NO:98; (e2) the HCDR1 comprises the amino acid sequence of SEQ ID NO: 99, the HCDR2 comprises the amino acid sequence of SEQ ID NO: 100, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO:98; (e3) the HCDR1 comprises the amino acid sequence of SEQ ID NO: 101, the HCDR2 comprises the amino acid sequence of SEQ ID NO: 102, the HCDR3 comprises the amino acid sequence of SEQ ID NO: 95, the LCDR1 comprises the amino acid sequence of SEQ ID NO:96 the LCDR2 comprises the amino acid sequence of SEQ ID NO:97, and the LCDR3 comprises the amino acid sequence of SEQ ID NO:98; (e4) the HCDR1 comprises the amino acid sequence of SEQ ID NO: 103, the HCDR2 comprises the amino acid sequence of SEQ ID NO:104, the HCDR3 comprises the amino acid sequence of SEQ ID NO: 105, the LCDR1 comprises the amino acid sequence of SEQ ID NO: 106, the LCDR2 comprises the amino acid sequence of SEQ ID NO: 107, and the LCDR3 comprises the amino acid sequence of SEQ ID NO: 108; (e5) the HCDR1 comprises the amino acid sequence of SEQ ID NO:109, the HCDR2 comprises the amino acid sequence of SEQ ID NO: 110, the HCDR3 comprises the amino acid sequence of SEQ ID NO:111, the LCDR1 comprises the amino acid sequence of SEQ ID NO: 112, the LCDR2 comprises the amino acid sequence of GAS, and the LCDR3 comprises the amino acid sequence of SEQ ID NO: 98; (f1) the HCDR1 comprises the amino acid sequence of SEQ ID NO:116, the HCDR2 comprises the amino acid sequence of SEQ ID NO: 117, the HCDR3 comprises the amino acid sequence of SEQ ID NO: 118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO: 120; (f2) the HCDR1 comprises the amino acid sequence of SEQ ID NO:121, the HCDR2 comprises the amino acid sequence of SEQ ID NO: 122, the HCDR3 comprises the amino acid sequence of SEQ ID NO: 118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO: 120; (f3) the HCDR1 comprises the amino acid sequence of SEQ ID NO: 123, the HCDR2 comprises the amino acid sequence of SEQ ID NO:124, the HCDR3 comprises the amino acid sequence of SEQ ID NO: 118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO: 120; (f4) the HCDR1 comprises the amino acid sequence of SEQ ID NO: 125, the HCDR2 comprises the amino acid sequence of SEQ ID NO: 126, the HCDR3 comprises the amino acid sequence of SEQ ID NO: 127, the LCDR1 comprises the amino acid sequence of SEQ ID NO:128, the LCDR2 comprises the amino acid sequence of SEQ ID NO:15, and the LCDR3 comprises the amino acid sequence of SEQ ID NO: 129; (f5) the HCDR1 comprises the amino acid sequence of SEQ ID NO: 130, the HCDR2 comprises the amino acid sequence of SEQ ID NO: 131, the HCDR3 comprises the amino acid sequence of SEQ ID NO: 132, the LCDR1 comprises the amino acid sequence of SEQ ID NO: 133, the LCDR2 comprises the amino acid sequence of GAS, and the LCDR3 comprises the amino acid sequence of SEQ ID NO: 120; (g1) the HCDR1 comprises the amino acid sequence of SEQ ID NO: 137, the HCDR2 comprises the amino acid sequence of SEQ ID NO: 138, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO: 142; (g2) the HCDR1 comprises the amino acid sequence of SEQ ID NO: 143, the HCDR2 comprises the amino acid sequence of SEQ ID NO: 144, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO: 142; (g3) the HCDR1 comprises the amino acid sequence of SEQ ID NO: 145, the HCDR2 comprises the amino acid sequence of SEQ ID NO:146, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO: 142; (g4) the HCDR1 comprises the amino acid sequence of SEQ ID NO: 147, the HCDR2 comprises the amino acid sequence of SEQ ID NO: 148, the HCDR3 comprises the amino acid sequence of SEQ ID NO:149, the LCDR1 comprises the amino acid sequence of SEQ ID NO: 150, the LCDR2 comprises the amino acid sequence of SEQ ID NO: 151, and the LCDR3 comprises the amino acid sequence of SEQ ID NO: 152; (g5) the HCDR1 comprises the amino acid sequence of SEQ ID NO: 153, the HCDR2 comprises the amino acid sequence of SEQ ID NO: 154, the HCDR3 comprises the amino acid sequence of SEQ ID NO: 155, the LCDR1 comprises the amino acid sequence of SEQ ID NO: 156, the LCDR2 comprises the amino acid sequence of AAS, and the LCDR3 comprises the amino acid sequence of SEQ ID NO: 142; the HCDR1 comprises the amino acid sequence of SEQ ID NO: 1, the HCDR2 comprises the amino acid sequence of SEQ ID NO: 160, the HCDR3 comprises the amino acid sequence of SEQ ID NO: 161, the LCDR1 comprises the amino acid sequence of SEQ ID NO: 162, the LCDR2 comprises the amino acid sequence of SEQ ID NO: 163, and the LCDR3 comprises the amino acid sequence of SEQ ID NO:76; the HCDR1 comprises the amino acid sequence of SEQ ID NO: 164, the HCDR2 comprises the amino acid sequence of SEQ ID NO:165, the HCDR3 comprises the amino acid sequence of SEQ ID NO: 161, the LCDR1 comprises the amino acid sequence of SEQ ID NO: 162 the LCDR2 comprises the amino acid sequence of SEQ ID NO: 163, and the LCDR3 comprises the amino acid sequence of SEQ ID NO:76; (h3) the HCDR1 comprises the amino acid sequence of SEQ ID NO: 166, the HCDR2 comprises the amino acid sequence of SEQ ID NO: 167, the HCDR3 comprises the amino acid sequence of SEQ ID NO: 161, the LCDR1 comprises the amino acid sequence of SEQ ID NO:162, the LCDR2 comprises the amino acid sequence of SEQ ID NO: 163, and the LCDR3 comprises the amino acid sequence of SEQ ID NO:76; the HCDR1 comprises the amino acid sequence of SEQ ID NO: 11, the HCDR2 comprises the amino acid sequence of SEQ ID NO: 168, the HCDR3 comprises the amino acid sequence of SEQ ID NO: 169, the LCDR1 comprises the amino acid sequence of SEQ ID NO:170, the LCDR2 comprises the amino acid sequence of SEQ ID NO: 171, and the LCDR3 comprises the amino acid sequence of SEQ ID NO:86; and (h5) the HCDR1 comprises the amino acid sequence of SEQ ID NO: 172, the HCDR2 comprises the amino acid sequence of SEQ ID NO: 173, the HCDR3 comprises the amino acid sequence of SEQ ID NO: 174, the LCDR1 comprises the amino acid sequence of SEQ ID NO:175, the LCDR2 comprises the amino acid sequence of VAS and the LCDR3 comprises the amino acid sequence of SEQ ID NO:76.

In some embodiments, the second antigen binding region that binds to CD3E comprises CDR sequences selected from the group consisting of: (a) the HCDR1 comprises the amino acid sequence of SEQ ID NO: 179, the HCDR2 comprises the amino acid sequence of SEQ ID NO: 180, the HCDR3 comprises the amino acid sequence of SEQ ID NO:181, the LCDR1 comprises the amino acid sequence of SEQ ID NO: 182, the LCDR2 comprises the amino acid sequence of SEQ ID NO: 183, and the LCDR3 comprises the amino acid sequence of SEQ ID NO:184; and (b) the HCDR1 comprises the amino acid sequence of SEQ ID NO:185, the HCDR2 comprises the amino acid sequence of SEQ ID NO: 186, the HCDR3 comprises the amino acid sequence of SEQ ID NO: 181, the LCDR1 comprises the amino acid sequence of SEQ ID NO: 182 the LCDR2 comprises the amino acid sequence of SEQ ID NO: 183, and the LCDR3 comprises the amino acid sequence of SEQ ID NO: 184; (c) the HCDR1 comprises the amino acid sequence of SEQ ID NO:187, the HCDR2 comprises the amino acid sequence of SEQ ID NO: 188, the HCDR3 comprises the amino acid sequence of SEQ ID NO:181, the LCDR1 comprises the amino acid sequence of SEQ ID NO: 182, the LCDR2 comprises the amino acid sequence of SEQ ID NO: 183, and the LCDR3 comprises the amino acid sequence of SEQ ID NO:184; or (d) the HCDR1 comprises the amino acid sequence of SEQ ID NO:189, the HCDR2 comprises the amino acid sequence of SEQ ID NO: 190, the HCDR3 comprises the amino acid sequence of SEQ ID NO:191, the LCDR1 comprises the amino acid sequence of SEQ ID NO: 192, the LCDR2 comprises the amino acid sequence of SEQ ID NO:193, and the LCDR3 comprises the amino acid sequence of SEQ ID NO: 194; and (e) the HCDR1 comprises the amino acid sequence of SEQ ID NO: 195, the HCDR2 comprises the amino acid sequence of SEQ ID NO: 196, the HCDR3 comprises the amino acid sequence of SEQ ID NO: 197, the LCDR1 comprises the amino acid sequence of SEQ ID NO: 198, the LCDR2 comprises the amino acid sequence of DSS and the LCDR3 comprises the amino acid sequence of SEQ ID NO: 184.

In some embodiments, (i) the first binding region comprises a VH domain comprising the amino acid sequence of SEQ ID NO:22, and a VL domain comprising the amino acid sequence of SEQ ID NO:23; and (ii) the second binding region comprises a VH domain comprising the amino acid sequence of SEQ ID NO:200, and a VL domain comprising the amino acid sequence of SEQ ID NO:201.

In some embodiments, the first antigen binding region comprises a Fab, and the second antigen binding region comprises a stapled scFv fragment (spFv). In some embodiments, the spFv comprises at least one disulfide bond between the VH or VL and the linker.

In some embodiments, the binding agent further comprises an immunoglobulin (Ig) constant region, a fragment of the Ig constant region, wherein optionally the fragment of the Ig constant region is an Fc region or an CH3 domain.

In one aspect, provided herein is a binding agent comprising (i) a first polypeptide comprising a spFv that binds CD3ε, a CH2 domain and a CH3 domain; (ii) a second polypeptide comprising a VH domain that binds ENPP3, a CH2 domain and a CH3 domain; and (iii) a third polypeptide comprising a VL domain that binds ENPP3, wherein the spFv that binds CD3E comprises a VH domain comprising a HCDR1, HCDR2, and HCDR3 of a VH as set forth in SEQ ID NO:200, and a VL domain comprising a LCDR1, LCDR2, and LCDR3 of a VL as set forth in SEQ ID NO:201; and wherein the second and third polypeptide comprising a VH and VL that bind to ENPP3, wherein the VH and VL that bind to ENPP3 are selected from the group consisting of: (a) a VH domain comprising a HCDR1, HCDR2, and HCDR3 of a VH as set forth in SEQ ID NO:22, and a VL domain comprising a LCDR1, LCDR2, and LCDR3 of a VL as set forth in SEQ ID NO:23; (b) a VH domain comprising a HCDR1, HCDR2, and HCDR3 of a VH as set forth in SEQ ID NO:45, and a VL domain comprising a LCDR1, LCDR2, and LCDR3 of a VL as set forth in SEQ ID NO:46; (c) a VH domain comprising a HCDR1, HCDR2, and HCDR3 of a VH as set forth in SEQ ID NO:68, and a VL domain comprising a LCDR1, LCDR2, and LCDR3 of a VL as set forth in SEQ ID NO:69 or SEQ ID NO:70; (d) a VH domain comprising a HCDR1, HCDR2, and HCDR3 of a VH as set forth in SEQ ID NO:91, and a VL domain comprising a LCDR1, LCDR2, and LCDR3 of a VL as set forth in SEQ ID NO:92; (e) a VH domain comprising a HCDR1, HCDR2, and HCDR3 of a VH as set forth in SEQ ID NO:113, and a VL domain comprising a LCDR1, LCDR2, and LCDR3 of a VL as set forth in SEQ ID NO: 114 or SEQ ID NO:115; (f) a VH domain comprising a HCDR1, HCDR2, and HCDR3 of a VH as set forth in SEQ ID NO: 134, and a VL domain comprising a LCDR1, LCDR2, and LCDR3 of a VL as set forth in SEQ ID NO:135 or SEQ ID NO:136; (g) a VH domain comprising a HCDR1, HCDR2, and HCDR3 of a VH as set forth in SEQ ID NO: 158, and a VL domain comprising a LCDR1, LCDR2, and LCDR3 of a VL as set forth in SEQ ID NO:159; and (h) a VH domain comprising a HCDR1, HCDR2, and HCDR3 of a VH as set forth in SEQ ID NO: 177, and a VL domain comprising a LCDR1, LCDR2, and LCDR3 of a VL as set forth in SEQ ID NO:178.

In some embodiments, (i) the spFv that binds CD3ε comprises CDRs selected from the group consisting of: (a) the HCDR1 comprises the amino acid sequence of SEQ ID NO:179, the HCDR2 comprises the amino acid sequence of SEQ ID NO: 180, the HCDR3 comprises the amino acid sequence of SEQ ID NO: 181, the LCDR1 comprises the amino acid sequence of SEQ ID NO: 182, the LCDR2 comprises the amino acid sequence of SEQ ID NO: 183, and the LCDR3 comprises the amino acid sequence of SEQ ID NO:184; (b) the HCDR1 comprises the amino acid sequence of SEQ ID NO: 185, the HCDR2 comprises the amino acid sequence of SEQ ID NO: 186, the HCDR3 comprises the amino acid sequence of SEQ ID NO: 181, the LCDR1 comprises the amino acid sequence of SEQ ID NO: 182 the LCDR2 comprises the amino acid sequence of SEQ ID NO: 183, and the LCDR3 comprises the amino acid sequence of SEQ ID NO: 184; (c) the HCDR1 comprises the amino acid sequence of SEQ ID NO: 187, the HCDR2 comprises the amino acid sequence of SEQ ID NO: 188, the HCDR3 comprises the amino acid sequence of SEQ ID NO:181, the LCDR1 comprises the amino acid sequence of SEQ ID NO: 182, the LCDR2 comprises the amino acid sequence of SEQ ID NO: 183, and the LCDR3 comprises the amino acid sequence of SEQ ID NO:184; (d) the HCDR1 comprises the amino acid sequence of SEQ ID NO: 189, the HCDR2 comprises the amino acid sequence of SEQ ID NO: 190, the HCDR3 comprises the amino acid sequence of SEQ ID NO: 191, the LCDR1 comprises the amino acid sequence of SEQ ID NO: 192, the LCDR2 comprises the amino acid sequence of SEQ ID NO: 193, and the LCDR3 comprises the amino acid sequence of SEQ ID NO: 194; and (e) the HCDR1 comprises the amino acid sequence of SEQ ID NO:195, the HCDR2 comprises the amino acid sequence of SEQ ID NO: 196, the HCDR3 comprises the amino acid sequence of SEQ ID NO: 197, the LCDR1 comprises the amino acid sequence of SEQ ID NO: 198, the LCDR2 comprises the amino acid sequence of DSS and the LCDR3 comprises the amino acid sequence of SEQ ID NO: 184; and (ii) the Fab that binds ENPP3 comprises: (a) in the VH domain that binds ENPP3, HCDR1 comprises the amino acid sequence of SEQ ID NO:1; HCDR2 comprises the amino acid sequence of SEQ ID NO:2; HCDR3 comprises the amino acid sequence of SEQ ID NO:3; and in the VL domain that binds ENPP3, LCDR1 comprises the amino acid sequence of SEQ ID NO:4; LCDR2 comprises the amino acid sequence of SEQ ID NO:5; and LCDR3 comprises the amino acid sequence of SEQ ID NO:6; (b) in the VH domain that binds ENPP3, HCDR1 comprises the amino acid sequence of SEQ ID NO: 7; HCDR2 comprises the amino acid sequence of SEQ ID NO:8; HCDR3 comprises the amino acid sequence of SEQ ID NO:3; and in the VL domain that binds ENPP3, LCDR1 comprises the amino acid sequence of SEQ ID NO:4; LCDR2 comprises the amino acid sequence of SEQ ID NO:5; and LCDR3 comprises the amino acid sequence of SEQ ID NO:6; (c) in the VH domain that binds ENPP3, HCDR1 comprises the amino acid sequence of SEQ ID NO: 9; HCDR2 comprises the amino acid sequence of SEQ ID NO: 10; HCDR3 comprises the amino acid sequence of SEQ ID NO:3; and in the VL domain that binds ENPP3, LCDR1 comprises the amino acid sequence of SEQ ID NO:4; LCDR2 comprises the amino acid sequence of SEQ ID NO:5; and LCDR3 comprises the amino acid sequence of SEQ ID NO:6; (d) in the VH domain that binds ENPP3, HCDR1 comprises the amino acid sequence of SEQ ID NO: 11; HCDR2 comprises the amino acid sequence of SEQ ID NO: 12; HCDR3 comprises the amino acid sequence of SEQ ID NO: 13; and in the VL domain that binds ENPP3, LCDR1 comprises the amino acid sequence of SEQ ID NO: 14; LCDR2 comprises the amino acid sequence of SEQ ID NO:15; and LCDR3 comprises the amino acid sequence of SEQ ID NO: 16; and (e) in the VH domain that binds ENPP3, HCDR1 comprises the amino acid sequence of SEQ ID NO: 17; HCDR2 comprises the amino acid sequence of SEQ ID NO:18; HCDR3 comprises the amino acid sequence of SEQ ID NO: 19; and in the VL domain that binds ENPP3, LCDR1 comprises the amino acid sequence of SEQ ID NO:20; LCDR2 comprises the amino acid sequence of GAS; and LCDR3 comprises the amino acid sequence of SEQ ID NO:6.

In some embodiments, the spFv that binds CD3ε comprises a VH domain comprising the amino acid sequence of SEQ ID NO:200, and a VL domain comprising the amino acid sequence of SEQ ID NO:201; the VH domain that binds ENPP3 comprises the amino acid sequence of SEQ ID NO:22, and the VL domain that binds ENPP3 comprises the amino acid sequence of SEQ ID NO:23.

In some embodiments, the spFv that binds CD3ε comprises the amino acid sequence of SEQ ID NO:248; the VH domain that binds ENPP3 comprises the amino acid sequence of SEQ ID NO:22, and the VL domain that binds ENPP3 comprises the amino acid sequence of SEQ ID NO:23.

In some embodiments, (i) the first polypeptide comprising a spFv that binds CD3ε, a CH2 domain and a CH3 domain comprises the amino acid sequence of SEQ ID NO:255; (ii) the second polypeptide comprising a VH domain that binds ENPP3, a CH2 domain and a CH3 domain comprises the amino acid sequence of SEQ ID NO:256; and (iii) the third polypeptide comprising a VL domain that binds ENPP3 comprises the amino acid sequence of SEQ ID NO: 257.

In some embodiments, the spFv comprises at least one disulfide bond between the VH or VL and the linker.

In one aspect, provided herein is a composition comprising any of the binding agents described herein and a pharmaceutically acceptable carrier.

In one aspect, provided herein is a polynucleotide comprising nucleotide sequences encoding a VH, a VL, or both a VH and a VL of any of the binding agents described herein.

In one aspect, provided herein is a vector comprising any of the polynucleotides described herein.

In one aspect, provided herein is a cell comprising any of the polynucleotides described herein. In one aspect, provided herein is a cell comprising any of the vectors described herein. In one aspect, provided herein is an isolated cell producing any of the binding agents described herein.

In one aspect, provided herein is a kit comprising any of the binding agents described herein.

In one aspect, provided herein is a method of making a binding agent which binds to an epitope of ENPP3, comprising culturing the cell described herein to express the binding agent. In one aspect, provided herein is a method of making a binding agent which binds to an epitope of ENPP3, comprising expressing a polynucleotide described herein.

In one aspect, provided herein is a method of directing a T cell to a target cell expressing ENPP3, comprising contacting the T cell with an effective amount of a ENPP3×CD3 binding agent described herein or a composition comprising the ENPP3×CD3 binding agent and a pharmaceutically acceptable carrier, wherein the antigen binding region that binds to CD3ε binds the T cell and the antigen binding region that binds to ENPP3 binds to the target cell.

In one aspect, provided herein is a method of treating a cancer or tumor in a subject in need thereof, comprising administering an effective amount a ENPP3×CD3 binding agent described herein or a composition comprising the ENPP3×CD3 binding agent and a pharmaceutically acceptable carrier to the subject. In some embodiments, the cancer or tumor is selected from the group consisting of a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.

### 4. BRIEF DESCRIPTION OF THE DRAWINGS

FIG. 1 depicts the screening funnel that was used for screening the ENPP3 binders.

FIG. 2 depicts in vitro killing with the select antibodies. The image was collected from Incucyte. Only the Red layer was selected with the scale set 1.0 min and 3.0 max. The assay was run in 3:1 Effector to Target ratio. The 3-fold titration series started at 3 μg/mL. There was a loss of cells with active compounds.

FIG. 3 depicts an in vitro killing time course with select antibodies. The time course was performed with 0.04 and 2.5 μg/mL treatment. The B56 antibody showed the best killing at low dose on all three ENPP3 expressing cell lines.

FIG. 4 depicts exemplary experimental results showing tumor area loss.

FIG. 5 depicts exemplary experimental results showing a tumor area AUC fold change time course. The plots show the results with ENPP3×CD3W245 molecules. The plots show the fold change of the AUC tumor area over time at two concentrations (0.04 and 1 ug/mL) for each cell line tested. As killing occurs the area decreases. The table shows the average value at 72 hours for each compound at the two concentrations. At the higher concentrations the value is lower. NPP3B276, NPP3B314 and NPP3B321 are still active at the lower dose. Tumor area does not decrease on ENPP3 HEPG2 KO cells. CD3 Null reference arms (ENPP3-Ref1 and ENPP3-Ref2) were not active on ENPP3 expressing arms. A704 cells have more receptors than HEPG2.

FIG. 6 depicts exemplary experimental results showing % tumor loss at 72 hours in mice bearing xenograft tumors (either A704 or HEPG2) and treated with bispecific ENPP3×CD3W245 antibodies.

FIG. 7 depicts exemplary experimental results showing that select ENPP3×CD3 exhibit potent in vitro killing and T-cell activation.

FIG. 8 depicts exemplary experimental results showing that select ENPP3×CD3 exhibit potent in vitro killing even at lower E:T ratios.

FIG. 9 depicts exemplary experimental results showing that surface binding of ENPP3 is specific to ENPP3 expressing cell lines. The data shows that cross-reactivity to other ENPP3 family members for the top 3 binders.

FIG. 10 depicts exemplary experimental results showing that select ENPP3×CD3 molecules demonstrated robust anti-tumor efficacy in the T-cell humanized VMRC-RCW CDX model.

FIG. 11 depicts exemplary experimental results showing that individual tumor volumes demonstrate efficacy with NPP3B56×CD3B2030-N106A.

FIG. 12 depicts a table demonstrating the efficacy of ENPP3×CD3 molecules on HepG2 xenografts in T cell humanized female NSG Mice. Study design: Inject 5e6 cells/animal +50% Matrigel/animal. Randomize into groups when tumors are ˜50-150 mm³˜16-21 days post implantation. 20e6 T cells from donor D204071 injected IP day after randomization. Antibodies dosed IP 2×/week following IVIG and Fc Block treatment day after T cells injected for 3 weeks. Tumors were measured with calipers once weekly until the group mean tumor volume reaches approximately 1500 mm³ and body weight was taken twice weekly. Serial blood sampling of whole blood collected through tail vein into EDTA tubes (10-15 μL) and diluted 1:10 in buffer at 4, 24, 72 hours post first dose and 24 and 72 hours post last dose. Samples are stored at −80 until all samples are collected, then submitted for bioanalysis.

FIG. 13 depicts exemplary experimental results demonstrating that treatment with NPP3B194 at 1 and 5 mg/kg and NPP3B239 at 1 and 10 mg/kg show significant tumor regression in NSG mice on day 52.

FIG. 14 depicts exemplary experimental results demonstrating that mean tumor graphs show sustained antitumor responses in groups treated with NPP3B194 at 1 and 5 mg/kg and NPP3B239 1 and 10 mg/kg compared to CD3×Null control in NSG mice bearing HepG2 xenograft tumors.

FIG. 15 depicts exemplary experimental results demonstrating that individual tumor volumes show complete responses in the groups treated with NPP3B194 at 1 and 5 mg/kg, and NPP3B239 at 1 and 10 mg/kg on day 61.

FIG. 16 depicts exemplary data showing the HDX-MS epitope mapping of NPP3W6 against three mAbs. The epitopes against NPP3B56 are residues 412-415 (PHDF; SEQ ID NO: 295), 673-676 (PSES; SEQ ID NO:296), 762-768 (PDEITKH; SEQ ID NO:297), and 773-775 (DVP). The epitopes against NPP3B62 are residues 572-576 (SLECF; SEQ ID NO:299), 582-586 (NSTQL; SEQ ID NO:300), and 673-681 (PSESQKCSF; SEQ ID NO:301). The epitopes against NPP3B98 are residues 556-562 (VSKFSVC; SEQ ID NO:302), 612-616 (RPRVL; SEQ ID NO:303), 621-624 (DHCL; SEQ ID NO:304), and 856-860 (QPVSE; SEQ ID NO: 305). Figure discloses SEQ ID NO: 327.

FIG. 17 shows a schematic of NPP3B815 (NPP3B56xCD3B2030-N106A), a bispecific antibody targeting CD3 and ENPP3. AAS, L234A, L235A, D265S; CD, cluster of differentiation; Fab, fragment antigen-binding; hole, T366S, L368A, Y407V; knob, T366W; ENPP3, ectonucleotide pyrophosphatase/phosphodiesterase family member 3; Ig, immunoglobulin; spFv, stapled single-chain fragment variable.

FIG. 18 shows ENPP3 expression (receptor density) in different cancer cell lines with variable expression levels. Flow-cytometry-based membrane ENPP3 detection and receptor occupancy quantification measured using a commercial ENPP3 antibody (i.e., clone NP4D6) on a panel of endogenous cancer cell lines. Representative histogram showing ENPP3 expression in a high, medium, and negative cell line. Abbreviations: ABC, antibody binding capacity; ENPP3, ectonucleotide pyrophosphatase/phosphodiesterase family member 3; HCC, hepatocellular carcinoma; LD, Live Dead; LLOD, lower limit of detection; Med, medium; Neg, negative; RCC, renal cell carcinoma; ULOD, upper limit of detection.

FIG. 19 shows ENPP3 expression in different in vivo CDX and PDX model systems from ex vivo tumors. Flow-cytometry (using commercial antibody clone NP4D6) and IHC-based evaluation (using commercial antibody clone E5M2W) of ENPP3 expression on 2 CDX models, i.e., VMRCRCW (RCC) and HepG2 (HCC), and a RCC PDX model, i.e., RXF488. Magnification is 30× for all images. Abbreviations: ENPP3, ectonucleotide pyrophosphatase/phosphodiesterase family member 3; CDX, cell-line-derived xenograft; HCC, hepatocellular carcinoma; IHC, immunohistochemistry; LD, Live Dead; PDX, patient-derived xenograft; RCC, renal cell carcinoma.

FIGS. 20A and 20B show the binding of NPP3B815 to endogenous ENPP3-expressing tumor cell lines and isolated T cells. FIG. 20A shows the binding of ENPP3×CD3 (NPP3B815) and Null×CD3 (CD3B2533) on A704 (ENPP3-high), VMRCRCW (ENPP3-medium), and HepG2 ENPP3KO (ENPP3-negative) cell lines was evaluated by flow cytometry. FIG. 20B shows the binding of ENPP3×CD3 (NPP3B815, NPP3B815) and isotype control (79C3B613) antibodies evaluated by flow cytometry on T cells isolated from 6 different healthy human donors. Solid lines denote NPP3B815 and dotted lines (bottom of graph) denote 79C3B613 binding. Abbreviations: CD, cluster of differentiation; ENPP3, ectonucleotide pyrophosphatase/phosphodiesterase family member 3; geomean, geometric mean.

FIG. 21 shows binding of NPP3B815 to ENPP1, ENPP2, and ENPP3 overexpressing cell lines. Binding of ENPP3×CD3 (NPP3B815) and isotype control (79C3B613) on CHO parental cell line or CHO cell lines overexpressing ENPP1, ENPP2 or ENPP3 was evaluated by flow cytometry. Abbreviations: CD, cluster of differentiation; ENPP1, ectonucleotide pyrophosphatase/phosphodiesterase family member 1; ENPP2, ectonucleotide pyrophosphatase/phosphodiesterase family member 2; ENPP3, ectonucleotide pyrophosphatase/phosphodiesterase family member 3.

FIG. 22 shows NPP3B815-induced tumor cell killing of a panel of tumor cell lines with endogenous ENPP3 expression. Incucyte-based assessment of tumor cell killing (i.e., loss of Nuclight-red-positive cells) was measured upon treatment with ENPP3×CD3 (NPP3B815, NPP3B815), ENPP3×Null (NPP3B812), and Null×CD3 (79C3B615) antibodies in the presence of isolated T cells from Donor 888668965 at E:T ratio of 3:1 on a panel of cell lines. Data plotted at 72 hours post treatment. Error bars are SEM. Abbreviations: CD, cluster of differentiation; ENPP3, ectonucleotide pyrophosphatase/phosphodiesterase family member 3; E:T ratio, effector-to-target cell ratio; SEM, standard error of the mean.

FIG. 23 shows NPP3B815-induced T cell activation in a panel of tumor cell lines with endogenous ENPP3 expression. T cell activation (i.e., CD25+ T cells) was measured by flow cytometry upon treatment with ENPP3×CD3 (NPP3B815, NPP3B815), ENPP3×Null (NPP3B812), and Null×CD3 (79C3B615) antibodies for 48 hours in the presence of isolated T cells from Donor 888668965 at E:T ratio of 3:1 with A704 (ENPP3-high), VMRCRCW (ENPP3-medium), HepG2 (ENPP3-medium), and HepG2 ENPP3 KO (ENPP3-negative) cell lines. Error bars are SEM. Abbreviations: CD, cluster of differentiation; ENPP3, ectonucleotide pyrophosphatase/phosphodiesterase family member 3; E:T ratio, effector-to-target cell ratio.

FIG. 24A, FIG. 24B, FIG. 24C, FIG. 24D, FIG. 24E, and FIG. 24F show NPP3B815-induced tumor cell killing and T cell activation in the presence of T cells isolated from multiple donors in ENPP3-positive and ENPP3-negative cell lines. (FIG. 24A, FIG. 24B, and FIG. 24C) Incucyte-based assessment of tumor cell killing (ie, loss of Nuclight-red-positive cells) measured upon treatment with ENPP3×CD3 (NPP3B815) antibody in the presence of T cells (E:T ratio of 3:1) isolated from 6 different healthy human donors tested with A704 (ENPP3-high), VMRCRCW (ENPP3-medium), and HepG2 ENPP3 KO (ENPP3-negative) cell lines. Data plotted at 72 hours post treatment. Error bars are SEM. (FIG. 24D, FIG. 24E, and FIG. 24F) T cell activation (ie, CD25+ T cells) was measured by flow cytometry upon treatment with ENPP3×CD3 (NPP3B815) for 48 hours in the presence of T cells (E:T ratio of 3:1) isolated from 6 different healthy human donors tested with A704 (ENPP3-high), VMRCRCW (ENPP3-medium), and HepG2 ENPP3 KO (ENPP3-negative) cell lines. Error bars are SEM. Abbreviations: CD, cluster of differentiation; ENPP3, ectonucleotide pyrophosphatase/phosphodiesterase family member 3; E:T ratio, effector-to-target cell ratio; SEM, standard error of the mean.

FIG. 25A, FIG. 25B, FIG. 25C, FIG. 25D, FIG. 25E, FIG. 25F, and FIG. 25G show NPP3B815-induced tumor cell killing in the presence of T cells isolated from multiple donors at different E:T ratios. (FIG. 25A, FIG. 25B, FIG. 25C, and FIG. 25D) Incucyte-based assessment of tumor cell killing (i.e., loss of Nuclight-red-positive cells) measured upon treatment with ENPP3×CD3 (NPP3B815) in the presence of T cells isolated from 6 different healthy human donors and added at 2 different E:T ratios of 1:1 and 1:3 to the assay with the ENPP3-high cell line, A704. Data plotted at 72- and 120-hours post-treatment. Error Bars are SEM. (FIG. 25E, FIG. 25F, and FIG. 25G) Incucyte-based assessment of tumor cell killing (ie, loss of Nuclight-red-positive cells) measured upon treatment with ENPP3×CD3 (NPP3B815) in the presence of T cells isolated from 6 different healthy human donors and added at 2 different E:T ratios of 1:1 and 1:3 to the assay with VMRCRCW (ENPP3-medium) cells. Data plotted at 72 and 120 hours post treatment. Error Bars are SEM. Abbreviations: CD, cluster of differentiation; Conc., concentration; D, donor; ENPP3, ectonucleotide pyrophosphatase/phosphodiesterase family member 3; E:T ratio, effector-to-target cell ratio; IFN, interferon; IL, interleukin; SEM, standard error of the mean; TNF, tumor necrosis factor.

FIG. 26 shows NPP3B815-induced cytokine release in the presence of T cells isolated from multiple donors in ENPP3-high cancer cell line A704. Cytokine release was measured by flow cytometry upon treatment with ENPP3×CD3 (NPP3B815) for 48 hours in the presence of T cells (E:T ratio of 3:1) isolated from 6 different healthy human donors tested with A704 (ENPP3-high) cells. Error bars are SEM. Abbreviations: CD, cluster of differentiation; Conc., concentration; D, donor; ENPP3, ectonucleotide pyrophosphatase/phosphodiesterase family member 3; E:T ratio, effector-to-target cell ratio; IFN, interferon; IL, interleukin; SEM, standard error of the mean; TNF, tumor necrosis factor.

FIG. 27 shows NPP3B815-induced tumor cell killing with PBMC donors at different E:T ratios. Incucyte-based assessment of tumor cell killing (i.e., loss of Nuclight-red-positive cells) measured upon treatment with ENPP3×CD3 (NPP3B815) and Null×CD3 (79C3B615) antibodies in the presence of PBMCs (NPP3B815: E:T ratio of 5:1, 3:1 or 1:1; 79C3B615: E:T ratio of 5:1) isolated from 6 different healthy human donors tested with A704 (ENPP3-high) cell line. Data plotted at 68 hours or 72 hours post treatment. Error bars are SEM. Abbreviations: CD, cluster of differentiation; Conc., concentration; D, donor; ENPP3, ectonucleotide pyrophosphatase/phosphodiesterase family member 3; E:T ratio, effector-to-target cell ratio; PBMC, peripheral blood mononuclear cell; SEM, standard error of the mean.

FIGS. 28A and 28B show binding of toom molecule NPP3B847 to cynomolgus ENPP3-expressing cells and T cells. (FIG. 28A) Binding of ENPP3×CD3 tool (NPP3B847; NPP3B56 paired with cyno cross reactive CD3B219), and isotype control (79C3B613) on the HepG2-huENPP3-KO cell line with cyno ENPP3-OE was evaluated by flow cytometry. Error Bars are SEM. (FIG. 28B) Binding of ENPP3×CD3 tool (NPP3B847) was evaluated on human and cyno T cells. Error Bars are SEM. Abbreviations: CD, cluster of differentiation; cyno, cynomolgus monkey; ENPP3, ectonucleotide pyrophosphatase/phosphodiesterase family member 3; Geomean, geometric mean; SEM, standard error of the mean.

FIG. 29 shows human and cynomolgus monkey T-cell activation and T-cell mediated cytotoxicity. Tumor cell killing and T-cell activation (i.e., CD25 T cells) was measured by flow cytometry upon treatment with ENPP3×CD3 (NPP3B815), matched Null×CD3 (79C3B615; with CD3B2030-N106A arm), ENPP3×CD3 tool (NPP3B847; NPP3B56 paired with cyno cross reactive CD3B219) and matched Null×CD3 (NPP3B41; with CD3B219 arm) antibodies for 48 hours in the presence of human and cyno T cells (E:T ratio of 3:1) tested with HepG2-huENPP3-KO cell line with cyno ENPP3-OE. Error Bars are SEM. Abbreviations: CD, cluster of differentiation; cyno, cynomolgus monkey; ENPP3, ectonucleotide pyrophosphatase/phosphodiesterase family member 3; E:T ratio, effector-to-target cell ratio; SEM, standard error of the mean.

FIGS. 30A and 30B show the effect of NPP3B815 on VMRCRCW established xenografts in mice (Study ONC2022-035). NSG mice bearing established VMRCRCW xenografts were IP dosed with NPP3B815 or Null×CD3 control antibody at the indicated doses. (FIG. 30A) Group tumor volumes are graphed as mean±SEM. Tumor cells were implanted on Day 0 and T cells were implanted on Day 10. Treatment with NPP3B815 or Null×CD3 control antibodies was on Days 11, 14, 18, 21, 25, 28, 31, and 34 (represented by line underneath X axis). (FIG. 30B) Individual tumor graphs for CD3×Null control antibody and NPP3B815 1 mg/kg treated groups. * Denotes significant difference of NPP3B815-treated groups on Day 39 (n=10/group) versus the control group. Abbreviations: CD, cluster of differentiation; IP, intraperitoneal; NSG, non-obese diabetic (NOD) severe combined immunodeficiency (scid) gamma or NOD.Cg-Prkdcscid Il2rgtm1 Wjl/SzJ; SEM, standard error of the mean.

FIGS. 31A and 31B show the effect of NPP3B194 (unstapled [scFv] ENPP3×CD3) on HepG2 established xenografts in mice (Study P764Y). NSG mice bearing established VMRCRCW xenografts were IP dosed with NPP3B194 or Null×CD3 control antibodies at the indicated doses. (FIG. 31A) Group tumor volumes are graphed as mean±SEM. Tumor cells were implanted on Day 0 and T cells were implanted on Day 21. Treatment with NPP3B194 or Null×CD3 control antibody was on Days 22, 25, 29, 32, 36, and 39 (represented by line underneath X axis). (FIG. 31B) Individual tumor graphs for groups. * Denotes significant difference of NPP3B194-treated groups on Day 52 (n≥7/group) versus the control group. Abbreviations: CD, cluster of differentiation; IP, intraperitoneal; NSG, non-obese diabetic (NOD) severe combined immunodeficiency (scid) gamma or NOD.Cg-Prkdcscid Il2rgtm1Wjl/SzJ; SEM, standard error of the mean.

FIG. 32 shows the effect of NPP3B815 on growth of HepG2 established xenografts in T-Cell-humanized Mice; * denotes significant difference of NPP3B815-treated groups on Day 45 versus the Null×CD3 control group. Tumor cells were implanted on Day 0 and T cells were implanted on Day 21. Treatment with NPP3B815 or Null×CD3 control antibodies was on Days 22, 25, 29, 32, 36, 39, 43, and 46.

FIG. 33 shows effect of NPP3B815 on growth of RXF 488 established xenografts in T-Cell-humanized Mice; * denotes significant difference of NPP3B815-treated groups on Day 45 versus the Null×CD3 control group. Tumor cells were implanted on Day 0 and T cells were implanted on Day 18. Treatment with NPP3B815 or Null×CD3 control antibodies was on Days 19, 22, 26, 29, 33, 36, 40, and 43.

### 5. DETAILED DESCRIPTION

Solid tumors have been a challenge for antibody-mediated T-cell redirection therapy, with very limited, if any, drugs yet approved by the US Food and Drug Administration (FDA). Limitation of the approach may be related to the lack of cancer specificity of targets leading to toxicity occurring at subefficacious doses. To mitigate this liability, the work described herein has focused on the identification of cancer-specific antigens with potentially greater therapeutic index based on apically expressed targets. Apical protein targets are unreachable by directed antibody through the blood stream in normal tissues, however in tumors characterized by disorganized expression, these proteins are reachable through the blood stream. Using this approach, ENPP3 was identified as a target with mainly apical expression in normal tissues and depolarized expression in cancer.

The invention provides monospecific and bispecific ENPP3 binding agents and methods of use thereof. In one embodiment, provided are compositions comprising the ENPP3 binding agents (monospecific or bispecific). The described compositions can be used to carry out the methods of using the ENPP3 binding fragments provided herein, or other methods known to those skilled in the art. In some embodiments, the described compositions can include bispecific agents for use in detecting the presence of multiple target antigens (e.g., ENPP3 and CD3) in a biological sample. Accordingly, the described compositions can include one or more of the binding agents (e.g., antibodies or proteins), or an antigen binding region(s) thereof, described herein. In one embodiment, provided is a bispecific binding agent comprising a first binding region specifically binding ENPP3 and a second binding region specifically binding CD3ε.

In some embodiments, the invention provides for the use of the compositions or bispecific binding agents of the invention for the diagnosis or treatment of a disease or disorder. In one embodiment, the disease or disorder is cancer.

In some embodiments, the invention provides for the use of the compositions or bispecific binding agents of the invention to target T cells to an ENPP3 expressing cell. In one embodiment, the ENPP3 expressing cell is a cancer cell.

### 5.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

### 5.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

Unless otherwise defined herein, technical and scientific terms used in the present description have the meanings that are commonly understood by those of ordinary skill in the art. Whenever appropriate, terms used in the singular will also include the plural and vice versa. In the event that any description of a term set forth conflicts with any document incorporated herein by reference, the description of the term set forth below shall control.

The term “binding agent” as used herein refers to a molecule that binds a specific antigen or target (e.g., ENPP3 and/or CD3). A binding agent may comprise a protein, peptide, nucleic acid, carbohydrate, lipid, or small molecular weight compound. In some embodiments, a binding agent comprises a full-length antibody. In some embodiments, a binding agent is an antigen binding fragment of an antibody. In some embodiments, a binding agent comprises an alternative protein scaffold or artificial scaffold (e.g., a non-immunoglobulin backbone). In some embodiments, a binding agent is a fusion protein comprising an antigen-binding site. In some embodiments, a binding agent is a bispecific molecule comprising at least two antigen-binding sites. In some embodiments, a binding agent is a multispecific molecule comprising at least three antigen-binding sites.

The terms “ENPP3” “ENPP3 protein” and “ENPP3 polypeptide” encompass a polypeptide (“polypeptide” and “protein” are used interchangeably herein), including any native polypeptide, from any vertebrate source, including mammals such as primates (e.g., humans and cynomolgus monkeys (cynomolgus)), dogs, and rodents (e.g., mice and rats), unless otherwise indicated. In certain embodiments, the terms include “related ENPP3 polypeptides,” including SNP variants thereof. The term “ENPP3” also encompasses “full-length,” unprocessed ENPP3 as well as any form that results from processing.

The term “Cluster of Differentiation 3 E” or “CD3ε” refers to a known protein which is also called “T-cell surface glycoprotein CD3 epsilon chain,” or “T3E.” CD3ε, together with CD3-gamma, -delta and -zeta, and the T-cell receptor alpha/beta and gamma/delta heterodimers, forms the T-cell receptor-CD3 complex. This complex plays an important role in coupling antigen recognition to several intracellular signal-transduction pathways. The CD3 complex mediates signal transduction, resulting in T cell activation and proliferation. CD3 is required for the immune response. The amino acid sequence of a full length CD3ε is shown in SEQ ID NO: 293. The amino acid sequence of the extracellular domain (ECD) of CD3ε is shown in SEQ ID NO: 294. Throughout the specification, “CD3ε-specific” or “specifically binds CD3ε” or “anti-CD3ε antibody” refers to antibodies that bind specifically to the CD3ε polypeptide (SEQ ID NO: 293), including antibodies that bind specifically to the CD3ε extracellular domain (ECD) (SEQ ID NO: 294);
Human CD3 epsilon:

(SEQ ID NO: 293)
MQSGTHWRVLGLCLLSVGVWGQDGNEEMGGITQTPYKVSISGTTV
ILTCPQYPGSEILWQHNDKNIGGDEDDKNIGSDEDHLSLKEFSEL
EQSGYYVCYPRGSKPEDANFYLYLRARVCENCMEMDVMSVATIVI
VDICITGGLLLLVYYWSKNRKAKAKPVTRGAGAGGRQRGQNKERP
PPVPNPDYEPIRKGQRDLYSGLNQRRI;

Human CD3 epsilon extracellular domain:

(SEQ ID NO: 294)
DGNEEMGGITQTPYKVSISGTTVILTCPQYPGSEILWQHNDKNIG
GDEDDKNIGSDEDHLSLKEFSELEQSGYYVCYPRGSKPEDANFYL
YLRARVCENCMEMD

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

The term “antibody,” “immunoglobulin,” or “Ig” is used interchangeably herein, and is used in the broadest sense and specifically covers, for example polyclonal antibodies, monoclonal antibodies (including agonist, antagonist, neutralizing antibodies, full-length monoclonal antibodies), antibody compositions with polyepitopic or monoepitopic specificity, recombinantly produced antibodies, single domain (e.g., VHH) antibodies, monospecific antibodies, multispecific antibodies (including bispecific antibodies), synthetic antibodies, chimeric antibodies, humanized antibodies, or human versions of antibodies having full-length heavy and/or light chains. Antibodies also include antibody fragments (and/or polypeptides that comprise antibody fragments) that retain binding characteristics of their parental antibodies. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable domain antibody, single variable domain, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region domain may be any suitable arrangement of immunoglobulin heavy (VH) and/or light (VL) variable domains. For example, antibodies also include tetrameric antibodies comprising two heavy chain and two light chain molecules, an antibody light chain monomer, and an antibody heavy chain monomer. Thus, for example, the V region domain may be dimeric and contain VHH-VHH, VH-VH, VH-VL, or VL-VL dimers that bind ENPP2. If desired, the VH and VL may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable regions”) can be obtained by constructing polynucleotides that encode one or more CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated, for example, into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23 (9): 1126-1136, 2005). In some embodiments, antibodies comprising a VH and/or VL contain a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions. A “functional fragment,” “binding fragment,” or “antigen binding fragment” of a therapeutic antibody will exhibit at least one if not some or all of the biological functions attributed to the intact antibody, the function comprising at least binding to the target antigen (e.g., a ENPP3 binding fragment or fragment that binds to ENPP3).

A typical 4-chain antibody unit is a heterotetrametric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat,” and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27 (1): 55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in the table below.

### Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26--H32 . . . 34 | H30--H35B | H27--H38 |
| | (Kabat | | | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “antigen binding domain” or “antigen binding region” refers to a binding agent or a portion of a binding agent as described herein (such as a protein or an antibody or fragment thereof) that binds an antigen. In some embodiments, an antigen binding region can comprise one or more fragments or portions of an intact antibody as described herein. The term “antigen binding domain” or “antigen binding region” can be an antibody fragment as described above.

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

The term “(scFv)₂” or “tandem scFv” or “bis-scFv” refers to a fusion protein comprising two light chain variable region (VL) and two heavy chain variable region (VH), wherein the two VL and the two VH are contiguously linked via polypeptide linkers, and capable of being expressed as a single chain polypeptide. The two VL and two VH are fused by peptide linkers to form a bivalent molecule VL_{A}-linker-VH_{A}-linker-VL_{B}-linker-VH_{B} to form two binding sites, capable of binding two different antigens or epitopes concurrently.

“Stapled single chain Fv” or “spFv” refers to a scFv that comprises one or more disulfide bonds between the VH and the linker or the VL and the linker. Typically the spFv may comprise one disulfide bond between the VH and the linker, one disulfide bond between the VL and the linker, or two disulfide bonds between the VH and the linker and the VL and the linker. scFv molecules which comprise disulfide bonds between the VH and the VL are excluded from the term “spFv”.

“Anchor point” refers to a scFv VH or a VL framework Cysteine (Cys) residue that can be mutagenized to Cys without adverse effect to the overall scFv structure and is capable of forming a disulfide bond with a Cys residing in the scFv linker.

“Staple” refers to the scFv linker that contains one or two Cys residues which are capable of forming a disulfide bond with the anchor point Cys.

The term “multispecific” refers to a molecule, such as an antibody that specifically binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

The term “bispecific” refers to a molecule (such as a protein or an antibody) that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

The terms “bispecific anti-ENPP3/anti-CD3 antibody,” “ENPP3/CD3 antibody,” “ENPP3×CD3 antibody,” “anti-ENPP3/anti-CD3 protein,” and the like refer to an antibody that binds ENPP3 and CD3, i.e., comprising at least one binding domain specifically binding ENPP3 and at least one binding domain specifically binding CD3. The domains specifically binding ENPP3 and CD3 are typically V_{H}/V_{L} pairs. The bispecific anti-ENPP3×CD3 antibody may be monovalent in terms of its binding to either ENPP3 or CD3.

The term “monoclonal antibody” as used herein refers to a substantially homogenous antibody population involved in the highly specific recognition and binding of a single antigenic determinant or epitope. The term “monoclonal antibody” encompasses intact and full-length antibodies as well as antibody fragments (e.g., Fab, Fab′, F(ab′)₂, Fv), single chain antibodies, scFv, fusion proteins comprising an antigen-binding antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of publicly available bioinformatic software tools. X-ray crystallography may be used to characterize an epitope on a target protein by analyzing the amino acid residue interactions of an antigen/antibody complex.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR amino acid residues are replaced by residues from corresponding CDRs from a non-human antibody (e.g., mouse, rat, rabbit, or non-human primate), wherein the non-human antibody has the desired specificity, affinity, and/or activity. In some embodiments, one or more framework region amino acid residues of the human heavy chain or light chain variable regions are replaced by corresponding residues from the non-human antibody. Furthermore, humanized antibodies can comprise amino acid residues that are not found in the human antibody or in the non-human antibody. In some embodiments, these modifications are made to further refine and/or optimize antibody characteristics. In some embodiments, the humanized antibody comprises at least a portion of a human immunoglobulin constant region (e.g., CH1, CH2, CH3, Fc, and/or hinge region).

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60, at least about 60-80 nucleotides or amino acids in length, or any integral value there between. In some embodiments, identity exists over a longer region than 60-80 nucleotides or amino acids, such as at least about 80-100 nucleotides or amino acids, and in some embodiments the sequences are substantially identical over the full length of the sequences being compared, for example, (i) the coding region of a nucleotide sequence or (ii) an amino acid sequence.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line.

The term “substantially pure” as used herein refers to material that is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, and the like.

The term “pharmaceutical composition” or “pharmaceutical formulation” as used herein refers to a preparation that is in such form as to permit the biological activity of the binding agent to be effective. A pharmaceutical formulation or composition generally comprises additional components, such as a pharmaceutically acceptable excipient, carrier, adjuvant, buffers, etc.

The term “effective amount” or “therapeutically effective amount” as used herein refers to the amount of an agent that is sufficient to reduce and/or ameliorate the severity and/or duration of (i) a disease, disorder or condition in a subject, and/or (ii) a symptom in a subject.

The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the binding agents provided herein).

The term “treat” or “treatment” or “treating” or “to treat” or “alleviate” or alleviation” or “alleviating” or “to alleviate” as used herein refers to therapeutic measures that aim to cure, slow down, lessen symptoms of, and/or halt progression of a pathologic condition or disorder. Thus, those in need of treatment include those already with the disorder.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, dendritic cells, etc.

As used herein, reference to “about” or “approximately” a value or parameter includes (and describes) embodiments that are directed to that value or parameter. For example, a description referring to “about X” includes description of “X.”

As used in the present disclosure and claims, the singular forms “a,” “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.3 ENPP3 Binding Agents

In one aspect, provided herein is a binding agent comprising an antigen binding region that binds ENPP3 (e.g., human ENPP3). In some embodiments, the present binding agent comprises at least one portion that is a polypeptide. In some embodiments, the present binding agent comprises at least one portion that is not a polypeptide. In some embodiments, the present binding agents are ENPP3 binding proteins.

In some embodiments, the present disclosure provides binding agents (e.g., antibodies or proteins) that bind ENPP3. In some embodiments, the ENPP3 binding agent binds a ENPP3 protein or a fragment thereof of a mammalian origin. In some embodiments, the ENPP3 binding agent binds a human ENPP3 protein or a fragment thereof. In some embodiments, the ENPP3 binding agent binds a ENPP3 protein or a fragment thereof originated from a non-human mammalian species. In some embodiments, the non-human mammalian species is a rodent (e.g., mice and rats). In some embodiments, the non-human mammalian species is a dog. In some embodiments, the non-human mammalian species is a cynomolgus monkeys (cynomolgus).

In some embodiments, the binding agent comprises an antigen binding region that binds to ENPP3. In some embodiments, the ENPP3 binding region in the present binding protein is an antibody or a binding domain derived from an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgG antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody. In some embodiments, the antibody comprises an IgG heavy chain. In some embodiments, the antibody comprises an IgG1 heavy chain. In some embodiments, the antibody comprises an IgG2 heavy chain. In some embodiments, the antibody comprises an IgG4 heavy chain. In some embodiments, the antibody comprises a kappa light chain. In some embodiments, the antibody comprises a kappa light chain constant region. In some embodiments, the antibody comprises a lambda light chain. In some embodiments, the antibody comprises a lambda light chain constant region. In some embodiments, the antibody is an antibody fragment comprising an antigen-binding site. In some embodiments, the antibody is an scFv. In some embodiments, the antibody is a disulfide-linked scFv. In some embodiments, the antibody is an spFv. In some embodiments, the antibody is a disulfide-linked sc(Fv)₂. In some embodiments, the antibody is a Fab, Fab′, or a F(ab)₂ antibody. In some embodiments, the antibody is a diabody. In some embodiments, the antibody is a nanobody. In some embodiments, the antibody is a monospecific antibody. In some embodiments, the antibody is a bispecific antibody. In some embodiments, the antibody is a trispecific antibody. In some embodiments, the antibody is a multispecific antibody. In some embodiments, the antibody is a monovalent antibody. In some embodiments, the antibody is a multivalent antibody. In some embodiments, the antibody is a bivalent antibody. In some embodiments, the antibody is a trivalent antibody. In some embodiments, the antibody is a tetravalent antibody.

In some embodiments, the ENPP3 binding region provided herein binds to ENPP3 (e.g., human ENPP3) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹ M to 10⁻¹³ M). In some embodiments, the ENPP3 binding region provided herein binds to ENPP3 with a dissociation constant of ≤0.1 nM. In some embodiments, the ENPP3 binding region provided herein binds to ENPP3 with a dissociation constant of ≤0.2 nM. In some embodiments, the ENPP3 binding region provided herein binds to ENPP3 with a dissociation constant of ≤0.3 nM. In some embodiments, the ENPP3 binding region provided herein binds to ENPP3 with a dissociation constant of ≤0.8 nM. In some embodiments, the ENPP3 binding region provided herein binds to ENPP3 with a dissociation constant of ≤3 nM. In some embodiments, the ENPP3 binding region provided herein binds to ENPP3 with a dissociation constant of ≤9 nM. A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-3000, or the Biacore®TM-8000 system.

In one aspect, provided herein is a binding agent that binds ENPP3. In one embodiment, the binding agent comprises a ENPP3 binding region. In some embodiments, the ENPP3 binding region is any one of those in Tables 1-11.

In some embodiments, the ENPP3 binding region provided herein comprises one or more CDR sequences of the VH or VL having the amino acid sequence set forth in any one of Tables 1-11.

In one embodiment, the ENPP3 binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:22. In another embodiment, the ENPP3 binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:23. In yet another embodiment, the ENPP3 binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:22, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:23. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the ENPP3 binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 1, 7, 9, 11 or 17; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 2, 8, 10, 12 or 18, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 3, 13 or 19; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:4, 14 or 20; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 5, 15 or 21; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 6 or 16. In some embodiments, the ENPP3 binding region is humanized. In some embodiments, the ENPP3 binding region comprises an acceptor human framework, e.g., a human immunoglobulin framework or a human consensus framework.

In some specific embodiments, the ENPP3 binding region provided herein comprises one or more CDRs in Table 1.

In some embodiments, the ENPP3 binding region provided herein comprises an HCDR1 comprising an amino acid sequence of any of SEQ ID NOs: 1, 7, 9, 11, and 17; (ii) an HCDR2 comprising an amino acid sequence of any of SEQ ID NOs: 2, 8, 10, 12, and 18, (iii) an HCDR3 comprising an amino acid sequence of SEQ ID NOs: 3, 13, and 19; (iv) a LCDR1 comprising an amino acid sequence of SEQ ID NOs: 4, 14, and 20; (v) a LCDR2 comprising an amino acid sequence of SEQ ID NOs: 5, 15, and 21; and/or (vi) a LCDR3 comprising an amino acid sequence of SEQ ID NOs: 6 and 16.

In some specific embodiments, in the ENPP3 binding region provided herein, the HCDR1 comprises the amino acid sequence of SEQ ID NO:1, the HCDR2 comprises the amino acid sequence of SEQ ID NO:2, the HCDR3 comprises the amino acid sequence of SEQ ID NO: 3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the ENPP3 binding region provided herein, the HCDR1 comprises the amino acid sequence of SEQ ID NO:7, the HCDR2 comprises the amino acid sequence of SEQ ID NO:8, the HCDR3 comprises the amino acid sequence of SEQ ID NO: 3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the ENPP3 binding region provided herein, the HCDR1 comprises the amino acid sequence of SEQ ID NO:9, the HCDR2 comprises the amino acid sequence of SEQ ID NO: 10, the HCDR3 comprises the amino acid sequence of SEQ ID NO: 3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the ENPP3 binding region provided herein, the HCDR1 comprises the amino acid sequence of SEQ ID NO:11, the HCDR2 comprises the amino acid sequence of SEQ ID NO: 12, the HCDR3 comprises the amino acid sequence of SEQ ID NO: 13, the LCDR1 comprises the amino acid sequence of SEQ ID NO: 14, the LCDR2 comprises the amino acid sequence of SEQ ID NO: 15, and the LCDR3 comprises the amino acid sequence of SEQ ID NO: 16.

In some specific embodiments, in the ENPP3 binding region provided herein, the HCDR1 comprises the amino acid sequence of SEQ ID NO:17, the HCDR2 comprises the amino acid sequence of SEQ ID NO: 18, the HCDR3 comprises the amino acid sequence of SEQ ID NO: 19, the LCDR1 comprises the amino acid sequence of SEQ ID NO:20, the LCDR2 comprises the amino acid sequence of GAS, and the LCDR3 comprises the amino acid sequence of SEQ ID NO:6.

In one embodiment, provided herein is a binding region that binds ENPP3, comprising a VH comprising a HCDR1, a HCDR2, and a HCDR3 having an amino acid sequence of SEQ ID NOs: 1, 2, and 3, respectively. In another embodiment, provided herein is a binding region that binds ENPP3, comprising a VL comprising a LCDR1, a LCDR2, and a LCDR3 having an amino acid sequence of SEQ ID NOs: 4, 5, and 6, respectively. In another embodiment, provided herein is a binding region that binds ENPP3, comprising: (i) a VH comprising a HCDR1, a HCDR2, and a HCDR3 having an amino acid sequence of SEQ ID NOs: 1, 2, and 3, respectively, and (ii) a VL comprising a LCDR1, a LCDR2, and a LCDR3 having an amino acid sequence of SEQ ID NOs: 4, 5, and 6, respectively.

In one embodiment, provided herein is a binding region that binds ENPP3, comprising a VH comprising a HCDR1, a HCDR2, and a HCDR3 having an amino acid sequence of SEQ ID NOs: 7, 8, and 3, respectively. In another embodiment, provided herein is a binding region that binds ENPP3, comprising a VL comprising a LCDR1, a LCDR2, and a LCDR3 having an amino acid sequence of SEQ ID NOs: 4, 5, and 6, respectively. In another embodiment, provided herein is a binding region that binds ENPP3, comprising: (i) a VH comprising a HCDR1, a HCDR2, and a HCDR3 having an amino acid sequence of SEQ ID NOs: 7, 8, and 3, respectively, and (ii) a VL comprising a LCDR1, a LCDR2, and a LCDR3 having an amino acid sequence of SEQ ID NOs: 4, 5, and 6, respectively.

In one embodiment, provided herein is a binding region that binds ENPP3, comprising a VH comprising a HCDR1, a HCDR2, and a HCDR3 having an amino acid sequence of SEQ ID NOs: 9, 10, and 3, respectively. In another embodiment, provided herein is a binding region that binds ENPP3, comprising a VL comprising a LCDR1, a LCDR2, and a LCDR3 having an amino acid sequence of SEQ ID NOs: 4, 5, and 6, respectively. In another embodiment, provided herein is a binding region that binds ENPP3, comprising: (i) a VH comprising a HCDR1, a HCDR2, and a HCDR3 having an amino acid sequence of SEQ ID NOs: 9, 10, and 3, respectively, and (ii) a VL comprising a LCDR1, a LCDR2, and a LCDR3 having an amino acid sequence of SEQ ID NOs: 4, 5, and 6, respectively.

In one embodiment, provided herein is a binding region that binds ENPP3, comprising a VH comprising a HCDR1, a HCDR2, and a HCDR3 having an amino acid sequence of SEQ ID NOs: 11, 12, and 13, respectively. In another embodiment, provided herein is a binding region that binds ENPP3, comprising a VL comprising a LCDR1, a LCDR2, and a LCDR3 having an amino acid sequence of SEQ ID NOs: 14, 15, and 16, respectively. In another embodiment, provided herein is a binding region that binds ENPP3, comprising: (i) a VH comprising a HCDR1, a HCDR2, and a HCDR3 having an amino acid sequence of SEQ ID NOs: 11, 12, and 13, respectively, and (ii) a VL comprising a LCDR1, a LCDR2, and a LCDR3 having an amino acid sequence of SEQ ID NOs: 14, 15, and 16, respectively.

In one embodiment, provided herein is a binding region that binds ENPP3, comprising a VH comprising a HCDR1, a HCDR2, and a HCDR3 having an amino acid sequence of SEQ ID NOs: 17, 18, and 19, respectively. In another embodiment, provided herein is a binding region that binds ENPP3, comprising a VL comprising a LCDR1, a LCDR2, and a LCDR3 having an amino acid sequence of SEQ ID NOs: 20, 21, and 6, respectively. In another embodiment, provided herein is a binding region that binds ENPP3, comprising: (i) a VH comprising a HCDR1, a HCDR2, and a HCDR3 having an amino acid sequence of SEQ ID NOs: 17, 18, and 19, respectively, and (ii) a VL comprising a LCDR1, a LCDR2, and a LCDR3 having an amino acid sequence of SEQ ID NOs: 20, 21, and 6, respectively.

In one embodiment, the ENPP3 binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:45. In another embodiment, the ENPP3 binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:46. In yet another embodiment, the ENPP3 binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:45, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:46. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the ENPP3 binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 24, 30, 32, 34, or 40; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 25, 31, 33, 35, or 41, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 26, 36, or 42; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 27, 37, or 43; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 28, 38, or 44; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 29 or 39. In some embodiments, the ENPP3 binding region is humanized. In some embodiments, the ENPP3 binding region comprises an acceptor human framework, e.g., a human immunoglobulin framework or a human consensus framework.

In some specific embodiments, the ENPP3 binding region provided herein comprises one or more CDRs in Table 2.

In some embodiments, the ENPP3 binding region provided herein comprises an HCDR1 comprising an amino acid sequence of any of SEQ ID NOs: 24, 30, 32, 34, or 50; (ii) an HCDR2 comprising an amino acid sequence of any of SEQ ID NOs: 25, 31, 33, 35, or 41, (iii) an HCDR3 comprising an amino acid sequence of SEQ ID NOs: 26, 36, or 42; (iv) a LCDR1 comprising an amino acid sequence of SEQ ID NOs: 27, 37, or 43; (v) a LCDR2 comprising an amino acid sequence of SEQ ID NOs: 28, 38, or 44; and/or (vi) a LCDR3 comprising an amino acid sequence of SEQ ID NOs: 29 or 39.

In some specific embodiments, in the ENPP3 binding region provided herein, the HCDR1 comprises the amino acid sequence of SEQ ID NO:24, the HCDR2 comprises the amino acid sequence of SEQ ID NO:25, the HCDR3 comprises the amino acid sequence of SEQ ID NO: 26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO:29.

In some specific embodiments, in the ENPP3 binding region provided herein, the HCDR1 comprises the amino acid sequence of SEQ ID NO:30, the HCDR2 comprises the amino acid sequence of SEQ ID NO:31, the HCDR3 comprises the amino acid sequence of SEQ ID NO: 26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO:29.

In some specific embodiments, in the ENPP3 binding region provided herein, the HCDR1 comprises the amino acid sequence of SEQ ID NO:32, the HCDR2 comprises the amino acid sequence of SEQ ID NO:33, the HCDR3 comprises the amino acid sequence of SEQ ID NO: 26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO:29.

In some specific embodiments, in the ENPP3 binding region provided herein, the HCDR1 comprises the amino acid sequence of SEQ ID NO:34, the HCDR2 comprises the amino acid sequence of SEQ ID NO:35, the HCDR3 comprises the amino acid sequence of SEQ ID NO: 36, the LCDR1 comprises the amino acid sequence of SEQ ID NO:37, the LCDR2 comprises the amino acid sequence of SEQ ID NO:38, and the LCDR3 comprises the amino acid sequence of SEQ ID NO:39.

In some specific embodiments, in the ENPP3 binding region provided herein, the HCDR1 comprises the amino acid sequence of SEQ ID NO:40, the HCDR2 comprises the amino acid sequence of SEQ ID NO:41, the HCDR3 comprises the amino acid sequence of SEQ ID NO: 42, the LCDR1 comprises the amino acid sequence of SEQ ID NO:43, the LCDR2 comprises the amino acid sequence of DAS, and the LCDR3 comprises the amino acid sequence of SEQ ID NO:29.

In one embodiment, provided herein is a binding region that binds ENPP3, comprising a VH comprising a HCDR1, a HCDR2, and a HCDR3 having an amino acid sequence of SEQ ID NOs: 24, 25, and 26, respectively. In another embodiment, provided herein is a binding region that binds ENPP3, comprising a VL comprising a LCDR1, a LCDR2, and a LCDR3 having an amino acid sequence of SEQ ID NOs: 27, 28, and 29, respectively. In another embodiment, provided herein is a binding region that binds ENPP3, comprising: (i) a VH comprising a HCDR1, a HCDR2, and a HCDR3 having an amino acid sequence of SEQ ID NOs: 24, 25, and 26, respectively, and (ii) a VL comprising a LCDR1, a LCDR2, and a LCDR3 having an amino acid sequence of SEQ ID NOs: 27, 28, and 29, respectively.

In one embodiment, provided herein is a binding region that binds ENPP3, comprising a VH comprising a HCDR1, a HCDR2, and a HCDR3 having an amino acid sequence of SEQ ID NOs: 30, 31, and 26, respectively. In another embodiment, provided herein is a binding region that binds ENPP3, comprising a VL comprising a LCDR1, a LCDR2, and a LCDR3 having an amino acid sequence of SEQ ID SEQ ID NOs: 27, 28, and 29, respectively. In another embodiment, provided herein is a binding region that binds ENPP3, comprising: (i) a VH comprising a HCDR1, a HCDR2, and a HCDR3 having an amino acid sequence of SEQ ID NOs: 30, 31, and 26, respectively, and (ii) a VL comprising a LCDR1, a LCDR2, and a LCDR3 having an amino acid sequence of SEQ ID SEQ ID NOs: 27, 28, and 29, respectively.

In one embodiment, provided herein is a binding region that binds ENPP3, comprising a VH comprising a HCDR1, a HCDR2, and a HCDR3 having an amino acid sequence of SEQ ID NOs: 32, 33, and 26, respectively. In another embodiment, provided herein is a binding region that binds ENPP3, comprising a VL comprising a LCDR1, a LCDR2, and a LCDR3 having an amino acid sequence of SEQ ID NOs: 27, 28, and 29, respectively. In another embodiment, provided herein is a binding region that binds ENPP3, comprising: (i) a VH comprising a HCDR1, a HCDR2, and a HCDR3 having an amino acid sequence of SEQ ID NOs: 32, 33, and 26, respectively, and (ii) a VL comprising a LCDR1, a LCDR2, and a LCDR3 having an amino acid sequence of SEQ ID NOs: 27, 28, and 29, respectively.

In one embodiment, provided herein is a binding region that binds ENPP3, comprising a VH comprising a HCDR1, a HCDR2, and a HCDR3 having an amino acid sequence of SEQ ID NOs: 34, 35, and 36, respectively. In another embodiment, provided herein is a binding region that binds ENPP3, comprising a VL comprising a LCDR1, a LCDR2, and a LCDR3 having an amino acid sequence of SEQ ID NOs: 37, 38, and 39, respectively. In another embodiment, provided herein is a binding region that binds ENPP3, comprising: (i) a VH comprising a HCDR1, a HCDR2, and a HCDR3 having an amino acid sequence of SEQ ID NOs: 34, 35, and 36, respectively, and (ii) a VL comprising a LCDR1, a LCDR2, and a LCDR3 having an amino acid sequence of SEQ ID NOs: 37, 38, and 39, respectively.

In one embodiment, provided herein is a binding region that binds ENPP3, comprising a VH comprising a HCDR1, a HCDR2, and a HCDR3 having an amino acid sequence of SEQ ID NOs: 40, 41, and 42, respectively. In another embodiment, provided herein is a binding region that binds ENPP3, comprising a VL comprising a LCDR1, a LCDR2, and a LCDR3 having an amino acid sequence of SEQ ID NOs: 43, 44, and 29, respectively. In another embodiment, provided herein is a binding region that binds ENPP3, comprising: (i) a VH comprising a HCDR1, a HCDR2, and a HCDR3 having an amino acid sequence of SEQ ID NOs: 40, 41, and 42, respectively, and (ii) a VL comprising a LCDR1, a LCDR2, and a LCDR3 having an amino acid sequence of SEQ ID NOs: 43, 44, and 29, respectively.

In one embodiment, the ENPP3 binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:68. In another embodiment, the ENPP3 binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:69 or 70. In yet another embodiment, the ENPP3 binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:68, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:69 or 70. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the ENPP3 binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 47, 53, 55, 57, or 63; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 48, 54, 56, 58, or 64, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 49, 59, or 65; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 50, 60, or 66; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 51, 61, or 67; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 52 or 62. In some embodiments, the ENPP3 binding region is humanized. In some embodiments, the ENPP3 binding region comprises an acceptor human framework, e.g., a human immunoglobulin framework or a human consensus framework.

In some specific embodiments, the ENPP3 binding region provided herein comprises one or more CDRs in Table 3 or Table 4.

In some embodiments, the ENPP3 binding region provided herein comprises an HCDR1 comprising an amino acid sequence of any of SEQ ID NOs: 47, 53, 55, 57, or 63; (ii) an HCDR2 comprising an amino acid sequence of any of SEQ ID NOs: 48, 54, 56, 58, or 64, (iii) an HCDR3 comprising an amino acid sequence of SEQ ID NOs: 49, 59, or 65; (iv) a LCDR1 comprising an amino acid sequence of SEQ ID NOs: 50, 60, or 66; (v) a LCDR2 comprising an amino acid sequence of SEQ ID NOs: 51, 61, or 67; and/or (vi) a LCDR3 comprising an amino acid sequence of SEQ ID NOs: 52 or 62.

In some specific embodiments, in the ENPP3 binding region provided herein, the HCDR1 comprises the amino acid sequence of SEQ ID NO:47, the HCDR2 comprises the amino acid sequence of SEQ ID NO:48, the HCDR3 comprises the amino acid sequence of SEQ ID NO: 49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO:52.

In some specific embodiments, in the ENPP3 binding region provided herein, the HCDR1 comprises the amino acid sequence of SEQ ID NO:53, the HCDR2 comprises the amino acid sequence of SEQ ID NO:54, the HCDR3 comprises the amino acid sequence of SEQ ID NO: 49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO:52.

In some specific embodiments, in the ENPP3 binding region provided herein, the HCDR1 comprises the amino acid sequence of SEQ ID NO:55, the HCDR2 comprises the amino acid sequence of SEQ ID NO:56, the HCDR3 comprises the amino acid sequence of SEQ ID NO: 49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO:52.

In some specific embodiments, in the ENPP3 binding region provided herein, the HCDR1 comprises the amino acid sequence of SEQ ID NO:57, the HCDR2 comprises the amino acid sequence of SEQ ID NO:58, the HCDR3 comprises the amino acid sequence of SEQ ID NO: 59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO:62.

In some specific embodiments, in the ENPP3 binding region provided herein, the HCDR1 comprises the amino acid sequence of SEQ ID NO:63, the HCDR2 comprises the amino acid sequence of SEQ ID NO:64, the HCDR3 comprises the amino acid sequence of SEQ ID NO: 65, the LCDR1 comprises the amino acid sequence of SEQ ID NO:66, the LCDR2 comprises the amino acid sequence of QIS, and the LCDR3 comprises the amino acid sequence of SEQ ID NO:52.

In one embodiment, provided herein is a binding region that binds ENPP3, comprising a VH comprising a HCDR1, a HCDR2, and a HCDR3 having an amino acid sequence of SEQ ID NOs: 47, 48, and 49, respectively. In another embodiment, provided herein is a binding region that binds ENPP3, comprising a VL comprising a LCDR1, a LCDR2, and a LCDR3 having an amino acid sequence of SEQ ID NOs: 50, 51, and 52, respectively. In another embodiment, provided herein is a binding region that binds ENPP3, comprising: (i) a VH comprising a HCDR1, a HCDR2, and a HCDR3 having an amino acid sequence of SEQ ID NOs: 47, 48, and 49, respectively, and (ii) a VL comprising a LCDR1, a LCDR2, and a LCDR3 having an amino acid sequence of SEQ ID NOs: 50, 51, and 52, respectively.

In one embodiment, provided herein is a binding region that binds ENPP3, comprising a VH comprising a HCDR1, a HCDR2, and a HCDR3 having an amino acid sequence of SEQ ID NOs: 53, 54, and 49, respectively. In another embodiment, provided herein is a binding region that binds ENPP3, comprising a VL comprising a LCDR1, a LCDR2, and a LCDR3 having an amino acid sequence of SEQ ID NOs: 50, 51, and 52, respectively. In another embodiment, provided herein is a binding region that binds ENPP3, comprising: (i) a VH comprising a HCDR1, a HCDR2, and a HCDR3 having an amino acid sequence of SEQ ID NOs: 53, 54, and 49, respectively, and (ii) a VL comprising a LCDR1, a LCDR2, and a LCDR3 having an amino acid sequence of SEQ ID NOs: 50, 51, and 52, respectively.

In one embodiment, provided herein is a binding region that binds ENPP3, comprising a VH comprising a HCDR1, a HCDR2, and a HCDR3 having an amino acid sequence of SEQ ID NOs: 55, 56, and 49, respectively. In another embodiment, provided herein is a binding region that binds ENPP3, comprising a VL comprising a LCDR1, a LCDR2, and a LCDR3 having an amino acid sequence of SEQ ID NOs: 50, 51, and 52, respectively. In another embodiment, provided herein is a binding region that binds ENPP3, comprising: (i) a VH comprising a HCDR1, a HCDR2, and a HCDR3 having an amino acid sequence of SEQ ID NOs: 55, 56, and 49, respectively, and (ii) a VL comprising a LCDR1, a LCDR2, and a LCDR3 having an amino acid sequence of SEQ ID NOs: 50, 51, and 52, respectively.

In one embodiment, provided herein is a binding region that binds ENPP3, comprising a VH comprising a HCDR1, a HCDR2, and a HCDR3 having an amino acid sequence of SEQ ID NOs: 57, 58, and 59, respectively. In another embodiment, provided herein is a binding region that binds ENPP3, comprising a VL comprising a LCDR1, a LCDR2, and a LCDR3 having an amino acid sequence of SEQ ID NOs: 60, 61, and 62, respectively. In another embodiment, provided herein is a binding region that binds ENPP3, comprising: (i) a VH comprising a HCDR1, a HCDR2, and a HCDR3 having an amino acid sequence of SEQ ID NOS: 57, 58, and 59, respectively, and (ii) a VL comprising a LCDR1, a LCDR2, and a LCDR3 having an amino acid sequence of SEQ ID NOs: 60, 61, and 62, respectively.

In one embodiment, provided herein is a binding region that binds ENPP3, comprising a VH comprising a HCDR1, a HCDR2, and a HCDR3 having an amino acid sequence of SEQ ID NOs: 63, 64, and 65, respectively. In another embodiment, provided herein is a binding region that binds ENPP3, comprising a VL comprising a LCDR1, a LCDR2, and a LCDR3 having an amino acid sequence of SEQ ID NOs: 66, 67, and 52, respectively. In another embodiment, provided herein is a binding region that binds ENPP3, comprising: (i) a VH comprising a HCDR1, a HCDR2, and a HCDR3 having an amino acid sequence of SEQ ID NOs: 63, 64, and 65, respectively, and (ii) a VL comprising a LCDR1, a LCDR2, and a LCDR3 having an amino acid sequence of SEQ ID NOs: 66, 67, and 52, respectively.

In one embodiment, the ENPP3 binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:91. In another embodiment, the ENPP3 binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:92. In yet another embodiment, the ENPP3 binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:91, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:92. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the ENPP3 binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 71, 77, 79, 81 or 87; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 72, 78, 80, 82 or 88, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 73, 83 or 89; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 74, 84 or 90; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 75, 85 or 21; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 76 or 86. In some embodiments, the ENPP3 binding region is humanized. In some embodiments, the ENPP3 binding region comprises an acceptor human framework, e.g., a human immunoglobulin framework or a human consensus framework.

In some specific embodiments, the ENPP3 binding region provided herein comprises one or more CDRs in Table 5.

In some embodiments, the ENPP3 binding region provided herein comprises an HCDR1 comprising an amino acid sequence of any of SEQ ID NOs: 71, 77, 79, 81 or 87; (ii) an HCDR2 comprising an amino acid sequence of any of SEQ ID NOs: 72, 78, 80, 82 or 88, (iii) an HCDR3 comprising an amino acid sequence of SEQ ID NOs: 73, 83 or 89; (iv) a LCDR1 comprising an amino acid sequence of SEQ ID NOs: 74, 84 or 90; (v) a LCDR2 comprising an amino acid sequence of SEQ ID NOs: 75, 85 or 21; and/or (vi) a LCDR3 comprising an amino acid sequence of SEQ ID NOs: 76 or 86.

In some specific embodiments, in the ENPP3 binding region provided herein, the HCDR1 comprises the amino acid sequence of SEQ ID NO:71, the HCDR2 comprises the amino acid sequence of SEQ ID NO: 72, the HCDR3 comprises the amino acid sequence of SEQ ID NO: 73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO:76.

In some specific embodiments, in the ENPP3 binding region provided herein, the HCDR1 comprises the amino acid sequence of SEQ ID NO:77, the HCDR2 comprises the amino acid sequence of SEQ ID NO: 78, the HCDR3 comprises the amino acid sequence of SEQ ID NO: 73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO:76.

In some specific embodiments, in the ENPP3 binding region provided herein, the HCDR1 comprises the amino acid sequence of SEQ ID NO:79, the HCDR2 comprises the amino acid sequence of SEQ ID NO:80, the HCDR3 comprises the amino acid sequence of SEQ ID NO: 73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO:76.

In some specific embodiments, in the ENPP3 binding region provided herein, the HCDR1 comprises the amino acid sequence of SEQ ID NO:81, the HCDR2 comprises the amino acid sequence of SEQ ID NO:82, the HCDR3 comprises the amino acid sequence of SEQ ID NO: 83, the LCDR1 comprises the amino acid sequence of SEQ ID NO:84, the LCDR2 comprises the amino acid sequence of SEQ ID NO:85, and the LCDR3 comprises the amino acid sequence of SEQ ID NO:86.

In some specific embodiments, in the ENPP3 binding region provided herein, the HCDR1 comprises the amino acid sequence of SEQ ID NO:87, the HCDR2 comprises the amino acid sequence of SEQ ID NO:88, the HCDR3 comprises the amino acid sequence of SEQ ID NO: 89, the LCDR1 comprises the amino acid sequence of SEQ ID NO:90, the LCDR2 comprises the amino acid sequence of GAS, and the LCDR3 comprises the amino acid sequence of SEQ ID NO:76.

In one embodiment, provided herein is a binding region that binds ENPP3, comprising a VH comprising a HCDR1, a HCDR2, and a HCDR3 having an amino acid sequence of SEQ ID NOs: 71 72, and 73, respectively. In another embodiment, provided herein is a binding region that binds ENPP3, comprising a VL comprising a LCDR1, a LCDR2, and a LCDR3 having an amino acid sequence of SEQ ID NOs: 74, 75, and 76, respectively. In another embodiment, provided herein is a binding region that binds ENPP3, comprising: (i) a VH comprising a HCDR1, a HCDR2, and a HCDR3 having an amino acid sequence of SEQ ID NOs: 71 72, and 73, respectively, and (ii) a VL comprising a LCDR1, a LCDR2, and a LCDR3 having an amino acid sequence of SEQ ID NOs: 74, 75, and 76, respectively.

In one embodiment, provided herein is a binding region that binds ENPP3, comprising a VH comprising a HCDR1, a HCDR2, and a HCDR3 having an amino acid sequence of SEQ ID NOs: 77, 78 and 73, respectively. In another embodiment, provided herein is a binding region that binds ENPP3, comprising a VL comprising a LCDR1, a LCDR2, and a LCDR3 having an amino acid sequence of SEQ ID NOs: 74, 75, and 76, respectively. In another embodiment, provided herein is a binding region that binds ENPP3, comprising: (i) a VH comprising a HCDR1, a HCDR2, and a HCDR3 having an amino acid sequence of SEQ ID NOs: 77, 78 and 73, respectively, and (ii) a VL comprising a LCDR1, a LCDR2, and a LCDR3 having an amino acid sequence of SEQ ID NOs: 74, 75, and 76, respectively.

In one embodiment, provided herein is a binding region that binds ENPP3, comprising a VH comprising a HCDR1, a HCDR2, and a HCDR3 having an amino acid sequence of SEQ ID NOs: 79, 80 and 73, respectively. In another embodiment, provided herein is a binding region that binds ENPP3, comprising a VL comprising a LCDR1, a LCDR2, and a LCDR3 having an amino acid sequence of SEQ ID NOs: 74, 75, and 76, respectively. In another embodiment, provided herein is a binding region that binds ENPP3, comprising: (i) a VH comprising a HCDR1, a HCDR2, and a HCDR3 having an amino acid sequence of SEQ ID NOs: 79, 80 and 73, respectively, and (ii) a VL comprising a LCDR1, a LCDR2, and a LCDR3 having an amino acid sequence of SEQ ID NOs: 74, 75, and 76, respectively.

In one embodiment, provided herein is a binding region that binds ENPP3, comprising a VH comprising a HCDR1, a HCDR2, and a HCDR3 having an amino acid sequence of SEQ ID NOs: 81, 82 and 83, respectively. In another embodiment, provided herein is a binding region that binds ENPP3, comprising a VL comprising a LCDR1, a LCDR2, and a LCDR3 having an amino acid sequence of SEQ ID NOs: 84, 85, and 86, respectively. In another embodiment, provided herein is a binding region that binds ENPP3, comprising: (i) a VH comprising a HCDR1, a HCDR2, and a HCDR3 having an amino acid sequence of SEQ ID NOs: 81, 82 and 83, respectively, and (ii) a VL comprising a LCDR1, a LCDR2, and a LCDR3 having an amino acid sequence of SEQ ID NOs: 84, 85, and 86, respectively.

In one embodiment, provided herein is a binding region that binds ENPP3, comprising a VH comprising a HCDR1, a HCDR2, and a HCDR3 having an amino acid sequence of SEQ ID NOs: 87, 88, and 89, respectively. In another embodiment, provided herein is a binding region that binds ENPP3, comprising a VL comprising a LCDR1, a LCDR2, and a LCDR3 having an amino acid sequence of SEQ ID NOs: 90, 21, and 76, respectively. In another embodiment, provided herein is a binding region that binds ENPP3, comprising: (i) a VH comprising a HCDR1, a HCDR2, and a HCDR3 having an amino acid sequence of SEQ ID NOs: 87, 88, and 89, respectively, and (ii) a VL comprising a LCDR1, a LCDR2, and a LCDR3 having an amino acid sequence of SEQ ID NOs: 90, 21, and 76, respectively.

In one embodiment, the ENPP3 binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:113. In another embodiment, the ENPP3 binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO: 114 or 115. In yet another embodiment, the ENPP3 binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:113, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:114 or 115. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the ENPP3 binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 93, 99, 101, 103, or 109; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 94, 100, 102, 104, and 110 (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 95, 105 or 111; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 96, 106 or 112; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 97, 107 or 21; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 98 or 108. In some embodiments, the ENPP3 binding region is humanized. In some embodiments, the ENPP3 binding region comprises an acceptor human framework, e.g., a human immunoglobulin framework or a human consensus framework.

In some specific embodiments, the ENPP3 binding region provided herein comprises one or more CDRs in Table 6 or Table 7.

In some embodiments, the ENPP3 binding region provided herein comprises an HCDR1 comprising an amino acid sequence of any of SEQ ID NOs: 93, 99, 101, 103 or 109; (ii) an HCDR2 comprising an amino acid sequence of any of SEQ ID NOs: 94, 100, 102, 104, and 110, (iii) an HCDR3 comprising an amino acid sequence of SEQ ID NOs: 95, 105 or 111; (iv) a LCDR1 comprising an amino acid sequence of SEQ ID NOs: 96, 106 or 112; (v) a LCDR2 comprising an amino acid sequence of SEQ ID NOs: 97, 107 or 21; and/or (vi) a LCDR3 comprising an amino acid sequence of SEQ ID NOs: 98 or 108.

In some specific embodiments, in the ENPP3 binding region provided herein, the HCDR1 comprises the amino acid sequence of SEQ ID NO:93, the HCDR2 comprises the amino acid sequence of SEQ ID NO:94, the HCDR3 comprises the amino acid sequence of SEQ ID NO: 95, the LCDR1 comprises the amino acid sequence of SEQ ID NO:96, the LCDR2 comprises the amino acid sequence of SEQ ID NO:97 and the LCDR3 comprises the amino acid sequence of SEQ ID NO:98.

In some specific embodiments, in the ENPP3 binding region provided herein, the HCDR1 comprises the amino acid sequence of SEQ ID NO:99, the HCDR2 comprises the amino acid sequence of SEQ ID NO: 100, the HCDR3 comprises the amino acid sequence of SEQ ID NO: 95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO:98.

In some specific embodiments, in the ENPP3 binding region provided herein, the HCDR1 comprises the amino acid sequence of SEQ ID NO: 101, the HCDR2 comprises the amino acid sequence of SEQ ID NO: 102, the HCDR3 comprises the amino acid sequence of SEQ ID NO:95, the LCDR1 comprises the amino acid sequence of SEQ ID NO:96 the LCDR2 comprises the amino acid sequence of SEQ ID NO:97, and the LCDR3 comprises the amino acid sequence of SEQ ID NO:98.

In some specific embodiments, in the ENPP3 binding region provided herein, the HCDR1 comprises the amino acid sequence of SEQ ID NO: 103, the HCDR2 comprises the amino acid sequence of SEQ ID NO: 104, the HCDR3 comprises the amino acid sequence of SEQ ID NO: 105, the LCDR1 comprises the amino acid sequence of SEQ ID NO: 106, the LCDR2 comprises the amino acid sequence of SEQ ID NO: 107, and the LCDR3 comprises the amino acid sequence of SEQ ID NO:108.

In some specific embodiments, in the ENPP3 binding region provided herein, the HCDR1 comprises the amino acid sequence of SEQ ID NO: 109, the HCDR2 comprises the amino acid sequence of SEQ ID NO: 110, the HCDR3 comprises the amino acid sequence of SEQ ID NO: 111, the LCDR1 comprises the amino acid sequence of SEQ ID NO:112, the LCDR2 comprises the amino acid sequence of GAS, and the LCDR3 comprises the amino acid sequence of SEQ ID NO:98.

In one embodiment, provided herein is a binding region that binds ENPP3, comprising a VH comprising a HCDR1, a HCDR2, and a HCDR3 having an amino acid sequence of SEQ ID NOs: 93, 94, and 95, respectively. In another embodiment, provided herein is a binding region that binds ENPP3, comprising a VL comprising a LCDR1, a LCDR2, and a LCDR3 having an amino acid sequence of SEQ ID NOs: 96, 97, and 98, respectively. In another embodiment, provided herein is a binding region that binds ENPP3, comprising: (i) a VH comprising a HCDR1, a HCDR2, and a HCDR3 having an amino acid sequence of SEQ ID NOs: 93, 94, and 95, respectively, and (ii) a VL comprising a LCDR1, a LCDR2, and a LCDR3 having an amino acid sequence of SEQ ID NOs: 96, 97, and 98, respectively.

In one embodiment, provided herein is a binding region that binds ENPP3, comprising a VH comprising a HCDR1, a HCDR2, and a HCDR3 having an amino acid sequence of SEQ ID NOs: 99, 100, and 95, respectively. In another embodiment, provided herein is a binding region that binds ENPP3, comprising a VL comprising a LCDR1, a LCDR2, and a LCDR3 having an amino acid sequence of SEQ ID NOs: 96, 97, and 98, respectively. In another embodiment, provided herein is a binding region that binds ENPP3, comprising: (i) a VH comprising a HCDR1, a HCDR2, and a HCDR3 having an amino acid sequence of SEQ ID NOs: 99, 100, and 95, respectively, and (ii) a VL comprising a LCDR1, a LCDR2, and a LCDR3 having an amino acid sequence of SEQ ID NOs: 96, 97, and 98, respectively.

In one embodiment, provided herein is a binding region that binds ENPP3, comprising a VH comprising a HCDR1, a HCDR2, and a HCDR3 having an amino acid sequence of SEQ ID NOs: 101, 102 and 95, respectively. In another embodiment, provided herein is a binding region that binds ENPP3, comprising a VL comprising a LCDR1, a LCDR2, and a LCDR3 having an amino acid sequence of SEQ ID NOs: 96, 97, and 98, respectively. In another embodiment, provided herein is a binding region that binds ENPP3, comprising: (i) a VH comprising a HCDR1, a HCDR2, and a HCDR3 having an amino acid sequence of SEQ ID NOs: 101, 102 and 95, respectively, and (ii) a VL comprising a LCDR1, a LCDR2, and a LCDR3 having an amino acid sequence of SEQ ID NOs: 96, 97, and 98, respectively.

In one embodiment, provided herein is a binding region that binds ENPP3, comprising a VH comprising a HCDR1, a HCDR2, and a HCDR3 having an amino acid sequence of SEQ ID NOs: 103, 104, and 105, respectively. In another embodiment, provided herein is a binding region that binds ENPP3, comprising a VL comprising a LCDR1, a LCDR2, and a LCDR3 having an amino acid sequence of SEQ ID NOs: 106, 107 and 108, respectively. In another embodiment, provided herein is a binding region that binds ENPP3, comprising: (i) a VH comprising a HCDR1, a HCDR2, and a HCDR3 having an amino acid sequence of SEQ ID NOs: 103, 104, and 105, respectively, and (ii) a VL comprising a LCDR1, a LCDR2, and a LCDR3 having an amino acid sequence of SEQ ID NOs: 106, 107 and 108, respectively.

In one embodiment, provided herein is a binding region that binds ENPP3, comprising a VH comprising a HCDR1, a HCDR2, and a HCDR3 having an amino acid sequence of SEQ ID NOs: 109, 110 and 111, respectively. In another embodiment, provided herein is a binding region that binds ENPP3, comprising a VL comprising a LCDR1, a LCDR2, and a LCDR3 having an amino acid sequence of SEQ ID NOs: 112, 21 and 98, respectively. In another embodiment, provided herein is a binding region that binds ENPP3, comprising: (i) a VH comprising a HCDR1, a HCDR2, and a HCDR3 having an amino acid sequence of SEQ ID NOs: 109, 110 and 111, respectively, and (ii) a VL comprising a LCDR1, a LCDR2, and a LCDR3 having an amino acid sequence of SEQ ID NOs: 112, 21 and 98, respectively.

In one embodiment, the ENPP3 binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO: 134. In another embodiment, the ENPP3 binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO: 135 or 136. In yet another embodiment, the ENPP3 binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:134, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO: 135 or 136. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the ENPP3 binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 116, 121, 123, 125 or 130; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 117, 122, 124, 126 or 131, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 118, 127 or 132; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 119, 127 or 132; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 5, 15 or 21; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 120 or 129. In some embodiments, the ENPP3 binding region is humanized. In some embodiments, the ENPP3 binding region comprises an acceptor human framework, e.g., a human immunoglobulin framework or a human consensus framework.

In some specific embodiments, the ENPP3 binding region provided herein comprises one or more CDRs in Table 8.

In some embodiments, the ENPP3 binding region provided herein comprises an HCDR1 comprising an amino acid sequence of any of SEQ ID NOs: 116, 121, 123, 125 or 130; (ii) an HCDR2 comprising an amino acid sequence of any of SEQ ID NOs: 117, 122, 124, 126 or 131, (iii) an HCDR3 comprising an amino acid sequence of SEQ ID NOs: 118, 127 or 132; (iv) a LCDR1 comprising an amino acid sequence of SEQ ID NOs: 119, 127 or 132; (v) a LCDR2 comprising an amino acid sequence of SEQ ID NOs: 5, 15 or 21; and/or (vi) a LCDR3 comprising an amino acid sequence of SEQ ID NOs: 120 or 129.

In some specific embodiments, in the ENPP3 binding region provided herein, the HCDR1 comprises the amino acid sequence of SEQ ID NO:116, the HCDR2 comprises the amino acid sequence of SEQ ID NO:117, the HCDR3 comprises the amino acid sequence of SEQ ID NO:118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120.

In some specific embodiments, in the ENPP3 binding region provided herein, the HCDR1 comprises the amino acid sequence of SEQ ID NO: 121, the HCDR2 comprises the amino acid sequence of SEQ ID NO: 122, the HCDR3 comprises the amino acid sequence of SEQ ID NO: 118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120.

In some specific embodiments, in the ENPP3 binding region provided herein, the HCDR1 comprises the amino acid sequence of SEQ ID NO: 123, the HCDR2 comprises the amino acid sequence of SEQ ID NO:124, the HCDR3 comprises the amino acid sequence of SEQ ID NO:118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120.

In some specific embodiments, in the ENPP3 binding region provided herein, the HCDR1 comprises the amino acid sequence of SEQ ID NO: 125, the HCDR2 comprises the amino acid sequence of SEQ ID NO: 126, the HCDR3 comprises the amino acid sequence of SEQ ID NO: 127, the LCDR1 comprises the amino acid sequence of SEQ ID NO: 128, the LCDR2 comprises the amino acid sequence of SEQ ID NO: 15, and the LCDR3 comprises the amino acid sequence of SEQ ID NO: 129.

In some specific embodiments, in the ENPP3 binding region provided herein, the HCDR1 comprises the amino acid sequence of SEQ ID NO: 130, the HCDR2 comprises the amino acid sequence of SEQ ID NO: 131, the HCDR3 comprises the amino acid sequence of SEQ ID NO: 132, the LCDR1 comprises the amino acid sequence of SEQ ID NO: 133, the LCDR2 comprises the amino acid sequence of GAS, and the LCDR3 comprises the amino acid sequence of SEQ ID NO:120.

In one embodiment, provided herein is a binding region that binds ENPP3, comprising a VH comprising a HCDR1, a HCDR2, and a HCDR3 having an amino acid sequence of SEQ ID NOs: 116, 117 and 118, respectively. In another embodiment, provided herein is a binding region that binds ENPP3, comprising a VL comprising a LCDR1, a LCDR2, and a LCDR3 having an amino acid sequence of SEQ ID NOs: 119, 5, and 120, respectively. In another embodiment, provided herein is a binding region that binds ENPP3, comprising: (i) a VH comprising a HCDR1, a HCDR2, and a HCDR3 having an amino acid sequence of SEQ ID NOs: 116, 117 and 118, respectively, and (ii) a VL comprising a LCDR1, a LCDR2, and a LCDR3 having an amino acid sequence of SEQ ID NOs: 119, 5, and 120, respectively.

In one embodiment, provided herein is a binding region that binds ENPP3, comprising a VH comprising a HCDR1, a HCDR2, and a HCDR3 having an amino acid sequence of SEQ ID NOs: 121, 122, and 118, respectively. In another embodiment, provided herein is a binding region that binds ENPP3, comprising a VL comprising a LCDR1, a LCDR2, and a LCDR3 having an amino acid sequence of SEQ ID NOs: 119, 5, and 120, respectively. In another embodiment, provided herein is a binding region that binds ENPP3, comprising: (i) a VH comprising a HCDR1, a HCDR2, and a HCDR3 having an amino acid sequence of SEQ ID NOs: 121, 122, and 118, respectively, and (ii) a VL comprising a LCDR1, a LCDR2, and a LCDR3 having an amino acid sequence of SEQ ID NOs: 119, 5, and 120, respectively.

In one embodiment, provided herein is a binding region that binds ENPP3, comprising a VH comprising a HCDR1, a HCDR2, and a HCDR3 having an amino acid sequence of SEQ ID NOs: 123, 124, and 118, respectively. In another embodiment, provided herein is a binding region that binds ENPP3, comprising a VL comprising a LCDR1, a LCDR2, and a LCDR3 having an amino acid sequence of SEQ ID NOs: 119, 5, and 120, respectively. In another embodiment, provided herein is a binding region that binds ENPP3, comprising: (i) a VH comprising a HCDR1, a HCDR2, and a HCDR3 having an amino acid sequence of SEQ ID NOs: 123, 124, and 118, respectively, and (ii) a VL comprising a LCDR1, a LCDR2, and a LCDR3 having an amino acid sequence of SEQ ID NOs: 119, 5, and 120, respectively.

In one embodiment, provided herein is a binding region that binds ENPP3, comprising a VH comprising a HCDR1, a HCDR2, and a HCDR3 having an amino acid sequence of SEQ ID NOs: 125, 126 and 127, respectively. In another embodiment, provided herein is a binding region that binds ENPP3, comprising a VL comprising a LCDR1, a LCDR2, and a LCDR3 having an amino acid sequence of SEQ ID NOs: 128, 15 and 129, respectively. In another embodiment, provided herein is a binding region that binds ENPP3, comprising: (i) a VH comprising a HCDR1, a HCDR2, and a HCDR3 having an amino acid sequence of SEQ ID NOs: 125, 126 and 127, respectively, and (ii) a VL comprising a LCDR1, a LCDR2, and a LCDR3 having an amino acid sequence of SEQ ID NOs: 128, 15 and 129, respectively.

In one embodiment, provided herein is a binding region that binds ENPP3, comprising a VH comprising a HCDR1, a HCDR2, and a HCDR3 having an amino acid sequence of SEQ ID NOs: 130, 131 and 132, respectively. In another embodiment, provided herein is a binding region that binds ENPP3, comprising a VL comprising a LCDR1, a LCDR2, and a LCDR3 having an amino acid sequence of SEQ ID NOs: 133, 21, and 120, respectively. In another embodiment, provided herein is a binding region that binds ENPP3, comprising: (i) a VH comprising a HCDR1, a HCDR2, and a HCDR3 having an amino acid sequence of SEQ ID NOs: 130, 131 and 132, respectively, and (ii) a VL comprising a LCDR1, a LCDR2, and a LCDR3 having an amino acid sequence of SEQ ID NOs: 133, 21, and 120, respectively.

In one embodiment, the ENPP3 binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:158. In another embodiment, the ENPP3 binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:159. In yet another embodiment, the ENPP3 binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO: 158, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:159. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the ENPP3 binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 137, 143, 145, 147 or 153; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 138, 144, 146, 148 or 154; (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 139, 149 or 155; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 140, 150 or 156; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 141, 151 or 157; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 142 or 152. In some embodiments, the ENPP3 binding region is humanized. In some embodiments, the ENPP3 binding region comprises an acceptor human framework, e.g., a human immunoglobulin framework or a human consensus framework.

In some specific embodiments, the ENPP3 binding region provided herein comprises one or more CDRs in Table 10.

In some embodiments, the ENPP3 binding region provided herein comprises an HCDR1 comprising an amino acid sequence of any of SEQ ID NOs: 137, 143, 145, 147 or 153; (ii) an HCDR2 comprising an amino acid sequence of any of SEQ ID NOs: 138, 144, 146, 148 or 154; (iii) an HCDR3 comprising an amino acid sequence of SEQ ID NOs: 139, 149 or 155; (iv) a LCDR1 comprising an amino acid sequence of SEQ ID NOs: 140, 150 or 156; (v) a LCDR2 comprising an amino acid sequence of SEQ ID NOs: 141, 151 or 157; and/or (vi) a LCDR3 comprising an amino acid sequence of SEQ ID NOs: 142 or 152.

In some specific embodiments, in the ENPP3 binding region provided herein, the HCDR1 comprises the amino acid sequence of SEQ ID NO:137, the HCDR2 comprises the amino acid sequence of SEQ ID NO: 138, the HCDR3 comprises the amino acid sequence of SEQ ID NO: 139, the LCDR1 comprises the amino acid sequence of SEQ ID NO:140, the LCDR2 comprises the amino acid sequence of SEQ ID NO:141, and the LCDR3 comprises the amino acid sequence of SEQ ID NO:142.

In some specific embodiments, in the ENPP3 binding region provided herein, the HCDR1 comprises the amino acid sequence of SEQ ID NO: 143, the HCDR2 comprises the amino acid sequence of SEQ ID NO:144, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO: 142.

In some specific embodiments, in the ENPP3 binding region provided herein, the HCDR1 comprises the amino acid sequence of SEQ ID NO: 145, the HCDR2 comprises the amino acid sequence of SEQ ID NO: 146, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO:141, and the LCDR3 comprises the amino acid sequence of SEQ ID NO:142.

In some specific embodiments, in the ENPP3 binding region provided herein, the HCDR1 comprises the amino acid sequence of SEQ ID NO:147, the HCDR2 comprises the amino acid sequence of SEQ ID NO: 148, the HCDR3 comprises the amino acid sequence of SEQ ID NO: 149, the LCDR1 comprises the amino acid sequence of SEQ ID NO:150, the LCDR2 comprises the amino acid sequence of SEQ ID NO:151, and the LCDR3 comprises the amino acid sequence of SEQ ID NO:152.

In some specific embodiments, in the ENPP3 binding region provided herein, the HCDR1 comprises the amino acid sequence of SEQ ID NO: 153, the HCDR2 comprises the amino acid sequence of SEQ ID NO:154, the HCDR3 comprises the amino acid sequence of SEQ ID NO: 155, the LCDR1 comprises the amino acid sequence of SEQ ID NO:156, the LCDR2 comprises the amino acid sequence of AAS, and the LCDR3 comprises the amino acid sequence of SEQ ID NO: 142.

In one embodiment, provided herein is a binding region that binds ENPP3, comprising a VH comprising a HCDR1, a HCDR2, and a HCDR3 having an amino acid sequence of SEQ ID NOs: 137, 138 and 139, respectively. In another embodiment, provided herein is a binding region that binds ENPP3, comprising a VL comprising a LCDR1, a LCDR2, and a LCDR3 having an amino acid sequence of SEQ ID NOs: 140, 141 and 142, respectively. In another embodiment, provided herein is a binding region that binds ENPP3, comprising: (i) a VH comprising a HCDR1, a HCDR2, and a HCDR3 having an amino acid sequence of SEQ ID NOs: 137, 138 and 139, respectively, and (ii) a VL comprising a LCDR1, a LCDR2, and a LCDR3 having an amino acid sequence of SEQ ID NOs: 140, 141 and 142, respectively.

In one embodiment, provided herein is a binding region that binds ENPP3, comprising a VH comprising a HCDR1, a HCDR2, and a HCDR3 having an amino acid sequence of SEQ ID NOs: 143, 144, and 139, respectively. In another embodiment, provided herein is a binding region that binds ENPP3, comprising a VL comprising a LCDR1, a LCDR2, and a LCDR3 having an amino acid sequence of SEQ ID NOs: 140, 141 and 142, respectively. In another embodiment, provided herein is a binding region that binds ENPP3, comprising: (i) a VH comprising a HCDR1, a HCDR2, and a HCDR3 having an amino acid sequence of SEQ ID NOs: 143, 144, and 139, respectively, and (ii) a VL comprising a LCDR1, a LCDR2, and a LCDR3 having an amino acid sequence of SEQ ID NOs: 140, 141 and 142, respectively.

In one embodiment, provided herein is a binding region that binds ENPP3, comprising a VH comprising a HCDR1, a HCDR2, and a HCDR3 having an amino acid sequence of SEQ ID NOs: 145, 146 and 139, respectively. In another embodiment, provided herein is a binding region that binds ENPP3, comprising a VL comprising a LCDR1, a LCDR2, and a LCDR3 having an amino acid sequence of SEQ ID NOs: 140, 141 and 142, respectively. In another embodiment, provided herein is a binding region that binds ENPP3, comprising: (i) a VH comprising a HCDR1, a HCDR2, and a HCDR3 having an amino acid sequence of SEQ ID NOs: 145, 146 and 139, respectively, and (ii) a VL comprising a LCDR1, a LCDR2, and a LCDR3 having an amino acid sequence of SEQ ID NOs: 140, 141 and 142, respectively.

In one embodiment, provided herein is a binding region that binds ENPP3, comprising a VH comprising a HCDR1, a HCDR2, and a HCDR3 having an amino acid sequence of SEQ ID NOs: 147, 148 and 149, respectively. In another embodiment, provided herein is a binding region that binds ENPP3, comprising a VL comprising a LCDR1, a LCDR2, and a LCDR3 having an amino acid sequence of SEQ ID NOs: 150, 151 and 152, respectively. In another embodiment, provided herein is a binding region that binds ENPP3, comprising: (i) a VH comprising a HCDR1, a HCDR2, and a HCDR3 having an amino acid sequence of SEQ ID NOs: 147, 148 and 149, respectively, and (ii) a VL comprising a LCDR1, a LCDR2, and a LCDR3 having an amino acid sequence of SEQ ID NOs: 150, 151 and 152, respectively.

In one embodiment, provided herein is a binding region that binds ENPP3, comprising a VH comprising a HCDR1, a HCDR2, and a HCDR3 having an amino acid sequence of SEQ ID NOs: 153, 154 and 155, respectively. In another embodiment, provided herein is a binding region that binds ENPP3, comprising a VL comprising a LCDR1, a LCDR2, and a LCDR3 having an amino acid sequence of SEQ ID NOs: 156, 157 and 142, respectively. In another embodiment, provided herein is a binding region that binds ENPP3, comprising: (i) a VH comprising a HCDR1, a HCDR2, and a HCDR3 having an amino acid sequence of SEQ ID NOs: 153, 154 and 155, respectively, and (ii) a VL comprising a LCDR1, a LCDR2, and a LCDR3 having an amino acid sequence of SEQ ID NOs: 156, 157 and 142, respectively.

In one embodiment, the ENPP3 binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:177. In another embodiment, the ENPP3 binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO: 178. In yet another embodiment, the ENPP3 binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO: 177, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:178. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the ENPP3 binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 1, 164, 166, 11, or 172; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 160, 165, 167, 168 or 173, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 161, 169 or 174; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 162, 170 or 175; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 163, 171 or 176; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs: 76 or 86. In some embodiments, the ENPP3 binding region is humanized. In some embodiments, the ENPP3 binding region comprises an acceptor human framework, e.g., a human immunoglobulin framework or a human consensus framework.

In some specific embodiments, the ENPP3 binding region provided herein comprises one or more CDRs in Table 11.

In some embodiments, the ENPP3 binding region provided herein comprises an HCDR1 comprising an amino acid sequence of any of SEQ ID NOs: 1, 164, 166, 11 or 172; (ii) an HCDR2 comprising an amino acid sequence of any of SEQ ID NOs: 160, 165, 167, 168 or 173, (iii) an HCDR3 comprising an amino acid sequence of SEQ ID NOs: 161, 169 or 174; (iv) a LCDR1 comprising an amino acid sequence of SEQ ID NOs: 162, 170 or 175; (v) a LCDR2 comprising an amino acid sequence of SEQ ID NOs: 163, 171 or 176; and/or (vi) a LCDR3 comprising an amino acid sequence of SEQ ID NOs: 76 or 86.

In some specific embodiments, in the ENPP3 binding region provided herein, the HCDR1 comprises the amino acid sequence of SEQ ID NO: 1, the HCDR2 comprises the amino acid sequence of SEQ ID NO: 160, the HCDR3 comprises the amino acid sequence of SEQ ID NO: 161, the LCDR1 comprises the amino acid sequence of SEQ ID NO:162, the LCDR2 comprises the amino acid sequence of SEQ ID NO:163, and the LCDR3 comprises the amino acid sequence of SEQ ID NO:76.

In some specific embodiments, in the ENPP3 binding region provided herein, the HCDR1 comprises the amino acid sequence of SEQ ID NO:164, the HCDR2 comprises the amino acid sequence of SEQ ID NO: 165, the HCDR3 comprises the amino acid sequence of SEQ ID NO: 161, the LCDR1 comprises the amino acid sequence of SEQ ID NO: 162 the LCDR2 comprises the amino acid sequence of SEQ ID NO:163, and the LCDR3 comprises the amino acid sequence of SEQ ID NO:76.

In some specific embodiments, in the ENPP3 binding region provided herein, the HCDR1 comprises the amino acid sequence of SEQ ID NO: 166, the HCDR2 comprises the amino acid sequence of SEQ ID NO: 167, the HCDR3 comprises the amino acid sequence of SEQ ID NO: 161, the LCDR1 comprises the amino acid sequence of SEQ ID NO:162, the LCDR2 comprises the amino acid sequence of SEQ ID NO: 163, and the LCDR3 comprises the amino acid sequence of SEQ ID NO:76.

In some specific embodiments, in the ENPP3 binding region provided herein, the HCDR1 comprises the amino acid sequence of SEQ ID NO: 11, the HCDR2 comprises the amino acid sequence of SEQ ID NO:168, the HCDR3 comprises the amino acid sequence of SEQ ID NO: 169, the LCDR1 comprises the amino acid sequence of SEQ ID NO: 170, the LCDR2 comprises the amino acid sequence of SEQ ID NO: 171, and the LCDR3 comprises the amino acid sequence of SEQ ID NO:86.

In some specific embodiments, in the ENPP3 binding region provided herein, the HCDR1 comprises the amino acid sequence of SEQ ID NO: 172, the HCDR2 comprises the amino acid sequence of SEQ ID NO: 173, the HCDR3 comprises the amino acid sequence of SEQ ID NO: 174, the LCDR1 comprises the amino acid sequence of SEQ ID NO:175, the LCDR2 comprises the amino acid sequence of VAS and the LCDR3 comprises the amino acid sequence of SEQ ID NO:76.

In one embodiment, provided herein is a binding region that binds ENPP3, comprising a VH comprising a HCDR1, a HCDR2, and a HCDR3 having an amino acid sequence of SEQ ID NOs: 1, 160 and 161, respectively. In another embodiment, provided herein is a binding region that binds ENPP3, comprising a VL comprising a LCDR1, a LCDR2, and a LCDR3 having an amino acid sequence of SEQ ID NOs: 162, 163 and 76, respectively. In another embodiment, provided herein is a binding region that binds ENPP3, comprising: (i) a VH comprising a HCDR1, a HCDR2, and a HCDR3 having an amino acid sequence of SEQ ID NOs: 1, 160 and 161, respectively, and (ii) a VL comprising a LCDR1, a LCDR2, and a LCDR3 having an amino acid sequence of SEQ ID NOs: 162, 163 and 76, respectively.

In one embodiment, provided herein is a binding region that binds ENPP3, comprising a VH comprising a HCDR1, a HCDR2, and a HCDR3 having an amino acid sequence of SEQ ID NOs: 164, 165 and 161, respectively. In another embodiment, provided herein is a binding region that binds ENPP3, comprising a VL comprising a LCDR1, a LCDR2, and a LCDR3 having an amino acid sequence of SEQ ID NOs: 162, 163 and 76, respectively. In another embodiment, provided herein is a binding region that binds ENPP3, comprising: (i) a VH comprising a HCDR1, a HCDR2, and a HCDR3 having an amino acid sequence of SEQ ID NOs: 164, 165 and 161, respectively, and (ii) a VL comprising a LCDR1, a LCDR2, and a LCDR3 having an amino acid sequence of SEQ ID NOs: 162, 163 and 76, respectively.

In one embodiment, provided herein is a binding region that binds ENPP3, comprising a VH comprising a HCDR1, a HCDR2, and a HCDR3 having an amino acid sequence of SEQ ID NOs: 166, 167 and 161, respectively. In another embodiment, provided herein is a binding region that binds ENPP3, comprising a VL comprising a LCDR1, a LCDR2, and a LCDR3 having an amino acid sequence of SEQ ID NOs: 162, 163 and 76, respectively. In another embodiment, provided herein is a binding region that binds ENPP3, comprising: (i) a VH comprising a HCDR1, a HCDR2, and a HCDR3 having an amino acid sequence of SEQ ID NOs: 166, 167 and 161, respectively, and (ii) a VL comprising a LCDR1, a LCDR2, and a LCDR3 having an amino acid sequence of SEQ ID NOs: 162, 163 and 76, respectively.

In one embodiment, provided herein is a binding region that binds ENPP3, comprising a VH comprising a HCDR1, a HCDR2, and a HCDR3 having an amino acid sequence of SEQ ID NOs: 11, 168, and 169, respectively. In another embodiment, provided herein is a binding region that binds ENPP3, comprising a VL comprising a LCDR1, a LCDR2, and a LCDR3 having an amino acid sequence of SEQ ID NOs: 170, 171 and 86, respectively. In another embodiment, provided herein is a binding region that binds ENPP3, comprising: (i) a VH comprising a HCDR1, a HCDR2, and a HCDR3 having an amino acid sequence of SEQ ID NOs: 11, 168, and 169, respectively, and (ii) a VL comprising a LCDR1, a LCDR2, and a LCDR3 having an amino acid sequence of SEQ ID NOs: 170, 171 and 86, respectively.

In one embodiment, provided herein is a binding region that binds ENPP3, comprising a VH comprising a HCDR1, a HCDR2, and a HCDR3 having an amino acid sequence of SEQ ID NOs: 172, 173 and 174, respectively. In another embodiment, provided herein is a binding region that binds ENPP3, comprising a VL comprising a LCDR1, a LCDR2, and a LCDR3 having an amino acid sequence of SEQ ID NOs: 175, 176 and 76, respectively. In another embodiment, provided herein is a binding region that binds ENPP3, comprising: (i) a VH comprising a HCDR1, a HCDR2, and a HCDR3 having an amino acid sequence of SEQ ID NOs: 172, 173 and 174, respectively, and (ii) a VL comprising a LCDR1, a LCDR2, and a LCDR3 having an amino acid sequence of SEQ ID NOs: 175, 176 and 76, respectively.

In some embodiments, the ENPP3 binding region further comprises one or more framework regions of the VH or VL having the amino acid sequence of any one of SEQ ID NO: 22, SEQ ID NO:23, SEQ ID NO:45, SEQ ID NO:46, SEQ ID NO:68, SEQ ID NO:69, SEQ ID NO: 70, SEQ ID NO:91, SEQ ID NO:92, SEQ ID NO: 113, SEQ ID NO: 114, SEQ ID NO: 115, SEQ ID NO: 134, SEQ ID NO: 135, SEQ ID NO: 136, SEQ ID NO: 158, SEQ ID NO: 159, SEQ ID NO: 177 and SEQ ID NO:178. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In one embodiment, provided herein is a binding region that binds ENPP3, comprising a VH having an amino acid sequence of SEQ ID NO:22. In another embodiment, provided herein is a binding region that binds ENPP3, comprising a VL having an amino acid sequence of SEQ ID NO:23. In another embodiment, provided herein is a binding region that binds ENPP3, comprising: (i) a VH having an amino acid sequence of SEQ ID NO:22, and (ii) a VL having an amino acid sequence of SEQ ID NO:23.

In one embodiment, provided herein is a binding region that binds ENPP3, comprising a VH having an amino acid sequence of SEQ ID NO:45. In another embodiment, provided herein is a binding region that binds ENPP3, comprising a VL having an amino acid sequence of SEQ ID NO:46. In another embodiment, provided herein is a binding region that binds ENPP3, comprising: (i) a VH having an amino acid sequence of SEQ ID NO:45, and (ii) a VL having an amino acid sequence of SEQ ID NO:46.

In one embodiment, provided herein is a binding region that binds ENPP3, comprising a VH having an amino acid sequence of SEQ ID NO:68. In another embodiment, provided herein is a binding region that binds ENPP3, comprising a VL having an amino acid sequence of SEQ ID NO:69. In another embodiment, provided herein is a binding region that binds ENPP3, comprising: (i) a VH having an amino acid sequence of SEQ ID NO:68, and (ii) a VL having an amino acid sequence of SEQ ID NO:69.

In one embodiment, provided herein is a binding region that binds ENPP3, comprising a VH having an amino acid sequence of SEQ ID NO:68. In another embodiment, provided herein is a binding region that binds ENPP3, comprising a VL having an amino acid sequence of SEQ ID NO:70. In another embodiment, provided herein is a binding region that binds ENPP3, comprising: (i) a VH having an amino acid sequence of SEQ ID NO:68, and (ii) a VL having an amino acid sequence of SEQ ID NO:70.

In one embodiment, provided herein is a binding region that binds ENPP3, comprising a VH having an amino acid sequence of SEQ ID NO:91. In another embodiment, provided herein is a binding region that binds ENPP3, comprising a VL having an amino acid sequence of SEQ ID NO:92. In another embodiment, provided herein is a binding region that binds ENPP3, comprising: (i) a VH having an amino acid sequence of SEQ ID NO:91, and (ii) a VL having an amino acid sequence of SEQ ID NO:92.

In one embodiment, provided herein is a binding region that binds ENPP3, comprising a VH having an amino acid sequence of SEQ ID NO:113. In another embodiment, provided herein is a binding region that binds ENPP3, comprising a VL having an amino acid sequence of SEQ ID NO:114. In another embodiment, provided herein is a binding region that binds ENPP3, comprising: (i) a VH having an amino acid sequence of SEQ ID NO:113, and (ii) a VL having an amino acid sequence of SEQ ID NO:114.

In one embodiment, provided herein is a binding region that binds ENPP3, comprising a VH having an amino acid sequence of SEQ ID NO:113. In another embodiment, provided herein is a binding region that binds ENPP3, comprising a VL having an amino acid sequence of SEQ ID NO:115. In another embodiment, provided herein is a binding region that binds ENPP3, comprising: (i) a VH having an amino acid sequence of SEQ ID NO: 113, and (ii) a VL having an amino acid sequence of SEQ ID NO:115.

In one embodiment, provided herein is a binding region that binds ENPP3, comprising a VH having an amino acid sequence of SEQ ID NO:134. In another embodiment, provided herein is a binding region that binds ENPP3, comprising a VL having an amino acid sequence of SEQ ID NO:135. In another embodiment, provided herein is a binding region that binds ENPP3, comprising: (i) a VH having an amino acid sequence of SEQ ID NO:134, and (ii) a VL having an amino acid sequence of SEQ ID NO:135.

In one embodiment, provided herein is a binding region that binds ENPP3, comprising a VH having an amino acid sequence of SEQ ID NO:134. In another embodiment, provided herein is a binding region that binds ENPP3, comprising a VL having an amino acid sequence of SEQ ID NO:136. In another embodiment, provided herein is a binding region that binds ENPP3, comprising: (i) a VH having an amino acid sequence of SEQ ID NO:134, and (ii) a VL having an amino acid sequence of SEQ ID NO:136.

In one embodiment, provided herein is a binding region that binds ENPP3, comprising a VH having an amino acid sequence of SEQ ID NO:158. In another embodiment, provided herein is a binding region that binds ENPP3, comprising a VL having an amino acid sequence of SEQ ID NO:159. In another embodiment, provided herein is a binding region that binds ENPP3, comprising: (i) a VH having an amino acid sequence of SEQ ID NO: 158, and (ii) a VL having an amino acid sequence of SEQ ID NO:159.

In one embodiment, provided herein is a binding region that binds ENPP3, comprising a VH having an amino acid sequence of SEQ ID NO:177. In another embodiment, provided herein is a binding region that binds ENPP3, comprising a VL having an amino acid sequence of SEQ ID NO:178. In another embodiment, provided herein is a binding region that binds ENPP3, comprising: (i) a VH having an amino acid sequence of SEQ ID NO:177, and (ii) a VL having an amino acid sequence of SEQ ID NO: 178.

In certain embodiments, the ENPP3 binding region provided herein comprises amino acid sequences with certain percent identity relative to any ENPP3 binding region provided herein (such as in Table 1, Table 2, Table 3, Table 4, Table 5, Table 6, Table 7, Table 8, Table 9, Table 10, and Table 11).

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the ENPP3 binding region provide herein contains substitutions (e.g., conservative substitutions), insertions, or deletions relative to the reference sequence, but the ENPP3 binding region comprising that sequence retains the ability to bind to ENPP3. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the ENPP3 binding region provided herein includes post-translational modifications of a reference sequence.

In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:22, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:23.

In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:45, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:46. In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:68, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 69. In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:68, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:70. In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:91, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 92. In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:113, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:114. In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:113, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 115. In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 134, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 135. In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 134, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 136. In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:158, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:159. In some embodiments, the ENPP3 binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:177, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 178.

In some embodiments, the ENPP3 binding region provided herein binds to the same epitope as an ENPP3 binding region comprising a VH comprising the amino acid sequence of SEQ ID NO:22, and a VL comprising the amino acid sequence of SEQ ID NO:23. In some embodiments, the ENPP3 binding region provided herein binds to the same epitope as an ENPP3 binding region comprising a VH comprising the amino acid sequence of SEQ ID NO:45, and a VL comprising the amino acid sequence of SEQ ID NO:46. In some embodiments, the ENPP3 binding region provided herein binds to the same epitope as an ENPP3 binding region comprising a VH comprising the amino acid sequence of SEQ ID NO:68, and a VL comprising the amino acid sequence of SEQ ID NO:69. In some embodiments, the ENPP3 binding region provided herein binds to the same epitope as an ENPP3 binding region comprising a VH comprising the amino acid sequence of SEQ ID NO:68, and a VL comprising the amino acid sequence of SEQ ID NO:70. In some embodiments, the ENPP3 binding region provided herein binds to the same epitope as an ENPP3 binding region comprising a VH comprising the amino acid sequence of SEQ ID NO:91, and a VL comprising the amino acid sequence of SEQ ID NO: 92. In some embodiments, the ENPP3 binding region provided herein binds to the same epitope as an ENPP3 binding region comprising a VH comprising the amino acid sequence of SEQ ID NO:113, and a VL comprising the amino acid sequence of SEQ ID NO: 114. In some embodiments, the ENPP3 binding region provided herein binds to the same epitope as an ENPP3 binding region comprising a VH comprising the amino acid sequence of SEQ ID NO: 113, and a VL comprising the amino acid sequence of SEQ ID NO:115. In some embodiments, the ENPP3 binding region provided herein binds to the same epitope as an ENPP3 binding region comprising a VH comprising the amino acid sequence of SEQ ID NO: 134, and a VL comprising the amino acid sequence of SEQ ID NO:135. In some embodiments, the ENPP3 binding region provided herein binds to the same epitope as an ENPP3 binding region comprising a VH comprising the amino acid sequence of SEQ ID NO:134, and a VL comprising the amino acid sequence of SEQ ID NO: 136. In some embodiments, the ENPP3 binding region provided herein binds to the same epitope as an ENPP3 binding region comprising a VH comprising the amino acid sequence of SEQ ID NO: 158, and a VL comprising the amino acid sequence of SEQ ID NO: 159. In some embodiments, the ENPP3 binding region provided herein binds to the same epitope as an ENPP3 binding region comprising a VH comprising the amino acid sequence of SEQ ID NO:177, and a VL comprising the amino acid sequence of SEQ ID NO: 178.

In some embodiments, the ENPP3 binding region provided herein specifically binds to ENPP3 competitively with an ENPP3 binding region comprising a VH comprising the amino acid sequence of SEQ ID NO:22, and a VL comprising the amino acid sequence of SEQ ID NO: 23. In some embodiments, the ENPP3 binding region provided herein specifically binds to ENPP3 competitively with an ENPP3 binding region comprising a VH comprising the amino acid sequence of SEQ ID NO:45, and a VL comprising the amino acid sequence of SEQ ID NO: 46. In some embodiments, the ENPP3 binding region provided herein specifically binds to ENPP3 competitively with an ENPP3 binding region comprising a VH comprising the amino acid sequence of SEQ ID NO:68, and a VL comprising the amino acid sequence of SEQ ID NO: 69. In some embodiments, the ENPP3 binding region provided herein specifically binds to ENPP3 competitively with an ENPP3 binding region comprising a VH comprising the amino acid sequence of SEQ ID NO:68, and a VL comprising the amino acid sequence of SEQ ID NO: 70. In some embodiments, the ENPP3 binding region provided herein specifically binds to ENPP3 competitively with an ENPP3 binding region comprising a VH comprising the amino acid sequence of SEQ ID NO:91, and a VL comprising the amino acid sequence of SEQ ID NO: 92. In some embodiments, the ENPP3 binding region provided herein specifically binds to ENPP3 competitively with an ENPP3 binding region comprising a VH comprising the amino acid sequence of SEQ ID NO: 113, and a VL comprising the amino acid sequence of SEQ ID NO: 114. In some embodiments, the ENPP3 binding region provided herein specifically binds to ENPP3 competitively with an ENPP3 binding region comprising a VH comprising the amino acid sequence of SEQ ID NO:113, and a VL comprising the amino acid sequence of SEQ ID NO: 115. In some embodiments, the ENPP3 binding region provided herein specifically binds to ENPP3 competitively with an ENPP3 binding region comprising a VH comprising the amino acid sequence of SEQ ID NO: 134, and a VL comprising the amino acid sequence of SEQ ID NO: 135. In some embodiments, the ENPP3 binding region provided herein specifically binds to ENPP3 competitively with an ENPP3 binding region comprising a VH comprising the amino acid sequence of SEQ ID NO: 134, and a VL comprising the amino acid sequence of SEQ ID NO: 136. In some embodiments, the ENPP3 binding region provided herein specifically binds to ENPP3 competitively with an ENPP3 binding region comprising a VH comprising the amino acid sequence of SEQ ID NO:158, and a VL comprising the amino acid sequence of SEQ ID NO: 159. In some embodiments, the ENPP3 binding region provided herein specifically binds to ENPP3 competitively with an ENPP3 binding region comprising a VH comprising the amino acid sequence of SEQ ID NO: 177, and a VL comprising the amino acid sequence of SEQ ID NO: 178.

TABLE 1
NPP3B56 Binding Region Sequences

| | Kabat | Chothia | AbM | Contact | IMGT |
|---|---|---|---|---|---|
| Heavy Chain | DYSMN | GFTFSDY | GFTFSDYSMN | SDYSMN | GFTFSDYS |
| variable region | (SEQ ID NO: 1) | (SEQ ID NO: 7) | (SEQ ID NO: 9) | (SEQ ID NO: 11) | (SEQ ID |
| CDR1 | | | | | NO: 17) |
| Heavy Chain | SISSISSYVKY | SSISSY | SISSISSYVK | WVSSISSISSYVK | ISSISSYV |
| variable region | ADSVKG | (SEQ ID NO: 8) | (SEQ ID NO: 10 | (SEQ ID NO: 12) | (SEQ ID |
| CDR2 | (SEQ ID NO: 2) | | | | NO: 18) |
| Heavy Chain | GHYFDY | GHYFDY | GHYFDY | ARGHYFD | ARGHYFDY |
| variable region | (SEQ ID NO: 3) | (SEQ ID NO: 3) | (SEQ ID NO: 3) | (SEQ ID NO: 13) | (SEQ ID |
| CDR3 | | | | | NO: 19) |
| Light Chain | RASQSVSSNL | RASQSVSSNLA | RASQSVSSNLA | SSNLAWY | QSVSSN |
| variable region | A | (SEQ ID NO: 4) | (SEQ ID NO: 4) | (SEQ ID NO: 14) | (SEQ ID |
| CDR1 | (SEQ ID NO: 4) | | | | NO: 20) |
| Light Chain | GASTRAT | GASTRAT | GASTRAT | LLIYGASTRA | GAS |
| variable region | (SEQ ID NO: 5) | (SEQ ID NO: 5) | (SEQ ID NO: 5) | (SEQ ID NO: 15) | |
| CDR2 | | | | | |
| Light Chain | QQYNNWPRT | QQYNNWPRT | QQYNNWPRT | QQYNNWPR | QQYNNWPR |
| variable region | (SEQ ID NO: 6) | (SEQ ID NO: 6) | (SEQ ID NO: 6) | (SEQ ID NO: 16) | T |
| CDR3 | | | | | (SEQ ID |
| | | | | | NO: 6) |

VH (SEQ ID NO: 22):
EVQLVESGGGRVKPGGSLRLSCAASGFTFSDYSMNWVRQAPGKGLEWVSSISSISSYVKYADSVKGRFTMSRDNAKNSLFLQMNS
LRDEDMAVYYCARGHYFDYWGQGTLVTVSS
DNA Sequence encoding VH (SEQ ID NO: 316)
GAGGTGCAGCTGGTTGAATCTGGCGGCGGAAGAGTGAAGCCTGGCGGATCTCTGAGACTGTCTTGTGCCGCCTCTGGCTTCACCT
TCTCCGACTACTCCATGAACTGGGTCCGACAGGCTCCTGGCAAAGGCCTGGAATGGGTGTCCTCTATCTCCTCCATCTCCAGCTA
CGTGAAGTACGCCGACTCCGTGAAGGGCAGATTCACCATGTCCAGAGACAACGCCAAGAACTCCCTGTTCCTGCAGATGAACAGC
CTGCGCGACGAGGACATGGCCGTGTACTATTGTGCCAGAGGCCACTACTTCGACTACTGGGGACAGGGCACACTGGTCACAGTCT
CTTCT
VL (SEQ ID NO: 23):
EIVMTQSPATLSVSPGERATLSCRASQSVSSNLAWYQQKPGLAPRLLIYGASTRATGIPARFSGSGSGTEFTLTISSLQSEDFAV
YYCQQYNNWPRTFGQGTKVEIK
DNA Sequence encoding VL (SEQ ID NO: 317)
GAGATCGTGATGACCCAGTCTCCTGCCACACTGTCTGTGTCTCCCGGCGAGAGAGCTACCCTGTCTTGTAGAGCCTCTCAGTCCG
TGTCCTCCAACCTGGCCTGGTATCAGCAGAAGCCTGGACTGGCTCCCCGGCTGTTGATCTATGGCGCTTCTACCAGAGCCACAGG
CATCCCCGCTAGATTCTCCGGCTCTGGCTCTGGCACAGAGTTTACCCTGACCATCTCCAGCCTGCAGTCCGAGGATTTCGCCGTG
TACTACTGCCAGCAGTACAACAACTGGCCCCGGACCTTTGGCCAGGGCACCAAGGTGGAAATCAAG

TABLE 2
NPP3B86 Binding Region Sequences

| | Kabat | Chothia | AbM | Contact | IMGT |
|---|---|---|---|---|---|
| Heavy Chain | NYYIF | GYTVTNY | GYTVTNYYIF | TNYYIF | GYTVTNYY |
| variable region | (SEQ ID NO: 24) | (SEQ ID NO: 30) | (SEQ ID NO: 32) | (SEQ ID NO: 34) | (SEQ ID |
| CDR1 | | | | | NO: 40) |
| Heavy Chain | WINPNSGGTN | NPNSGG | WINPNSGGTN | WMGWINPNSGGT | INPNSGGT |
| variable region | YAQKFQG | (SEQ ID NO: 31) | (SEQ ID NO: 33) | N | (SEQ ID |
| CDR2 | (SEQ ID NO: 25) | | | (SEQ ID NO: 35) | NO: 41) |
| Heavy Chain | GGYSGYALSS | GGYSGYALSSHYG | GGYSGYALSSHYG | ARGGYSGYALSS | ARGGYSGY |
| variable region | HYGMDV | MDV | MDV | HYGMD | ALSSHYGMD |
| CDR3 | (SEQ ID NO: 26) | (SEQ ID NO: 26) | (SEQ ID NO: 26) | (SEQ ID NO: 36) | V |
| | | | | | (SEQ ID |
| | | | | | NO: 42) |
| Light Chain | RASQSVSSSY | RASQSVSSSYLA | RASQSVSSSYLA | SSSYLAWY | QSVSSSY |
| variable region | LA | (SEQ ID NO: 27) | (SEQ ID NO: 27) | (SEQ ID NO: 37) | (SEQ ID |
| CDR1 | (SEQ ID NO: 27) | | | | NO: 43) |
| Light Chain | DASSRAT | DASSRAT | DASSRAT | LLIYDASSRA | DAS |
| variable region | (SEQ ID NO: 28) | (SEQ ID NO: 28) | (SEQ ID NO: 28) | (SEQ ID NO: 38) | |
| CDR2 | | | | | |
| Light Chain | QQYDSSPLT | QQYDSSPLT | QQYDSSPLT | QQYDSSPL | QQYDSSPLT |
| variable region | (SEQ ID NO: 29) | (SEQ ID NO: 29) | (SEQ ID NO: 29) | (SEQ ID NO: 39) | (SEQ ID |
| CDR3 | | | | | NO: 29) |

VH (SEQ ID NO: 45)
QVQLVQSGAEVKKPGASVKVSCKASGYTVTNYYIFWVRQAPGQGLEWMGWINPNSGGTNYAQKFQGRVTMTRDTSINTAYMKLSRLGSDDTA
VYYCARGGYSGYALSSHYGMDVWGQGTMVTVSS
VL (SEQ ID NO: 46)
EIVLTQSPGTLSLSPGERATLSCRASQSVSSSYLAWYQQKPGQAPRLLIYDASSRATGIPDRFSGSGSGTDFTLIISRLEPEDFAVYYCQQY
DSSPLTFGGGTKVEIK

TABLE 3
NPP3B64 Binding Region Sequences

| | Kabat | Chothia | AbM | Contact | IMGT |
|---|---|---|---|---|---|
| Heavy Chain | TYYIH | GYTFTTY | GYTFTTYYIH | TTYYIH | GYTFTTYY |
| variable region | (SEQ ID NO: 47) | (SEQ ID NO: 53) | (SEQ ID NO: 55) | (SEQ ID NO: 57) | (SEQ ID |
| CDR1 | | | | | NO: 63) |
| Heavy Chain | IINPSGGSTNY | NPSGGS | IINPSGGSTN | WMGIINPSGGSTN | INPSGGST |
| variable region | AQKFQG | (SEQ ID NO: 54) | (SEQ ID NO: 56) | (SEQ ID NO: 58) | (SEQ ID |
| CDR2 | (SEQ ID NO: 48) | | | | NO: 64) |
| Heavy Chain | DYYYYYYGM | DYYYYYYGMDV | DYYYYYYGMDV | ARDYYYYYYGM | ARDYYYYY |
| variable region | DV | (SEQ ID NO: 49) | (SEQ ID NO: 49) | D | YGMDV |
| CDR3 | (SEQ ID NO: 49) | | | (SEQ ID NO: 59) | (SEQ ID |
| | | | | | NO: 65) |
| Light Chain | RSSQSL VRNG | RSSQSLVRNGGYT | RSSQSLVRNGGYT | VRNGGYTYLNWL | QSLVRNGGY |
| variable region | GYTYLN | YLN | YLN | (SEQ ID NO: 60) | TY |
| CDR1 | (SEQ ID NO: 50) | (SEQ ID NO: 50) | (SEQ ID NO: 50) | | (SEQ ID |
| | | | | | NO: 66) |
| Light Chain | QISNRFS | QISNRFS | QISNRFS | LLIYQISNRF | QIS |
| variable region | (SEQ ID NO: 51) | (SEQ ID NO: 51) | (SEQ ID NO: 51) | (SEQ ID NO: 61) | |
| CDR2 | | | | | |
| Light Chain | MQATQFPHT | MQATQFPHT | MQATQFPHT | MQATQFPH | MQATQFPHT |
| variable region | (SEQ ID NO: 52) | (SEQ ID NO: 52) | (SEQ ID NO: 52) | (SEQ ID NO: 62) | (SEQ ID |
| CDR3 | | | | | NO: 52) |

VH (SEQ ID NO: 68)
QVQLVQSGAEVKKPGASVKVSCKASGYTFTTYYIHWVRQAPGQGLEWMGIINPSGGSTNYAQKFQGRVTMTRDTSTRIVYMELGSLRSEDTAVY
YCARDYYYYYYGMDVWGQGTTVTVSS
VL (SEQ ID NO: 69)
DIVMTQSPLSLPVTLGQPASISCRSSQSLVRNGGYTYLNWLQQRPGQPPRLLIYQISNRFSGVPDRFSGSGAGTDFTLTISRVEAEDVGVYYCM
QATQFPHTFGQGTKLEIK

TABLE 4
NPP3B68 Binding Region Sequences

| | Kabat | Chothia | AbM | Contact | IMGT |
|---|---|---|---|---|---|
| Heavy Chain | TYYIH | GYTFTTY | GYTFTTYYIH | TTYYIH | GYTFTTYY |
| variable region | (SEQ ID NO: 47) | (SEQ ID NO: 53) | (SEQ ID NO: 55) | (SEQ ID NO: 57) | (SEQ ID |
| CDR1 | | | | | NO: 63) |
| Heavy Chain | IINPSGGSTNY | NPSGGS | IINPSGGSTN | WMGIINPSGGSTN | INPSGGST |
| variable region | AQKFQG | (SEQ ID NO: 54) | (SEQ ID NO: 56) | (SEQ ID NO: 58) | (SEQ ID |
| CDR2 | (SEQ ID NO: 48) | | | | NO: 64) |
| Heavy Chain | DYYYYYYGM | DYYYYYYGMDV | DYYYYYYGMDV | ARDYYYYYYGM | ARDYYYYY |
| variable region | DV | (SEQ ID NO: 49) | (SEQ ID NO: 49) | D | YGMDV |
| CDR3 | (SEQ ID NO: 49) | | | (SEQ ID NO: 59) | (SEQ ID |
| | | | | | NO: 65) |
| Light Chain | GYTYLN | YLN | YLN | VRNGGYTYLNWL | QSLVRNGGY |
| variable region | (SEQ ID NO: 50) | (SEQ ID NO: 50) | (SEQ ID NO: 50) | (SEQ ID NO: 60) | TY |
| CDR1 | RSSQSLVRNG | \|RSSQSLVRNGGYT | RSSQSL VRNGGYT | | (SEQ ID |
| | | | | | NO: 66) |
| Light Chain | QISNRFS | QISNRFS | QISNRFS | LLIYQISNRF | QIS |
| variable region | (SEQ ID NO: 51) | (SEQ ID NO: 51) | (SEQ ID NO: 51) | (SEQ ID NO: 61) | |
| CDR2 | | | | | |
| Light Chain | MQATQFPHT | MQATQFPHT | MQATQFPHT | MQATQFPH | MQATQFPHT |
| variable region | (SEQ ID NO: 52) | (SEQ ID NO: 52) | (SEQ ID NO: 52) | (SEQ ID NO: 62) | (SEQ ID |
| CDR3 | | | | | NO: 52) |

VH (SEQ ID NO: 68)
QVQLVQSGAEVKKPGASVKVSCKASGYTFTTYYIHWVRQAPGQGLEWMGIINPSGGSTNYAQKFQGRVTMTRDTSTRIVYMELGSLR
SEDTAVYYCARDYYYYYYGMDVWGQGTTVTVSS
VL (SEQ ID NO: 70)
DIVMTQTPLSSPVTLGQPASISCRSSQSLVRNGGYTYLNWLQQRPGQPPRLLIYQISNRFSGVPDRFSGSGAGTDFTLTISRVEAED
VGVYYCMQATQFPHTFGQGTKLEIK

TABLE 5
NPP3B60 Binding Region Sequences

| | Kabat | Chothia | AbM | Contact | IMGT |
|---|---|---|---|---|---|
| Heavy Chain | NYWIG | GYGFTNY | GYGFTNYWIG | TNYWIG | GYGFTNYW |
| variable region | (SEQ ID NO: 71) | (SEQ ID NO: 77) | (SEQ ID NO: 79) | (SEQ ID NO: 81) | (SEQ ID |
| CDR1 | | | | | NO: 87) |
| Heavy Chain | IIYPSDSNTRY | YPSDSN | IIYPSDSNTR | WMGIIYPSDSNTR | IYPSDSNT |
| variable region | SPSFQG | (SEQ ID NO: 78) | (SEQ ID NO: 80) | (SEQ ID NO: 82) | (SEQ ID |
| CDR2 | (SEQ ID NO: 72) | | | | NO: 88) |
| Heavy Chain | QNYYYYTMD | QNYYYYTMDV | QNYYYYTMDV | TRONYYYYTMD | TRONYYYYT |
| variable region | V | (SEQ ID NO: 73) | (SEQ ID NO: 73) | (SEQ ID NO: 83) | MDV |
| CDR3 | (SEQ ID NO: 73) | | | | (SEQ ID |
| | | | | | NO: 89) |
| Light Chain | RASQVIRSYL | RASQVIRSYLA | RASQVIRSYLA | RSYLAWY | QVIRSY |
| variable region | A | (SEQ ID NO: 74) | (SEQ ID NO: 74) | (SEQ ID NO: 84) | (SEQ ID |
| CDR1 | (SEQ ID NO: 74) | | | | NO: 90) |
| Light Chain | GASTLQS | GASTLOS | GASTLOS | LLIYGASTLQ | GAS |
| variable region | (SEQ ID NO: 75) | (SEQ ID NO: 75) | (SEQ ID NO: 75) | (SEQ ID NO: 85) | |
| CDR2 | | | | | |
| Light Chain | QQLNSYPRT | QQLNSYPRT | QQLNSYPRT | QQLNSYPR | QQLNSYPRT |
| variable region | (SEQ ID NO: 76) | (SEQ ID NO: 76) | (SEQ ID NO: 76) | (SEQ ID NO: 86) | (SEQ ID |
| CDR3 | | | | | NO: 76) |

VH (SEQ ID NO: 91)
QVQLVQSGAEVKKPGESLKISCKGSGYGFTNYWIGWVRQMPGKGLEWMGIIYPSDSNTRYSPSFQGQVTISADKSINTA
YLQWSSLKASDTAMYYCTRQNYYYYTMDVWGQGTTVTVSS
VL (SEQ ID NO: 92)
DIQMTQSPSFLSASVGDRVTITCRASQVIRSYLAWYQQKPGKAPKLLIYGASTLQSGFPSRFSGSGSGTEFTLTISSLQ
PEDFATYYCQQLNSYPRTFGQGTKVEIK

TABLE 6
NPP3B61 Binding Region Sequences

| | Kabat | Chothia | AbM | Contact | IMGT |
|---|---|---|---|---|---|
| Heavy Chain | SYYWS | GGSISSY | GGSISSYYWS | SSYYWS | GGSISSYY |
| variable region | (SEQ ID NO: 93) | (SEQ ID NO: 99) | (SEQ ID NO: 101) | (SEQ ID NO: 103) | (SEQ ID |
| CDR1 | | | | | NO: 109) |
| Heavy Chain | RIYTTGRIYTS | YTTGRIYTSGY | RIYTTGRIYTSGYT | WIGRIYTTGRIYTS | IYTTGRIYTS |
| variable region | GYTNYNPSLK | (SEQ ID NO: 100) | N | GYTN | GYT |
| CDR2 | S | | (SEQ ID NO: 102) | (SEQ ID NO: 104) | (SEQ ID |
| | (SEQ ID NO: 94) | | | | NO: 110) |
| Heavy Chain | GGELFTDGFDI | GGELFTDGFDI | GGELFTDGFDI | ARGGELFTDGFD | ARGGELFTD |
| variable region | (SEQ ID NO: 95) | (SEQ ID NO: 95) | (SEQ ID NO: 95) | (SEQ ID NO: 105) | GFDI |
| CDR3 | | | | | (SEQ ID |
| | | | | | NO: 111) |
| Light Chain | RASQSVISTYL | RASQSVISTYLA | RASQSVISTYLA | ISTYLAWY | QSVISTY |
| variable region | A | (SEQ ID NO: 96) | (SEQ ID NO: 96) | (SEQ ID NO: 106) | (SEQ ID |
| CDR1 | (SEQ ID NO: 96) | | | | NO: 112) |
| Light Chain | GASSRAT | GASSRAT | GASSRAT | LLIYGASSRA | GAS) |
| variable region | (SEQ ID NO: 97) | (SEQ ID NO: 97) | (SEQ ID NO: 97) | (SEQ ID NO: 107) | |
| CDR2 | | | | | |
| Light Chain | QQYDTSPIT | QQYDTSPIT | QQYDTSPIT | QQYDTSPI | QQYDTSPIT |
| variable region | (SEQ ID NO: 98) | (SEQ ID NO: 98) | (SEQ ID NO: 98) | (SEQ ID NO: 108) | (SEQ ID |
| CDR3 | | | | | NO: 98) |

VH (SEQ ID NO: 113)
QVQLQESGPGLVRPSETLSLTCTVSGGSISSYYWSWIRQPAGKGLEWIGRIYTTGRIYTSGYTNYNPSLKSRVTMSIDTS
KIQFSLRLNSVTAADTAVYWCARGGELFTDGFDIWGQGTMVTVSS
VL (SEQ ID NO: 114)
EIVMTQSPGTLSLSPGERATLSCRASQSVISTYLAWYQQNPGQAPRLLIYGASSRATGIPDRESGSGSGTDFTLTISRLE
PEDFAVYYCQQYDTSPITFGQGTRLEIK

TABLE 7
NPP3B62 Binding Region Sequences

| | Kabat | Chothia | AbM | Contact | IMGT |
|---|---|---|---|---|---|
| Heavy Chain | SYYWS | GGSISSY | GGSISSYYWS | SSYYWS | GGSISSYY |
| variable region | (SEQ ID NO: 93) | (SEQ ID NO: 99) | (SEQ ID NO: 101) | (SEQ ID NO: 103) | (SEQ ID |
| CDR1 | | | | | NO: 109) |
| Heavy Chain | RIYTTGRIYTS | YTTGRIYTSGY | RIYTTGRIYTSGYT | WIGRIYTTGRIYTS | IYTTGRIYTS |
| variable region | GYTNYNPSLK | (SEQ ID NO: 100) | N (SEQ ID NO: 102) | GYTN | GYT |
| CDR2 | S | | | (SEQ ID NO: 104) | (SEQ ID |
| | (SEQ ID NO: 94) | | | | NO: 110) |
| Heavy Chain | GGELFTDGFDI | GGELFTDGFDI | GGELFTDGFDI | ARGGELFTDGFD | ARGGELFTD |
| variable region | (SEQ ID NO: 95) | (SEQ ID NO: 95) | (SEQ ID NO: 95) | (SEQ ID NO: 105) | GFDI |
| CDR3 | | | | | (SEQ ID |
| | | | | | NO: 111) |
| Light Chain | RASQSVISTYL | RASQSVISTYLA | RASQSVISTYLA | ISTYLAWY | QSVISTY |
| variable region | A | (SEQ ID NO: 96) | (SEQ ID NO: 96) | (SEQ ID NO: 106) | (SEQ ID |
| CDR1 | (SEQ ID NO: 96) | | | | NO: 112) |
| Light Chain | GASSRAT | GASSRAT | GASSRAT | LLIYGASSRA | GAS |
| variable region | (SEQ ID NO: 97) | (SEQ ID NO: 97) | (SEQ ID NO: 97) | (SEQ ID NO: 107) | |
| CDR2 | | | | | |
| Light Chain | QQYDTSPIT | QQYDTSPIT | QQYDTSPIT | QQYDTSPI | QQYDTSPIT |
| variable region | (SEQ ID NO: 98) | (SEQ ID NO: 98) | (SEQ ID NO: 98) | (SEQ ID NO: 108) | (SEQ ID |
| CDR3 | | | | | NO: 98) |

VH (SEQ ID NO: 113)
QVQLQESGPGLVRPSETLSLTCTVSGGSISSYYWSWIRQPAGKGLEWIGRIYTTGRIYTSGYTNYNPSLKSRVTMSIDTSKIQF
SLRLNSVTAADTAVYWCARGGELFTDGFDIWGQGTMVTVSS
VL (SEQ ID NO: 115)
EIVMTQSPGTLSLSPGERATLSCRASQSVISTYLAWYQQNPGQAPRLLIYGASSRATGIPDRFSGSGSGTDFTLTISRLEPEDF
VVYYCQQYDTSPITFGQGTRLEIK

TABLE 8
NPP3B101 Binding Region Sequences

| | Kabat | Chothia | AbM | Contact | IMGT |
|---|---|---|---|---|---|
| Heavy Chain | SHYWS | GGSISSH | GGSISSHYWS | SSHYWS | GGSISSHY |
| variable | (SEQ ID | (SEQ ID NO: 121) | (SEQ ID NO: 123) | (SEQ ID NO: 125) | (SEQ ID |
| region | NO: 116) | | | | NO: 130) |
| CDR1 | | | | | |
| Heavy Chain | NIFYSGSTNY | FYSGS | NIFYSGSTN | WIGNIFYSGSTN | IFYSGST |
| variable | NPSLKS | (SEQ ID NO: 122) | (SEQ ID NO: 124) | (SEQ ID NO: 126) | (SEQ ID |
| region | (SEQ ID | | | | NO: 131) |
| CDR2 | NO: 117) | | | | |
| Heavy Chain | HDHVWFDP | HDHVWFDP | HDHVWFDP | ARHDHVWFD | ARHDHVWF |
| variable | (SEQ ID | (SEQ ID NO: 118) | (SEQ ID NO: 118) | (SEQ ID NO: 127) | DP |
| region | NO: 118) | | | | (SEQ ID |
| CDR3 | | | | | NO: 132) |
| Light Chain | RASQSVSRNL | RASQSVSRNLV | RASQSVSRNLV | SRNLVWY | QSVSRN |
| variable | V | (SEQ ID NO: 119) | (SEQ ID NO: 119) | (SEQ ID NO: 128) | (SEQ ID |
| region | (SEQ ID | | | | NO: 133) |
| CDR1 | NO: 119) | | | | |
| Light Chain | GASTRAT | GASTRAT | GASTRAT | LLIYGASTRA | GAS |
| variable | (SEQ ID NO: 5) | (SEQ ID NO: 5) | (SEQ ID NO: 5) | (SEQ ID NO: 15) | |
| region | | | | | |
| CDR2 | | | | | |
| Light Chain | QQYNNWPPW | QQYNNWPPWT | QQYNNWPPWT | QQYNNWPPW | QQYNNWPP |
| variable | T | (SEQ ID NO: 120) | (SEQ ID NO: 120) | (SEQ ID NO: 129) | WT |
| region | (SEQ ID | | | | (SEQ ID |
| CDR3 | NO: 120) | | | | NO: 120) |

VH (SEQ ID NO: 134)
QVQLQESGPGLVKTSETLSLTCTVSGGSISSHYWSWIRQPPGKGLEWIGNIFYSGSTNYNPSLKSRVTMSVDTSKNHFSLKLISVTTAD
TAIYYCARHDHVWFDPWGQGTLVTVSS
VL (SEQ ID NO: 135)
EIVLTQSPGTLSVFPGERATLSCRASQSVSRNLVWYQQKPGQAPRLLIYGASTRATGIPDRFSGSGSGTEFTLTISSLQSEDFAVYYCQ
QYNNWPPWTFGQGTKVEIK

TABLE 9
NPP3B100 Binding Region Sequences

| | Kabat | Chothia | AbM | Contact | IMGT |
|---|---|---|---|---|---|
| Heavy Chain | SHYWS | GGSISSH | GGSISSHYWS | SSHYWS | GGSISSHY |
| variable region | (SEQ ID | (SEQ ID NO: 121) | (SEQ ID NO: 123) | (SEQ ID NO: 125) | (SEQ ID |
| CDR1 | NO: 116) | | | | NO: 130) |
| Heavy Chain | NIFYSGSTNY | FYSGS | NIFYSGSTN | WIGNIFYSGSTN | IFYSGST |
| variable region | NPSLKS | (SEQ ID NO: 122) | (SEQ ID NO: 124) | (SEQ ID NO: 126) | (SEQ ID |
| CDR2 | (SEQ ID | | | | NO: 131) |
| | NO: 117) | | | | |
| Heavy Chain | HDHVWFDP | HDHVWFDP | HDHVWFDP | ARHDHVWFD | ARHDHVWF |
| variable region | (SEQ ID | (SEQ ID NO: 118) | (SEQ ID NO: 118) | (SEQ ID NO: 127) | DP |
| CDR3 | NO: 118) | | | | (SEQ ID |
| | | | | | NO: 132) |
| Light Chain | RASQSVSRNL | RASQSVSRNLV | RASQSVSRNLV | SRNLVWY | QSVSRN |
| variable region | V | (SEQ ID NO: 119) | (SEQ ID NO: 119) | (SEQ ID NO: 128) | (SEQ ID |
| CDR1 | (SEQ ID | | | | NO: 133) |
| | NO: 119) | | | | |
| Light Chain | GASTRAT | GASTRAT | GASTRAT | LLIYGASTRA | GAS |
| variable region | (SEQ ID NO: 5) | (SEQ ID NO: 5) | (SEQ ID NO: 5) | (SEQ ID NO: 15) | |
| CDR2 | | | | | |
| Light Chain | QQYNNWPPW | QQYNNWPPWT | QQYNNWPPWT | QQYNNWPPW | QQYNNWPP |
| variable region | T | (SEQ ID NO: 120) | (SEQ ID NO: 120) | (SEQ ID NO: 129) | WT |
| CDR3 | (SEQ ID | | | | (SEQ ID |
| | NO: 120) | | | | NO: 120) |

VH (SEQ ID NO: 134)
QVQLQESGPGLVKTSETLSLTCTVSGGSISSHYWSWIRQPPGKGLEWIGNIFYSGSTNYNPSLKSRVTMSVDTSKNHFSLKLISVTTADTA
IYYCARHDHVWFDPWGQGTLVTVSS
VL (SEQ ID NO: 136)
EIVLTQSPATLSVFPGERATLSCRASQSVSRNLVWYQQKPGQAPRLLIYGASTRATGIPDRESGSGSGTEFTLTISSLQSEDFAVYYCQQY
NNWPPWTFGQGTKVEIK

TABLE 10
NPP3B98 Binding Region Sequences

| | Kabat | Chothia | AbM | Contact | IMGT |
|---|---|---|---|---|---|
| Heavy Chain | NYYWS | GGSISNY | GGSISNYYWS | SNYYWS | GGSISNYY |
| variable | (SEQ ID | (SEQ ID NO: 143) | (SEQ ID NO: 145) | (SEQ ID NO: 147) | (SEQ ID |
| region | NO: 137) | | | | NO: 153) |
| CDR1 | | | | | |
| Heavy Chain | RIYASGSTNY | YASGS | RIYASGSTN | WIGRIYASGSTN | IYASGST |
| variable | NPSLKS | (SEQ ID NO: 144) | (SEQ ID NO: 146) | (SEQ ID NO: 148) | (SEQ ID |
| region | (SEQ ID | | | | NO: 154) |
| CDR2 | NO: 138) | | | | |
| Heavy Chain | EGYSSTLYDN | EGYSSTLYDNAFD | EGYSSTLYDNAFD | AREGYSSTLYDN | AREGYSSTL |
| variable | AFDF | F (SEQ ID NO: 139) | F (SEQ ID NO: 139) | AFD | YDNAFDF |
| region | (SEQ ID | | | (SEQ ID NO: 149) | (SEQ ID |
| CDR3 | NO: 139) | | | | NO: 155) |
| Light Chain | RASQGISTWL | RASQGISTWLA | RASQGISTWLA | STWLAWY | QGISTW |
| variable | A | (SEQ ID NO: 140) | (SEQ ID NO: 140) | (SEQ ID NO: 150) | (SEQ ID |
| region | (SEQ ID | | | | NO: 156) |
| CDR1 | NO: 140) | | | | |
| Light Chain | AASSLOS | AASSLOS | AASSLOS | LLIYAASSLQ | AAS |
| variable | (SEQ ID | (SEQ ID NO: 141) | (SEQ ID NO: 141) | (SEQ ID NO: 151) | |
| region | NO: 141) | | | | |
| CDR2 | | | | | |
| Light Chain | QQANSFPLT | QQANSFPLT | QQANSFPLT | QQANSFPL | QQANSFPLT |
| variable | (SEQ ID | (SEQ ID NO: 142) | (SEQ ID NO: 142) | (SEQ ID NO: 152) | (SEQ ID |
| region | NO: 142) | | | | NO: 142) |
| CDR3 | | | | | |

VH (SEQ ID NO: 158)
QVQLQESGPGLVKPSETLSLTCTVSGGSISNYYWSWIRQPAGKGLEWIGRIYASGSTNYNPSLKSRVTMSVDTSKNQFSLRLR
SVTATDTAVYYCAREGYSSTLYDNAFDFWGQGTMVTVSS
VL (SEQ ID NO: 159)
DIQMTQSPSSVSASVGGRVTITCRASQGISTWLAWYQQKPGKAPKLLIYAASSLQSGVPSRFSGSGSGTDFTLTISSLQPEDF
ATYYCQQANSFPLTFGGGTKVEIK

TABLE 11
NPP3B107 Binding Region Sequences

| | Kabat | Chothia | AbM | Contact | IMGT |
|---|---|---|---|---|---|
| Heavy Chain | DYSMN | GFIFSDY | GFIFSDYSMN | SDYSMN | GFIFSDYS |
| variable region | (SEQ ID NO: 1) | (SEQ ID NO: 164) | (SEQ ID NO: 166) | (SEQ ID NO: 11) | (SEQ ID |
| CDR1 | | | | | NO: 172) |
| Heavy Chain | SISSGSSYIYY | SSGSSY | SISSGSSYIY | WVSSISSGSSYIY | ISSGSSYI |
| variable region | ADSLKG | (SEQ ID NO: 165) | (SEQ ID NO: 167) | (SEQ ID NO: 168) | (SEQ ID |
| CDR2 | (SEQ ID | | | | NO: 173) |
| | NO: 160) | | | | |
| Heavy Chain | GLDYFDY | GLDYFDY | GLDYFDY | VRGLDYFD | VRGLDYFDY |
| variable region | (SEQ ID | (SEQ ID NO: 161) | (SEQ ID NO: 161) | (SEQ ID NO: 169) | (SEQ ID |
| CDR3 | NO: 161) | | | | NO: 174) |
| Light Chain | RASQDISNYL | RASQDISNYLA | RASQDISNYLA | SNYLAWY | QDISNY |
| variable region | A | (SEQ ID NO: 162) | (SEQ ID NO: 162) | (SEQ ID NO: 170) | (SEQ ID |
| CDR1 | (SEQ ID | | | | NO: 175) |
| | NO: 162) | | | | |
| Light Chain | VASTLOS | VASTLQS | VASTLQS | LLIYVASTLQ | VAS |
| variable region | (SEQ ID | (SEQ ID NO: 163) | (SEQ ID NO: 163) | (SEQ ID NO: 171) | |
| CDR2 | NO: 163) | | | | |
| Light Chain | QQLNSYPRT | QQLNSYPRT | QQLNSYPRT | QQLNSYPR | QQLNSYPRT |
| variable region | (SEQ ID NO: 76) | (SEQ ID NO: 76) | (SEQ ID NO: 76) | (SEQ ID NO: 86) | (SEQ ID |
| CDR3 | | | | | NO: 76) |

VH (SEQ ID NO: 177)
EVQLVESGGGLVKPGGSLRLSCAASGFIFSDYSMNWVRQAPGKGLEWVSSISSGSSYIYYADSLKGRFTISRDNAKNSLYL
QMSSLRAEDTAVYYCVRGLDYFDYWGQGALVTVSS
VL (SEQ ID NO: 178)
DIQMTQSPSFLSASVGDRVTITCRASQDISNYLAWYQQKPGRAPKLLIYVASTLQSGVPSRFSGSGSGTEFTLTISSLQPE
DFATYYCQQLNSYPRTFGQGTKVEIK

CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, upon binding to the ENPP3 molecule, the present ENPP3-binding molecule binds to the cell expressing the ENPP3 protein. In some embodiments, the ENPP3-expressing cell is a cancer cell. In some embodiments, the cancer is a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.

In some embodiments, the ENPP3 binding region provided herein binds to the same epitope as an ENPP3 binding region comprising a VH comprising the amino acid sequence of SEQ ID NO:22, and a VL comprising the amino acid sequence of SEQ ID NO:23. In some embodiments, the ENPP3 binding region provided herein binds to the same epitope as an ENPP3 binding region comprising a VH comprising the amino acid sequence of SEQ ID NO:45, and a VL comprising the amino acid sequence of SEQ ID NO:46. In some embodiments, the ENPP3 binding region provided herein binds to the same epitope as an ENPP3 binding region comprising a VH comprising the amino acid sequence of SEQ ID NO:68, and a VL comprising the amino acid sequence of SEQ ID NO:69. In some embodiments, the ENPP3 binding region provided herein binds to the same epitope as an ENPP3 binding region comprising a VH comprising the amino acid sequence of SEQ ID NO:68, and a VL comprising the amino acid sequence of SEQ ID NO:70. In some embodiments, the ENPP3 binding region provided herein binds to the same epitope as an ENPP3 binding region comprising a VH comprising the amino acid sequence of SEQ ID NO:91, and a VL comprising the amino acid sequence of SEQ ID NO: 92. In some embodiments, the ENPP3 binding region provided herein binds to the same epitope as an ENPP3 binding region comprising a VH comprising the amino acid sequence of SEQ ID NO:113, and a VL comprising the amino acid sequence of SEQ ID NO:114. In some embodiments, the ENPP3 binding region provided herein binds to the same epitope as an ENPP3 binding region comprising a VH comprising the amino acid sequence of SEQ ID NO:113, and a VL comprising the amino acid sequence of SEQ ID NO:115. In some embodiments, the ENPP3 binding region provided herein binds to the same epitope as an ENPP3 binding region comprising a VH comprising the amino acid sequence of SEQ ID NO:134, and a VL comprising the amino acid sequence of SEQ ID NO:135. In some embodiments, the ENPP3 binding region provided herein binds to the same epitope as an ENPP3 binding region comprising a VH comprising the amino acid sequence of SEQ ID NO: 158, and a VL comprising the amino acid sequence of SEQ ID NO: 159. In some embodiments, the ENPP3 binding region provided herein binds to the same epitope as an ENPP3 binding region comprising a VH comprising the amino acid sequence of SEQ ID NO:177, and a VL comprising the amino acid sequence of SEQ ID NO: 178.

### 5.4 Antibodies

In some embodiments, the ENPP3 binding agents described herein are anti-ENPP3 antibodies. In some embodiments, the ENPP3 binding agents described herein comprises one or more domain or fragments derived from an antibody (e.g., an anti-ENPP3 antibody). In some embodiments, the ENPP3 binding agents described herein can be generated with methods and processes for the generation, selection, modification, and fragmentation, etc. of antibody molecules that are known in the art.

### 5.4.1 Polyclonal Antibodies

In some embodiments, the anti-ENPP3 antibodies of the present disclosure may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a ENPP3 polypeptide or a fusion protein thereof. It may be useful to conjugate the immunizing agent to a protein known to be immunogenic in the mammal being immunized or to immunize the mammal with the protein and one or more adjuvants. Examples of such immunogenic proteins include, but are not limited to, keyhole limpet hemocyanin, serum albumin, bovine thyroglobulin, and soybean trypsin inhibitor. Examples of adjuvants which may be employed include Ribi, CpG, Poly 1C, Freund's complete adjuvant, and MPL-TDM adjuvant (monophosphoryl Lipid A, synthetic trehalose dicorynomycolate). The immunization protocol may be selected by one skilled in the art without undue experimentation. The mammal can then be bled, and the serum assayed for ENPP3 antibody titer. If desired, the mammal can be boosted until the antibody titer increases or plateaus. Additionally or alternatively, lymphocytes may be obtained from the immunized animal for fusion and preparation of monoclonal antibodies from hybridoma as described below.

### 5.4.2 Monoclonal Antibodies

The antibodies of the present disclosure may alternatively be monoclonal antibodies. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. In some embodiments, the immunizing antigen is a human protein or a fragment thereof. In some embodiments, the immunizing antigen is a mouse protein or a fragment thereof. In some embodiments, the immunizing antigen is a cyno protein or a fragment thereof. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Pluckthun, 1992, Immunol. Revs. 130:151-88.

In some embodiments, an antibody that binds an ENPP3 epitope comprises an amino acid sequence of a VH domain and/or an amino acid sequence of a VL domain encoded by a nucleotide sequence that hybridizes to (1) the complement of a nucleotide sequence encoding any one of the VH and/or VL domain described herein under stringent conditions (e.g., hybridization to filter-bound DNA in 6× sodium chloride/sodium citrate (SSC) at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art. See, e.g., Current Protocols in Molecular Biology Vol. I, 6.3.1-6.3.6 and 2.10.3 (Ausubel et al. eds., 1989).

In some embodiments, an antibody that binds an ENPP3 epitope comprises an amino acid sequence of a HCDR or an amino acid sequence of a LCDR depicted in Tables 1-11 under stringent conditions (e.g., hybridization to filter-bound DNA in 6×SSC at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art (see, e.g., Ausubel et al., supra).

In a further embodiment, monoclonal antibodies or antibody fragments can be isolated from antibody phage libraries generated using the techniques described in, for example, Antibody Phage Display: Methods and Protocols (O'Brien and Aitken eds., 2002). In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, ENPP3 (e.g., a ENPP3 polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Anti-ENPP3 antibodies can be obtained by designing a suitable antigen screening procedure to select for the phage clone of interest followed by construction of a full length anti-ENPP3 antibody clone using VH and/or VL sequences (e.g., the Fv sequences), or various CDR sequences from VH and VL sequences, from the phage clone of interest and suitable constant region (e.g., Fc) sequences described in Kabat et al., supra.

In another embodiment, anti-ENPP3 antibody is generated by using methods as described in Bowers et al., 2011, Proc Natl Acad Sci USA. 108:20455-60, e.g., the SHM-XHL™ platform (AnaptysBio, San Diego, CA). Briefly, in this approach, a fully human library of IgGs is constructed in a mammalian cell line (e.g., HEK293) as a starting library. Mammalian cells displaying immunoglobulin that binds to a target peptide or epitope are selected (e.g., by FACS sorting), then activation-induced cytidine deaminase (AID)-triggered somatic hypermutation is reproduced in vitro to expand diversity of the initially selected pool of antibodies. After several rounds of affinity maturation by coupling mammalian cell surface display with in vitro somatic hypermutation, high affinity, high specificity anti-ENPP3 antibodies are generated. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

### 5.4.3 Antibody Fragments

The present disclosure provides antibodies and antibody fragments that bind to ENPP3. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, scFv and spFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). In certain embodiments, an antibody is a stapled single chain Fv fragment (spFv) (see, e.g., WO2021030657). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50:98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, immunoglobulin molecules and immunologically active portions of immunoglobulin molecules, for example, molecules that contain an antigen binding site that bind to a ENPP3 epitope. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule.

Variants and derivatives of antibodies include antibody functional fragments that retain the ability to bind to a ENPP3 epitope. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a stapled scFv, or spFv (e.g., an scFv comprising at least one disulfide bond between the VH or VL and the linker); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); and a triabody (e.g., a trimerized scFv, formed in a manner similar to a diabody, but in which three antigen-binding domains are created in a single complex; the three antigen-binding domains may be directed towards the same or different epitopes).

In some embodiments, an ENPP3 binding region is an antigen binding fragment of an antibody. In some embodiments, the ENPP3 binding region is a portion of an intact antibody. In some embodiments, the ENPP3 binding region is a Fab, Fab′, F(ab′)₂, Fv, single chain antibody molecules (e.g., scFv), stapled single chain antibody molecules (e.g., spFv), disulfide-linked scFv (dsscFv). In some embodiments, the ENPP3 binding region is a Fab. In other specific embodiments, an ENPP3 binding region comprises two Fabs. In other specific embodiments, an ENPP3 binding region comprises two Fabs in tandem. In some embodiments, the ENPP3 binding region is a scFv. In some embodiments, the ENPP3 binding region is selected from nanobodies, diabodies, tribodies, tetrabodies, minibodies, dual variable domain antibodies (DVD), single variable domain antibodies (e.g., camelid antibodies). Any of the VH and the VL domains described herein that bind ENPP3 can be engineered into the binding molecule in the various formats described above, and their binding to ENPP3 and thermostability may be assessed using the assays described herein.

In specific embodiments, an ENPP3 binding region comprises an scFv that binds ENPP3. Any of the VH and the VL domains described herein that bind ENPP3 may be engineered into scFv format in either VH-linker-VL or VL-linker-VH orientation. Alternatively, the VH and VL domains may be engineered into scFv format without the use of a linker in either the VH-VL or VL-VH orientation. Accordingly, in some embodiments, the ENPP3 binding agent described herein comprises an scFv that binds ENPP3 in the format of VH-linker-VL. In some embodiments, the ENPP3 binding agent described herein comprises an scFv that binds ENPP3 in the format of VL-linker-VH. In some embodiments, the ENPP3 binding agent described herein comprises an scFv that binds ENPP3 in the format of VH-VL. In some embodiments, the ENPP3 binding agent described herein comprises an scFv that binds ENPP3 in the format of VL-VH.

In particular embodiments, the VH forming any of the scFv described herein is selected from the VH sequences disclosed in any of Tables 1 to 11. In particular embodiments, the VH forming any of the scFv described herein is selected from SEQ ID NOs: 22, 45, 68, 91, 113, 134, 158, and 177. In particular embodiments, the VL forming any of the scFv described herein is selected from the VL sequences disclosed in any of Tables 1 to 11. In particular embodiments, the VL forming any of the scFv described herein is selected from SEQ ID NOs: 23, 46, 69, 70, 92, 114, 115, 135, 136, and 178. In particular embodiments, the VH forming any of the scFv described herein is selected from SEQ ID NOs: 22, 45, 68, 91, 113, 134, 158, and 177, and the VL forming any of the scFv described herein is selected from SEQ ID NOs: 23, 46, 69, 70, 92, 114, 115, 135, 136, and 178.

In particular embodiments, the scFv comprises the VH and VL sequences disclosed in any of Tables 1 to 11. In some embodiments, the scFv comprises the VH comprising SEQ ID NO: 22 and the VL comprising SEQ ID NO: 23. In particular embodiments, the VH comprising SEQ ID NO:22 and the VL comprising SEQ ID NO:23 are fused directly with one another without a linker in the scFv. In some embodiments, the VH comprising SEQ ID NO:22 is fused to the N terminus of the VL comprising SEQ ID NO:23 in the VH-VL orientation. In other embodiments, the VH comprising SEQ ID NO:22 is fused to the C terminus of the VL comprising SEQ ID NO:23 in the VL-VH orientation. In some embodiments, the VH comprising SEQ ID NO:22 and the VL comprising SEQ ID NO:23 are linked with a linker in the scFv. In some embodiments, the VH comprising SEQ ID NO:22 and the VL comprising SEQ ID NO: 23 and the linker are fused in the VH-linker-VL orientation in the scFv. In other embodiments, the VH comprising SEQ ID NO:22 and the VL comprising SEQ ID NO:23 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO: 45 and the VL comprising SEQ ID NO: 46. In particular embodiments, the VH comprising SEQ ID NO: 45 and the VL comprising SEQ ID NO:46 are fused directly with one another without a linker in the scFv. In some embodiments, the VH comprising SEQ ID NO:45 is fused to the N terminus of the VL comprising SEQ ID NO:46 in the VH-VL orientation. In other embodiments, the VH comprising SEQ ID NO:45 is fused to the C terminus of the VL comprising SEQ ID NO:46 in the VL-VH orientation. In some embodiments, the VH comprising SEQ ID NO:45 and the VL comprising SEQ ID NO:46 are linked with a linker in the scFv. In some embodiments, the VH comprising SEQ ID NO:45 and the VL comprising SEQ ID NO: 46 and the linker are fused in the VH-linker-VL orientation in the scFv. In other embodiments, the VH comprising SEQ ID NO:45 and the VL comprising SEQ ID NO:46 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO:68 and the VL comprising SEQ ID NO:69. In particular embodiments, the VH comprising SEQ ID NO: 68 and the VL comprising SEQ ID NO:69 are fused directly with one another without a linker in the scFv. In some embodiments, the VH comprising SEQ ID NO:68 is fused to the N terminus of the VL comprising SEQ ID NO:69 in the VH-VL orientation. In other embodiments, the VH comprising SEQ ID NO:68 is fused to the C terminus of the VL comprising SEQ ID NO:69 in the VL-VH orientation. In some embodiments, the VH comprising SEQ ID NO:68 and the VL comprising SEQ ID NO:69 are linked with a linker in the scFv. In some embodiments, the VH comprising SEQ ID NO:68 and the VL comprising SEQ ID NO: 69 and the linker are fused in the VH-linker-VL orientation in the scFv. In other embodiments, the VH comprising SEQ ID NO:68 and the VL comprising SEQ ID NO:69 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO:68 and the VL comprising SEQ ID NO:70. In particular embodiments, the VH comprising SEQ ID NO:68 and the VL comprising SEQ ID NO:70 are fused directly with one another without a linker in the scFv. In some embodiments, the VH comprising SEQ ID NO:68 is fused to the N terminus of the VL comprising SEQ ID NO:70 in the VH-VL orientation. In other embodiments, the VH comprising SEQ ID NO:68 is fused to the C terminus of the VL comprising SEQ ID NO: 70 in the VL-VH orientation. In some embodiments, the VH comprising SEQ ID NO:68 and the VL comprising SEQ ID NO: 70 are linked with a linker in the scFv. In some embodiments, the VH comprising SEQ ID NO:68 and the VL comprising SEQ ID NO:70 and the linker are fused in the VH-linker-VL orientation in the scFv. In other embodiments, the VH comprising SEQ ID NO:68 and the VL comprising SEQ ID NO:70 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO:91 and the VL comprising SEQ ID NO:92. In particular embodiments, the VH comprising SEQ ID NO:91 and the VL comprising SEQ ID NO:92 are fused directly with one another without a linker in the scFv. In some embodiments, the VH comprising SEQ ID NO:91 is fused to the N terminus of the VL comprising SEQ ID NO:92 in the VH-VL orientation. In other embodiments, the VH comprising SEQ ID NO:91 is fused to the C terminus of the VL comprising SEQ ID NO:92 in the VL-VH orientation. In some embodiments, the VH comprising SEQ ID NO:91 and the VL comprising SEQ ID NO:92 are linked with a linker in the scFv. In some embodiments, the VH comprising SEQ ID NO:91 and the VL comprising SEQ ID NO:92 and the linker are fused in the VH-linker-VL orientation in the scFv. In other embodiments, the VH comprising SEQ ID NO:91 and the VL comprising SEQ ID NO:92 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO: 113 and the VL comprising SEQ ID NO: 114. In particular embodiments, the VH comprising SEQ ID NO: 113 and the VL comprising SEQ ID NO:114 are fused directly with one another without a linker in the scFv. In some embodiments, the VH comprising SEQ ID NO: 113 is fused to the N terminus of the VL comprising SEQ ID NO: 114 in the VH-VL orientation. In other embodiments, the VH comprising SEQ ID NO: 113 is fused to the C terminus of the VL comprising SEQ ID NO:114 in the VL-VH orientation. In some embodiments, the VH comprising SEQ ID NO: 113 and the VL comprising SEQ ID NO: 114 are linked with a linker in the scFv. In some embodiments, the VH comprising SEQ ID NO: 113 and the VL comprising SEQ ID NO: 114 and the linker are fused in the VH-linker-VL orientation in the scFv. In other embodiments, the VH comprising SEQ ID NO:113 and the VL comprising SEQ ID NO:114 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO:113 and the VL comprising SEQ ID NO: 115. In particular embodiments, the VH comprising SEQ ID NO: 113 and the VL comprising SEQ ID NO: 115 are fused directly with one another without a linker in the scFv. In some embodiments, the VH comprising SEQ ID NO: 113 is fused to the N terminus of the VL comprising SEQ ID NO: 115 in the VH-VL orientation. In other embodiments, the VH comprising SEQ ID NO: 113 is fused to the C terminus of the VL comprising SEQ ID NO: 115 in the VL-VH orientation. In some embodiments, the VH comprising SEQ ID NO:113 and the VL comprising SEQ ID NO: 115 are linked with a linker in the scFv. In some embodiments, the VH comprising SEQ ID NO:113 and the VL comprising SEQ ID NO: 115 and the linker are fused in the VH-linker-VL orientation in the scFv. In other embodiments, the VH comprising SEQ ID NO:113 and the VL comprising SEQ ID NO: 115 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO: 134 and the VL comprising SEQ ID NO: 135. In particular embodiments, the VH comprising SEQ ID NO: 134 and the VL comprising SEQ ID NO: 135 are fused directly with one another without a linker in the scFv. In some embodiments, the VH comprising SEQ ID NO: 134 is fused to the N terminus of the VL comprising SEQ ID NO: 135 in the VH-VL orientation. In other embodiments, the VH comprising SEQ ID NO: 134 is fused to the C terminus of the VL comprising SEQ ID NO: 135 in the VL-VH orientation. In some embodiments, the VH comprising SEQ ID NO: 134 and the VL comprising SEQ ID NO:135 are linked with a linker in the scFv. In some embodiments, the VH comprising SEQ ID NO: 134 and the VL comprising SEQ ID NO: 135 and the linker are fused in the VH-linker-VL orientation in the scFv. In other embodiments, the VH comprising SEQ ID NO: 134 and the VL comprising SEQ ID NO: 135 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO: 134 and the VL comprising SEQ ID NO: 136. In particular embodiments, the VH comprising SEQ ID NO: 134 and the VL comprising SEQ ID NO: 136 are fused directly with one another without a linker in the scFv. In some embodiments, the VH comprising SEQ ID NO: 134 is fused to the N terminus of the VL comprising SEQ ID NO: 136 in the VH-VL orientation. In other embodiments, the VH comprising SEQ ID NO: 134 is fused to the C terminus of the VL comprising SEQ ID NO: 136 in the VL-VH orientation. In some embodiments, the VH comprising SEQ ID NO: 134 and the VL comprising SEQ ID NO: 136 are linked with a linker in the scFv. In some embodiments, the VH comprising SEQ ID NO: 134 and the VL comprising SEQ ID NO: 136 and the linker are fused in the VH-linker-VL orientation in the scFv. In other embodiments, the VH comprising SEQ ID NO: 134 and the VL comprising SEQ ID NO: 136 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO: 158 and the VL comprising SEQ ID NO:159. In particular embodiments, the VH comprising SEQ ID NO: 158 and the VL comprising SEQ ID NO: 159 are fused directly with one another without a linker in the scFv. In some embodiments, the VH comprising SEQ ID NO: 158 is fused to the N terminus of the VL comprising SEQ ID NO: 159 in the VH-VL orientation. In other embodiments, the VH comprising SEQ ID NO: 158 is fused to the C terminus of the VL comprising SEQ ID NO: 159 in the VL-VH orientation. In some embodiments, the VH comprising SEQ ID NO:158 and the VL comprising SEQ ID NO: 159 are linked with a linker in the scFv. In some embodiments, the VH comprising SEQ ID NO: 158 and the VL comprising SEQ ID NO: 159 and the linker are fused in the VH-linker-VL orientation in the scFv. In other embodiments, the VH comprising SEQ ID NO:158 and the VL comprising SEQ ID NO: 159 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the scFv comprises the VH comprising SEQ ID NO:177 and the VL comprising SEQ ID NO: 178. In particular embodiments, the VH comprising SEQ ID NO: 177 and the VL comprising SEQ ID NO: 178 are fused directly with one another without a linker in the scFv. In some embodiments, the VH comprising SEQ ID NO: 177 is fused to the N terminus of the VL comprising SEQ ID NO: 178 in the VH-VL orientation. In other embodiments, the VH comprising SEQ ID NO: 177 is fused to the C terminus of the VL comprising SEQ ID NO:178 in the VL-VH orientation. In some embodiments, the VH comprising SEQ ID NO: 177 and the VL comprising SEQ ID NO: 178 are linked with a linker in the scFv. In some embodiments, the VH comprising SEQ ID NO: 177 and the VL comprising SEQ ID NO: 178 and the linker are fused in the VH-linker-VL orientation in the scFv. In other embodiments, the VH comprising SEQ ID NO:177 and the VL comprising SEQ ID NO: 178 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the VH and the VL domains identified herein may be incorporated into a scFv format and the binding and thermostability of the resulting scFv to ENPP3 can be assessed using known methods. In some embodiments, binding of the resulting scFv to ENPP3 can be assessed using ProteOn XPR36, Biacore 3000 or KinExA instrumentation, ELISA or competitive binding assays known to those skilled in the art. In some embodiments, binding of the resulting scFv to ENPP3 can be evaluated using purified scFvs or E. coli supernatants or lysed cells containing the expressed scFv. In some embodiments, the measured affinity of a test scFv to ENPP3 may vary if measured under different conditions (e.g., osmolarity, pH). Thus, measurements of affinity and other binding parameters (e.g., KD, Kon, Koff) are typically made with standardized conditions and standardized buffers. Thermostability may be evaluated by heating the test scFv at elevated temperatures, such as at 50° C., 55° C. or 60° C. for a period of time, such as 5 minutes (min), 10 min, 15 min, 20 min, 25 min or 30 min and measuring binding of the test scFv to ENPP3. The scFvs retaining comparable binding to ENPP3 when compared to a non-heated scFv sample are referred to as being thermostable.

In recombinant expression systems, the linker is a peptide linker and may include any naturally occurring amino acid. Exemplary amino acids that can be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to ENPP3.

In some embodiments, the linker is about 5-50 amino acids long. In other embodiments, the linker is about 10-40 amino acids long. In other embodiments, the linker is about 10-35 amino acids long. In other embodiments, the linker is about 10-30 amino acids long. In other embodiments, the linker is about 10-25 amino acids long. In other embodiments, the linker is about 10-20 amino acids long. In other embodiments, the linker is about 15-20 amino acids long. In other embodiments, the linker is about 16-19 amino acids long. In other embodiments, the linker is 6 amino acids long. In other embodiments, the linker is 7 amino acids long. In other embodiments, the linker is 8 amino acids long. In other embodiments, the linker is 9 amino acids long. In other embodiments, the linker is 10 amino acids long. In other embodiments, the linker is 11 amino acids long. In other embodiments, the linker is 12 amino acids long. In other embodiments, the linker is 13 amino acids long. In other embodiments, the linker is 14 amino acids long. In other embodiments, the linker is 15 amino acids long. In other embodiments, the linker is 16 amino acids long. In other embodiments, the linker is 17 amino acids long. In other embodiments, the linker is 18 amino acids long. In other embodiments, the linker is 19 amino acids long. In other embodiments, the linker is 20 amino acids long. In other embodiments, the linker is 21 amino acids long. In other embodiments, the linker is 22 amino acids long. In other embodiments, the linker is 23 amino acids long. In other embodiments, the linker is 24 amino acids long. In other embodiments, the linker is 25 amino acids long. In other embodiments, the linker is 26 amino acids long. In other embodiments, the linker is 27 amino acids long. In other embodiments, the linker is 28 amino acids long. In other embodiments, the linker is 29 amino acids long. In other embodiments, the linker is 30 amino acids long. In other embodiments, the linker is 31 amino acids long. In other embodiments, the linker is 32 amino acids long. In other embodiments, the linker is 33 amino acids long. In other embodiments, the linker is 34 amino acids long. In other embodiments, the linker is 35 amino acids long. In other embodiments, the linker is 36 amino acids long. In other embodiments, the linker is 37 amino acids long. In other embodiments, the linker is 38 amino acids long. In other embodiments, the linker is 39 amino acids long. In other embodiments, the linker is 40 amino acids long. Exemplary linkers that can be used are Gly rich linkers, Gly and Ser containing linkers, Gly and Ala containing linkers, Ala and Ser containing linkers, and other flexible linkers.

Other linker sequences can include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers. Exemplary linkers that may be used are shown in Table 12. Additional linkers are described for example in Int. Pat. Publ. No. WO2019/060695.

TABLE 12
Examples of Linker Sequences

| | | SEQ |
|---|---|---|
| Linker name | Amino acid sequence | ID NO: |
| Linker 1 | GGSEGKSSGSGSESKSTGGS | 258 |
| Linker 2 | GGGSGGGS | 259 |
| Linker 3 | GGGSGGGSGGGS | 260 |
| Linker 4 | GGGSGGGSGGGSGGGS | 261 |
| Linker 5 | GGGSGGGSGGGSGGGSGGGS | 262 |
| Linker 6 | GGGGSGGGGSGGGGS | 263 |
| Linker 7 | GGGGSGGGGSGGGGGGGGS | 264 |
| Linker 8 | GGGGSGGGGSGGGGSGGGGSGGGGS | 265 |
| Linker 9 | GSTSGSGKPGSGEGSTKG | 266 |
| Linker 10 | IRPRAIGGSKPRVA | 267 |
| Linker 11 | GKGGSGKGGSGKGGS | 268 |
| Linker 12 | GGKGSGGKGSGGKGS | 269 |
| Linker 13 | GGGKSGGGKSGGGKS | 270 |
| Linker 14 | GKGKSGKGKSGKGKS | 271 |
| Linker 15 | GGGKSGGKGSGKGGS | 272 |
| Linker 16 | GKPGSGKPGSGKPGS | 273 |
| Linker 17 | GKPGSGKPGSGKPGSGKPGS | 274 |
| Linker 18 | GKGKSGKGKSGKGKSGKGKS | 275 |
| Linker 19 | STAGDTHLGGEDFD | 276 |
| Linker 20 | GEGGSGEGGSGEGGS | 277 |
| Linker 21 | GGEGSGGEGSGGEGS | 278 |
| Linker 22 | GEGESGEGESGEGES | 279 |
| Linker 23 | GGGESGGEGSGEGGS | 280 |
| Linker 24 | GEGESGEGESGEGESGEGES | 281 |
| Linker 25 | GSTSGSGKPGSGEGSTKG | 282 |
| Linker 26 | PRGASKSGSASQTGSAPGS | 283 |
| Linker 27 | GTAAAGAGAAGGAAAGAAG | 284 |
| Linker 28 | GTSGSSGSGSGGSGSGGGG | 285 |
| Linker 29 | GKPGSGKPGSGKPGSGKPGS | 286 |
| Linker 30 | GSGS | 287 |
| Linker 31 | APAPAPAPAP | 288 |
| Linker 32 | APAPAPAPAPAPAPAPAPAP | 289 |
| Linker 33 | AEAAAKEAAAKEAAAAKEAAAAKEAA | 290 |
| | AAKAAA | |
| Linker 34 | GTEGKSSGSGSESKST | 291 |
| Linker 35 | GGGSGGSGGCPPCGGSGG | 292 |

In particular embodiments, the scFv comprises, from the N- to C-terminus, a VH, a first linker (L1) and a VL (VH-L1-VL). In particular embodiments, the scFv comprises, from the N- to C-terminus, the VL, the L1 and the VH (VL-L1-VH). In particular embodiments, the L1 comprises the amino acid sequence of SEQ ID NO: 258. In particular embodiments, the L1 comprises the amino acid sequence of SEQ ID NO: 259. In particular embodiments, the L1 comprises the amino acid sequence of SEQ ID NO: 260. In particular embodiments, the L1 comprises the amino acid sequence of SEQ ID NO: 261. In particular embodiments, the L1 comprises the amino acid sequence of SEQ ID NO: 262. In particular embodiments, the L1 comprises the amino acid sequence of SEQ ID NO: 263. In particular embodiments, the L1 comprises the amino acid sequence of SEQ ID NO: 264. In particular embodiments, the L1 comprises the amino acid sequence of SEQ ID NO: 265. In particular embodiments, the L1 comprises the amino acid sequence of SEQ ID NO: 266. In particular embodiments, the L1 comprises the amino acid sequence of SEQ ID NO: 267. In particular embodiments, the L1 comprises the amino acid sequence of SEQ ID NO: 268. In particular embodiments, the L1 comprises the amino acid sequence of SEQ ID NO: 269. In particular embodiments, the L1 comprises the amino acid sequence of SEQ ID NO: 270. In particular embodiments, the L1 comprises the amino acid sequence of SEQ ID NO: 271. In particular embodiments, the L1 comprises the amino acid sequence of SEQ ID NO: 272. In particular embodiments, the L1 comprises the amino acid sequence of SEQ ID NO: 273. In particular embodiments, the L1 comprises the amino acid sequence of SEQ ID NO: 274. In particular embodiments, the L1 comprises the amino acid sequence of SEQ ID NO: 275. In particular embodiments, the L1 comprises the amino acid sequence of SEQ ID NO: 276. In particular embodiments, the L1 comprises the amino acid sequence of SEQ ID NO: 277. In particular embodiments, the L1 comprises the amino acid sequence of SEQ ID NO: 278. In particular embodiments, the L1 comprises the amino acid sequence of SEQ ID NO: 279. In particular embodiments, the L1 comprises the amino acid sequence of SEQ ID NO: 280. In particular embodiments, the L1 comprises the amino acid sequence of SEQ ID NO: 281. In particular embodiments, the L1 comprises the amino acid sequence of SEQ ID NO: 282. In particular embodiments, the L1 comprises the amino acid sequence of SEQ ID NO: 283. In particular embodiments, the L1 comprises the amino acid sequence of SEQ ID NO: 284. In particular embodiments, the L1 comprises the amino acid sequence of SEQ ID NO: 285. In particular embodiments, the L1 comprises the amino acid sequence of SEQ ID NO: 286. In particular embodiments, the L1 comprises the amino acid sequence of SEQ ID NO: 287. In particular embodiments, the L1 comprises the amino acid sequence of SEQ ID NO: 288. In particular embodiments, the L1 comprises the amino acid sequence of SEQ ID NO: 289. In particular embodiments, the L1 comprises the amino acid sequence of SEQ ID NO: 290. In particular embodiments, the L1 comprises the amino acid sequence of SEQ ID NO: 291. In particular embodiments, the L1 comprises the amino acid sequence of SEQ ID NO: 292.

In particular embodiments, the spFv comprises a linker comprising at least one Cys residue which functions as an anchor point for forming a disulfide bond between the linker and the VH, the VL or both of the VH and VL. In one embodiment, the linker comprises the amino acid sequence of SEQ ID NO: 292.

### 5.4.4 Multivalent Antibodies

Without being bound by any theory, it is contemplated that a multivalent antibody may be internalized (and/or catabolized) faster than a monovalent antibody by a cell expressing an antigen to which the antibodies bind, and a multivalent antibody with three or more antigen binding sites may be internalized (and/or catabolized) faster than a bivalent antibody. In some embodiments, the binding agents of the present disclosure can be a bivalent antibody with two antigen binding sites. In some embodiments, the binding agents of the present disclosure can be multivalent antibodies (which are other than of the IgM class) with three or more antigen binding sites (e.g., tetravalent antibodies). In some embodiments, the multivalent antibodies can be readily produced by recombinant expression of nucleic acid encoding the polypeptide chains of the antibody. The multivalent antibody can comprise a dimerization domain and three or more antigen binding sites. In certain embodiments, the dimerization domain comprises (or consists of) an Fc region or a hinge region. In this scenario, the antibody will comprise an Fc region and three or more antigen binding sites amino-terminal to the Fc region. In certain embodiments, a multivalent antibody comprises (or consists of) three to about eight antigen binding sites. In one such embodiment, a multivalent antibody comprises (or consists of) four antigen binding sites. In some embodiments, the multivalent antibody comprises at least one polypeptide chain (e.g., two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In specific embodiments, the multivalent binding agents disclosed herein comprises one or more bivalent sc(Fv)₂ structures. In particular embodiments, any of the VH and the VL domains identified herein may also be used to generate bivalent sc(Fv)₂ structures, such as in the bivalent format of VH-linker-VL-linker-VL-linker-VH, VH-linker-VL-linker-VH-linker-VL, VH-linker-VH-linker-VL-linker-VL, VL-linker-VH-linker-VH-linker-VL, VL-linker-VH-linker-VL-linker-VH, or VL-linker-VL-linker-VH-linker-VH. In specific embodiments, the ENPP3 binding agent described herein comprises an sc(Fv)₂ that binds ENPP3 in the format of VH-linker-VL-linker-VL-linker-VH. In some embodiments, the ENPP3 binding agent described herein comprises an sc(Fv)₂ that binds ENPP3 in the format of VH-linker-VL-linker-VH-linker-VL. In some embodiments, the ENPP3 binding agent described herein comprises an sc(Fv)₂ that binds ENPP3 in the format of VH-linker-VH-linker-VL-linker-VL. In some embodiments, the ENPP3 binding agent described herein comprises an sc(Fv)₂ that binds ENPP3 in the format of VL-linker-VH-linker-VH-linker-VL. In some embodiments, the ENPP3 binding agent described herein comprises an sc(Fv)₂ that binds ENPP3 in the format of VL-linker-VH-linker-VL-linker-VH. In some embodiments, the ENPP3 binding agent described herein comprises an sc(Fv)₂ that binds ENPP3 in the format of VL-linker-VL-linker-VH-linker-VH.

Alternatively, the use of one or more linkers in the sc(Fv)₂ structures described herein may be avoided, and any two of the VH and VL domains may be joint directly. Accordingly, in some embodiments, the ENPP3 binding agent described herein comprises an sc(Fv)₂ that binds ENPP3 in the format of VH-VL-linker-VL-linker-VH. In some embodiments, the ENPP3 binding agent described herein comprises an sc(Fv)₂ that binds ENPP3 in the format of VH-linker-VL-VL-linker-VH. In some embodiments, the ENPP3 binding agent described herein comprises an sc(Fv)₂ that binds ENPP3 in the format of VH-linker-VL-linker-VL-VH. In some embodiments, the ENPP3 binding agent described herein comprises an sc(Fv)₂ that binds ENPP3 in the format of VH-VL-VL-linker-VH. In some embodiments, the ENPP3 binding agent described herein comprises an sc(Fv)₂ that binds ENPP3 in the format of VH-VL-linker-VL-VH. In some embodiments, the ENPP3 binding agent described herein comprises an sc(Fv)₂ that binds ENPP3 in the format of VH-linker-VL-VL-VH. In some embodiments, the ENPP3 binding agent described herein comprises an sc(Fv)₂ that binds ENPP3 in the format of VH-VL-VL-VH.

In some embodiments, the ENPP3 binding agent described herein comprises an sc(Fv)₂ that binds ENPP3 in the format of VH-VL-linker-VH-linker-VL. In some embodiments, the ENPP3 binding agent described herein comprises an sc(Fv)₂ that binds ENPP3 in the format of VH-linker-VL-VH-linker-VL. In some embodiments, the ENPP3 binding agent described herein comprises an sc(Fv)₂ that binds ENPP3 in the format of VH-linker-VL-linker-VH-VL. In some embodiments, the ENPP3 binding agent described herein comprises an sc(Fv)₂ that binds ENPP3 in the format of VH-VL-VH-linker-VL. In some embodiments, the ENPP3 binding agent described herein comprises an sc(Fv)₂ that binds ENPP3 in the format of VH-VL-linker-VH-VL. In some embodiments, the ENPP3 binding agent described herein comprises an sc(Fv)₂ that binds ENPP3 in the format of VH-linker-VL-VH-VL. In some embodiments, the ENPP3 binding agent described herein comprises an sc(Fv)₂ that binds ENPP3 in the format of VH-VL-VH-VL.

In some embodiments, the ENPP3 binding agent described herein comprises an sc(Fv)₂ that binds ENPP3 in the format of VH-VH-linker-VL-linker-VL. In some embodiments, the ENPP3 binding agent described herein comprises an sc(Fv)₂ that binds ENPP3 in the format of VH-linker-VH-VL-linker-VL. In some embodiments, the ENPP3 binding agent described herein comprises an sc(Fv)₂ that binds ENPP3 in the format of VH-linker-VH-linker-VL-VL. In some embodiments, the ENPP3 binding agent described herein comprises an sc(Fv)₂ that binds ENPP3 in the format of VH-VH-VL-linker-VL. In some embodiments, the ENPP3 binding agent described herein comprises an sc(Fv)₂ that binds ENPP3 in the format of VH-VH-linker-VL-VL. In some embodiments, the ENPP3 binding agent described herein comprises an sc(Fv)₂ that binds ENPP3 in the format of VH-linker-VH-VL-VL. In some embodiments, the ENPP3 binding agent described herein comprises an sc(Fv)₂ that binds ENPP3 in the format of VH-VH-VL-VL.

In some embodiments, the ENPP3 binding agent described herein comprises an sc(Fv)₂ that binds ENPP3 in the format of VL-VH-linker-VH-linker-VL. In some embodiments, the ENPP3 binding agent described herein comprises an sc(Fv)₂ that binds ENPP3 in the format of VL-linker-VH-VH-linker-VL. In some embodiments, the ENPP3 binding agent described herein comprises an sc(Fv)₂ that binds ENPP3 in the format of VL-linker-VH-linker-VH-VL. In some embodiments, the ENPP3 binding agent described herein comprises an sc(Fv)₂ that binds ENPP3 in the format of VL-VH-VH-linker-VL. In some embodiments, the ENPP3 binding agent described herein comprises an sc(Fv)₂ that binds ENPP3 in the format of VL-VH-linker-VH-VL. In some embodiments, the ENPP3 binding agent described herein comprises an sc(Fv)₂ that binds ENPP3 in the format of VL-linker-VH-VH-VL. In some embodiments, the ENPP3 binding agent described herein comprises an sc(Fv)₂ that binds ENPP3 in the format of VL-VH-VH-VL.

In some embodiments, the ENPP3 binding agent described herein comprises an sc(Fv)₂ that binds ENPP3 in the format of VL-VH-linker-VL-linker-VH. In some embodiments, the ENPP3 binding agent described herein comprises an sc(Fv)₂ that binds ENPP3 in the format of VL-linker-VH-VL-linker-VH. In some embodiments, the ENPP3 binding agent described herein comprises an sc(Fv)₂ that binds ENPP3 in the format of VL-linker-VH-linker-VL-VH. In some embodiments, the ENPP3 binding agent described herein comprises an sc(Fv)₂ that binds ENPP3 in the format of VL-VH-VL-linker-VH. In some embodiments, the ENPP3 binding agent described herein comprises an sc(Fv)₂ that binds ENPP3 in the format of VL-VH-linker-VL-VH. In some embodiments, the ENPP3 binding agent described herein comprises an sc(Fv)₂ that binds ENPP3 in the format of VL-linker-VH-VL-VH. In some embodiments, the ENPP3 binding agent described herein comprises an sc(Fv)₂ that binds ENPP3 in the format of VL-VH-VL-VH.

In some embodiments, the ENPP3 binding agent described herein comprises an sc(Fv)₂ that binds ENPP3 in the format of VL-VL-linker-VH-linker-VH. In some embodiments, the ENPP3 binding agent described herein comprises an sc(Fv)₂ that binds ENPP3 in the format of VL-linker-VL-VH-linker-VH. In some embodiments, the ENPP3 binding agent described herein comprises an sc(Fv)₂ that binds ENPP3 in the format of VL-linker-VL-linker-VH-VH. In some embodiments, the ENPP3 binding agent described herein comprises an sc(Fv)₂ that binds ENPP3 in the format of VL-VL-VH-linker-VH. In some embodiments, the ENPP3 binding agent described herein comprises an sc(Fv)₂ that binds ENPP3 in the format of VL-VL-linker-VH-VH. In some embodiments, the ENPP3 binding agent described herein comprises an sc(Fv)₂ that binds ENPP3 in the format of VL-linker-VL-VH-VH. In some embodiments, the ENPP3 binding agent described herein comprises an sc(Fv)₂ that binds ENPP3 in the format of VL-VL-VH-VH.

In some embodiments, the two VH domains in an sc(Fv)₂ described herein are the same. In some embodiments, the two VH domains in an sc(Fv)₂ described herein are different. In some embodiments, the two VL domains in an sc(Fv)₂ described herein are the same. In some embodiments, the two VL domains in an sc(Fv)₂ described herein are different. In particular embodiments, the two VH domains forming any of the sc(Fv)₂ structures described herein are independently selected from the VH sequences disclosed in any of Tables 1 to 11.

In particular embodiments, the two VH domains forming any of the sc(Fv)₂ structures described herein are independently selected from SEQ ID NOs: 22, 45, 68, 91, 113, 134, 158, and 177. In particular embodiments, the two VL domains forming any of the sc(Fv)₂ structures described herein are independently selected from the VL sequences disclosed in any of Tables 1 to 11. In particular embodiments, the two VL domains forming any of the sc(Fv)₂ structures described herein are independently selected from SEQ ID NOs: 23, 46, 69, 70, 92, 114, 115, 135, 136, and 178. In particular embodiments, the two VH domains forming any of the sc(Fv)₂ structures described herein are independently selected from SEQ ID NOs: 22, 45, 68, 91, 113, 134, 158, and 177, and the two VL domains forming any of the sc(Fv)₂ structures described herein are independently selected from SEQ ID NOs: 23, 46, 69, 70, 92, 114, 115, 135, 136, and 178.

In recombinant expression systems, the linker is a peptide linker and may include any naturally occurring amino acid. Exemplary amino acids that can be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to ENPP3.

In some embodiments, one or more linkers used in the sc(Fv)₂ structures described herein is about 5-50 amino acids long. In other embodiments, the one or more linkers is about 10-40 amino acids long. In other embodiments, the one or more linkers is about 10-35 amino acids long. In other embodiments, the one or more linkers is about 10-30 amino acids long. In other embodiments, the linker is about 10-25 amino acids long. In other embodiments, the one or more linkers is about 10-20 amino acids long. In other embodiments, the one or more linkers is about 15-20 amino acids long. In other embodiments, the one or more linkers is about 16-19 amino acids long. In other embodiments, the one or more linkers is 6 amino acids long. In other embodiments, the one or more linkers is 7 amino acids long. In other embodiments, the one or more linkers is 8 amino acids long. In other embodiments, the one or more linkers is 9 amino acids long. In other embodiments, the one or more linkers is 10 amino acids long. In other embodiments, the one or more linkers is 11 amino acids long. In other embodiments, the one or more linkers is 12 amino acids long. In other embodiments, the one or more linkers is 13 amino acids long. In other embodiments, the one or more linkers is 14 amino acids long. In other embodiments, the one or more linkers is 15 amino acids long. In other embodiments, the one or more linkers is 16 amino acids long. In other embodiments, the one or more linkers is 17 amino acids long. In other embodiments, the one or more linkers is 18 amino acids long. In other embodiments, the one or more linkers is 19 amino acids long. In other embodiments, the one or more linkers is 20 amino acids long. In other embodiments, the one or more linkers is 21 amino acids long. In other embodiments, the one or more linkers is 22 amino acids long. In other embodiments, the one or more linkers is 23 amino acids long. In other embodiments, the one or more linkers is 24 amino acids long. In other embodiments, the one or more linkers is 25 amino acids long. In other embodiments, the one or more linkers is 26 amino acids long. In other embodiments, the one or more linkers is 27 amino acids long. In other embodiments, the one or more linkers is 28 amino acids long. In other embodiments, the one or more linkers is 29 amino acids long. In other embodiments, the one or more linkers is 30 amino acids long. In other embodiments, the one or more linkers is 31 amino acids long. In other embodiments, the one or more linkers is 32 amino acids long. In other embodiments, the one or more linkers is 33 amino acids long. In other embodiments, the one or more linkers is 34 amino acids long. In other embodiments, the one or more linkers is 35 amino acids long. In other embodiments, the one or more linkers is 36 amino acids long. In other embodiments, the one or more linkers is 37 amino acids long. In other embodiments, the one or more linkers is 38 amino acids long. In other embodiments, the one or more linkers is 39 amino acids long. In other embodiments, the one or more linkers is 40 amino acids long. Exemplary linkers that can be used are Gly rich linkers, Gly and Ser containing linkers, Gly and Ala containing linkers, Ala and Ser containing linkers, and other flexible linkers.

Other linker sequences can include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers. Exemplary linkers that may be used are shown in the following Table. Additional linkers are described for example in Int. Pat. Publ. No. WO2019/060695.

In specific embodiments, the one or more linkers used in the sc(Fv)₂ structures described herein are independently selected from any one of SEQ ID NO:258 to 292. In particular embodiments, at least one of the linkers in the sc(Fv)₂ structure comprises the amino acid sequence of SEQ ID NO: 258. In particular embodiments, at least one of the linkers in the sc(Fv)₂ structure comprises the amino acid sequence of SEQ ID NO: 259. In particular embodiments, at least one of the linkers in the sc(Fv)₂ structure comprises the amino acid sequence of SEQ ID NO: 260. In particular embodiments, at least one of the linkers in the sc(Fv)₂ structure comprises the amino acid sequence of SEQ ID NO: 261. In particular embodiments, at least one of the linkers in the sc(Fv)₂ structure comprises the amino acid sequence of SEQ ID NO: 262. In particular embodiments, at least one of the linkers in the sc(Fv)₂ structure comprises the amino acid sequence of SEQ ID NO: 263. In particular embodiments, at least one of the linkers in the sc(Fv)₂ structure comprises the amino acid sequence of SEQ ID NO: 264. In particular embodiments, at least one of the linkers in the sc(Fv)₂ structure comprises the amino acid sequence of SEQ ID NO: 265. In particular embodiments, at least one of the linkers in the sc(Fv)₂ structure comprises the amino acid sequence of SEQ ID NO: 266. In particular embodiments, at least one of the linkers in the sc(Fv)₂ structure comprises the amino acid sequence of SEQ ID NO: 267. In particular embodiments, at least one of the linkers in the sc(Fv)₂ structure comprises the amino acid sequence of SEQ ID NO: 268. In particular embodiments, at least one of the linkers in the sc(Fv)₂ structure comprises the amino acid sequence of SEQ ID NO: 269. In particular embodiments, at least one of the linkers in the sc(Fv)₂ structure comprises the amino acid sequence of SEQ ID NO: 270. In particular embodiments, at least one of the linkers in the sc(Fv)₂ structure comprises the amino acid sequence of SEQ ID NO: 271. In particular embodiments, at least one of the linkers in the sc(Fv)₂ structure comprises the amino acid sequence of SEQ ID NO: 272. In particular embodiments, at least one of the linkers in the sc(Fv)₂ structure comprises the amino acid sequence of SEQ ID NO: 273. In particular embodiments, at least one of the linkers in the sc(Fv)₂ structure comprises the amino acid sequence of SEQ ID NO: 274. In particular embodiments, at least one of the linkers in the sc(Fv)₂ structure comprises the amino acid sequence of SEQ ID NO: 275. In particular embodiments, at least one of the linkers in the sc(Fv)₂ structure comprises the amino acid sequence of SEQ ID NO: 276. In particular embodiments, at least one of the linkers in the sc(Fv)₂ structure comprises the amino acid sequence of SEQ ID NO: 277. In particular embodiments, at least one of the linkers in the sc(Fv)₂ structure comprises the amino acid sequence of SEQ ID NO: 278. In particular embodiments, at least one of the linkers in the sc(Fv)₂ structure comprises the amino acid sequence of SEQ ID NO: 279. In particular embodiments, at least one of the linkers in the sc(Fv)₂ structure comprises the amino acid sequence of SEQ ID NO: 280. In particular embodiments, at least one of the linkers in the sc(Fv)₂ structure comprises the amino acid sequence of SEQ ID NO: 281. In particular embodiments, at least one of the linkers in the sc(Fv)₂ structure comprises the amino acid sequence of SEQ ID NO: 282. In particular embodiments, at least one of the linkers in the sc(Fv)₂ structure comprises the amino acid sequence of SEQ ID NO: 283. In particular embodiments, at least one of the linkers in the sc(Fv)₂ structure comprises the amino acid sequence of SEQ ID NO: 284. In particular embodiments, at least one of the linkers in the sc(Fv)₂ structure comprises the amino acid sequence of SEQ ID NO: 285. In particular embodiments, at least one of the linkers in the sc(Fv)₂ structure comprises the amino acid sequence of SEQ ID NO: 286. In particular embodiments, at least one of the linkers in the sc(Fv)₂ structure comprises the amino acid sequence of SEQ ID NO: 287. In particular embodiments, at least one of the linkers in the sc(Fv)₂ structure comprises the amino acid sequence of SEQ ID NO: 288. In particular embodiments, at least one of the linkers in the sc(Fv)₂ structure comprises the amino acid sequence of SEQ ID NO: 289. In particular embodiments, at least one of the linkers in the sc(Fv)₂ structure comprises the amino acid sequence of SEQ ID NO: 290. In particular embodiments, at least one of the linkers in the sc(Fv)₂ structure comprises the amino acid sequence of SEQ ID NO: 291. In particular embodiments, at least one of the linkers in the sc(Fv)₂ structure comprises the amino acid sequence of SEQ ID NO: 292.

### 5.4.5 Humanized Antibodies

In some embodiments, antibodies provided herein can be humanized antibodies that bind ENPP3, including human and/or cynomolgus ENPP3. For example, humanized antibodies of the present disclosure may comprise one or more CDRs as shown in Tables 1-11. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, V_{L}6 subgroup I (V_{L}6I) and V_{H} subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

### 5.4.6 Human Antibodies

Human anti-ENPP3 antibodies can be constructed by combining Fv clone variable domain sequence(s) selected from human-derived phage display libraries with known human constant domain sequences(s). Alternatively, human monoclonal anti-ENPP3 antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6 (5): 561-66; Brüggemann and Taussing, 1997, Curr. Opin. Biotechnol. 8 (4): 455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147 (1): 86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human HCDR3 is commonly retained, as this CDR may be at the center of the antigen-binding site and may be the most important region of the antibody for antigen recognition. In some instances, however, HCDR3 and LCDR3, as well as HCDR2, LCDR2, and LCDR1 of the non-human antibody may be retained.

### 5.4.7 Fc Engineering

In some embodiments, the antigen binding domains that bind ENPP3 of the disclosure are conjugated to an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). In some embodiments, the antigen binding domains that bind ENPP3 of the disclosure are used to make a fusion protein, wherein the fusion protein comprises the antigen binding domains that bind ENPP3 and an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). The Ig constant region or the fragment of the Ig constant region functions also as a half-life extending moiety as discussed herein. The antigen binding domains that bind ENPP3 of the disclosure may be engineered into conventional full-length antibodies using standard methods. The full-length antibodies comprising the antigen binding domain that binds ENPP3 may further be engineered as described herein.

Immunoglobulin heavy chain constant region comprised of subdomains CH1, hinge, CH2 and CH3. The CH1 domain spans residues A118-V215, the CH2 domain residues A231-K340 and the CH3 domain residues G341-K447 on the heavy chain, residue numbering according to the EU Index. In some instances, G341 is referred as a CH2 domain residue. Hinge is generally defined as including E216 and terminating at P230 of human IgG1. In some embodiments, the Ig Fc region comprises at least the CH2 and the CH3 domains of the Ig constant region, and therefore comprises at least a region from about A231 to K447 of Ig heavy chain constant region.

In some embodiments, the C-terminal lysine (CTL) is removed from the Ig constant region. Accordingly, in some embodiments, the Ig Fc region comprises at least a region from about A231 to G446 of Ig heavy chain constant region. In specific embodiments, the CTL is removed from the Ig constant region by endogenous circulating carboxypeptidases in the blood stream (Cai et al., (2011) Biotechnol Bioeng 108:404-412). In some embodiments, during manufacturing, CTL removal may be controlled to less than the maximum level by control of concentration of extracellular Zn²⁺, EDTA or EDTA-Fe³⁺ as described in U.S. Patent Publ. No. US20140273092. CTL content of proteins may be measured using known methods.

In other embodiments, the antigen binding fragment that binds ENPP3 fused to the Ig constant region has a C-terminal lysine content from about 10% to about 90%. In other embodiments, the C-terminal lysine content is from about 20% to about 80%. In other embodiments, the C-terminal lysine content is from about 40% to about 70%. In other embodiments, the C-terminal lysine content is from about 55% to about 70%. In other embodiments, the C-terminal lysine content is about 60%.

The present disclosure also provides an antigen binding domain that binds ENPP3 fused or conjugated to an immunoglobulin (Ig) constant region or a fragment of the Ig constant region. In some embodiments, the Ig constant region is a heavy chain constant region. In some embodiments, the Ig constant region is a light chain constant region. In some embodiments, the fragment of the Ig constant region comprises a Fc region. In some embodiments, the fragment of the Ig constant region comprises a CH2 domain. In some embodiments, the fragment of the Ig constant region comprises a CH3 domain. In some embodiments, the fragment of the Ig constant region comprises the CH2 domain and the CH3 domain. In some embodiments, the fragment of the Ig constant region comprises at least portion of a hinge, the CH2 domain and the CH3 domain. Portion of the hinge refers to one or more amino acid residues of the Ig hinge. In some embodiments, the fragment of the Ig constant region comprises the hinge, the CH2 domain and the CH3 domain.

In some embodiments, the antigen binding domain that binds ENPP3 is fused or conjugated to the N-terminus of the Ig constant region or the fragment of the Ig constant region. In some embodiments, the antigen binding domain that binds ENPP3 is fused or conjugated to the C-terminus of the Ig constant region or the fragment of the Ig constant region. In some embodiments, the antigen binding domain that binds ENPP3 is fused or conjugated to the Ig constant region or the fragment of the Ig constant region via a second linker (L2). In particular embodiments, the L2 comprises the amino acid sequence of SEQ ID NO: 258. In particular embodiments, the L2 comprises the amino acid sequence of SEQ ID NO: 259. In particular embodiments, the L2 comprises the amino acid sequence of SEQ ID NO: 260. In particular embodiments, the L2 comprises the amino acid sequence of SEQ ID NO: 261. In particular embodiments, the L2 comprises the amino acid sequence of SEQ ID NO: 262. In particular embodiments, the L2 comprises the amino acid sequence of SEQ ID NO: 263. In particular embodiments, the L2 comprises the amino acid sequence of SEQ ID NO: 264. In particular embodiments, the L2 comprises the amino acid sequence of SEQ ID NO: 265. In particular embodiments, the L2 comprises the amino acid sequence of SEQ ID NO: 266. In particular embodiments, the L2 comprises the amino acid sequence of SEQ ID NO: 267. In particular embodiments, the L2 comprises the amino acid sequence of SEQ ID NO: 268. In particular embodiments, the L2 comprises the amino acid sequence of SEQ ID NO: 269. In particular embodiments, the L2 comprises the amino acid sequence of SEQ ID NO: 270. In particular embodiments, the L2 comprises the amino acid sequence of SEQ ID NO: 271. In particular embodiments, the L2 comprises the amino acid sequence of SEQ ID NO: 272. In particular embodiments, the L2 comprises the amino acid sequence of SEQ ID NO: 273. In particular embodiments, the L2 comprises the amino acid sequence of SEQ ID NO: 274. In particular embodiments, the L2 comprises the amino acid sequence of SEQ ID NO: 275. In particular embodiments, the L2 comprises the amino acid sequence of SEQ ID NO: 276. In particular embodiments, the L2 comprises the amino acid sequence of SEQ ID NO: 277. In particular embodiments, the L2 comprises the amino acid sequence of SEQ ID NO: 278. In particular embodiments, the L2 comprises the amino acid sequence of SEQ ID NO: 279. In particular embodiments, the L2 comprises the amino acid sequence of SEQ ID NO: 280. In particular embodiments, the L2 comprises the amino acid sequence of SEQ ID NO: 281. In particular embodiments, the L2 comprises the amino acid sequence of SEQ ID NO: 282. In particular embodiments, the L2 comprises the amino acid sequence of SEQ ID NO: 283. In particular embodiments, the L2 comprises the amino acid sequence of SEQ ID NO: 284. In particular embodiments, the L2 comprises the amino acid sequence of SEQ ID NO: 285. In particular embodiments, the L2 comprises the amino acid sequence of SEQ ID NO: 286. In particular embodiments, the L2 comprises the amino acid sequence of SEQ ID NO: 287. In particular embodiments, the L2 comprises the amino acid sequence of SEQ ID NO: 288. In particular embodiments, the L2 comprises the amino acid sequence of SEQ ID NO: 289. In particular embodiments, the L2 comprises the amino acid sequence of SEQ ID NO: 290. In particular embodiments, the L2 comprises the amino acid sequence of SEQ ID NO: 291. In particular embodiments, the L2 comprises the amino acid sequence of SEQ ID NO: 292.

The antigen binding domains that bind ENPP3 of the disclosure fused or conjugated to Ig constant region or the fragment of the Ig constant region may be assessed for their functionality using several known assays. Binding to ENPP3 can be assessed using methods described herein. Altered properties imparted by the Ig constant domain or the fragment of the Ig constant region such as Fc region may be assayed in Fc receptor binding assays using soluble forms of the receptors, such as the FcγRI, FcγRII, FcγRIII or FcRn receptors, or using cell-based assays measuring for example ADCC, CDC or ADCP.

ADCC can be assessed using an in vitro assay using ENPP3 expressing cells as target cells and NK cells as effector cells. Cytolysis may be detected by the release of label (e.g. radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

ADCP can be evaluated by using monocyte-derived macrophages as effector cells and any ENPP3 expressing cells as target cells which are engineered to express GFP or other labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11⁺CD14⁺ macrophages using standard methods.

CDC of cells may be measured for example by plating Daudi cells at 1×10⁵ cells/well (50 μL/well) in RPMI-B (RPMI supplemented with 1% BSA), adding 50 μL of test protein to the wells at final concentration between 0-100 μg/mL, incubating the reaction for 15 min at room temperature, adding 11 μL of pooled human serum to the wells, and incubation the reaction for 45 min at 37° C. Percentage (%) lysed cells may be detected as % propidium iodide stained cells in FACS assay using standard methods.

In some embodiments, it may be desirable to modify an anti-ENPP3 antibody provided herein by Fc engineering. In certain embodiments, the modification to the Fc region of the antibody results in the decrease or elimination of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. For example, substitutions into human IgG1 using IgG2 residues at positions 233-236 and IgG4 residues at positions 327, 330, and 331 were shown to greatly reduce ADCC and CDC (see, e.g., Armour et al., 1999, Eur. J. Immunol. 29 (8): 2613-24; and Shields et al., 2001, J. Biol. Chem. 276 (9): 6591-604). Other Fc variants are provided elsewhere herein.

To increase the serum half life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

In some embodiments, the Ig constant region or the fragment of the Ig constant region comprises at least one mutation that modulates a half-life of the ENPP3 binding agent. In some embodiments, the at least one mutation that modulates the half-life of the ENPP3 binding agent is selected from the group consisting of H435A, P257I/N434H, D376V/N434H, M252Y/S254T/T256E/H433K/N434F, T308P/N434A, and H435R, wherein residue numbering is according to the EU index. In some embodiments, the ENPP3 binding agent comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the ENPP3 binding agent independently selected from the group consisting of H435A, P257I/N434H, D376V/N434H, M252Y/S254T/T256E/H433K/N434F, T308P/N434A, and H435R, wherein residue numbering is according to the EU index.

In some embodiments, the Ig constant region or the fragment of the Ig constant region comprises at least one mutation that results in reduced binding of the ENPP3 binding agent to a FcγR.

In some embodiments, the at least one mutation that results in reduced binding of the ENPP3 binding agent to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index. In some embodiments, the ENPP3 binding agent comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the ENPP3 binding agent independently selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the Ig constant region or the fragment of the Ig constant region comprises at least one mutation that results in enhanced binding of the ENPP3 binding agent to a FcγR.

In some embodiments, the at least one mutation that results in enhanced binding of the ENPP3 binding agent to the FcγR is selected from the group consisting of S239D/1332E, S298A/E333A/K334A, F243L/R292P/Y300L, F243L/R292P/Y300L/P396L, F243L/R292P/Y300L/V305I/P396L and G236A/S239D/1332E, wherein residue numbering is according to the EU index. In some embodiments, the FcγR is FcγRI, FcγRIIA, FcγRIIB or FcγRIII, or any combination thereof. In some embodiments, the ENPP3 binding agent comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the ENPP3 binding agent independently selected from the group consisting of S239D/1332E, S298A/E333A/K334A, F243L/R292P/Y300L, F243L/R292P/Y300L/P396L, F243L/R292P/Y300L/V305I/P396L and G236A/S239D/1332E, wherein residue numbering is according to the EU index.

In some embodiments, the ENPP3 binding agent comprises at least one mutation in a CH3 domain of a first Ig constant region or in a CH3 domain of the fragment of the first Ig constant region and/or at least one mutation in a CH3 domain of a second Ig constant region or in a CH3 domain of the fragment of the second Ig constant region. In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, T366L, F405W, K392L, T394W, T394S, Y407T, Y407A, H435R, Y436F, T366S/L368A/Y407V, L351Y/F405A/Y407V, T366I/K392M/T394W, T366L/K392L/T394W, F405A/Y407V, T366L/K392M/T394W, L351Y/Y407A, L351Y/Y407V, T366A/K409F, L351Y/Y407A, T366V/K409F, T366A/K409F, T350V/L351Y/F405A/Y407V, T350V/T366L/K392L/T394W, and H435R/L436F wherein residue numbering is according to the EU index. In some embodiments, at least one mutation in the CH3 domain is selected from the group consisting of H435R, Y436F and H435R/L436F, wherein residue numbering is according to the EU index.

In some embodiments, the first Ig constant region or the fragment of the first Ig constant region and the second Ig constant region or the fragment of the second Ig constant region comprise the following mutations L234A_L235A_D265S_T350V_L351Y_F405A_Y407V in the first Ig constant region and L234A_L235A_D265S_T350V_T366L_K392L_T394W in the second Ig constant region; or L234A_L235A_D265S_T350V_T366L_K392L_T394W in the first Ig constant region and L234A_L235A_D265S_T350V_L351Y_F405A_Y407V in the second Ig constant region.

### 5.4.8 Alternative Binding Agents

The present disclosure encompasses non-immunoglobulin binding agents that specifically bind to the same epitope as an anti-ENPP3 antibody disclosed herein. In some embodiments, a non-immunoglobulin binding agent is identified as an agent that displaces or is displaced by an anti-ENPP3 antibody of the present disclosure in a competitive binding assay. These alternative binding agents may include, for example, any of the engineered protein scaffolds known in the art. Such scaffolds may comprise one or more CDRs as shown in Tables 1-11. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352:95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

### 5.5 Multispecific Binding Proteins

In one aspect of the present disclosure, the ENPP3 binding agents described herein are multispecific (e.g., bispecific or trispecific) binding molecules that can bind to one or more other antigens in addition to ENPP3.

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating bispecific antibodies, see, for example, Bispecific Antibodies (Kontermann ed., 2011).

In some embodiments, the multispecific protein is bispecific. In some embodiments, the multispecific protein is trispecific. In some embodiments, the multispecific protein is tetraspecific.

In specific embodiments, the ENPP3 binding agents described herein are bispecific antibodies. In some embodiments, bispecific antibodies described herein are monoclonal antibodies that have binding specificities for at least two different antigens. In certain embodiments, bispecific antibodies are human or humanized antibodies. In certain embodiments, one of the binding specificities is for ENPP3 and the other is for any other antigen. In some embodiments, one of the binding specificities is for ENPP3, and the other is for another surface antigen expressed on cells expressing ENPP3. In certain embodiments, bispecific antibodies may bind to two different epitopes of ENPP3 (i.e., bivalent for ENPP3). Bispecific antibodies can be prepared as full length antibodies or antibody fragments (e.g., F(ab′)₂ bispecific antibodies).

In other embodiments, the multispecific protein is monovalent for binding to ENPP3. In other embodiments, the multispecific protein is bivalent for binding to ENPP3. In other embodiments, the multispecific protein is monovalent for binding to the second antigen. In other embodiments, the multispecific protein is bivalent for binding to second antigen. In other embodiments, the multispecific protein is monovalent for binding to ENPP3, and is monovalent for binding to the second antigen. In other embodiments, the multispecific protein is bivalent for binding to ENPP3, and is monovalent for binding to the second antigen. In other embodiments, the multispecific protein is monovalent for binding to ENPP3, and is bivalent for binding to the second antigen. In other embodiments, the multispecific protein is bivalent for binding to ENPP3, and is bivalent for binding to the second antigen.

In some embodiments, the first antigen binding domain that binds ENPP3 and/or the second antigen binding domain that binds the second antigen are independently selected from a scFv, a spFv, a (scFv)₂, a Fv, a Fab, a F(ab′)₂, a Fd, a dAb or a VHH. In some embodiments, the first antigen binding domain that binds ENPP3 and/or the second antigen binding domain that binds the second antigen comprise the Fab. In some embodiments, the first antigen binding domain that binds ENPP3 and/or the second antigen binding domain that binds the second antigen comprise the F(ab′)₂. In some embodiments, the first antigen binding domain that binds ENPP3 and/or the second antigen binding domain that binds the second antigen comprise the VHH. In some embodiments, the first antigen binding domain that binds ENPP3 and/or the second antigen binding domain that binds the second antigen comprise the Fv. In some embodiments, the first antigen binding domain that binds ENPP3 and/or the second antigen binding domain that binds the second antigen comprise the Fd. In some embodiments, the first antigen binding domain that binds ENPP3 and/or the second antigen binding domain that binds the second antigen comprise the scFv. In some embodiments, the first antigen binding domain that binds ENPP3 and/or the second antigen binding domain that binds the second antigen comprise the spFv. In some embodiments, the first antigen binding domain that binds ENPP3 comprises the Fab, and the second antigen binding domain that binds the second antigen comprises the scFv. In some embodiments, the first antigen binding domain that binds ENPP3 comprises the Fab, and the second antigen binding domain that binds the second antigen comprises the spFv. In specific embodiments, the second antigen is CD3ε.

In some embodiments, the first antigen binding domain that binds ENPP3 is fused or conjugated to a first immunoglobulin (Ig) constant region or a fragment of the first Ig constant region and/or the second antigen binding domain that binds the tumor antigen is fused or conjugated to a second immunoglobulin (Ig) constant region or a fragment of the second Ig constant region.

In some embodiments, the fragment of the first Ig constant region and/or the fragment of the second Ig constant region comprises a Fc region. In some embodiments, the fragment of the first Ig constant region and/or the fragment of the second Ig constant region comprises a CH2 domain. In some embodiments, the fragment of the first Ig constant region and/or the fragment of the second Ig constant region comprises a CH3 domain. In some embodiments, the fragment of the first Ig constant region and/or the fragment of the second Ig constant region comprises the CH2 domain and the CH3 domain.

In some embodiments, the fragment of the first Ig constant region and/or the fragment of the second Ig constant region comprises at least portion of a hinge, the CH2 domain and the CH3 domain. In some embodiments, the fragment of the Ig constant region comprises the hinge, the CH2 domain and the CH3 domain.

In some embodiments, the multispecific protein further comprises a second linker (L2) between the first antigen binding domain that binds ENPP3 and the first Ig constant region or the fragment of the first Ig constant region and the second antigen binding domain that binds the second antigen and the second Ig constant region or the fragment of the second Ig constant region. In some embodiments, the L2 comprises the amino acid sequence of any one of SEQ ID NO: 258 to 292.

In some embodiments, the first Ig constant region or the fragment of the first Ig constant region and the second Ig constant region or the fragment of the second Ig constant region is an IgG1, an IgG2, and IgG3 or an IgG4 isotype. In some embodiments, the first Ig constant region or the fragment of the first Ig constant region and the second Ig constant region or the fragment of the second Ig constant region is an IgG1 isotype. In some embodiments, the first Ig constant region or the fragment of the first Ig constant region and the second Ig constant region or the fragment of the second Ig constant region is an IgG2 isotype. In some embodiments, the first Ig constant region or the fragment of the first Ig constant region and the second Ig constant region or the fragment of the second Ig constant region is an IgG3 isotype. In some embodiments, the first Ig constant region or the fragment of the first Ig constant region and the second Ig constant region or the fragment of the second Ig constant region is an IgG4 isotype.

The first Ig constant region or the fragment of the first Ig constant region and the second Ig constant region or the fragment of the second Ig constant region can further be engineered as described herein. In some embodiments, the first Ig constant region or the fragment of the first Ig constant region and the second Ig constant region or the fragment of the second Ig constant region comprises at least one mutation that results in reduced binding of the multispecific protein to a FcγR.

In some embodiments, the at least one mutation that results in reduced binding of the multispecific protein to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the first Ig constant region or the fragment of the first Ig constant region and the second Ig constant region or the fragment of the second Ig constant region comprises at least one mutation that results in enhanced binding of the multispecific protein to a Fcγ receptor (FcγR).

In some embodiments, the at least one mutation that results in enhanced binding of the multispecific protein to the FcγR is selected from the group consisting of S239D/1332E, S298A/E333A/K334A, F243L/R292P/Y300L, F243L/R292P/Y300L/P396L, F243L/R292P/Y300L/V305I/P396L and G236A/S239D/1332E, wherein residue numbering is according to the EU index. In some embodiments, the FcγR is FcγRI, FcγRIIA, FcγRIIB or FcγRIII, or any combination thereof.

In some embodiments, the first Ig constant region or the fragment of the first Ig constant region and the second Ig constant region or the fragment of the second Ig constant region comprises at least one mutation that modulates a half-life of the multispecific protein. In some embodiments, the at least one mutation that modulates the half-life of the multispecific protein is selected from the group consisting of H435A, P257I/N434H, D376V/N434H, M252Y/S254T/T256E/H433K/N434F, T308P/N434A, and H435R, wherein residue numbering is according to the EU index.

In some embodiments, the multispecific protein comprises at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region. In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, T366L, F405W, K392L, T394W, T394S, Y407T, Y407A, T366S/L368A/Y407V, L351Y/F405A/Y407V, T366I/K392M/T394W, T366L/K392L/T394W, F405A/Y407V, T366L/K392M/T394W, L351Y/Y407A, L351Y/Y407V, T366A/K409F, L351Y/Y407A, T366V/K409F, T366A/K409F, T350V/L351Y/F405A/Y407V and T350V/T366L/K392L/T394W, wherein residue numbering is according to the EU index.

In some embodiments, the first Ig constant region or the fragment of the first Ig constant region and the second Ig constant region or the fragment of the second Ig constant region comprise the following mutations L234A_L235A_D265S_T350V_L351Y_F405A_Y407V in the first Ig constant region and L234A_L235A_D265S_T350V_T366L_K392L_T394W in the second Ig constant region; or L234A_L235A_D265S_T350V_T366L_K392L_T394W in the first Ig constant region and L234A_L235A_D265S_T350V_L351Y_F405A_Y407V in the second Ig constant region.

In some embodiments, the multispecific binding proteins provided herein are antibodies having a full-length antibody structure. “Full length antibody” refers to an antibody having two full length antibody heavy chains and two full length antibody light chains. A full-length antibody heavy chain (HC) consists of well-known heavy chain variable and constant domains VH, CH1, hinge, CH2, and CH3. A full-length antibody light chain (LC) consists of well-known light chain variable and constant domains VL and CL. The full-length antibody can be lacking the C-terminal lysine (K) in either one or both heavy chains. “Fab-arm” or “half molecule” refers to one heavy chain-light chain pair that specifically binds an antigen.

Full length bispecific antibodies can be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms can be engineered to favor heterodimerization over homodimerization. The resulting product is a bispecific antibody having two Fab arms or half molecules which each bind a distinct epitope, i.e. an epitope on ENPP3 and an epitope on CD3.

“Homodimerization” refers to an interaction of two heavy chains having identical CH3 amino acid sequences. “Homodimer” refers to an antibody having two heavy chains with identical CH3 amino acid sequences. “Heterodimerization” refers to an interaction of two heavy chains having non-identical CH3 amino acid sequences. “Heterodimer” refers to an antibody having two heavy chains with non-identical CH3 amino acid sequences.

In some embodiments, the binding proteins provided herein include designs such as the Triomab/Quadroma (Trion Pharma/Fresenius Biotech), Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), the Biclonic (Merus) and the DuoBody (Genmab A/S).

In some embodiments, a multispecific binding protein provided herein is in the knob-and-hole format. In some embodiments, a multispecific binding protein provided herein is in a DuoBody format.

The Triomab quadroma technology can be used to generate full length bispecific antibodies provided herein. Triomab technology promotes Fab arm exchange between two parental chimeric antibodies, one parental mAb having IgG2a and the second parental mAb having rat IgG2b constant regions, yielding chimeric bispecific antibodies.

The “knob-in-hole” strategy (see, e.g., International Publication No. WO 2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

The CrossMAb technology can be used to generate full length bispecific antibodies provided herein. CrossMAbs, in addition to utilizing the “knob-in-hole” strategy to promoter Fab arm exchange, have in one of the half arms the CH1 and the CL domains exchanged to ensure correct light chain pairing of the resulting bispecific antibody (see e.g. U.S. Pat. No. 8,242,247).

Other cross-over strategies can be used to generate full length bispecific antibodies provided herein by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in International Publication Nos. WO 2009/080254, WO 2009/080251, WO 2009/018386 and WO 2009/080252.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849. Other strategies that can facilitate the removal of homodimer and/or half-molecule species include incorporating both substitutions H435R and Y436F in a heavy chain CH3 domain.

LUZ-Y technology can be utilized to generate bispecific antibodies provided herein. In this technology, a leucine zipper is added into the C terminus of the CH3 domains to drive the heterodimer assembly from parental mAbs that is removed post-purification as described in Wranik et al., (2012) J Biol Chem 287 (52): 42221-9.

SEEDbody technology can be utilized to generate bispecific antibodies provided herein. SEEDbodies have, in their constant domains, select IgG residues substituted with IgA residues to promote heterodimerization as described in U.S. Patent No. US20070287170.

In addition to methods described above, binding agents provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO 2011/131746.

In some embodiments described herein, the bispecific ENPP3 binding agent described herein comprises a first binding region binding ENPP3 and a second binding region binding a second antigen, and comprises at least one substitution in an antibody CH3 constant domain. Substitutions are typically made at the DNA level to a molecule such as the constant domain of the antibody using standard methods.

The antibodies provided herein can be engineered into various well-known antibody forms.

In some embodiments, the bispecific antibody is a diabody or a cross-body.

In some embodiments, the bispecific antibody includes IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (ImClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual (ScFv)₂-Fab (National Research Center for Antibody Medicine—China).

In some embodiments, Fab fusion bispecific antibodies include F(ab) 2 (Medarex/AMGEN), Dual-Action or Bis-Fab (Genentech), Dock-and-Lock (DNL) (ImmunoMedics), Bivalent Bispecific (Biotecnoland Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12:276 and in Nunez-Prado et al., (2015) Drug Discovery Today 20 (5): 588-594.

### 5.6 Bispecific ENPP3×CD3 Binding Proteins

As described herein, any antigen other than ENPP3 can be selected as the second antigen target for the present multispecific ENPP3 binding agent. In specific embodiments, the multispecific binding agent is a multispecific binding protein. In particular embodiments, the second antigen is expressed on an immune cell. In some embodiments, the second antigen is CD3. In specific embodiments, the second antigen is CD3ε.

In specific embodiments, the multispecific binding protein described herein is a bispecific ENPP3×CD3 binding protein comprising a first antigen binding region that binds ENPP3 and a second antigen binding region that binds CD3ε. In some embodiments, the ENPP3 binding region of multispecific binding protein can be any ENPP3 binding region or ENPP3 binding protein described herein.

In particular embodiments, the CD3ε binding region of the multispecific binding protein comprises one or more binding sequences set forth in Tables 13A to 15. In some embodiments, the CD3ε binding region comprises one or more CDR sequences set forth in SEQ ID NOs: 159 and 160. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CD3ε binding region is humanized. In some embodiments, the CD3ε binding region comprises an acceptor human framework, e.g., a human immunoglobulin framework or a human consensus framework.

In some embodiments, the CD3ε binding region provided herein comprises a VH comprising an HCDR1, HCDR2, and HCDR3 of a VH as set forth in SEQ ID NO:200 or 202. In some embodiments, the CD3ε binding region provided herein comprises a VL comprising an LCDR1, LCDR2, and LCDR3 of a VL as set forth in SEQ ID NO:201 or 203. In some embodiments, the CD3ε binding region provided herein comprises a VH comprising an HCDR1, HCDR2, and HCDR3 of a VH as set forth in SEQ ID NO:200 or 202, and a VL comprising an LCDR1, LCDR2, and LCDR3 of a VL as set forth in SEQ ID NO:201 or 203. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the CD3ε binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:179, 185, 187, 189, or 195; (ii) an HCDR2 comprising an amino acid sequence having at least 75%, 80%, 85%, 86%, 87%, 88%, 89%. 90%, 91%, 92%, 93%, 94%, 95%, 96%, 97%, 98%, 99%, or 100% sequence identity to any of SEQ ID NO: 180, 186, 188, 190 or 196, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO: 181, 191 or 197; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO: 182, 192 or 198; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO: 183, 193 or 199; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NO: 184 or 194. In some embodiments, the CD3ε binding region is humanized. In some embodiments, the CD3ε binding region comprises an acceptor human framework, e.g., a human immunoglobulin framework or a human consensus framework.

In some specific embodiments, the CD3 binding region provided herein comprises one or more CDRs in Table 13A or Table 13B.

In some embodiments, the CD3ε binding region provided herein comprises an HCDR1 comprising an amino acid sequence of any of SEQ ID NO: 179, 185, 187, 189, or 195; (ii) an HCDR2 comprising an amino acid sequence of any of SEQ ID NO:180, 186, 188, 190 or 196, (iii) an HCDR3 comprising an amino acid sequence SEQ ID NO: 181, 191 or 197; (iv) a LCDR1 comprising an amino acid sequence of SEQ ID NO: 182, 192 or 198; (v) a LCDR2 comprising an amino acid sequence of SEQ ID NO: 183, 193 or 199; and/or (vi) a LCDR3 comprising an amino acid sequence of SEQ ID NO: 184 or 194.

In some specific embodiments, in the CD3ε binding region provided herein, the HCDR1 comprises the amino acid sequence of SEQ ID NO: 179, the HCDR2 comprises the amino acid sequence of SEQ ID NO: 180, the HCDR3 comprises the amino acid sequence of SEQ ID NO: 181, the LCDR1 comprises the amino acid sequence of SEQ ID NO: 182, the LCDR2 comprises the amino acid sequence of SEQ ID NO: 183, and the LCDR3 comprises the amino acid sequence of SEQ ID NO: 184.

In some specific embodiments, in the CD3ε binding region provided herein, the HCDR1 comprises the amino acid sequence of SEQ ID NO: 185, the HCDR2 comprises the amino acid sequence of SEQ ID NO: 186, the HCDR3 comprises the amino acid sequence of SEQ ID NO: 181, the LCDR1 comprises the amino acid sequence of SEQ ID NO: 182 the LCDR2 comprises the amino acid sequence of SEQ ID NO: 183, and the LCDR3 comprises the amino acid sequence of SEQ ID NO: 184.

In some specific embodiments, in the CD3ε binding region provided herein, the HCDR1 comprises the amino acid sequence of SEQ ID NO: 187, the HCDR2 comprises the amino acid sequence of SEQ ID NO: 188, the HCDR3 comprises the amino acid sequence of SEQ ID NO: 181, the LCDR1 comprises the amino acid sequence of SEQ ID NO: 182, the LCDR2 comprises the amino acid sequence of SEQ ID NO: 183, and the LCDR3 comprises the amino acid sequence of SEQ ID NO: 184.

In some specific embodiments, in the CD3ε binding region provided herein, the HCDR1 comprises the amino acid sequence of SEQ ID NO: 189, the HCDR2 comprises the amino acid sequence of SEQ ID NO: 190, the HCDR3 comprises the amino acid sequence of SEQ ID NO: 191, the LCDR1 comprises the amino acid sequence of SEQ ID NO: 192, the LCDR2 comprises the amino acid sequence of SEQ ID NO: 193, and the LCDR3 comprises the amino acid sequence of SEQ ID NO: 194.

In some specific embodiments, in the CD3ε binding region provided herein, the HCDR1 comprises the amino acid sequence of SEQ ID NO: 195, the HCDR2 comprises the amino acid sequence of SEQ ID NO: 196, the HCDR3 comprises the amino acid sequence of SEQ ID NO: 197, the LCDR1 comprises the amino acid sequence of SEQ ID NO: 198, the LCDR2 comprises the amino acid sequence of DSS and the LCDR3 comprises the amino acid sequence of SEQ ID NO: 184.

In one embodiment, provided herein is a binding region that binds CD3ε, comprising a VH comprising a HCDR1, a HCDR2, and a HCDR3 having an amino acid sequence of SEQ ID NOs: 179, 180 and 181, respectively. In another embodiment, provided herein is a binding region that binds CD3ε, comprising a VL comprising a LCDR1, a LCDR2, and a LCDR3 having an amino acid sequence of SEQ ID NOs: 182, 183 and 184, respectively. In another embodiment, provided herein is a binding region that binds CD3ε, comprising: (i) a VH comprising a HCDR1, a HCDR2, and a HCDR3 having an amino acid sequence of SEQ ID NOs: 179, 180 and 181, respectively, and (ii) a VL comprising a LCDR1, a LCDR2, and a LCDR3 having an amino acid sequence of SEQ ID NOs: 182, 183 and 184, respectively.

In one embodiment, provided herein is a binding region that binds CD3ε, comprising a VH comprising a HCDR1, a HCDR2, and a HCDR3 having an amino acid sequence of SEQ ID NOs: 185, 186 and 181, respectively. In another embodiment, provided herein is a binding region that binds CD3ε, comprising a VL comprising a LCDR1, a LCDR2, and a LCDR3 having an amino acid sequence of SEQ ID NOs: 182, 183 and 184, respectively. In another embodiment, provided herein is a binding region that binds CD3ε, comprising: (i) a VH comprising a HCDR1, a HCDR2, and a HCDR3 having an amino acid sequence of SEQ ID NOs: 185, 186 and 181, respectively, and (ii) a VL comprising a LCDR1, a LCDR2, and a LCDR3 having an amino acid sequence of SEQ ID NOs: 182, 183 and 184, respectively.

In one embodiment, provided herein is a binding region that binds CD3ε, comprising a VH comprising a HCDR1, a HCDR2, and a HCDR3 having an amino acid sequence of SEQ ID NOs: 187, 188 and 181, respectively. In another embodiment, provided herein is a binding region that binds CD3ε, comprising a VL comprising a LCDR1, a LCDR2, and a LCDR3 having an amino acid sequence of SEQ ID NOs: 182, 183 and 184, respectively. In another embodiment, provided herein is a binding region that binds CD3ε, comprising: (i) a VH comprising a HCDR1, a HCDR2, and a HCDR3 having an amino acid sequence of SEQ ID NOs: 187, 188 and 181, respectively, and (ii) a VL comprising a LCDR1, a LCDR2, and a LCDR3 having an amino acid sequence of SEQ ID NOs: 182, 183 and 184, respectively.

In one embodiment, provided herein is a binding region that binds CD3ε, comprising a VH comprising a HCDR1, a HCDR2, and a HCDR3 having an amino acid sequence of SEQ ID NOs: 189, 190, and 191, respectively. In another embodiment, provided herein is a binding region that binds CD3ε, comprising a VL comprising a LCDR1, a LCDR2, and a LCDR3 having an amino acid sequence of SEQ ID NOs: 192, 193 and 194, respectively. In another embodiment, provided herein is a binding region that binds CD3ε, comprising: (i) a VH comprising a HCDR1, a HCDR2, and a HCDR3 having an amino acid sequence of SEQ ID NOs: 189, 190, and 191, respectively, and (ii) a VL comprising a LCDR1, a LCDR2, and a LCDR3 having an amino acid sequence of SEQ ID NOs: 192, 193 and 194, respectively.

In one embodiment, provided herein is a binding region that binds CD3ε, comprising a VH comprising a HCDR1, a HCDR2, and a HCDR3 having an amino acid sequence of SEQ ID NOs: 195, 196 and 197, respectively. In another embodiment, provided herein is a binding region that binds CD3ε, comprising a VL comprising a LCDR1, a LCDR2, and a LCDR3 having an amino acid sequence of SEQ ID NOs: 198, 199 and 184, respectively. In another embodiment, provided herein is a binding region that binds CD3ε, comprising: (i) a VH comprising a HCDR1, a HCDR2, and a HCDR3 having an amino acid sequence of SEQ ID NOs: 195, 196 and 197, respectively, and (ii) a VL comprising a LCDR1, a LCDR2, and a LCDR3 having an amino acid sequence of SEQ ID NOs: 198, 199 and 184, respectively.

In some embodiments, the CD3ε binding region further comprises one or more framework regions. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system. In some embodiments, the CD3ε binding region further comprises one or more framework regions of SEQ ID NO:200, 201, 202 or 203.

In some embodiments, the CD3ε binding region provided herein comprises a VH comprising the amino acid sequence of SEQ ID NO:200, and a VL comprising the amino acid sequence of SEQ ID NO:201. In some embodiments, the CD3ε binding region provided herein comprises a VH comprising the amino acid sequence of SEQ ID NO:202, and a VL comprising the amino acid sequence of SEQ ID NO:203.

In certain embodiments, the CD3ε binding region provided herein comprises amino acid sequences with certain percent identity relative to any CD3ε binding region provided herein. The determination of percent identity can be accomplished using mathematical algorithms known in the art or described herein.

In some embodiments, the CD3ε binding region provided herein contains substitutions (e.g., conservative substitutions), insertions, or deletions relative to the reference sequence, but the CD3ε binding region comprising that sequence retains the ability to bind to CD3ε. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the CD3ε binding region provided herein includes post-translational modifications of a reference sequence.

TABLE 13A
CD3B2030-N106A (NPP3B815-CD3 arm) Binding Region Sequences

| | Kabat | Chothia | AbM | Contact | IMGT |
|---|---|---|---|---|---|
| Heavy Chain | RSTMH | GYTFTRS | GYTFTRSTMH | TRSTMH | GYTFTRST |
| variable region | (SEQ ID | (SEQ ID | (SEQ ID | (SEQ ID | (SEQ ID |
| CDR1 | NO: 179) | NO: 185) | NO: 187) | NO: 189) | NO: 195) |
| Heavy Chain | YINPSSAYTNY | NPSSAY | YINPSSAYTN | WIGYINPSSAYTN | INPSSAYT |
| variable region | NQKFQG | (SEQ ID | (SEQ ID | (SEQ ID | (SEQ ID |
| CDR2 | (SEQ ID | NO: 186) | NO: 188) | NO: 190) | NO: 196) |
| | NO: 180) | | | | |
| Heavy Chain | PQVHYDYAGF | PQVHYDYAGFPY | PQVHYDYAGFPY | ASPQVHYDYAGF | ASPQVHYDY |
| variable region | PY | (SEQ ID | (SEQ ID | P | AGFPY |
| CDR3 | (SEQ ID | NO: 181) | NO: 181) | (SEQ ID | (SEQ ID |
| | NO: 181) | | | NO: 191) | NO: 197) |
| Light Chain | SASSSVSYMN | SASSSVSYMN | SASSSVSYMN | SYMNWY | SSVSY |
| variable region | (SEQ ID | (SEQ ID | (SEQ ID | (SEQ ID | (SEQ ID |
| CDR1 | NO: 182) | NO: 182) | NO: 182) | NO: 192) | NO: 198) |
| Light Chain | DSSKLAS | DSSKLAS | DSSKLAS | RWIYDSSKLA | DSS |
| variable region | (SEQ ID | (SEQ ID | (SEQ ID | (SEQ ID | ) |
| CDR2 | NO: 183) | NO: 183) | NO: 183) | NO: 193) | |
| Light Chain | QQWSRNPPT | QQWSRNPPT | QQWSRNPPT | QQWSRNPP | QQWSRNPPT |
| variable region | (SEQ ID | (SEQ ID | (SEQ ID | (SEQ ID | (SEQ ID |
| CDR3 | NO: 184) | NO: 184) | NO: 184) | NO: 194) | NO: 184) |

VH (SEQ ID NO: 200)
QVQLVQSGAEVKKPGSSVKVSCKASGYTFTRSTMHWVKQAPGQGLEWIGYINPSSAYTNYNQKFQGRVT
LTADKSTSTAYMELSSLRSEDTAVYYCASPQVHYDYAGFPYWGCGTLVTVSS
DNA sequence encoding VH (SEQ ID NO: 318)
CAGGTTCAGCTGGTTCAGTCTGGCGCCGAAGTGAAGAAACCTGGCTCCTCCGTGAAAGTGTCCTGCAA
GGCTTCCGGCTACACCTTTACCAGATCCACCATGCACTGGGTCAAGCAGGCCCCTGGACAAGGCTTGG
AGTGGATCGGCTACATCAACCCCAGCTCCGCCTACACCAACTACAACCAGAAATTCCAGGGCAGAGT
GACCCTGACCGCCGACAAGTCTACCTCCACCGCCTACATGGAACTGTCCAGCCTGAGATCTGAGGACA
CCGCCGTGTACTACTGCGCCTCTCCTCAGGTTCACTACGACTACGCCGGCTTTCCTTATTGGGGCTGTG
GCACCCTGGTCACCGTTTCTTCT
VL (SEQ ID NO: 201)
EIVLTQSPATLSASPGERVTLSCSASSSVSYMNWYQQKPGCAPRRWIYDSSKLASGVPARFSGSGSGRDYT
LTISSLEPEDFAVYYCQQWSRNPPTFGGGTKVEIK
DNA sequence encoding VL (SEQ ID NO: 319)
GAGATCGTGCTGACCCAGTCTCCTGCCACACTGAGTGCTTCTCCAGGCGAGAGAGTGACCCTGTCCTG
CTCCGCTTCCTCCTCCGTGTCCTACATGAACTGGTATCAGCAGAAGCCCGGCTGCGCCCCTAGAAGAT
GGATCTACGACTCCTCCAAGCTGGCCTCTGGCGTGCCTGCTAGATTTTCCGGCTCTGGCTCTGGCAGAG
ACTATACCCTGACAATCTCCAGCCTGGAACCTGAGGACTTCGCCGTGTACTACTGCCAGCAGTGGTCT
AGGAACCCTCCTACCTTTGGCGGAGGCACCAAGGTGGAAATCAAG

TABLE 13B
CD3B2030-N106A Binding Region Sequences

| | Kabat | Chothia | AbM | Contact | IMGT |
|---|---|---|---|---|---|
| Heavy Chain | RSTMH | GYTFTRS | GYTFTRSTM | TRSTMH | GYTFTRST |
| Variable | (SEQ ID | (SEQ ID | H | (SEQ ID | (SEQ ID |
| Region CDR1 | NO: 179) | NO: 185) | (SEQ ID | NO: 189) | NO: 195) |
| | | | NO: 187) | | |
| Heavy Chain | YINPSSAYTNYN | NPSSAY | YINPSSAYT | WIGYINPSSA | INPSSAYT |
| Variable | QKFQG | (SEQ ID | N | YTN | (SEQ ID |
| Region CDR2 | (SEQ ID | NO: 186) | (SEQ ID | (SEQ ID | NO: 196) |
| | NO: 180) | | NO: 188) | NO: 190) | |
| Heavy Chain | PQVHYDYAGFP | PQVHYDYA | PQVHYDYA | ASPQVHYDY | ASPQVHYDY |
| Variable | Y | GFPY | GFPY | AGFP | AGFPY |
| Region CDR3 | (SEQ ID | (SEQ ID | (SEQ ID | (SEQ ID | (SEQ ID |
| | NO: 181) | NO: 181) | NO: 181) | NO: 191) | NO: 197) |
| Light Chain | SASSSVSYMN | SASSSVSYM | SASSSVSYM | SYMNWY | SSVSY |
| Variable | (SEQ ID | N | N | (SEQ ID | (SEQ ID |
| Region CDR1 | NO: 182) | (SEQ ID | (SEQ ID | NO: 192) | NO: 198) |
| | | NO: 182) | NO: 182) | | |
| Light Chain | DSSKLAS | DSSKLAS | DSSKLAS | RWIYDSSKL | DSS |
| Variable | (SEQ ID | (SEQ ID | (SEQ ID | A | ) |
| Region CDR2 | NO: 183) | NO: 183) | NO: 183) | (SEQ ID | |
| | | | | NO: 193) | |
| Light Chain | QQWSRNPPT | QQWSRNPP | QQWSRNPP | QQWSRNPP | QQWSRNPPT |
| Variable | (SEQ ID | T | T | (SEQ ID | (SEQ ID |
| Region CDR3 | NO: 184) | (SEQ ID | (SEQ ID | NO: 194) | NO: 184) |
| | | NO: 184) | NO: 184) | | |

VH (SEQ ID NO: 202)
QVQLVQSGAEVKKPGSSVKVSCKASGYTFTRSTMHWVKQAPGQGLEWIGYINPSSAYT
NYNQKFQGRVTLTADKSTSTAYMELSSLRSEDTAVYYCASPQVHYDYAGFPYWGQGT
LVTVSS
VL (SEQ ID NO: 203)
EIVLTQSPATLSASPGERVTLSCSASSSVSYMNWYQQKPGQAPRRWIYDSSKLASGVPA
RFSGSGSGRDYTLTISSLEPEDFAVYYCQQWSRNPPTFGGGTKVEIK

In some embodiments, the CD3ε binding region provided herein comprises HCDR1, HCDR2, and HCDR3 as set forth in SEQ ID NO:220. In some embodiments, the CD3ε binding region provided herein comprises LCDR1, LCDR2, and LCDR3 as set forth in SEQ ID NO:221. In some embodiments, the CD3 binding region provided herein comprises HCDR1, HCDR2, and HCDR3 as set forth in SEQ ID NO:220, and LCDR1, LCDR2, and LCDR3 as set forth in SEQ ID NO:221. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the CD3ε binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:204, 210 or 216; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:205, 211 or 217, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:206, 212 or 218; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:207, 213 or 219; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:208 or 214; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NO:209 or 215. In some embodiments, the CD3ε binding region is humanized. In some embodiments, the CD3ε binding region comprises an acceptor human framework, e.g., a human immunoglobulin framework or a human consensus framework.

In some specific embodiments, the CD3 binding region provided herein comprises one or more CDRs in Table 13C.

In some embodiments, the CD3ε binding region provided herein comprises an HCDR1 comprising an amino acid sequence of any of SEQ ID NO:204, 210 or 216; (ii) an HCDR2 comprising an amino acid sequence of any of SEQ ID NO:205, 211 or 217, (iii) an HCDR3 comprising an amino acid sequence SEQ ID NO:206, 212 or 218; (iv) a LCDR1 comprising an amino acid sequence SEQ ID NO: 207, 213 or 219; (v) a LCDR2 comprising an amino acid sequence of SEQ ID NO:208 or 214; and/or (vi) a LCDR3 comprising an amino acid sequence of SEQ ID NO:209 or 215.

In some specific embodiments, in the CD3ε binding region provided herein, the HCDR1 comprises the amino acid sequence of SEQ ID NO:204, the HCDR2 comprises the amino acid sequence of SEQ ID NO:205, the HCDR3 comprises the amino acid sequence of SEQ ID NO:206, the LCDR1 comprises the amino acid sequence of SEQ ID NO:207, the LCDR2 comprises the amino acid sequence of SEQ ID NO:208, and the LCDR3 comprises the amino acid sequence of SEQ ID NO:209.

In some specific embodiments, in the CD3ε binding region provided herein, the HCDR1 comprises the amino acid sequence of SEQ ID NO:210, the HCDR2 comprises the amino acid sequence of SEQ ID NO:211, the HCDR3 comprises the amino acid sequence of SEQ ID NO:212, the LCDR1 comprises the amino acid sequence of SEQ ID NO:213, the LCDR2 comprises the amino acid sequence of YAS, and the LCDR3 comprises the amino acid sequence of SEQ ID NO:215.

In some specific embodiments, in the CD3ε binding region provided herein, the HCDR1 comprises the amino acid sequence of SEQ ID NO:216, the HCDR2 comprises the amino acid sequence of SEQ ID NO:217, the HCDR3 comprises the amino acid sequence of SEQ ID NO:218, the LCDR1 comprises the amino acid sequence of SEQ ID NO:219, the LCDR2 comprises the amino acid sequence of YAS, and the LCDR3 comprises the amino acid sequence of SEQ ID NO:209.

In one embodiment, provided herein is a binding region that binds CD3ε, comprising a VH comprising a HCDR1, a HCDR2, and a HCDR3 having an amino acid sequence of SEQ ID NOs: 204, 205 and 206, respectively. In another embodiment, provided herein is a binding region that binds CD3ε, comprising a VL comprising a LCDR1, a LCDR2, and a LCDR3 having an amino acid sequence of SEQ ID NOs: 207, 208 and 209, respectively. In another embodiment, provided herein is a binding region that binds CD3ε, comprising: (i) a VH comprising a HCDR1, a HCDR2, and a HCDR3 having an amino acid sequence of SEQ ID NOs: 204, 205 and 206, respectively, and (ii) a VL comprising a LCDR1, a LCDR2, and a LCDR3 having an amino acid sequence of SEQ ID NOs: 207, 208 and 209, respectively.

In one embodiment, provided herein is a binding region that binds CD3ε, comprising a VH comprising a HCDR1, a HCDR2, and a HCDR3 having an amino acid sequence of SEQ ID NOs: 210, 211 and 212, respectively. In another embodiment, provided herein is a binding region that binds CD3ε, comprising a VL comprising a LCDR1, a LCDR2, and a LCDR3 having an amino acid sequence of SEQ ID NOs: 213, 214 and 214, respectively. In another embodiment, provided herein is a binding region that binds CD3ε, comprising: (i) a VH comprising a HCDR1, a HCDR2, and a HCDR3 having an amino acid sequence of SEQ ID NOs: 210, 211 and 212, respectively, and (ii) a VL comprising a LCDR1, a LCDR2, and a LCDR3 having an amino acid sequence of SEQ ID NOs: 213, 214 and 214, respectively.

In one embodiment, provided herein is a binding region that binds CD3ε, comprising a VH comprising a HCDR1, a HCDR2, and a HCDR3 having an amino acid sequence of SEQ ID NOs: 216, 217 and 218, respectively. In another embodiment, provided herein is a binding region that binds CD3ε, comprising a VL comprising a LCDR1, a LCDR2, and a LCDR3 having an amino acid sequence of SEQ ID NOs: 219, 214 and 209, respectively. In another embodiment, provided herein is a binding region that binds CD3ε, comprising: (i) a VH comprising a HCDR1, a HCDR2, and a HCDR3 having an amino acid sequence of SEQ ID NOs: 216, 217 and 218, respectively, and (ii) a VL comprising a LCDR1, a LCDR2, and a LCDR3 having an amino acid sequence of SEQ ID NOs: 219, 214 and 209, respectively.

TABLE 13C
CD3W245 Binding Region Sequences

| | Kabat | Chothia | IMGT |
|---|---|---|---|
| Heavy Chain variable | RYNMN | GFTFSRY | GFTFSRYN |
| region CDR1 | (SEQ ID NO: 204) | (SEQ ID NO: 210) | (SEQ ID NO: 216) |
| Heavy Chain variable | SISTSSNYIYYADSV | STSSNY | ISTSSNYI |
| region CDR2 | KG | (SEQ ID NO: 211) | (SEQ ID NO: 217) |
| | (SEQ ID NO: 205) | | |
| Heavy Chain variable | GWGPFDY | GWGPFD | TRGWGPFDY |
| region CDR3 | (SEQ ID NO: 206) | (SEQ ID NO: 212) | (SEQ ID NO: 218) |
| Light Chain variable | RARQSIGTAIH | RQSIGTA | QSIGTA |
| region CDR1 | (SEQ ID NO: 207) | (SEQ ID NO: 213) | (SEQ ID NO: 219) |
| Light Chain variable | YASESIS | YAS | YAS |
| region CDR2 | (SEQ ID NO: 208) | | |
| Light Chain variable | QQSGSWPYT | SGSWPY | QQSGSWPYT |
| region CDR3 | (SEQ ID NO: 209) | (SEQ ID NO: 215) | (SEQ ID NO: 209) |

VH (SEQ ID NO: 220)
EVQLVESGGGLVKPGGSLRLSCAASGFTFSRYNMNWVRQAPGKGLEWVSSIST
SSNYIYYADSVKGRFTFSRDNAKNSLDLQMSGLRAEDTAIYYCTRGWGPFDYW
GQGTLVTVSS
VL (SEQ ID NO: 221)
DIQMTQSPSSLSASVGDRVTITCRARQSIGTAIHWYQQKPGKAPKLLIKYASESIS
GVPSRFSGSGSGTDFTLTISSLQPEDFATYYCQQSGSWPYTFGQGTKLEIK

In some embodiments, the CD3ε binding region provided herein comprises HCDR1, HCDR2, and HCDR3 as set forth in SEQ ID NO:228. In some embodiments, the CD3ε binding region provided herein comprises LCDR1, LCDR2, and LCDR3 as set forth in SEQ ID NO:229. In some embodiments, the CD3ε binding region provided herein comprises HCDR1, HCDR2, and HCDR3 as set forth in SEQ ID NO:228, and LCDR1, LCDR2, and LCDR3 as set forth in SEQ ID NO:229. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the CD3ε binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:222; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:223, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:224; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:225; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:226; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 227. In some embodiments, the CD3ε binding region is humanized. In some embodiments, the CD3ε binding region comprises an acceptor human framework, e.g., a human immunoglobulin framework or a human consensus framework.

In some specific embodiments, the CD3 binding region provided herein comprises one or more CDRs in Table 13D.

In some specific embodiments, in the CD3ε binding region provided herein, the HCDR1 comprises the amino acid sequence of SEQ ID NO:222, the HCDR2 comprises the amino acid sequence of SEQ ID NO:223, the HCDR3 comprises the amino acid sequence of SEQ ID NO:224, the LCDR1 comprises the amino acid sequence of SEQ ID NO:225, the LCDR2 comprises the amino acid sequence of SEQ ID NO:226, and the LCDR3 comprises the amino acid sequence of SEQ ID NO:227.

In one embodiment, provided herein is a binding region that binds CD3ε, comprising a VH comprising a HCDR1, a HCDR2, and a HCDR3 having an amino acid sequence of SEQ ID NOs: 222, 223 and 225, respectively. In another embodiment, provided herein is a binding region that binds CD3ε, comprising a VL comprising a LCDR1, a LCDR2, and a LCDR3 having an amino acid sequence of SEQ ID NOs: 225, 226 and 227, respectively. In another embodiment, provided herein is a binding region that binds CD3ε, comprising: (i) a VH comprising a HCDR1, a HCDR2, and a HCDR3 having an amino acid sequence of SEQ ID NOs: 222, 223 and 225, respectively, and (ii) a VL comprising a LCDR1, a LCDR2, and a LCDR3 having an amino acid sequence of SEQ ID NOs: 225, 226 and 227, respectively.

TABLE 13D
CD3B450 Binding Region Sequences

| | Kabat |
|---|---|
| Heavy Chain Variable | NNNAAWS |
| Region CDR1 | (SEQ ID NO: 222) |
| Heavy Chain Variable | RTYYRSKWLYDYAVSVKS |
| Region CDR2 | (SEQ ID NO: 223) |
| Heavy Chain Variable | GYSSSFDY |
| Region CDR3 | (SEQ ID NO: 224) |
| Light Chain Variable | TGTSSNIGTYKFVS |
| Region CDR1 | (SEQ ID NO: 225) |
| Light Chain Variable | EVSKRPS |
| Region CDR2 | (SEQ ID NO: 226) |
| Light Chain Variable | VSYAGSGTLL |
| Region CDR3 | (SEQ ID NO: 227) |

VH (SEQ ID NO: 228)
QVQLQQSGPGLVKPSQTLSLTCAISGDSVENNNAAWSWIRQSPSRGLEW
LGRTYYRSKWLYDYAVSVKSRITINPDTSKNQFSLQLNSVTPEDTAVYY
CARGYSSSFDYWGQGTLVTVSS
VL (SEQ ID NO: 229)
QSALTQPASVSGSPGQSITISCTGTSSNIGTYKFVSWYQQHPGKAPKVM
IYEVSKRPSGVSNRFSGSKSGNTASLTISGLQAEDEADYYCVSYAGSGT
LLFGGGTKLTVL

In some embodiments, the CD3ε binding region provided herein comprises HCDR1, HCDR2, and HCDR3 as set forth in SEQ ID NO:246. In some embodiments, the CD3ε binding region provided herein comprises LCDR1, LCDR2, and LCDR3 as set forth in SEQ ID NO:247. In some embodiments, the CD3ε binding region provided herein comprises HCDR1, HCDR2, and HCDR3 as set forth in SEQ ID NO:246, and LCDR1, LCDR2, and LCDR3 as set forth in SEQ ID NO:247. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the CD3ε binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:230, 236 and 242; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:231, 237 and 243, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:232, 238 and 244; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:233, 239 and 245; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NO:234 and 240; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NO:235 or 241. In some embodiments, the CD3ε binding region is humanized. In some embodiments, the CD3ε binding region comprises an acceptor human framework, e.g., a human immunoglobulin framework or a human consensus framework.

In some specific embodiments, the CD3 binding region provided herein comprises one or more CDRs in Table 13E.

In some embodiments, the CD3ε binding region provided herein comprises an HCDR1 comprising an amino acid sequence of any of SEQ ID NO:230, 236 and 242; (ii) an HCDR2 comprising an amino acid sequence of any of SEQ ID NO:231, 237 and 243, (iii) an HCDR3 comprising an amino acid sequence SEQ ID NO:232, 238 and 244; (iv) a LCDR1 comprising an amino acid sequence SEQ ID NO:233, 239 and 245; (v) a LCDR2 comprising an amino acid sequence of SEQ ID NO:234 and 240; and/or (vi) a LCDR3 comprising an amino acid sequence of SEQ ID NO:235 or 241.

In some specific embodiments, in the CD3ε binding region provided herein, the HCDR1 comprises the amino acid sequence of SEQ ID NO:230, the HCDR2 comprises the amino acid sequence of SEQ ID NO:231, the HCDR3 comprises the amino acid sequence of SEQ ID NO:232, the LCDR1 comprises the amino acid sequence of SEQ ID NO:233, the LCDR2 comprises the amino acid sequence of SEQ ID NO:234, and the LCDR3 comprises the amino acid sequence of SEQ ID NO:235.

In some specific embodiments, in the CD3ε binding region provided herein, the HCDR1 comprises the amino acid sequence of SEQ ID NO:236, the HCDR2 comprises the amino acid sequence of SEQ ID NO:237, the HCDR3 comprises the amino acid sequence of SEQ ID NO:238, the LCDR1 comprises the amino acid sequence of SEQ ID NO:239, the LCDR2 comprises the amino acid sequence of DSS, and the LCDR3 comprises the amino acid sequence of SEQ ID NO:241.

In some specific embodiments, in the CD3ε binding region provided herein, the HCDR1 comprises the amino acid sequence of SEQ ID NO:242, the HCDR2 comprises the amino acid sequence of SEQ ID NO:243, the HCDR3 comprises the amino acid sequence of SEQ ID NO:244, the LCDR1 comprises the amino acid sequence of SEQ ID NO:245 the LCDR2 comprises the amino acid sequence of DSS, and the LCDR3 comprises the amino acid sequence of SEQ ID NO:235.

In one embodiment, provided herein is a binding region that binds CD3ε, comprising a VH comprising a HCDR1, a HCDR2, and a HCDR3 having an amino acid sequence of SEQ ID NOs: 230, 231 and 232, respectively. In another embodiment, provided herein is a binding region that binds CD3ε, comprising a VL comprising a LCDR1, a LCDR2, and a LCDR3 having an amino acid sequence of SEQ ID NOs: 233, 234 and 235, respectively. In another embodiment, provided herein is a binding region that binds CD3ε, comprising: (i) a VH comprising a HCDR1, a HCDR2, and a HCDR3 having an amino acid sequence of SEQ ID NOs: 230, 231 and 232, respectively, and (ii) a VL comprising a LCDR1, a LCDR2, and a LCDR3 having an amino acid sequence of SEQ ID NOs: 233, 234 and 235, respectively.

In one embodiment, provided herein is a binding region that binds CD3ε, comprising a VH comprising a HCDR1, a HCDR2, and a HCDR3 having an amino acid sequence of SEQ ID NOs: 236, 237 and 238, respectively. In another embodiment, provided herein is a binding region that binds CD3ε, comprising a VL comprising a LCDR1, a LCDR2, and a LCDR3 having an amino acid sequence of SEQ ID NOs: 239, 240 and 241, respectively. In another embodiment, provided herein is a binding region that binds CD3ε, comprising: (i) a VH comprising a HCDR1, a HCDR2, and a HCDR3 having an amino acid sequence of SEQ ID NOs: 236, 237 and 238, respectively, and (ii) a VL comprising a LCDR1, a LCDR2, and a LCDR3 having an amino acid sequence of SEQ ID NOs: 239, 240 and 241, respectively.

In one embodiment, provided herein is a binding region that binds CD3ε, comprising a VH comprising a HCDR1, a HCDR2, and a HCDR3 having an amino acid sequence of SEQ ID NOs: 242, 243 and 244, respectively. In another embodiment, provided herein is a binding region that binds CD3ε, comprising a VL comprising a LCDR1, a LCDR2, and a LCDR3 having an amino acid sequence of SEQ ID NOs: 245, 240 and 235, respectively. In another embodiment, provided herein is a binding region that binds CD3ε, comprising: (i) a VH comprising a HCDR1, a HCDR2, and a HCDR3 having an amino acid sequence of SEQ ID NOs: 242, 243 and 244, respectively, and (ii) a VL comprising a LCDR1, a LCDR2, and a LCDR3 having an amino acid sequence of SEQ ID NOs: 245, 240 and 235, respectively.

TABLE 13E
CD3B2051-N106A Binding Region Sequences

| | Kabat | Chothia | IMGT |
|---|---|---|---|
| Heavy Chain Variable | RSTMH | GYTFTRS | GYTFTRST |
| Region CDR1 | (SEQ ID NO: 230) | (SEQ ID NO: 236) | (SEQ ID NO: 242) |
| Heavy Chain Variable | YINPSSAYTNYNQKF | NPSSAY | INPSSAYT |
| Region CDR2 | QG | (SEQ ID NO: 237) | (SEQ ID NO: 243) |
| | (SEQ ID NO: 231) | | |
| Heavy Chain Variable | PQVHYDYAGFPY | PQVHYDYAGFP | ASPQVHYDYAGF |
| Region CDR3 | (SEQ ID NO: 232) | (SEQ ID NO: 238) | PY |
| | | | (SEQ ID NO: 244) |
| Light Chain Variable | SASSSVSYMN | SSSVSY | SSVSY |
| Region CDR1 | (SEQ ID NO: 233) | (SEQ ID NO: 239) | (SEQ ID NO: 245) |
| Light Chain Variable | DSSKLAS | DSS | DSS |
| Region CDR2 | (SEQ ID NO: 234) | | |
| Light Chain Variable | QQWSRNPPT | WSRNPP | QQWSRNPPT |
| Region CDR3 | (SEQ ID NO: 235) | (SEQ ID NO: 241) | (SEQ ID NO: 235) |

VH (SEQ ID NO: 246)
QVQLVQSGAEVKKPGSSVKVSCKASGYTFTRSTMHWVKQAPGQGLEWMGYIN
PSSAYTNYNQKFQGRVTLTADKSTSTAYMELSSLRSEDTAVYYCASPQVHYDY
AGFPYWGQGTLVTVSS
VL (SEQ ID NO: 247)
EIVLTQSPATLSASPGERVTLSCSASSSVSYMNWYQQKPGQAPRRLIYDSSKLAS
GVPARFSGSGSGRDYTLTISSLEPEDFAVYYCQQWSRNPPTFGGGTKVEIK

In some embodiments, the CD3ε binding region provided herein comprises HCDR1, HCDR2, and HCDR3 as set forth in SEQ ID NO:312. In some embodiments, the CD3ε binding region provided herein comprises LCDR1, LCDR2, and LCDR3 as set forth in SEQ ID NO:313. In some embodiments, the CD3ε binding region provided herein comprises HCDR1, HCDR2, and HCDR3 as set forth in SEQ ID NO:312, and LCDR1, LCDR2, and LCDR3 as set forth in SEQ ID NO:313.

In other embodiments, the CD3ε binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:306; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:307, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:308; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:309; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:310; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 311. In some embodiments, the CD3ε binding region is humanized. In some embodiments, the CD3ε binding region comprises an acceptor human framework, e.g., a human immunoglobulin framework or a human consensus framework.

In some specific embodiments, the CD3 binding region provided herein comprises one or more CDRs in Table 13F.

In some embodiments, the CD3ε binding region provided herein comprises an HCDR1 comprising an amino acid sequence of SEQ ID NO:306; (ii) an HCDR2 comprising an amino acid sequence of SEQ ID NO:307, (iii) an HCDR3 comprising an amino acid sequence of SEQ ID NO:308; (iv) a LCDR1 comprising an amino acid sequence of SEQ ID NO:309; (v) a LCDR2 comprising an amino acid sequence of SEQ ID NO:310; and/or (vi) a LCDR3 comprising an amino acid sequence of SEQ ID NO:311.

In some specific embodiments, in the CD3ε binding region provided herein, the HCDR1 comprises the amino acid sequence of SEQ ID NO:306, the HCDR2 comprises the amino acid sequence of SEQ ID NO:307, the HCDR3 comprises the amino acid sequence of SEQ ID NO:308, the LCDR1 comprises the amino acid sequence of SEQ ID NO:309, the LCDR2 comprises the amino acid sequence of SEQ ID NO:310, and the LCDR3 comprises the amino acid sequence of SEQ ID NO:311.

In one embodiment, provided herein is a binding region that binds CD3ε, comprising a VH comprising a HCDR1, a HCDR2, and a HCDR3 having an amino acid sequence of SEQ ID NOs: 306, 307 and 308, respectively. In another embodiment, provided herein is a binding region that binds CD3ε, comprising a VL comprising a LCDR1, a LCDR2, and a LCDR3 having an amino acid sequence of SEQ ID NOs: 309, 310 and 311, respectively. In another embodiment, provided herein is a binding region that binds CD3ε, comprising: (i) a VH comprising a HCDR1, a HCDR2, and a HCDR3 having an amino acid sequence of SEQ ID NOs: 306, 307 and 308, respectively, and (ii) a VL comprising a LCDR1, a LCDR2, and a LCDR3 having an amino acid sequence of SEQ ID NOs: 309, 310 and 311, respectively.

TABLE 13F
CD3B376 Binding Region Sequences

| | |
|---|---|
| Heavy Chain Variable | NNNAAWS |
| Region CDR1 | (SEQ ID NO: 306) |
| Heavy Chain Variable | RTYYRSKWLYDYAVSVKS |
| Region CDR2 | (SEQ ID NO: 307) |
| Heavy Chain Variable | GYSSSFDY |
| Region CDR3 | (SEQ ID NO: 308) |
| Light Chain Variable | TGTSSNIGTYKFVS |
| Region CDR1 | (SEQ ID NO: 309) |
| Light Chain Variable | EVSKRPS |
| Region CDR2 | (SEQ ID NO: 310) |
| Light Chain Variable | VSYAGSGTLL |
| Region CDR3 | (SEQ ID NO: 311) |

VH (SEQ ID NO: 312)
QVQLQQSGPRLVRPSQTLSLTCAISGDSVENNNAAWSWIRQSPSRGLEW
LGRTYYRSKWLYDYAVSVKSRITVNPDTSRNQFTLQLNSVTPEDTALYY
CARGYSSSFDYWGQGTLVTVSS
VL (SEQ ID NO: 313)
QSALTQPASVSGSPGQSITISCTGTSSNIGTYKFVSWYQQHPDKAPKVL
LYEVSKRPSGVSSRFSGSKSGNTASLTISGLQAEDQADYHCVSYAGSGT
LLFGGGTKLTVL
HC (SEQ ID NO: 314)
QVQLQQSGPRLVRPSQTLSLTCAISGDSVENNNAAWSWIRQSPSRGLEW
LGRTYYRSKWLYDYAVSVKSRITVNPDTSRNQFTLQLNSVTPEDTALYY
CARGYSSSFDYWGQGTLVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPPCPAPEAAGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFLLYSKLTVDKSRWQEGNVFSCSVMHEALHNHYTQKSL
SLSLGK
LC (SEQ ID NO: 315)
QSALTQPASVSGSPGQSITISCTGTSSNIGTYKFVSWYQQHPDKAPKVL
LYEVSKRPSGVSSRFSGSKSGNTASLTISGLQAEDQADYHCVSYAGSGT
LLFGGGTKLTVLGQPKAAPSVTLFPPSSEELQANKATLVCLISDFYPGA
VTVAWKADSSPVKAGVETTTPSKQSNNKYAASSYLSLTPEQWKSHRSYS
CQVTHEGSTVEKTVAPTECS

In some embodiments, the CD3ε binding region further comprises one or more framework regions. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system. In some embodiments, the CD3ε binding region further comprises one or more framework regions of SEQ ID NOs: 220, 221, 228, 229, 246 or 247.

In some embodiments, the CD3ε binding region provided herein comprises a VH comprising the amino acid sequence of SEQ ID NO:220, and a VL comprising the amino acid sequence of SEQ ID NO:221.

In some embodiments, the CD3ε binding region provided herein comprises a VH comprising the amino acid sequence of SEQ ID NO:228, and a VL comprising the amino acid sequence of SEQ ID NO:229.

In some embodiments, the CD3ε binding region provided herein comprises a VH comprising the amino acid sequence of SEQ ID NO:246, and a VL comprising the amino acid sequence of SEQ ID NO:247.

In some embodiments, the CD3ε binding region provided herein comprises a VH comprising the amino acid sequence of SEQ ID NO:312, and a VL comprising the amino acid sequence of SEQ ID NO:313.

In some embodiments, the CD3ε binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:200, 202, 220, 228, 246 or 312, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:201, 203, 221, 229, 247 or 313.

In some embodiments, the CD3ε binding region provided herein binds to the same epitope as a CD3ε binding region comprising a VH comprising the amino acid sequence of SEQ ID NO: 201, and a VL comprising the amino acid sequence of SEQ ID NO:202.

In some embodiments, the CD3ε binding region provided herein binds to the same epitope as a CD3ε binding region comprising a VH comprising the amino acid sequence of SEQ ID NO: 203, and a VL comprising the amino acid sequence of SEQ ID NO:204.

In some embodiments, the CD3ε binding region provided herein binds to the same epitope as a CD3ε binding region comprising a VH comprising the amino acid sequence of SEQ ID NO: 220, and a VL comprising the amino acid sequence of SEQ ID NO:221.

In some embodiments, the CD3ε binding region provided herein binds to the same epitope as a CD3ε binding region comprising a VH comprising the amino acid sequence of SEQ ID NO: 228, and a VL comprising the amino acid sequence of SEQ ID NO:229.

In some embodiments, the CD3ε binding region provided herein binds to the same epitope as a CD3ε binding region comprising a VH comprising the amino acid sequence of SEQ ID NO: 246, and a VL comprising the amino acid sequence of SEQ ID NO:247.

In some embodiments, the CD3ε binding region provided herein binds to the same epitope as a CD3ε binding region comprising a VH comprising the amino acid sequence of SEQ ID NO: 312, and a VL comprising the amino acid sequence of SEQ ID NO:313.

In some specific embodiments, the CD3ε binding region is a scFv. In some embodiments, the CD3ε binding scFv comprises one or more amino acid substitutions, such as those stabilizing scFv.

In some specific embodiments, the CD3ε binding region is an spFv. In some embodiments, the CD3ε binding spFv comprises a linker comprising at least one Cys residue that serves as an anchor point for forming a disulfide bond between the VH and the linker. In some embodiments, the CD3ε binding spFv comprises a linker comprising at least one Cys residue that serves as an anchor point for forming a disulfide bond between the VL and the linker. In some embodiments, the CD3 binding spFv comprises a linker comprising at least two Cys residues that serve as anchor points for forming a disulfide bonds between the VH and the linker and the VL and the linker.

In some embodiments, the CD3ε binding scFv comprises the VH and VL sequences disclosed in any of Tables 13B to 13F. In some embodiments, the CD3ε binding scFv comprises a VH comprising the sequence of SEQ ID NO: 202, 220, 228, 246 or 312. In some embodiments, the CD3ε binding scFv comprises a VL comprising the sequence of SEQ ID NO: 203, 221, 229, 247 or 313. In some embodiments, the CD3ε binding scFv comprises a VH comprising the sequence of SEQ ID NO:202 and a VL comprising the sequence of SEQ ID NO: 203. In some embodiments, the CD3ε binding scFv comprises a VH comprising the sequence of SEQ ID NO:220 and a VL comprising the sequence of SEQ ID NO:221. In some embodiments, the CD3ε binding scFv comprises a VH comprising the sequence of SEQ ID NO: 228 and a VL comprising the sequence of SEQ ID NO:229. In some embodiments, the CD3ε binding scFv comprises a VH comprising the sequence of SEQ ID NO:246 and a VL comprising the sequence of SEQ ID NO:247. In some embodiments, the CD3ε binding scFv comprises a VH comprising the sequence of SEQ ID NO:312 and a VL comprising the sequence of SEQ ID NO: 313.

In some embodiments, the CD3ε binding spFv comprises a VH comprising the sequence of SEQ ID NO:200 and a VL comprising the sequence of SEQ ID NO:201.

In specific embodiments, any of the VH and the VL domains described herein that bind CD3ε may be engineered into scFv or spFv format in either VH-linker-VL or VL-linker-VH orientation. Alternatively, the VH and VL domains may be engineered into scFv format without the use of a linker in either the VH-VL or VL-VH orientation.

In specific embodiments, the CD3ε binding scFv or spFv comprises the VH comprising SEQ ID NO:200 and the VL comprising SEQ ID NO:201. In particular embodiments, the VH comprising SEQ ID NO:200 and the VL comprising SEQ ID NO:201 are fused directly with one another without a linker in the scFv. In some embodiments, the VH comprising SEQ ID NO:200 is fused to the N terminus of the VL comprising SEQ ID NO:201 in the VH-VL orientation. In other embodiments, the VH comprising SEQ ID NO:200 is fused to the C terminus of the VL comprising SEQ ID NO:201 in the VL-VH orientation. In some embodiments, the VH comprising SEQ ID NO:200 and the VL comprising SEQ ID NO:201 are linked with a linker in the scFv or spFv. In some embodiments, the VH comprising SEQ ID NO: 200 and the VL comprising SEQ ID NO:201 and the linker are fused in the VH-linker-VL orientation in the scFv or spFv. In other embodiments, the VH comprising SEQ ID NO:201 and the VL comprising SEQ ID NO:200 and the linker are fused in the VL-linker-VH orientation in the scFv or spFv. In some embodiments described in the present paragraph, the linker connecting the VH and VL in the scFv comprises a sequence of any one of SEQ ID NOs: 258-292. In some embodiments described in the present paragraph, the linker connecting the VH and VL in the spFv comprises at least one Cys residue that serves as an anchor point for forming a disulfide bond between the VH and the linker. In some embodiments, the linker connecting the VH and VL in the spFv comprises at least one Cys residue that serves as an anchor point for forming a disulfide bond between the VL and the linker. In some embodiments, the linker connecting the VH and VL in the spFv comprises a first Cys residues that serve as an anchor point for forming a disulfide bond between the VH and the linker and a second Cys residues that serve as an anchor point for forming a disulfide bond between the VL and the linker. In one embodiment the linker comprises an amino acid sequence of SEQ ID NO:292.

In specific embodiments, the CD3ε binding scFv comprises the VH comprising SEQ ID NO: 203 and the VL comprising SEQ ID NO:204. In particular embodiments, the VH comprising SEQ ID NO:203 and the VL comprising SEQ ID NO:204 are fused directly with one another without a linker in the scFv. In some embodiments, the VH comprising SEQ ID NO:203 is fused to the N terminus of the VL comprising SEQ ID NO:204 in the VH-VL orientation. In other embodiments, the VH comprising SEQ ID NO:203 is fused to the C terminus of the VL comprising SEQ ID NO:204 in the VL-VH orientation. In some embodiments, the VH comprising SEQ ID NO:203 and the VL comprising SEQ ID NO:204 are linked with a linker in the scFv. In some embodiments, the VH comprising SEQ ID NO:203 and the VL comprising SEQ ID NO:204 and the linker are fused in the VH-linker-VL orientation in the scFv. In other embodiments, the VH comprising SEQ ID NO:203 and the VL comprising SEQ ID NO:204 and the linker are fused in the VL-linker-VH orientation in the scFv. In some embodiments described in the present paragraph, the linker connecting the VH and VL in the scFv comprises a sequence selected from SEQ ID NOs: 258-292.

In specific embodiments, the CD3ε binding scFv comprises the VH comprising SEQ ID NO: 220 and the VL comprising SEQ ID NO:221. In particular embodiments, the VH comprising SEQ ID NO:220 and the VL comprising SEQ ID NO:221 are fused directly with one another without a linker in the scFv. In some embodiments, the VH comprising SEQ ID NO:220 is fused to the N terminus of the VL comprising SEQ ID NO:221 in the VH-VL orientation. In other embodiments, the VH comprising SEQ ID NO:220 is fused to the C terminus of the VL comprising SEQ ID NO:221 in the VL-VH orientation. In some embodiments, the VH comprising SEQ ID NO:220 and the VL comprising SEQ ID NO:221 are linked with a linker in the scFv. In some embodiments, the VH comprising SEQ ID NO:220 and the VL comprising SEQ ID NO:221 and the linker are fused in the VH-linker-VL orientation in the scFv. In other embodiments, the VH comprising SEQ ID NO:220 and the VL comprising SEQ ID NO:221 and the linker are fused in the VL-linker-VH orientation in the scFv. In some embodiments described in the present paragraph, the linker connecting the VH and VL in the scFv comprises a sequence selected from SEQ ID NOs: 258-292.

In specific embodiments, the CD3ε binding scFv comprises the VH comprising SEQ ID NO: 228 and the VL comprising SEQ ID NO:229. In particular embodiments, the VH comprising SEQ ID NO:228 and the VL comprising SEQ ID NO:229 are fused directly with one another without a linker in the scFv. In some embodiments, the VH comprising SEQ ID NO:228 is fused to the N terminus of the VL comprising SEQ ID NO:229 in the VH-VL orientation. In other embodiments, the VH comprising SEQ ID NO:228 is fused to the C terminus of the VL comprising SEQ ID NO:229 in the VL-VH orientation. In some embodiments, the VH comprising SEQ ID NO:228 and the VL comprising SEQ ID NO:229 are linked with a linker in the scFv. In some embodiments, the VH comprising SEQ ID NO:228 and the VL comprising SEQ ID NO:229 and the linker are fused in the VH-linker-VL orientation in the scFv. In other embodiments, the VH comprising SEQ ID NO:228 and the VL comprising SEQ ID NO:229 and the linker are fused in the VL-linker-VH orientation in the scFv. In some embodiments described in the present paragraph, the linker connecting the VH and VL in the scFv comprises a sequence selected from SEQ ID NOs: 258-292.

In specific embodiments, the CD3ε binding scFv comprises the VH comprising SEQ ID NO: 246 and the VL comprising SEQ ID NO:247. In particular embodiments, the VH comprising SEQ ID NO:246 and the VL comprising SEQ ID NO:247 are fused directly with one another without a linker in the scFv. In some embodiments, the VH comprising SEQ ID NO:246 is fused to the N terminus of the VL comprising SEQ ID NO:247 in the VH-VL orientation. In other embodiments, the VH comprising SEQ ID NO:246 is fused to the C terminus of the VL comprising SEQ ID NO:247 in the VL-VH orientation. In some embodiments, the VH comprising SEQ ID NO:246 and the VL comprising SEQ ID NO:247 are linked with a linker in the scFv. In some embodiments, the VH comprising SEQ ID NO:246 and the VL comprising SEQ ID NO:247 and the linker are fused in the VH-linker-VL orientation in the scFv. In other embodiments, the VH comprising SEQ ID NO:246 and the VL comprising SEQ ID NO:247 and the linker are fused in the VL-linker-VH orientation in the scFv. In some embodiments described in the present paragraph, the linker connecting the VH and VL in the scFv comprises a sequence selected from SEQ ID NOs: 258-292.

In specific embodiments, the CD3ε binding scFv comprises the VH comprising SEQ ID NO: 312 and the VL comprising SEQ ID NO:313. In particular embodiments, the VH comprising SEQ ID NO:312 and the VL comprising SEQ ID NO:313 are fused directly with one another without a linker in the scFv. In some embodiments, the VH comprising SEQ ID NO:312 is fused to the N terminus of the VL comprising SEQ ID NO:313 in the VH-VL orientation. In other embodiments, the VH comprising SEQ ID NO:312 is fused to the C terminus of the VL comprising SEQ ID NO:313 in the VL-VH orientation. In some embodiments, the VH comprising SEQ ID NO:312 and the VL comprising SEQ ID NO:313 are linked with a linker in the scFv. In some embodiments, the VH comprising SEQ ID NO:312 and the VL comprising SEQ ID NO:313 and the linker are fused in the VH-linker-VL orientation in the scFv. In other embodiments, the VH comprising SEQ ID NO:312 and the VL comprising SEQ ID NO:313 and the linker are fused in the VL-linker-VH orientation in the scFv. In some embodiments described in the present paragraph, the linker connecting the VH and VL in the scFv comprises a sequence selected from SEQ ID NOs: 258-292.

In some embodiments, the CD3ε binding scFv or spFv is in the orientation of VL-linker-VH. In some embodiments, the scFv or spFv comprises an amino acid sequence of SEQ ID NO: 248. In some embodiments, the scFv comprises an amino acid sequence of SEQ ID NO: 250. In some embodiments, the scFv comprises an amino acid sequence of SEQ ID NO:252. In some embodiments, the scFv comprises an amino acid sequence of SEQ ID NO:254.

In particular embodiments, the CD3ε binding scFv or spFv is in the orientation of VH-linker-VL. In some embodiments, the scFv comprises an amino acid sequence of SEQ ID NO: 249. In some embodiments, the scFv comprises an amino acid sequence of SEQ ID NO:251. In some embodiments, the scFv comprises an amino acid sequence of SEQ ID NO:253.

TABLE 14
CD3ϵ-Binding spFv

| Binding domain | |
|---|---|
| name | spFv-LH amino acid Sequence |
| CD3B2030- | EIVLTQSPATLSASPGERVTLSCSASSSVSY |
| N106A spFv | MNWYQQKPGCAPRRWIYDSSKLASGVPARFS |
| | GSGSGRDYTLTISSLEPEDFAVYYCQQWSRN |
| | PPTFGGGTKVEIKGGGSGGSGGCPPCGGSGG |
| | QVQLVQSGAEVKKPGSSVKVSCKASGYTFTR |
| | STMHWVKQAPGQGLEWIGYINPSSAYTNYNQ |
| | KFQGRVTLTADKSTSTAYMELSSLRSEDTAV |
| | YYCASPQVHYDYAGFPYWGCGTLVTVSS |
| | (SEQ ID NO: 248) |

TABLE 15
CD38-Binding scFvs

| Binding | scFv-HL | scFv-LH |
|---|---|---|
| domain name | amino acid Sequence | amino acid Sequence |
| CD3B2030- | QVQLVQSGAEVKKPGSSVK | EIVLTQSPATLSASPGERVTLSCSASS |
| N106A | VSCKASGYTFTRSTMHWV | SVSYMNWYQQKPGQAPRRWIYDSS |
| | KQAPGQGLEWIGYINPSSA | KLASGVPARFSGSGSGRDYTLTISSL |
| | YTNYNQKFQGRVTLTADKS | EPEDFAVYYCQQWSRNPPTFGGGT |
| | TSTAYMELSSLRSEDTAVY | KVEIKGGSEGKSSGSGSESKSTGGSQ |
| | YCASPQVHYDYAGFPYWG | VQLVQSGAEVKKPGSSVKVSCKAS |
| | QGTLVTVSSGGSEGKSSGS | GYTFTRSTMHWVKQAPGQGLEWIG |
| | GSESKSTGGSEIVLTQSPAT | YINPSSAYTNYNQKFQGRVTLTADK |
| | LSASPGERVTLSCSASSSVS | STSTAYMELSSLRSEDTAVYYCASP |
| | YMNWYQQKPGQAPRRWIY | QVHYDYAGFPYWGQGTLVTVSS |
| | DSSKLASGVPARFSGSGSGR | (SEQ ID NO: 250) |
| | DYTLTISSLEPEDFAVYYCQ | |
| | QWSRNPPTFGGGTKVEIK | |
| | (SEQ ID NO: 249) | |
| CD3W245 | EVQLVESGGGLVKPGGSLR | DIQMTQSPSSLSASVGDRVTITCRAR |
| | LSCAASGFTFSRYNMNWVR | QSIGTAIHWYQQKPGKAPKLLIKYA |
| | QAPGKGLEWVSSISTSSNYI | SESISGVPSRFSGSGSGTDFTLTISSL |
| | YYADSVKGRFTFSRDNAKN | QPEDFATYYCQQSGSWPYTFGQGT |
| | SLDLQMSGLRAEDTAIYYC | KLEIKGGSEGKSSGSGSESKSTGGSE |
| | TRGWGPFDYWGQGTLVTV | VQLVESGGGLVKPGGSLRLSCAASG |
| | SSGGSEGKSSGSGSESKSTG | FTFSRYNMNWVRQAPGKGLEWVSS |
| | GSDIQMTQSPSSLSASVGDR | ISTSSNYIYYADSVKGRFTFSRDNAK |
| | VTITCRARQSIGTAIHWYQQ | NSLDLQMSGLRAEDTAIYYCTRGW |
| | KPGKAPKLLIKYASESISGV | GPFDYWGQGTLVTVSS |
| | PSRFSGSGSGTDFTLTISSLQ | (Seq ID NO: 252) |
| | PEDFATYYCQQSGSWPYTF | |
| | GQGTKLEIK | |
| | (Seq ID NO: 251) | |
| CD3B2051- | QVQLVQSGAEVKKPGSSVK | EIVLTQSPATLSASPGERVTLSCSASS |
| N106A | VSCKASGYTFTRSTMHWV | SVSYMNWYQQKPGQAPRRLIYDSS |
| | KQAPGQGLEWMGYINPSSA | KLASGVPARFSGSGSGRDYTLTISSL |
| | YTNYNQKFQGRVTLTADKS | EPEDFAVYYCQQWSRNPPTFGGGT |
| | TSTAYMELSSLRSEDTAVY | KVEIKGGSEGKSSGSGSESKSTGGSQ |
| | YCASPQVHYDYAGFPYWG | VQLVQSGAEVKKPGSSVKVSCKAS |
| | QGTLVTVSSGGSEGKSSGS | GYTFTRSTMHWVKQAPGQGLEWM |
| | GSESKSTGGSEIVLTQSPAT | GYINPSSAYTNYNQKFQGRVTLTAD |
| | LSASPGERVTLSCSASSSVS | KSTSTAYMELSSLRSEDTAVYYCAS |
| | YMNWYQQKPGQAPRRLIY | PQVHYDYAGFPYWGQGTLVTVSS |
| | DSSKLASGVPARFSGSGSGR | (SEQ ID NO: 254) |
| | DYTLTISSLEPEDFAVYYCQ | |
| | QWSRNPPTFGGGTKVEIK | |
| | (SEQ ID NO: 253) | |

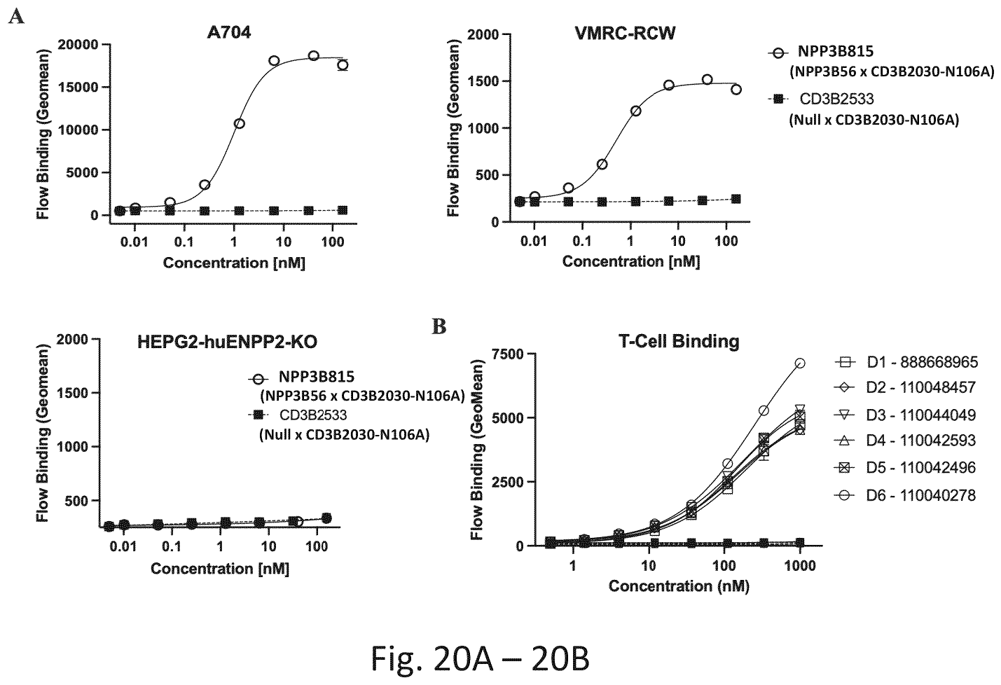

In some embodiments, the ENPP3×CD3 binding protein provided herein has the structure as illustrated in FIG. 20. In specific embodiments, the ENPP3×CD3 binding protein provided herein comprises a scFv that binds CD3ε and a Fab that binds ENPP3, and the binding protein further comprises a Fc region. In specific embodiments, the ENPP3×CD3 binding protein provided herein comprises: (i) a first polypeptide comprising a scFv or spFv that binds CD3ε that is linked to one arm of an antibody Fc region, (ii) a second polypeptide comprising the VH domain that binds ENPP3 that is linked to the other arm of an antibody Fc region, and (iii) a third polypeptide comprising the VL domain that binds ENPP3, wherein the VH domain and the VL domain forms a Fab that binds ENPP3, and the first polypeptide and the second polypeptide forms a Fc region.

In other specific embodiments, the ENPP3×CD3 binding protein provided herein comprises: (i) a first polypeptide comprising a scFv or spFv that binds ENPP3 that is linked to one arm of an antibody Fc region, (ii) a second polypeptide comprising the VH domain that binds CD3ε that is linked to the other arm of an antibody Fc region, and (iii) a third polypeptide comprising the VL domain that binds CD3ε, wherein the VH domain and the VL domain forms a Fab that binds ENPP3, and the first polypeptide and the second polypeptide forms a Fc region.

In some embodiments, the ENPP3×CD3 binding protein provided herein comprises one or more constant region mutation.

In some embodiments, the Fc regions comprise one or more modifications that facilitate heterodimerization of the first and second polypeptides. In some embodiments, one arm of the Fc region comprises T366W substitution, and the other arm of the Fc region comprises the T366S, L368A, Y407V substitutions. In some embodiments, the Fc region comprises one or more mutations that reduces binding affinity of the Fc domain to Fc receptors. In particular embodiments, one arm of the Fc region comprises L234A, L235A, and D265S (AAS) mutations. In particular embodiments, the second arm of the Fc region comprises L234A, L235A, and D265S (AAS) mutations. In particular embodiments, each the first and second arms of the Fc region comprises L234A, L235A, and D265S (AAS) mutations.

In some embodiments, the ENPP3×CD3 binding protein provided herein comprises: (i) a first polypeptide comprising a scFv or spFv that binds CD3ε that is linked to one arm of an antibody Fc region, (ii) a second polypeptide comprising the VH domain that binds ENPP3 that is linked to the other arm of an antibody Fc region, and (iii) a third polypeptide comprising the VL domain that binds ENPP3, wherein the VH domain and the VL domain forms a Fab that binds ENPP3, and the first polypeptide and the second polypeptide forms a Fc region; further wherein the Fc region of the first polypeptide comprises the T366S, L368A, Y407V substitutions, and the Fc region of the second polypeptide comprises the T366W substitution. In specific embodiments described in this paragraph, the Fc region of the first polypeptide further comprises the L234A, L235A, and D265S (AAS) mutations. In specific embodiments described in this paragraph, the Fc region of the second polypeptide further comprises the L234A, L235A, and D265S (AAS) mutations. In specific embodiments described in this paragraph, both Fc regions of the first and second polypeptides further comprise the L234A, L235A, and D265S (AAS) mutations.

In alternative embodiments, the ENPP3×CD3 binding protein provided herein comprises: (i) a first polypeptide comprising a scFv or spFv that binds CD3ε that is linked to one arm of an antibody Fc region, (ii) a second polypeptide comprising the VH domain that binds ENPP3 that is linked to the other arm of an antibody Fc region, and (iii) a third polypeptide comprising the VL domain that binds ENPP3, wherein the VH domain and the VL domain forms a Fab that binds ENPP3, and the first polypeptide and the second polypeptide forms a Fc region; further wherein the Fc region of the first polypeptide comprises the T366W substitution and the Fc region of the second polypeptide comprises the T366S, L368A, Y407V substitutions. In specific embodiments described in this paragraph, the Fc region of the first polypeptide further comprises the L234A, L235A, and D265S (AAS) mutations. In specific embodiments described in this paragraph, the Fc region of the second polypeptide further comprises the L234A, L235A, and D265S (AAS) mutations. In specific embodiments described in this paragraph, both Fc regions of the first and second polypeptides further comprise the L234A, L235A, and D265S (AAS) mutations.

In specific embodiments, the ENPP3×CD3 binding protein provided herein comprises: (i) a first polypeptide comprising a scFv or spFv that binds CD3ε that is linked to one arm of an antibody Fc region, (ii) a second polypeptide comprising the VH domain that binds ENPP3 that is linked to the other arm of an antibody Fc region, and (iii) a third polypeptide comprising the VL domain that binds ENPP3, wherein the VH domain and the VL domain forms a Fab that binds ENPP3, and the first polypeptide and the second polypeptide forms a Fc region. In some embodiments, the first polypeptide comprises an amino acid sequence of SEQ ID NO: 255. In some embodiments, the second polypeptide comprising an amino acid sequence of SEQ ID NO:256. In some embodiments, the third polypeptide comprising an amino acid sequence of SEQ ID NO:257.

In a specific embodiment, the ENPP3×CD3 binding protein provided herein comprises: (i) a first polypeptide comprising a scFv or spFv that binds CD3ε that is linked to one arm of an antibody Fc region, (ii) a second polypeptide comprising the VH domain that binds ENPP3 that is linked to the other arm of an antibody Fc region, and (iii) a third polypeptide comprising the VL domain that binds ENPP3, wherein the VH domain and the VL domain forms a Fab that binds ENPP3, and the first polypeptide and the second polypeptide forms a Fc region, wherein the first polypeptide comprises an amino acid sequence of SEQ ID NO:255, the second polypeptide comprises an amino acid sequence of SEQ ID NO:256, and the third polypeptide comprises an amino acid sequence of SEQ ID NO:257 (see Table 16A). In some embodiments, the ENPP3×CD3 binding protein provided herein is encoded by nucleic acid sequences shown in Table 16B. In some embodiments, the ENPP3×CD3 binding protein provided herein is encoded by nucleic acid sequences shown in Table 16C.

TABLE 16A
Sequences of NPP3B815

| | Amino Acids |
|---|---|
| Heavy Chain 1 | EIVLTQSPATLSASPGERVTLSCSASSSVSYMNWYQQKPGCAPRRW |
| (1^{st} polypeptide) | IYDSSKLASGVPARFSGSGSGRDYTLTISSLEPEDFAVYYCQQWSR |
| | NPPTFGGGTKVEIKGGGSGGSGGCPPCGGSGGQVQLVQSGAEVKK |
| | PGSSVKVSCKASGYTFTRSTMHWVKQAPGQGLEWIGYINPSSAYT |
| | NYNQKFQGRVTLTADKSTSTAYMELSSLRSEDTAVYYCASPQVHY |
| | DYAGFPYWGCGTLVTVSSEPKSSDKTHTCPPCPAPEAAGGPSVFLF |
| | PPKPKDTLMISRTPEVTCVVVSVSHEDPEVKFNWYVDGVEVHNAK |
| | TKPREEQYNSTYRVVSVLTVLHQDWLNGKEYKCKVSNKALPAPIE |
| | KTISKAKGQPREPQVYTLPPSREEMTKNQVSLWCLVKGFYPSDIAV |
| | EWESNGQPENNYKTTPPVLDSDGSFFLYSKLTVDKSRWQQGNVFS |
| | CSVMHEALHNHYTQKSLSLSPGK (SEQ ID NO: 255) |
| Heavy Chain 2 | EVQLVESGGGRVKPGGSLRLSCAASGFTFSDYSMNWVRQAPGKG |
| (2^{nd} polypeptide) | LEWVSSISSISSYVKYADSVKGRFTMSRDNAKNSLFLQMNSLRDED |
| | MAVYYCARGHYFDYWGQGTLVTVSSASTKGPSVFPLAPSSKSTSG |
| | GTAALGCLVKDYFPEPVTVSWNSGALTSGVHTFPAVLQSSGLYSLS |
| | SVVTVPSSSLGTQTYICNVNHKPSNTKVDKKVEPKSCDKTHTCPPC |
| | PAPEAAGGPSVFLFPPKPKDTLMISRTPEVTCVVVSVSHEDPEVKFN |
| | WYVDGVEVHNAKTKPREEQYNSTYRVVSVLTVLHQDWLNGKEY |
| | KCKVSNKALPAPIEKTISKAKGQPREPQVYTLPPSREEMTKNQVSL |
| | SCAVKGFYPSDIAVEWESNGQPENNYKTTPPVLDSDGSFFLVSKLT |
| | VDKSRWQQGNVFSCSVMHEALHNRFTQKSLSLSPGK (SEQ ID |
| | NO: 256) |
| Light Chain | EIVMTQSPATLSVSPGERATLSCRASQSVSSNLAWYQQKPGLAPRL |
| (3^{rd} polypeptide) | LIYGASTRATGIPARFSGSGSGTEFTLTISSLQSEDFAVYYCQQYNN |
| | WPRTFGQGTKVEIKRTVAAPSVFIFPPSDEQLKSGTASVVCLLNNF |
| | YPREAKVQWKVDNALQSGNSQESVTEQDSKDSTYSLSSTLTLSKA |
| | DYEKHKVYACEVTHQGLSSPVTKSFNRGEC (SEQ ID NO: 257) |

TABLE 16B
DNA Sequences Encoding NPP3B815

| | Polynucleotide |
|---|---|
| Heavy Chain 1 | GAGATCGTGCTGACCCAGTCTCCTGCCACACTGAGTGCTTCTCC |
| (1^{st} poly- | AGGCGAGAGAGTGACCCTGTCCTGCTCCGCTTCCTCCTCCGTGT |
| nucleotide) | CCTACATGAACTGGTATCAGCAGAAGCCCGGCTGCGCCCCTAGA |
| | AGATGGATCTACGACTCCTCCAAGCTGGCCTCTGGCGTGCCTGC |
| | TAGATTTTCCGGCTCTGGCTCTGGCAGAGACTATACCCTGACAA |
| | TCTCCAGCCTGGAACCTGAGGACTTCGCCGTGTACTACTGCCAG |
| | CAGTGGTCTAGGAACCCTCCTACCTTTGGCGGAGGCACCAAGGT |
| | GGAAATCAAGGGCGGAGGTTCTGGTGGATCTGGCGGATGTCCTC |
| | CTTGTGGTGGAAGCGGCGGACAGGTTCAGCTGGTTCAGTCTGGC |
| | GCCGAAGTGAAGAAACCTGGCTCCTCCGTGAAAGTGTCCTGCAA |
| | GGCTTCCGGCTACACCTTTACCAGATCCACCATGCACTGGGTCA |
| | AGCAGGCCCCTGGACAAGGCTTGGAGTGGATCGGCTACATCAA |
| | CCCCAGCTCCGCCTACACCAACTACAACCAGAAATTCCAGGGCA |
| | GAGTGACCCTGACCGCCGACAAGTCTACCTCCACCGCCTACATG |
| | GAACTGTCCAGCCTGAGATCTGAGGACACCGCCGTGTACTACTG |
| | CGCCTCTCCTCAGGTTCACTACGACTACGCCGGCTTTCCTTATTG |
| | GGGCTGTGGCACCCTGGTCACCGTTTCTTCTGAGCCCAAATCTA |
| | GCGACAAAACTCACACATGCCCACCGTGCCCAGCACCTGAAGC |
| | CGCCGGGGGACCGTCAGTCTTCCTCTTCCCCCCAAAACCCAAGG |
| | ACACCCTCATGATCTCCCGGACCCCTGAGGTCACATGCGTGGTG |
| | GTGAGCGTGAGCCACGAAGACCCTGAGGTCAAGTTCAACTGGT |
| | ACGTGGACGGCGTGGAGGTGCATAATGCCAAGACAAAGCCGCG |
| | GGAGGAGCAGTACAACAGCACGTACCGTGTGGTCAGCGTCCTC |
| | ACCGTCCTGCACCAGGACTGGCTGAATGGCAAGGAGTACAAGT |
| | GCAAGGTGTCGAACAAAGCCCTCCCAGCCCCCATCGAGAAAAC |
| | CATCTCCAAAGCCAAAGGGCAGCCCCGAGAACCACAGGTGTAC |
| | ACCCTGCCCCCATCCCGGGAGGAGATGACCAAGAACCAGGTCA |
| | GCCTGTGGTGCCTGGTCAAAGGCTTCTATCCCAGCGACATCGCC |
| | GTGGAGTGGGAGAGCAATGGGCAGCCGGAGAACAACTACAAGA |
| | CCACGCCTCCCGTGCTGGACTCCGACGGCTCCTTCTTCCTCTACA |
| | GCAAGCTCACCGTGGACAAGAGCAGATGGCAGCAGGGGAACGT |
| | CTTCTCATGCTCCGTGATGCATGAGGCTCTGCACAACCACTACA |
| | CGCAGAAGTCTCTCTCCCTGTCTCCGGGAAAA (SEQ ID NO: 320) |
| Heavy Chain 2 | GAGGTGCAGCTGGTTGAATCTGGCGGCGGAAGAGTGAAGCCTG |
| (2^{nd} poly- | GCGGATCTCTGAGACTGTCTTGTGCCGCCTCTGGCTTCACCTTCT |
| nucleotide) | CCGACTACTCCATGAACTGGGTCCGACAGGCTCCTGGCAAAGGC |
| | CTGGAATGGGTGTCCTCTATCTCCTCCATCTCCAGCTACGTGAA |
| | GTACGCCGACTCCGTGAAGGGCAGATTCACCATGTCCAGAGAC |
| | AACGCCAAGAACTCCCTGTTCCTGCAGATGAACAGCCTGCGCGA |
| | CGAGGACATGGCCGTGTACTATTGTGCCAGAGGCCACTACTTCG |
| | ACTACTGGGGACAGGGCACACTGGTCACAGTCTCTTCTGCCTCC |
| | ACCAAGGGCCCATCGGTCTTCCCCCTGGCACCCTCCTCCAAGAG |
| | CACCTCTGGGGGCACAGCGGCCCTGGGCTGCCTGGTCAAGGACT |
| | ACTTCCCCGAACCGGTGACGGTGTCGTGGAACTCAGGCGCCCTG |
| | ACCAGCGGCGTGCACACCTTCCCGGCTGTCCTACAGTCCTCAGG |
| | ACTCTACTCCCTCAGCAGCGTGGTGACCGTGCCCTCCAGCAGCT |
| | TGGGCACCCAGACCTACATCTGCAACGTGAATCACAAGCCCAGC |
| | AACACCAAGGTGGACAAGAAAGTTGAGCCCAAATCTTGTGACA |
| | AAACTCACACATGCCCACCGTGCCCAGCACCTGAAGCCGCCGG |
| | GGGACCGTCAGTCTTCCTCTTCCCCCCAAAACCCAAGGACACCC |
| | TCATGATCTCCCGGACCCCTGAGGTCACATGCGTGGTGGTGAGC |
| | GTGAGCCACGAAGACCCTGAGGTCAAGTTCAACTGGTACGTGG |
| | ACGGCGTGGAGGTGCATAATGCCAAGACAAAGCCGCGGGAGGA |
| | GCAGTACAACAGCACGTACCGTGTGGTCAGCGTCCTCACCGTCC |
| | TGCACCAGGACTGGCTGAATGGCAAGGAGTACAAGTGCAAGGT |
| | GTCGAACAAAGCCCTCCCAGCCCCCATCGAGAAAACCATCTCCA |
| | AAGCCAAAGGGCAGCCCCGAGAACCACAGGTGTACACCCTGCC |
| | CCCATCCCGGGAGGAGATGACCAAGAACCAGGTCAGCCTGTCC |
| | TGCGCCGTCAAAGGCTTCTATCCCAGCGACATCGCCGTGGAGTG |
| | GGAGAGCAATGGGCAGCCGGAGAACAACTACAAGACCACGCCT |
| | CCCGTGCTGGACTCCGACGGCTCCTTCTTCCTCGTGAGCAAGCT |
| | CACCGTGGACAAGAGCAGATGGCAGCAGGGGAACGTCTTCTCA |
| | TGCTCCGTGATGCATGAGGCTCTGCACAACCGGTTCACGCAGAA |
| | GTCTCTCTCCCTGTCTCCGGGAAAA (SEQ ID NO: 321) |
| Light Chain (3^{rd} | GAGATCGTGATGACCCAGTCTCCTGCCACACTGTCTGTGTCTCC |
| poly-nucleotide) | CGGCGAGAGAGCTACCCTGTCTTGTAGAGCCTCTCAGTCCGTGT |
| | CCTCCAACCTGGCCTGGTATCAGCAGAAGCCTGGACTGGCTCCC |
| | CGGCTGTTGATCTATGGCGCTTCTACCAGAGCCACAGGCATCCC |
| | CGCTAGATTCTCCGGCTCTGGCTCTGGCACAGAGTTTACCCTGA |
| | CCATCTCCAGCCTGCAGTCCGAGGATTTCGCCGTGTACTACTGC |
| | CAGCAGTACAACAACTGGCCCCGGACCTTTGGCCAGGGCACCA |
| | AGGTGGAAATCAAGCGTACTGTGGCTGCACCATCTGTCTTCATC |
| | TTCCCGCCATCTGATGAGCAGTTGAAATCTGGAACTGCCTCTGT |
| | TGTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACACA |
| | AAGTCTACGCCTGCGAAGTCACCCATCAGGGCCTGAGCTCGCCC |
| | GTCACAAAGAGCTTCAACAGGGGAGAGTGT (SEQ ID NO: 322) |

TABLE 16C
codon optimized DNA Sequences Encoding NPP3B815

| | Polynucleotide sequence |
|---|---|
| Heavy Chain 1 | GAGATCGTGCTGACCCAGTCTCCTGCCACACTGAGTGCTTCTCC |
| (^{1st} poly- | AGGCGAGAGAGTGACCCTGTCCTGCTCCGCTTCCTCCTCCGTGT |
| nucleotide) | CCTACATGAACTGGTATCAGCAGAAGCCCGGCTGCGCCCCTAGA |
| | AGATGGATCTACGACTCCTCCAAGCTGGCCTCTGGCGTGCCTGC |
| | TAGATTTTCCGGCTCTGGCTCTGGCAGAGACTATACCCTGACAA |
| | TCTCCAGCCTGGAACCTGAGGACTTCGCCGTGTACTACTGCCAG |
| | CAGTGGTCTAGGAACCCTCCTACCTTTGGCGGAGGCACCAAGGT |
| | GGAAATCAAGGGCGGAGGTTCTGGTGGATCTGGCGGATGTCCTC |
| | CTTGTGGTGGAAGCGGCGGACAGGTTCAGCTGGTTCAGTCTGGC |
| | GCCGAAGTGAAGAAACCTGGCTCCTCCGTGAAAGTGTCCTGCAA |
| | GGCTTCCGGCTACACCTTTACCAGATCCACCATGCACTGGGTCA |
| | AGCAGGCCCCTGGACAAGGCTTGGAGTGGATCGGCTACATCAA |
| | CCCCAGCTCCGCCTACACCAACTACAACCAGAAATTCCAGGGCA |
| | GAGTGACCCTGACCGCCGACAAGTCTACCTCCACCGCCTACATG |
| | GAACTGTCCAGCCTGAGATCTGAGGACACCGCCGTGTACTACTG |
| | CGCCTCTCCTCAGGTTCACTACGACTACGCCGGCTTTCCTTATTG |
| | GGGCTGTGGCACCCTGGTCACCGTTTCTTCTGAGCCCAAATCTA |
| | GCGACAAAACTCACACATGCCCACCGTGCCCAGCACCTGAAGC |
| | CGCCGGGGGACCGTCAGTCTTCCTCTTCCCCCCAAAACCCAAGG |
| | ACACCCTCATGATCTCCCGGACCCCTGAGGTCACATGCGTGGTG |
| | GTGAGCGTGAGCCACGAAGACCCTGAGGTCAAGTTCAACTGGT |
| | ACGTGGACGGCGTGGAGGTGCATAATGCCAAGACAAAGCCGCG |
| | GGAGGAGCAGTACAACAGCACGTACCGTGTGGTCAGCGTCCTC |
| | ACCGTCCTGCACCAGGACTGGCTGAATGGCAAGGAGTACAAGT |
| | GCAAGGTGTCGAACAAAGCCCTCCCAGCCCCCATCGAGAAAAC |
| | CATCTCCAAAGCCAAAGGGCAGCCCCGAGAACCACAGGTGTAC |
| | ACCCTGCCCCCATCCCGGGAGGAGATGACCAAGAACCAGGTCA |
| | GCCTGTGGTGCCTGGTCAAAGGCTTCTATCCCAGCGACATCGCC |
| | GTGGAGTGGGAGAGCAATGGGCAGCCGGAGAACAACTACAAGA |
| | CCACGCCTCCCGTGCTGGACTCCGACGGCTCCTTCTTCCTCTACA |
| | GCAAGCTCACCGTGGACAAGAGCAGATGGCAGCAGGGGAACGT |
| | CTTCTCATGCTCCGTGATGCATGAGGCTCTGCACAACCACTACA |
| | CGCAGAAGTCTCTCTCCCTGTCTCCGGGAAAATGA (SEQ ID |
| | NO: 324) |
| Heavy Chain 2 | GAGGTGCAGCTGGTTGAATCTGGCGGCGGAAGAGTGAAGCCTG |
| (2^{nd} poly- | GCGGATCTCTGAGACTGTCTTGTGCCGCCTCTGGCTTCACCTTCT |
| nucleotide) | CCGACTACTCCATGAACTGGGTCCGACAGGCTCCTGGCAAAGGC |
| | CTGGAATGGGTGTCCTCTATCTCCTCCATCTCCAGCTACGTGAA |
| | GTACGCCGACTCCGTGAAGGGCAGATTCACCATGTCCAGAGAC |
| | AACGCCAAGAACTCCCTGTTCCTGCAGATGAACAGCCTGCGCGA |
| | CGAGGACATGGCCGTGTACTATTGTGCCAGAGGCCACTACTTCG |
| | ACTACTGGGGACAGGGCACACTGGTCACAGTCTCTTCTGCCTCC |
| | ACCAAGGGCCCATCGGTCTTCCCCCTGGCACCCTCCTCCAAGAG |
| | CACCTCTGGGGGCACAGCGGCCCTGGGCTGCCTGGTCAAGGACT |
| | ACTTCCCCGAACCGGTGACGGTGTCGTGGAACTCAGGCGCCCTG |
| | ACCAGCGGCGTGCACACCTTCCCGGCTGTCCTACAGTCCTCAGG |
| | ACTCTACTCCCTCAGCAGCGTGGTGACCGTGCCCTCCAGCAGCT |
| | TGGGCACCCAGACCTACATCTGCAACGTGAATCACAAGCCCAGC |
| | AACACCAAGGTGGACAAGAAAGTTGAGCCCAAATCTTGTGACA |
| | AAACTCACACATGCCCACCGTGCCCAGCACCTGAAGCCGCCGG |
| | GGGACCGTCAGTCTTCCTCTTCCCCCCAAAACCCAAGGACACCC |
| | TCATGATCTCCCGGACCCCTGAGGTCACATGCGTGGTGGTGAGC |
| | GTGAGCCACGAAGACCCTGAGGTCAAGTTCAACTGGTACGTGG |
| | ACGGCGTGGAGGTGCATAATGCCAAGACAAAGCCGCGGGAGGA |
| | GCAGTACAACAGCACGTACCGTGTGGTCAGCGTCCTCACCGTCC |
| | TGCACCAGGACTGGCTGAATGGCAAGGAGTACAAGTGCAAGGT |
| | GTCGAACAAAGCCCTCCCAGCCCCCATCGAGAAAACCATCTCCA |
| | AAGCCAAAGGGCAGCCCCGAGAACCACAGGTGTACACCCTGCC |
| | CCCATCCCGGGAGGAGATGACCAAGAACCAGGTCAGCCTGTCC |
| | TGCGCCGTCAAAGGCTTCTATCCCAGCGACATCGCCGTGGAGTG |
| | GGAGAGCAATGGGCAGCCGGAGAACAACTACAAGACCACGCCT |
| | CCCGTGCTGGACTCCGACGGCTCCTTCTTCCTCGTGAGCAAGCT |
| | CACCGTGGACAAGAGCAGATGGCAGCAGGGGAACGTCTTCTCA |
| | TGCTCCGTGATGCATGAGGCTCTGCACAACCGGTTCACGCAGAA |
| | GTCTCTCTCCCTGTCTCCGGGAAAATGA (SEQ ID NO: 325) |
| Light Chain (3^{rd} | GAGATCGTGATGACCCAGTCTCCTGCCACACTGTCTGTGTCTCC |
| poly-nucleotide) | CGGCGAGAGAGCTACCCTGTCTTGTAGAGCCTCTCAGTCCGTGT |
| | CCTCCAACCTGGCCTGGTATCAGCAGAAGCCTGGACTGGCTCCC |
| | CGGCTGTTGATCTATGGCGCTTCTACCAGAGCCACAGGCATCCC |
| | CGCTAGATTCTCCGGCTCTGGCTCTGGCACAGAGTTTACCCTGA |
| | CCATCTCCAGCCTGCAGTCCGAGGATTTCGCCGTGTACTACTGC |
| | CAGCAGTACAACAACTGGCCCCGGACCTTTGGCCAGGGCACCA |
| | AGGTGGAAATCAAGCGTACTGTGGCTGCACCATCTGTCTTCATC |
| | TTCCCGCCATCTGATGAGCAGTTGAAATCTGGAACTGCCTCTGT |
| | TGTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACACA |
| | AAGTCTACGCCTGCGAAGTCACCCATCAGGGCCTGAGCTCGCCC |
| | GTCACAAAGAGCTTCAACAGGGGAGAGTGTTGA (SEQ ID NO: |
| | 326) |

In specific embodiments, the ENPP3×CD3 binding protein provided herein comprises: (i) a first polypeptide comprising a scFv or spFv that binds a CD3ε, wherein the scFv or spFv is linked with a first VH (or first VL) domain that binds ENPP3 on one end, and is further linked to one arm of an antibody Fc region on the other end; (ii) a second polypeptide comprising a first VL (or first VH) domain that binds ENPP3, wherein the first VH (or first VL) and the first VL (or first VH) domains form a Fab that binds ENPP3; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the ENPP3×CD3 binding protein provided herein comprises: (i) a first polypeptide comprising a scFv or spFv that binds a CD3ε, wherein the scFv or spFv is linked with a first VH (or first VL) domain that binds ENPP3 on one end, and is further linked to one arm of an antibody Fc region on the other end; (ii) a second polypeptide comprising a first VL (or first VH) domain that binds ENPP3, wherein the first VH (or first VL) and the first VL (or first VH) domains form a Fab that binds ENPP3; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the ENPP3×CD3 binding protein provided herein comprises: (i) a first polypeptide comprising a scFv or spFv that binds a ENPP3, wherein the scFv or spFv is linked with a first VH (or first VL) domain that binds CD3ε on one end, and is further linked to one arm of an antibody Fc region on the other end; (ii) a second polypeptide comprising a first VL (or first VH) domain that binds CD3ε, wherein the first VH (or first VL) and the first VL (or first VH) domains form a Fab that binds CD3ε; (iii) a third polypeptide comprising a second VH domain that binds ENPP3, wherein the second VH domain is linked to the other arm of an antibody Fc region, and (iv) a fourth polypeptide comprising the second VL domain that binds ENPP3, wherein the second VH domain and the second VL domain form a Fab that binds ENPP3, and wherein the first polypeptide and the third polypeptide forms a Fc region.

In other specific embodiments, the ENPP3×CD3 binding protein provided herein comprises: (i) a first polypeptide comprising a scFv or spFv that binds a CD3ε, wherein the scFv or spFv is linked with a first VH (or first VL) domain that binds CD3ε on one end, and is further linked to one arm of an antibody Fc region on the other end; (ii) a second polypeptide comprising a first VL (or first VH) domain that binds CD3ε, wherein the first VH (or first VL) and the first VL (or first VH) domains form a Fab that binds CD3ε; (iii) a third polypeptide comprising a second VH domain that binds ENPP3, wherein the second VH domain is linked to the other arm of an antibody Fc region, and (iv) a fourth polypeptide comprising the second VL domain that binds ENPP3, wherein the second VH domain and the second VL domain form a Fab that binds ENPP3, and wherein the first polypeptide and the third polypeptide forms a Fc region.

In other specific embodiments, the ENPP3×CD3 binding protein provided herein comprises: (i) a first polypeptide comprising a scFv or spFv that binds a ENPP3, wherein the scFv or spFv is linked with a first VH (or first VL) domain that binds ENPP3 on one end, and is further linked to one arm of an antibody Fc region on the other end; (ii) a second polypeptide comprising a first VL (or first VH) domain that binds ENPP3, wherein the first VH (or first VL) and the first VL (or first VH) domains form a Fab that binds ENPP3; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the ENPP3×CD3 binding protein provided herein comprises: (i) a first polypeptide comprising a scFv or spFv that binds a ENPP3, wherein the scFv or spFv is linked with a first VH (or first VL) domain that binds CD3ε on one end, and is further linked to one arm of an antibody Fc region on the other end; (ii) a second polypeptide comprising a first VL (or first VH) domain that binds CD3ε, wherein the first VH (or first VL) and the first VL (or first VH) domains form a Fab that binds CD3ε; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the ENPP3×CD3 binding protein provided herein comprises: (i) a first polypeptide comprising a scFv or spFv that binds a CD3ε, wherein the scFv or spFv is linked with a first VH (or first VL) domain that binds ENPP3 on one end, and is further linked to one arm of an antibody Fc region on the other end; (ii) a second polypeptide comprising a first VL (or first VH) domain that binds ENPP3, wherein the first VH (or first VL) and the first VL (or first VH) domains form a Fab that binds ENPP3; (iii) a third polypeptide comprising a second VH domain that binds ENPP3, wherein the second VH domain is linked to the other arm of an antibody Fc region, and (iv) a fourth polypeptide comprising the second VL domain that binds ENPP3, wherein the second VH domain and the second VL domain form a Fab that binds ENPP3, and wherein the first polypeptide and the third polypeptide forms a Fc region.

In some embodiments, the ENPP3×CD3 binding protein provided is in the Central-scFv format as described in U.S. Pat. No. 10,889,653, the content of which is herein incorporated by reference in its entirety.

In some embodiments, the ENPP3×CD3 binding protein provided is in the spFv format as described in International Patent Application Publication No. WO2021030657, the content of which is herein incorporated by reference in its entirety.

In specific embodiments, the ENPP3×CD3 binding protein provided herein comprises: (i) a first polypeptide comprising a spFV that binds a CD3ε, wherein the spFv is linked with a first VH (or first VL) domain that binds ENPP3 on one end, and is further linked to one arm of an antibody Fc region on the other end; (ii) a second polypeptide comprising a first VL (or first VH) domain that binds ENPP3, wherein the first VH (or first VL) and the first VL (or first VH) domains form a Fab that binds ENPP3; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the ENPP3×CD3 binding protein provided herein comprises: (i) a first polypeptide comprising a spFV that binds a CD3ε, wherein the spFv is linked with a first VH (or first VL) domain that binds ENPP3 on one end, and is further linked to one arm of an antibody Fc region on the other end; (ii) a second polypeptide comprising a first VL (or first VH) domain that binds ENPP3, wherein the first VH (or first VL) and the first VL (or first VH) domains form a Fab that binds ENPP3; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the ENPP3×CD3 binding protein provided herein comprises: (i) a first polypeptide comprising a spFV that binds a ENPP3, wherein the spFv is linked with a first VH (or first VL) domain that binds CD3ε on one end, and is further linked to one arm of an antibody Fc region on the other end; (ii) a second polypeptide comprising a first VL (or first VH) domain that binds CD3ε, wherein the first VH (or first VL) and the first VL (or first VH) domains form a Fab that binds CD3ε; (iii) a third polypeptide comprising a second VH domain that binds ENPP3, wherein the second VH domain is linked to the other arm of an antibody Fc region, and (iv) a fourth polypeptide comprising the second VL domain that binds ENPP3, wherein the second VH domain and the second VL domain form a Fab that binds ENPP3, and wherein the first polypeptide and the third polypeptide forms a Fc region.

In other specific embodiments, the ENPP3×CD3 binding protein provided herein comprises: (i) a first polypeptide comprising a spFV that binds a CD3ε, wherein the spFv is linked with a first VH (or first VL) domain that binds CD3ε on one end, and is further linked to one arm of an antibody Fc region on the other end; (ii) a second polypeptide comprising a first VL (or first VH) domain that binds CD3ε, wherein the first VH (or first VL) and the first VL (or first VH) domains form a Fab that binds CD3ε; (iii) a third polypeptide comprising a second VH domain that binds ENPP3, wherein the second VH domain is linked to the other arm of an antibody Fc region, and (iv) a fourth polypeptide comprising the second VL domain that binds ENPP3, wherein the second VH domain and the second VL domain form a Fab that binds ENPP3, and wherein the first polypeptide and the third polypeptide forms a Fc region.

In other specific embodiments, the ENPP3×CD3 binding protein provided herein comprises: (i) a first polypeptide comprising a spFV that binds a ENPP3, wherein the spFv is linked with a first VH (or first VL) domain that binds ENPP3 on one end, and is further linked to one arm of an antibody Fc region on the other end; (ii) a second polypeptide comprising a first VL (or first VH) domain that binds ENPP3, wherein the first VH (or first VL) and the first VL (or first VH) domains form a Fab that binds ENPP3; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the ENPP3×CD3 binding protein provided herein comprises: (i) a first polypeptide comprising a spFV that binds a ENPP3, wherein the spFv is linked with a first VH (or first VL) domain that binds CD3ε on one end, and is further linked to one arm of an antibody Fc region on the other end; (ii) a second polypeptide comprising a first VL (or first VH) domain that binds CD3ε, wherein the first VH (or first VL) and the first VL (or first VH) domains form a Fab that binds CD3ε; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the ENPP3×CD3 binding protein provided herein comprises: (i) a first polypeptide comprising a spFV that binds a CD3ε, wherein the spFv is linked with a first VH (or first VL) domain that binds ENPP3 on one end, and is further linked to one arm of an antibody Fc region on the other end; (ii) a second polypeptide comprising a first VL (or first VH) domain that binds ENPP3, wherein the first VH (or first VL) and the first VL (or first VH) domains form a Fab that binds ENPP3; (iii) a third polypeptide comprising a second VH domain that binds ENPP3, wherein the second VH domain is linked to the other arm of an antibody Fc region, and (iv) a fourth polypeptide comprising the second VL domain that binds ENPP3, wherein the second VH domain and the second VL domain form a Fab that binds ENPP3, and wherein the first polypeptide and the third polypeptide forms a Fc region.

In some embodiments, the Fc region comprises one or more amino acid mutations that facilitate the dimerization of the two arms of the Fc region. In some embodiments, the ENPP3×CD3 binding protein provided herein comprises: (i) a first polypeptide comprising a scFv that binds CD3ε, a CH2, and a CH3, (ii) a second polypeptide comprising the VH domain that binds ENPP3, a CH2, and a CH3, and (iii) a third polypeptide comprising the VL domain that binds ENPP3, wherein the VH domain and the VL domain forms a Fab that binds ENPP3, and the first polypeptide and the second polypeptide forms a Fc region.

In other embodiments, the ENPP3×CD3 binding protein provided herein comprises a scFv that binds CD3 and two Fabs each bind ENPP3, and the binding agent further comprises a Fc region. In some embodiments, the two Fabs are identical and are linked to each other.

In other embodiments, the ENPP3×CD3 binding protein provided herein comprises a spFv that binds CD3 and two Fabs each bind ENPP3, and the binding agent further comprises a Fc region. In some embodiments, the two Fabs are identical and are linked to each other.

In some embodiments, the ENPP3×CD3 binding protein provided herein comprises: (i) a first polypeptide comprising a scFv or spFv that binds CD3ε that is linked to one arm of an antibody Fc region, (ii) a second polypeptide comprising two identical VH domains in tandem each bind ENPP3 that is linked to the other arm of an antibody Fc region, (iii) a third polypeptide comprising a VL domain that binds ENPP3, and (iv) a fourth polypeptide comprising a VL domain that binds ENPP3, wherein the two VH domains and the two VL domains form two Fabs that bind ENPP3, and the first polypeptide and the second polypeptide forms a Fc region. In some embodiments, the Fc region comprises one or more amino acid mutations that facilitate the dimerization of the two arms of the Fc region.

In some embodiments, the ENPP3×CD3 binding protein provided herein comprises: (i) a first polypeptide comprising a scFv or spFv that binds CD3ε, a CH2, and a CH3, (ii) a second polypeptide comprising a first VH domain that binds ENPP3, a second VH domain that binds ENPP3, a CH2, and a CH3, (iii) a third polypeptide comprising a first VL domain that binds ENPP3, and (iv) a fourth polypeptide comprising a second VH domain that binds ENPP3, wherein the first VH domain and the first VL domain forms a first Fab that binds ENPP3, the second VH domain and the second VL domain forms a second Fab that binds ENPP3, and the first polypeptide and the second polypeptide forms a Fc region.

### 5.7 Polynucleotides, Vectors, and Host Cells

Also disclosed are isolated polynucleotides that encode the ENPP3×CD3 binding protein provided herein. The isolated polynucleotides capable of encoding the ENPP3×CD3 binding protein provided herein may be included on the same, or different, vectors to produce antibodies or antigen-binding fragments of the invention.

In some embodiments, the polynucleotides of the invention include a polynucleotide encoding a leader sequence. Any leader sequence known in the art may be employed. The polynucleotide encoding the leader sequence may include a restriction endonuclease cleavage site or a translation initiation site.

Also provided are vectors comprising the polynucleotides of the invention. The vectors can be expression vectors. The expression vector may contain one or more additional sequences such as but not limited to regulatory sequences (e.g., promoter, enhancer), a selection marker, and a polyadenylation signal.

Recombinant expression vectors within the scope of the description include synthetic, or cDNA-derived nucleic acid fragments that encode at least one recombinant protein which may be operably linked to suitable regulatory elements.

The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

In some embodiments, the antibody- or antigen-binding fragment-coding sequence is placed under control of a powerful constitutive promoter. In addition, many viral promoters function constitutively in eukaryotic cells and are suitable for use with the described embodiments. In one embodiment, the coding sequence of the bispecific antibody of the invention or an antigen-binding fragment thereof is placed under control of an inducible promoter.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites, which may be upstream or downstream of any of the aforementioned nucleic acid sequences.

The vectors may comprise selection markers. A nucleic acid sequence encoding a selection marker or the cloning site may be upstream or downstream of a nucleic acid sequence encoding a polypeptide of interest or cloning site.

The vectors described herein may be used to transform various cells with the genes encoding the described antibodies or antigen-binding fragments. For example, the vectors may be used to generate bispecific ENPP3×CD3 antibodies or antigen-binding fragment-producing cells. Thus, the invention also provides a host cell comprising the vectors of the invention.

Numerous techniques are known in the art for the introduction of foreign genes into cells and may be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

Cells suitable for use in the expression of the antibodies or antigen-binding fragments described herein are preferably eukaryotic cells, more preferably cells of plant, rodent, or human origin. In addition, expression of antibodies may be accomplished using hybridoma cells. Methods for producing hybridomas are well established in the art.

Cells transformed with expression vectors of the invention may be selected or screened for recombinant expression of the antibodies or antigen-binding fragments of the invention. Recombinant-positive cells are expanded and screened for subclones exhibiting a desired phenotype, such as high-level expression, enhanced growth properties, or the ability to yield proteins with desired biochemical characteristics, for example, due to protein modification or altered post-translational modifications. These phenotypes may be due to inherent properties of a given subclone or to mutation. Mutations may be effected through the use of chemicals, UV-wavelength light, radiation, viruses, insertional mutagens, inhibition of DNA mismatch repair, or a combination of such methods.

Table 1 shows polynucleotide sequences encoding the VH/VL amino acid sequences for an exemplary ENPP3 binding arm. Table 13A shows polynucleotide sequences encoding the VH/VL amino acid sequences for an exemplary CD3ε binding arm. Table 16B shows DNA sequences encoding the NPP3B815 antibody.

In some embodiments, the ENPP3 binding arm comprises a VH encoded by a polynucleotide sequence as set forth in SEQ ID NO:316. In some embodiments, the ENPP3 binding arm comprises a VL encoded by a polynucleotide sequence as set forth in SEQ ID NO: 317.

In some embodiments, the CD3ε binding arm comprises a VH encoded by a polynucleotide sequence as set forth in SEQ ID NO:318. In some embodiments, the CD3ε binding arm comprises a VL encoded by a polynucleotide sequence as set forth in SEQ ID NO: 319.

In some embodiments, the bispecific ENPP3×CD3ε binding molecule comprises an ENPP3 binding arm comprising a VH encoded by a polynucleotide sequence as set forth in SEQ ID NO: 316 and a VL encoded by a polynucleotide sequence as set forth in SEQ ID NO:317 and a CD3ε binding arm comprising a VH encoded by a polynucleotide sequence as set forth in SEQ ID NO: 318 and a VL encoded by a polynucleotide sequence as set forth in SEQ ID NO:319.

In some embodiments, the binding agent is encoded by a first polynucleotide encoding a spFv that binds CD3ε, a CH2 domain and a CH3 domain; a second polynucleotide encoding a VH domain that binds ENPP3, a CH2 domain and a CH3 domain; and a third polynucleotide encoding a VL domain that binds ENPP3. In some embodiments, the first polynucleotide encoding a spFv that binds CD3ε comprises SEQ ID NO:320. In some embodiments, the second polynucleotide encoding a VH domain that binds ENPP3, a CH2 domain and a CH3 domain comprises SEQ ID NO:321. In some embodiments, the third polynucleotide encoding a VL domain that binds ENPP3 comprises SEQ ID NO:322.

### 5.8 Pharmaceutical Compositions

In another general aspect, provided is a pharmaceutical composition comprising the ENPP3 binding agent (monospecific or multispecific) provided herein and a pharmaceutically acceptable excipient. In some embodiments, the ENPP3 binding agent is a ENPP3 binding protein. In another general aspect, provided is a pharmaceutical composition comprising a nucleic acid encoding the ENPP3 binding protein (monospecific or multispecific) provided herein or a fragment or a portion thereof and a pharmaceutically acceptable excipient. In another general aspect, provided is a pharmaceutical composition comprising the ENPP3 binding protein (monospecific or multispecific) provided herein and a pharmaceutically acceptable excipient.

In another general aspect, provided herein is a method of producing a pharmaceutical composition comprising a binding agent or an antigen binding region thereof provided herein, comprising combining a binding agent or an antigen binding region thereof with a pharmaceutically acceptable carrier to obtain the pharmaceutical composition.

### 5.9 Methods of Use

The functional activity of ENPP3 binding agents (monospecific or multispecific) provided herein can be characterized by methods known in the art and as described herein. Methods for characterizing binding agents include, but are not limited to, affinity and specificity assays including Biacore, ELISA, and OctetRed analysis; binding assays to detect the binding of antibodies to target cells by FACS; binding assays to detect the binding of antibodies to the target antigen on cells. According to particular embodiments, the methods for characterizing binding agents include those described below.

An ENPP3 binding agent (monospecific or multispecific) of the disclosure is useful in a variety of applications including, but not limited to, therapeutic treatment methods, such as treatment of cancer. In some embodiments, the cancer is a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.

In some embodiments, the therapeutic treatment methods comprise immunotherapy for cancer. In some embodiments, the ENPP3 binding agent is useful for activating, promoting, increasing, and/or enhancing an immune response to a cancer or cancer cell. In some embodiments, the ENPP3 binding agent is useful for activating, promoting, increasing, and/or enhancing an immune response to a tumor or tumor cell. In some embodiments, the ENPP3 binding agent is useful for activating, promoting, increasing, and/or enhancing a T cell response to a cancer or cancer cells. In some embodiments, the ENPP3 binding agent is useful for activating, promoting, increasing, and/or enhancing a T cell response to a tumor or tumor cell. The methods of use may be in vitro, ex vivo, or in vivo methods.

In one aspect, provided herein is a method of directing a T cell to a target cell expressing ENPP3 by contacting the T cell with an effective amount of an ENPP3×CD3 binding agent provided herein. In some embodiments, wherein the CD3 binding region binds the T cell. In some embodiments, the ENPP3 binding region binds the target cell. In some embodiments, the target cell is a tumor or cancer cell. In some embodiments, the cancer is a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.

In another aspect, provided herein is a method of directing a T cell to a cancer or tumor cell, comprising contacting the T cell with an effective amount of a pharmaceutical composition comprising an ENPP3×CD3 binding agent provided herein, wherein the CD3 binding region binds the T cell. In some embodiments, the directed T cell incudes cytokine release. In some embodiments, cytokine release is increased compared to a reference. In some embodiments, said cytokine release is determined by measuring IL-1β, IL-2, IL-4, IL-6, IL-8, IL-10, IL-12, IL-13, TNF-α, IFN-γ, or any combination thereof. In some embodiments, the directed T cell induces cytokine release, and release of one or more of IL-1B, IL-2, IL-4, IL-6, IL-8, IL-10, IL-12, IL-13, TNF-α, and IFN-γ is increased as compared to a reference. In some embodiments, a cytokine reference is: (a) a cytokine measured in a corresponding normal cell or issue; (b) a cytokine measured in a neighboring non-cancerous cell or tissue in the same subject; or (c) a cytokine measured in a corresponding cell or tissue measured in a cohort of healthy subjects. In some embodiments, the directed T cell induces apoptosis in the cancer or tumor cell. In some embodiments, when the T cell is directed to the cancer or tumor cell, the T cell induces differential cytotoxicity and cytokine release. That is, a method of directing a T cell to a cancer or tumor cell results in T-cell dependent cytotoxicity (TDCC) that is inversely related to T cell cytokine release. For example, in some embodiments, TDCC is increased compared to a reference and cytokine release is decreased compared to a reference. In some embodiments, said TDCC reference is: (a) TDCC measured in a corresponding normal cell or tissue; (b) TDCC measured in a neighboring non-cancerous cell or tissue in the same subject; or (c) TDCC measured in a corresponding cell or tissue measured in a cohort of healthy subjects. In some embodiments, said TDCC is determined by measuring apoptosis. In some embodiments, caspase mediated apoptosis is increased. In some embodiments, a cytokine reference is: (a) a cytokine measured in a corresponding normal cell or issue; (b) a cytokine measured in a neighboring non-cancerous cell or tissue in the same subject; or (c) a cytokine measured in a corresponding cell or tissue measured in a cohort of healthy subjects. In some embodiments, said cytokine release is determined by measuring IL-1β, IL-2, IL-4, IL-6, IL-8, IL-10, IL-12, IL-13, TNF-α, IFN-γ, or any combination thereof.

In some embodiments, the cancer cell expresses ENPP3. In some embodiments, the cancer cell expresses high levels of ENPP3 compared to a reference expression level. In some embodiments, the cancer cell expresses low levels of ENPP3 compared to a reference expression level. In some embodiments, said reference expression level of ENPP3 is: (a) a predetermined expression level of ENPP3; (b) an ENPP3 expression level in a corresponding normal cell or issue; (c) an ENPP3 expression level measured in a neighboring non-cancerous cell or tissue in the same subject; or (d) an ENPP3 expression level in a corresponding cell or tissue measured in a cohort of healthy subjects. In some embodiments, said expression level of ENPP3 is determined by measuring the protein expression level of ENPP3.

In one aspect, provided herein is a method of activating a T cell, comprising contacting the T cell with an effective amount of the ENPP3×CD3 binding protein provided herein, wherein the CD3 binding region binds the T cell. In another aspect, provided herein is a method of activating a T cell, comprising contacting the T cell with a pharmaceutical composition comprising an ENPP3×CD3 binding protein provided herein.

In some embodiments, T cell activation is measured by expression of certain markers by the T cells. In some embodiments, T cell activation is measured by the percentage of T cells positive for a particular marker in a population of T cells. In some embodiments, the T cell activation marker is CD25. In some embodiments, the T cell activation marker is CD69. In some embodiments, the method of activating the T cell increases expression of CD25, CD69 or both CD25 and CD69 in the T cells. In some embodiments, the method of activating the T cell increases the percentage of CD25+ T cells, CD69+ T cells, and/or CD25+/CD69+ T cell in the population of T cells.

In one aspect, provided herein is a method of targeting an antigen on the surface of a target cell, the method comprising contacting the target cell with an effective amount of an ENPP3×CD3 binding protein provided herein, wherein the ENPP3 binding region binds to the target cell. In another aspect, provided herein is a method of targeting an antigen on the surface of a target cell, the method comprising contacting the target cell with an effective amount of a pharmaceutical composition comprising a ENPP3×CD3 binding protein provided herein, wherein the ENPP3 binding region binds to the target cell. In some embodiments, provided herein is a method of targeting an antigen on the surface of a target cell, the method comprising contacting the target cell with an effective amount of a pharmaceutical composition comprising a ENPP3×CD3 binding protein provided herein. In some embodiments, the target cell is a cancer or tumor cell that expresses ENPP3. In some embodiments, the target cell expresses high levels of ENPP3 compared to a reference expression level. In some embodiments, the target cell expresses low levels of ENPP3 compared to a reference expression level. In some embodiments, said reference expression level of ENPP3 is: (a) a predetermined expression level of ENPP3; (b) an ENPP3 expression level in a corresponding normal cell or issue; (c) an ENPP3 expression level measured in a neighboring non-cancerous cell or tissue in the same subject; or (d) an ENPP3 expression level in a corresponding cell or tissue measured in a cohort of healthy subjects. In some embodiments, said expression level of ENPP3 is determined by measuring the protein expression level of ENPP3. In some embodiments, the cancer is a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.

In one aspect, provided herein is a method of killing or inhibiting the proliferation of a cancer or tumor cell, comprising contacting the cancer or tumor cell with a ENPP3×CD3 binding protein provided herein. In another aspect, provided herein is a method of killing or inhibiting the proliferation of a cancer or tumor cell, comprising contacting the cancer or tumor cell with a pharmaceutical composition comprising a ENPP3×CD3 binding protein provided herein. In some embodiments, the ENPP3×CD3 binding protein activates a T cell. In some embodiments, the CD3 binding region activates the T cell. In some embodiments, the activated T cell induces apoptosis in the cancer or tumor cell. In some embodiments, the cancer is a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.

In some embodiments, the cancer or tumor cell expresses high levels of ENPP3 compared to a reference expression level. In some embodiments, the cancer or tumor cell expresses low levels of ENPP3 compared to a reference expression level. In some embodiments, said reference expression level of ENPP3 is: (a) a predetermined expression level of ENPP3; (b) an ENPP3 expression level in a corresponding normal cell or issue; (c) an ENPP3 expression level measured in a neighboring non-cancerous cell or tissue in the same subject; or (d) an ENPP3 expression level in a corresponding cell or tissue measured in a cohort of healthy subjects. In some embodiments, said expression level of ENPP3 is determined by measuring the protein expression level of ENPP3.

In one aspect, provided herein is a method of treating a cancer or tumor in a subject, comprising administering an effective amount of a ENPP3×CD3 binding protein provided herein. In another aspect, provided herein is a method of treating a cancer or tumor in a subject, comprising administering an effective amount of a pharmaceutical composition comprising a ENPP3×CD3 binding protein provided herein or the pharmaceutical composition provided herein In some embodiments, the cancer is a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.

In some embodiments, the cancer or tumor cell expresses ENPP3. In some embodiments, the cancer or tumor cell expresses high levels of ENPP3 compared to a reference expression level. In some embodiments, the cancer or tumor cell expresses low levels of ENPP3 compared to a reference expression level. In some embodiments, said reference expression level of ENPP3 is: (a) a predetermined expression level of ENPP3; (b) an ENPP3 expression level in a corresponding normal cell or issue; (c) an ENPP3 expression level measured in a neighboring non-cancerous cell or tissue in the same subject; or (d) an ENPP3 expression level in a corresponding cell or tissue measured in a cohort of healthy subjects. In some embodiments, said expression level of ENPP3 is determined by measuring the protein expression level of ENPP3.

In another aspect, provided herein is a use of the ENPP3×CD3 binding protein provided herein in the manufacture of a medicament for treatment of a cancer or tumor in a subject thereof. In yet a further aspect, provided herein is a binding agent for use in the treatment of a cancer or tumor. In some embodiments, the cancer is a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.

In some embodiments, the cancer or tumor cell expresses ENPP3. In some embodiments, the cancer or tumor cell expresses high levels of ENPP3 compared to a reference expression level. In some embodiments, the cancer or tumor cell expresses low levels of ENPP3 compared to a reference expression level. In some embodiments, said reference expression level of ENPP3 is: (a) a predetermined expression level of ENPP3; (b) an ENPP3 expression level in a corresponding normal cell or issue; (c) an ENPP3 expression level measured in a neighboring non-cancerous cell or tissue in the same subject; or (d) an ENPP3 expression level in a corresponding cell or tissue measured in a cohort of healthy subjects. In some embodiments, said expression level of ENPP3 is determined by measuring the protein expression level of ENPP3.

In one aspect, provided herein is a method of treating a subject having a ENPP3-expressing cancer or tumor, comprising administering or providing for administration of an effective amount of the ENPP3×CD3 binding protein to the subject wherein the subject has a ENPP3-expressing cancer or tumor. In some embodiments, the cancer is a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma. In some embodiments, said expression level of ENPP3 is determined by measuring the protein expression level of ENPP3. In some embodiments, the subject is a subject in need thereof. In some embodiments, the subject is a human. In specific embodiments, the subject is administered an effective amount.

According to particular embodiments, the pharmaceutical compositions described herein are formulated to be suitable for the intended route of administration to a subject. For example, the pharmaceutical compositions described herein can be formulated to be suitable for intravenous, subcutaneous, or intramuscular administration.

In case of conflict, the specification, including definitions, will control. As used herein and in the appended claims, the singular forms “a,” “an,” and “the” include plural referents unless the context clearly dictates otherwise. Thus, for example, reference to “a peptide sequence” or “a treatment,” includes a plurality of such sequences, treatments, and so forth. It is further noted that the claims can be drafted to exclude any optional element. As such, this statement is intended to serve as antecedent basis for use of such exclusive terminology such as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges, unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1000, 1000-2500, 2500-5000, 5000-25,000, or 5000-50,000 includes any numerical value or range within or encompassing such values, e.g., 25, 26, 27, 28, 29 . . . 250, 251, 252, 253, 254 . . . 500, 501, 502, 503, 504 . . . , etc. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### 6. EMBODIMENTS

1. A binding agent comprising an antigen binding region that binds to an epitope of ENPP3.
2. The binding agent of embodiment 1, wherein the epitope of ENPP3 is selected from the group consisting of SEQ ID NO:295-297, DVP, and SEQ ID NO:299-305.
3. The binding agent of embodiment 1, wherein the antigen binding region comprises a VH and VL domain selected from the group consisting of:
   (a) a VH domain comprising a HCDR1, HCDR2, and HCDR3 of a VH as set forth in SEQ ID NO:22, and a VL domain comprising a LCDR1, LCDR2, and LCDR3 of a VL as set forth in SEQ ID NO:23;
   (b) a VH domain comprising a HCDR1, HCDR2, and HCDR3 of a VH as set forth in SEQ ID NO:45, and a VL domain comprising a LCDR1, LCDR2, and LCDR3 of a VL as set forth in SEQ ID NO:46;
   (c) a VH domain comprising a HCDR1, HCDR2, and HCDR3 of a VH as set forth in SEQ ID NO:68, and a VL domain comprising a LCDR1, LCDR2, and LCDR3 of a VL as set forth in SEQ ID NO:69 or SEQ ID NO:70;
   (d) a VH domain comprising a HCDR1, HCDR2, and HCDR3 of a VH as set forth in SEQ ID NO:91, and a VL domain comprising a LCDR1, LCDR2, and LCDR3 of a VL as set forth in SEQ ID NO:92;
   (e) a VH domain comprising a HCDR1, HCDR2, and HCDR3 of a VH as set forth in SEQ ID NO:113, and a VL domain comprising a LCDR1, LCDR2, and LCDR3 of a VL as set forth in SEQ ID NO: 114 or SEQ ID NO:115;
   (f) a VH domain comprising a HCDR1, HCDR2, and HCDR3 of a VH as set forth in SEQ ID NO:134, and a VL domain comprising a LCDR1, LCDR2, and LCDR3 of a VL as set forth in SEQ ID NO: 135 or SEQ ID NO:136;
   (g) a VH domain comprising a HCDR1, HCDR2, and HCDR3 of a VH as set forth in SEQ ID NO: 158, and a VL domain comprising a LCDR1, LCDR2, and LCDR3 of a VL as set forth in SEQ ID NO: 159; and
   (h) a VH domain comprising a HCDR1, HCDR2, and HCDR3 of a VH as set forth in SEQ ID NO:177, and a VL domain comprising a LCDR1, LCDR2, and LCDR3 of a VL as set forth in SEQ ID NO:178.
4. The binding agent of embodiment 3, wherein the CDR sequences are selected from the group consisting of:
   (a1) the HCDR1 comprises the amino acid sequence of SEQ ID NO:1, the HCDR2 comprises the amino acid sequence of SEQ ID NO:2, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
   (a2) the HCDR1 comprises the amino acid sequence of SEQ ID NO:7, the HCDR2 comprises the amino acid sequence of SEQ ID NO:8, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
   (a3) the HCDR1 comprises the amino acid sequence of SEQ ID NO:9, the HCDR2 comprises the amino acid sequence of SEQ ID NO: 10, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
   (a4) the HCDR1 comprises the amino acid sequence of SEQ ID NO: 11, the HCDR2 comprises the amino acid sequence of SEQ ID NO: 12, the HCDR3 comprises the amino acid sequence of SEQ ID NO: 13, the LCDR1 comprises the amino acid sequence of SEQ ID NO: 14, the LCDR2 comprises the amino acid sequence of SEQ ID NO: 15, and the LCDR3 comprises the amino acid sequence of SEQ ID NO: 16;
   (a5) the HCDR1 comprises the amino acid sequence of SEQ ID NO: 17, the HCDR2 comprises the amino acid sequence of SEQ ID NO: 18, the HCDR3 comprises the amino acid sequence of SEQ ID NO: 19, the LCDR1 comprises the amino acid sequence of SEQ ID NO:20, the LCDR2 comprises the amino acid sequence of GAS, and the LCDR3 comprises the amino acid sequence of SEQ ID NO:6;
   (b1) the HCDR1 comprises the amino acid sequence of SEQ ID NO:24, the HCDR2 comprises the amino acid sequence of SEQ ID NO:25, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
   (b2) the HCDR1 comprises the amino acid sequence of SEQ ID NO:30, the HCDR2 comprises the amino acid sequence of SEQ ID NO:31, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
   (b3) the HCDR1 comprises the amino acid sequence of SEQ ID NO:32, the HCDR2 comprises the amino acid sequence of SEQ ID NO:33, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
   (b4) the HCDR1 comprises the amino acid sequence of SEQ ID NO:34, the HCDR2 comprises the amino acid sequence of SEQ ID NO:35, the HCDR3 comprises the amino acid sequence of SEQ ID NO:36, the LCDR1 comprises the amino acid sequence of SEQ ID NO:37, the LCDR2 comprises the amino acid sequence of SEQ ID NO:38, and the LCDR3 comprises the amino acid sequence of SEQ ID NO: 39;
   (b5) the HCDR1 comprises the amino acid sequence of SEQ ID NO:40, the HCDR2 comprises the amino acid sequence of SEQ ID NO:41, the HCDR3 comprises the amino acid sequence of SEQ ID NO:42, the LCDR1 comprises the amino acid sequence of SEQ ID NO:43, the LCDR2 comprises the amino acid sequence of DAS, and the LCDR3 comprises the amino acid sequence of SEQ ID NO:29;
   (c1) the HCDR1 comprises the amino acid sequence of SEQ ID NO:47, the HCDR2 comprises the amino acid sequence of SEQ ID NO:48, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
   (c2) the HCDR1 comprises the amino acid sequence of SEQ ID NO:53, the HCDR2 comprises the amino acid sequence of SEQ ID NO:54, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
   (c3) the HCDR1 comprises the amino acid sequence of SEQ ID NO:55, the HCDR2 comprises the amino acid sequence of SEQ ID NO:56, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
   (c4) the HCDR1 comprises the amino acid sequence of SEQ ID NO:57, the HCDR2 comprises the amino acid sequence of SEQ ID NO:58, the HCDR3 comprises the amino acid sequence of SEQ ID NO:59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO: 62;
   (c5) the HCDR1 comprises the amino acid sequence of SEQ ID NO:63, the HCDR2 comprises the amino acid sequence of SEQ ID NO:64, the HCDR3 comprises the amino acid sequence of SEQ ID NO:65, the LCDR1 comprises the amino acid sequence of SEQ ID NO:66, the LCDR2 comprises the amino acid sequence of QIS, and the LCDR3 comprises the amino acid sequence of SEQ ID NO:52;
   (d1) the HCDR1 comprises the amino acid sequence of SEQ ID NO:71, the HCDR2 comprises the amino acid sequence of SEQ ID NO:72, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
   (d2) the HCDR1 comprises the amino acid sequence of SEQ ID NO:77, the HCDR2 comprises the amino acid sequence of SEQ ID NO:78, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
   (d3) the HCDR1 comprises the amino acid sequence of SEQ ID NO:79, the HCDR2 comprises the amino acid sequence of SEQ ID NO:80, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
   (d4) the HCDR1 comprises the amino acid sequence of SEQ ID NO:81, the HCDR2 comprises the amino acid sequence of SEQ ID NO:82, the HCDR3 comprises the amino acid sequence of SEQ ID NO:83, the LCDR1 comprises the amino acid sequence of SEQ ID NO:84, the LCDR2 comprises the amino acid sequence of SEQ ID NO:85, and the LCDR3 comprises the amino acid sequence of SEQ ID NO: 86;
   (d5) the HCDR1 comprises the amino acid sequence of SEQ ID NO:87, the HCDR2 comprises the amino acid sequence of SEQ ID NO:88, the HCDR3 comprises the amino acid sequence of SEQ ID NO:89, the LCDR1 comprises the amino acid sequence of SEQ ID NO:90, the LCDR2 comprises the amino acid sequence of GAS, and the LCDR3 comprises the amino acid sequence of SEQ ID NO:76;
   (e1) the HCDR1 comprises the amino acid sequence of SEQ ID NO:93, the HCDR2 comprises the amino acid sequence of SEQ ID NO:94, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97 and the LCDR3 comprises the amino acid sequence of SEQ ID NO: 98;
   (e2) the HCDR1 comprises the amino acid sequence of SEQ ID NO:99, the HCDR2 comprises the amino acid sequence of SEQ ID NO: 100, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO: 98;
   (e3) the HCDR1 comprises the amino acid sequence of SEQ ID NO:101, the HCDR2 comprises the amino acid sequence of SEQ ID NO: 102, the HCDR3 comprises the amino acid sequence of SEQ ID NO:95, the LCDR1 comprises the amino acid sequence of SEQ ID NO:96 the LCDR2 comprises the amino acid sequence of SEQ ID NO:97, and the LCDR3 comprises the amino acid sequence of SEQ ID NO: 98;
   (e4) the HCDR1 comprises the amino acid sequence of SEQ ID NO: 103, the HCDR2 comprises the amino acid sequence of SEQ ID NO: 104, the HCDR3 comprises the amino acid sequence of SEQ ID NO:105, the LCDR1 comprises the amino acid sequence of SEQ ID NO:106, the LCDR2 comprises the amino acid sequence of SEQ ID NO:107, and the LCDR3 comprises the amino acid sequence of SEQ ID NO:108;
   (e5) the HCDR1 comprises the amino acid sequence of SEQ ID NO: 109, the HCDR2 comprises the amino acid sequence of SEQ ID NO:110, the HCDR3 comprises the amino acid sequence of SEQ ID NO:111, the LCDR1 comprises the amino acid sequence of SEQ ID NO:112, the LCDR2 comprises the amino acid sequence of GAS, and the LCDR3 comprises the amino acid sequence of SEQ ID NO: 98;
   (f1) the HCDR1 comprises the amino acid sequence of SEQ ID NO: 116, the HCDR2 comprises the amino acid sequence of SEQ ID NO:117, the HCDR3 comprises the amino acid sequence of SEQ ID NO:118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120;
   (f2) the HCDR1 comprises the amino acid sequence of SEQ ID NO: 121, the HCDR2 comprises the amino acid sequence of SEQ ID NO:122, the HCDR3 comprises the amino acid sequence of SEQ ID NO: 118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120;
   (f3) the HCDR1 comprises the amino acid sequence of SEQ ID NO: 123, the HCDR2 comprises the amino acid sequence of SEQ ID NO:124, the HCDR3 comprises the amino acid sequence of SEQ ID NO: 118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO:120;
   (f4) the HCDR1 comprises the amino acid sequence of SEQ ID NO: 125, the HCDR2 comprises the amino acid sequence of SEQ ID NO: 126, the HCDR3 comprises the amino acid sequence of SEQ ID NO: 127, the LCDR1 comprises the amino acid sequence of SEQ ID NO: 128, the LCDR2 comprises the amino acid sequence of SEQ ID NO:15, and the LCDR3 comprises the amino acid sequence of SEQ ID NO:129;
   (f5) the HCDR1 comprises the amino acid sequence of SEQ ID NO: 130, the HCDR2 comprises the amino acid sequence of SEQ ID NO:131, the HCDR3 comprises the amino acid sequence of SEQ ID NO: 132, the LCDR1 comprises the amino acid sequence of SEQ ID NO: 133, the LCDR2 comprises the amino acid sequence of GAS, and the LCDR3 comprises the amino acid sequence of SEQ ID NO: 120;
   (g1) the HCDR1 comprises the amino acid sequence of SEQ ID NO: 137, the HCDR2 comprises the amino acid sequence of SEQ ID NO: 138, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO:141, and the LCDR3 comprises the amino acid sequence of SEQ ID NO: 142;
   (g2) the HCDR1 comprises the amino acid sequence of SEQ ID NO: 143, the HCDR2 comprises the amino acid sequence of SEQ ID NO: 144, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO:141, and the LCDR3 comprises the amino acid sequence of SEQ ID NO:142;
   (g3) the HCDR1 comprises the amino acid sequence of SEQ ID NO: 145, the HCDR2 comprises the amino acid sequence of SEQ ID NO: 146, the HCDR3 comprises the amino acid sequence of SEQ ID NO:139, the LCDR1 comprises the amino acid sequence of SEQ ID NO: 140, the LCDR2 comprises the amino acid sequence of SEQ ID NO:141, and the LCDR3 comprises the amino acid sequence of SEQ ID NO:142;
   (g4) the HCDR1 comprises the amino acid sequence of SEQ ID NO: 147, the HCDR2 comprises the amino acid sequence of SEQ ID NO: 148, the HCDR3 comprises the amino acid sequence of SEQ ID NO: 149, the LCDR1 comprises the amino acid sequence of SEQ ID NO: 150, the LCDR2 comprises the amino acid sequence of SEQ ID NO:151, and the LCDR3 comprises the amino acid sequence of SEQ ID NO:152;
   (g5) the HCDR1 comprises the amino acid sequence of SEQ ID NO: 153, the HCDR2 comprises the amino acid sequence of SEQ ID NO:154, the HCDR3 comprises the amino acid sequence of SEQ ID NO: 155, the LCDR1 comprises the amino acid sequence of SEQ ID NO: 156, the LCDR2 comprises the amino acid sequence of AAS, and the LCDR3 comprises the amino acid sequence of SEQ ID NO: 142;
   (h1) the HCDR1 comprises the amino acid sequence of SEQ ID NO: 1, the HCDR2 comprises the amino acid sequence of SEQ ID NO:160, the HCDR3 comprises the amino acid sequence of SEQ ID NO: 161, the LCDR1 comprises the amino acid sequence of SEQ ID NO: 162, the LCDR2 comprises the amino acid sequence of SEQ ID NO: 163, and the LCDR3 comprises the amino acid sequence of SEQ ID NO:76;
   (h2) the HCDR1 comprises the amino acid sequence of SEQ ID NO: 164, the HCDR2 comprises the amino acid sequence of SEQ ID NO: 165, the HCDR3 comprises the amino acid sequence of SEQ ID NO:161, the LCDR1 comprises the amino acid sequence of SEQ ID NO: 162 the LCDR2 comprises the amino acid sequence of SEQ ID NO: 163, and the LCDR3 comprises the amino acid sequence of SEQ ID NO: 76;
   (h3) the HCDR1 comprises the amino acid sequence of SEQ ID NO: 166, the HCDR2 comprises the amino acid sequence of SEQ ID NO: 167, the HCDR3 comprises the amino acid sequence of SEQ ID NO:161, the LCDR1 comprises the amino acid sequence of SEQ ID NO: 162, the LCDR2 comprises the amino acid sequence of SEQ ID NO: 163, and the LCDR3 comprises the amino acid sequence of SEQ ID NO:76;
   (h4) the HCDR1 comprises the amino acid sequence of SEQ ID NO: 11, the HCDR2 comprises the amino acid sequence of SEQ ID NO: 168, the HCDR3 comprises the amino acid sequence of SEQ ID NO:169, the LCDR1 comprises the amino acid sequence of SEQ ID NO:170, the LCDR2 comprises the amino acid sequence of SEQ ID NO:171, and the LCDR3 comprises the amino acid sequence of SEQ ID NO:86; and
   (h5) the HCDR1 comprises the amino acid sequence of SEQ ID NO: 172, the HCDR2 comprises the amino acid sequence of SEQ ID NO:173, the HCDR3 comprises the amino acid sequence of SEQ ID NO:174, the LCDR1 comprises the amino acid sequence of SEQ ID NO:175, the LCDR2 comprises the amino acid sequence of VAS and the LCDR3 comprises the amino acid sequence of SEQ ID NO: 76.
5. The binding agent of any one of embodiments 1 to 4, wherein the antigen binding region comprises a VH domain and a VL domain selected from the group consisting of:
   (a) a VH domain comprising the amino acid sequence of SEQ ID NO:22, and a VL domain comprising the amino acid sequence of SEQ ID NO:23;
   (b) a VH domain comprising the amino acid sequence of SEQ ID NO:45, and a VL domain comprising the amino acid sequence of SEQ ID NO:46;
   (c) a VH domain comprising the amino acid sequence of SEQ ID NO:68, and a VL domain comprising the amino acid sequence of SEQ ID NO:69;
   (c′) a VH domain comprising the amino acid sequence of SEQ ID NO:68, and a VL domain comprising the amino acid sequence of SEQ ID NO:70;
   (d) a VH domain comprising the amino acid sequence of SEQ ID NO:91, and a VL domain comprising the amino acid sequence of SEQ ID NO:92;
   (e) a VH domain comprising the amino acid sequence of SEQ ID NO:113, and a VL domain comprising the amino acid sequence of SEQ ID NO:114;
   (e′) a VH domain comprising the amino acid sequence of SEQ ID NO: 113, and a VL domain comprising the amino acid sequence of SEQ ID NO:115;
   (f) a VH domain comprising the amino acid sequence of SEQ ID NO:134, and a VL domain comprising the amino acid sequence of SEQ ID NO: 135;
   (f′) a VH domain comprising the amino acid sequence of SEQ ID NO:134, and a VL domain comprising the amino acid sequence of SEQ ID NO: 136;
   (g) a VH domain comprising the amino acid sequence of SEQ ID NO: 158, and a VL domain comprising the amino acid sequence of SEQ ID NO: 159; and
   (h) a VH domain comprising the amino acid sequence of SEQ ID NO: 177, and a VL domain comprising the amino acid sequence of SEQ ID NO:178.
6. The binding agent of any one of embodiments 1 to 5, wherein the binding agent is a bispecific protein or a multi-specific protein.
7. The binding agent of any one of embodiments 1 to 6, further comprising an immunoglobulin (Ig) constant region, or a fragment of the Ig constant region, wherein optionally the fragment of the Ig constant region is an Fc region or an CH3 domain.
8 The binding agent of embodiment 7, wherein the Ig constant region, the fragment of the Ig constant region, the Fc region, or the CH3 domain comprises at least one mutation.
9. The binding agent of embodiment 8, wherein the at least one mutation is selected from the group consisting of L234A/L235A/D265S, F234A/L235A, L234A/L235A, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.
10. The binding agent of embodiment 8, wherein the at least one mutation is selected from the group consisting of T366S/L368A/Y407V, T366W, T350V, L351Y, F405A, Y407V, T366Y, T366L, F405W, T394W, K392L, T394S, Y407T, Y407A, L351Y/F405A/Y407V, T366I/K392M/T394W, F405A/Y407V, T366L/K392M/T394W, T366L/K392L/T394W, L351Y/Y407A, L351Y/Y407V, T366A/K409F, T366V/K409F, T366A/K409F, T350V/L351Y/F405A/Y407V and T350V/T366L/K392L/T394W, wherein residue numbering is according to the EU index.
11. The binding agent of embodiment 8, wherein the binding agent comprises knob-in-hole mutations, wherein the knob mutations comprise T366S/L368A/Y407V, and the hole mutation comprises T366W.
12. The binding agent of any one of embodiments 1 to 11, wherein the agent comprises a bispecific protein comprising an antigen binding region that binds a second antigen other than ENPP3.
13. The binding agent of embodiment 12, wherein the second antigen is cluster of differentiation 3ε (CD3ε).
14. A binding agent comprising a first antigen binding region that binds to ENPP3 and a second antigen binding region that binds to CD3ε, wherein the first antigen binding region that binds to ENPP3 comprises a VH and VL selected from the group consisting of:
   (a) a VH domain comprising a HCDR1, HCDR2, and HCDR3 of a VH as set forth in SEQ ID NO:22, and a VL domain comprising a LCDR1, LCDR2, and LCDR3 of a VL as set forth in SEQ ID NO:23;
   (b) a VH domain comprising a HCDR1, HCDR2, and HCDR3 of a VH as set forth in SEQ ID NO:45, and a VL domain comprising a LCDR1, LCDR2, and LCDR3 of a VL as set forth in SEQ ID NO:46;
   (c) a VH domain comprising a HCDR1, HCDR2, and HCDR3 of a VH as set forth in SEQ ID NO:68, and a VL domain comprising a LCDR1, LCDR2, and LCDR3 of a VL as set forth in SEQ ID NO:69 or SEQ ID NO:70;
   (d) a VH domain comprising a HCDR1, HCDR2, and HCDR3 of a VH as set forth in SEQ ID NO:91, and a VL domain comprising a LCDR1, LCDR2, and LCDR3 of a VL as set forth in SEQ ID NO:92;
   (e) a VH domain comprising a HCDR1, HCDR2, and HCDR3 of a VH as set forth in SEQ ID NO:113, and a VL domain comprising a LCDR1, LCDR2, and LCDR3 of a VL as set forth in SEQ ID NO:114 or SEQ ID NO:115;
   (f) a VH domain comprising a HCDR1, HCDR2, and HCDR3 of a VH as set forth in SEQ ID NO:134, and a VL domain comprising a LCDR1, LCDR2, and LCDR3 of a VL as set forth in SEQ ID NO: 135 or SEQ ID NO:136;
   (g) a VH domain comprising a HCDR1, HCDR2, and HCDR3 of a VH as set forth in SEQ ID NO:158, and a VL domain comprising a LCDR1, LCDR2, and LCDR3 of a VL as set forth in SEQ ID NO: 159; and
   (h) a VH domain comprising a HCDR1, HCDR2, and HCDR3 of a VH as set forth in SEQ ID NO: 177, and a VL domain comprising a LCDR1, LCDR2, and LCDR3 of a VL as set forth in SEQ ID NO: 178.
15. The binding agent of embodiment 14, wherein in the second antigen binding region that binds to CD3ε comprises a VH domain comprising a HCDR1, HCDR2, and HCDR3 of a VH as set forth in SEQ ID NO:200, and a VL domain comprising a LCDR1, LCDR2, and LCDR3 of a VL as set forth in SEQ ID NO:201.
16. The binding agent of embodiment 14, wherein the first antigen binding region that binds to ENPP3 comprises CDR sequences selected from the group consisting of:
   (a1) the HCDR1 comprises the amino acid sequence of SEQ ID NO: 1, the HCDR2 comprises the amino acid sequence of SEQ ID NO:2, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
   (a2) the HCDR1 comprises the amino acid sequence of SEQ ID NO:7, the HCDR2 comprises the amino acid sequence of SEQ ID NO:8, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
   (a3) the HCDR1 comprises the amino acid sequence of SEQ ID NO:9, the HCDR2 comprises the amino acid sequence of SEQ ID NO: 10, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
   (a4) the HCDR1 comprises the amino acid sequence of SEQ ID NO:11, the HCDR2 comprises the amino acid sequence of SEQ ID NO: 12, the HCDR3 comprises the amino acid sequence of SEQ ID NO:13, the LCDR1 comprises the amino acid sequence of SEQ ID NO:14, the LCDR2 comprises the amino acid sequence of SEQ ID NO:15, and the LCDR3 comprises the amino acid sequence of SEQ ID NO: 16;
   (a5) the HCDR1 comprises the amino acid sequence of SEQ ID NO: 17, the HCDR2 comprises the amino acid sequence of SEQ ID NO:18, the HCDR3 comprises the amino acid sequence of SEQ ID NO: 19, the LCDR1 comprises the amino acid sequence of SEQ ID NO:20, the LCDR2 comprises the amino acid sequence of GAS, and the LCDR3 comprises the amino acid sequence of SEQ ID NO:6;
(b1) the HCDR1 comprises the amino acid sequence of SEQ ID NO:24, the HCDR2 comprises the amino acid sequence of SEQ ID NO:25, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
(b2) the HCDR1 comprises the amino acid sequence of SEQ ID NO:30, the HCDR2 comprises the amino acid sequence of SEQ ID NO:31, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
(b3) the HCDR1 comprises the amino acid sequence of SEQ ID NO:32, the HCDR2 comprises the amino acid sequence of SEQ ID NO:33, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
   (b4) the HCDR1 comprises the amino acid sequence of SEQ ID NO:34, the HCDR2 comprises the amino acid sequence of SEQ ID NO:35, the HCDR3 comprises the amino acid sequence of SEQ ID NO:36, the LCDR1 comprises the amino acid sequence of SEQ ID NO:37, the LCDR2 comprises the amino acid sequence of SEQ ID NO:38, and the LCDR3 comprises the amino acid sequence of SEQ ID NO: 39;
   (b5) the HCDR1 comprises the amino acid sequence of SEQ ID NO:40, the HCDR2 comprises the amino acid sequence of SEQ ID NO:41, the HCDR3 comprises the amino acid sequence of SEQ ID NO:42, the LCDR1 comprises the amino acid sequence of SEQ ID NO:43, the LCDR2 comprises the amino acid sequence of DAS, and the LCDR3 comprises the amino acid sequence of SEQ ID NO:29;
   (c1) the HCDR1 comprises the amino acid sequence of SEQ ID NO:47, the HCDR2 comprises the amino acid sequence of SEQ ID NO:48, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
   (c2) the HCDR1 comprises the amino acid sequence of SEQ ID NO:53, the HCDR2 comprises the amino acid sequence of SEQ ID NO:54, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
   (c3) the HCDR1 comprises the amino acid sequence of SEQ ID NO:55, the HCDR2 comprises the amino acid sequence of SEQ ID NO:56, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
   (c4) the HCDR1 comprises the amino acid sequence of SEQ ID NO:57, the HCDR2 comprises the amino acid sequence of SEQ ID NO:58, the HCDR3 comprises the amino acid sequence of SEQ ID NO:59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO: 62;
   (c5) the HCDR1 comprises the amino acid sequence of SEQ ID NO:63, the HCDR2 comprises the amino acid sequence of SEQ ID NO:64, the HCDR3 comprises the amino acid sequence of SEQ ID NO:65, the LCDR1 comprises the amino acid sequence of SEQ ID NO:66, the LCDR2 comprises the amino acid sequence of QIS, and the LCDR3 comprises the amino acid sequence of SEQ ID NO:52;
   (d1) the HCDR1 comprises the amino acid sequence of SEQ ID NO:71, the HCDR2 comprises the amino acid sequence of SEQ ID NO:72, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
   (d2) the HCDR1 comprises the amino acid sequence of SEQ ID NO: 77, the HCDR2 comprises the amino acid sequence of SEQ ID NO:78, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
   (d3) the HCDR1 comprises the amino acid sequence of SEQ ID NO:79, the HCDR2 comprises the amino acid sequence of SEQ ID NO:80, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
   (d4) the HCDR1 comprises the amino acid sequence of SEQ ID NO:81, the HCDR2 comprises the amino acid sequence of SEQ ID NO:82, the HCDR3 comprises the amino acid sequence of SEQ ID NO:83, the LCDR1 comprises the amino acid sequence of SEQ ID NO:84, the LCDR2 comprises the amino acid sequence of SEQ ID NO:85, and the LCDR3 comprises the amino acid sequence of SEQ ID NO: 86;
   (d5) the HCDR1 comprises the amino acid sequence of SEQ ID NO:87, the HCDR2 comprises the amino acid sequence of SEQ ID NO:88, the HCDR3 comprises the amino acid sequence of SEQ ID NO:89, the LCDR1 comprises the amino acid sequence of SEQ ID NO:90, the LCDR2 comprises the amino acid sequence of GAS, and the LCDR3 comprises the amino acid sequence of SEQ ID NO:76;
   (e1) the HCDR1 comprises the amino acid sequence of SEQ ID NO:93, the HCDR2 comprises the amino acid sequence of SEQ ID NO:94, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97 and the LCDR3 comprises the amino acid sequence of SEQ ID NO: 98;
   (e2) the HCDR1 comprises the amino acid sequence of SEQ ID NO:99, the HCDR2 comprises the amino acid sequence of SEQ ID NO: 100, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO: 98;
   (e3) the HCDR1 comprises the amino acid sequence of SEQ ID NO: 101, the HCDR2 comprises the amino acid sequence of SEQ ID NO:102, the HCDR3 comprises the amino acid sequence of SEQ ID NO:95, the LCDR1 comprises the amino acid sequence of SEQ ID NO:96 the LCDR2 comprises the amino acid sequence of SEQ ID NO:97, and the LCDR3 comprises the amino acid sequence of SEQ ID NO: 98;
   (e4) the HCDR1 comprises the amino acid sequence of SEQ ID NO: 103, the HCDR2 comprises the amino acid sequence of SEQ ID NO: 104, the HCDR3 comprises the amino acid sequence of SEQ ID NO: 105, the LCDR1 comprises the amino acid sequence of SEQ ID NO: 106, the LCDR2 comprises the amino acid sequence of SEQ ID NO:107, and the LCDR3 comprises the amino acid sequence of SEQ ID NO:108;
   (e5) the HCDR1 comprises the amino acid sequence of SEQ ID NO: 109, the HCDR2 comprises the amino acid sequence of SEQ ID NO:110, the HCDR3 comprises the amino acid sequence of SEQ ID NO:111, the LCDR1 comprises the amino acid sequence of SEQ ID NO: 112, the LCDR2 comprises the amino acid sequence of GAS, and the LCDR3 comprises the amino acid sequence of SEQ ID NO: 98;
   (f1) the HCDR1 comprises the amino acid sequence of SEQ ID NO: 116, the HCDR2 comprises the amino acid sequence of SEQ ID NO: 117, the HCDR3 comprises the amino acid sequence of SEQ ID NO: 118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120;
   (f2) the HCDR1 comprises the amino acid sequence of SEQ ID NO: 121, the HCDR2 comprises the amino acid sequence of SEQ ID NO: 122, the HCDR3 comprises the amino acid sequence of SEQ ID NO:118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO:120;
   (f3) the HCDR1 comprises the amino acid sequence of SEQ ID NO: 123, the HCDR2 comprises the amino acid sequence of SEQ ID NO: 124, the HCDR3 comprises the amino acid sequence of SEQ ID NO:118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO:120;
   (f4) the HCDR1 comprises the amino acid sequence of SEQ ID NO: 125, the HCDR2 comprises the amino acid sequence of SEQ ID NO: 126, the HCDR3 comprises the amino acid sequence of SEQ ID NO:127, the LCDR1 comprises the amino acid sequence of SEQ ID NO: 128, the LCDR2 comprises the amino acid sequence of SEQ ID NO:15, and the LCDR3 comprises the amino acid sequence of SEQ ID NO:129;
   (f5) the HCDR1 comprises the amino acid sequence of SEQ ID NO: 130, the HCDR2 comprises the amino acid sequence of SEQ ID NO: 131, the HCDR3 comprises the amino acid sequence of SEQ ID NO: 132, the LCDR1 comprises the amino acid sequence of SEQ ID NO: 133, the LCDR2 comprises the amino acid sequence of GAS, and the LCDR3 comprises the amino acid sequence of SEQ ID NO: 120;
   (g1) the HCDR1 comprises the amino acid sequence of SEQ ID NO: 137, the HCDR2 comprises the amino acid sequence of SEQ ID NO:138, the HCDR3 comprises the amino acid sequence of SEQ ID NO: 139, the LCDR1 comprises the amino acid sequence of SEQ ID NO:140, the LCDR2 comprises the amino acid sequence of SEQ ID NO: 141, and the LCDR3 comprises the amino acid sequence of SEQ ID NO:142;
   (g2) the HCDR1 comprises the amino acid sequence of SEQ ID NO: 143, the HCDR2 comprises the amino acid sequence of SEQ ID NO: 144, the HCDR3 comprises the amino acid sequence of SEQ ID NO:139, the LCDR1 comprises the amino acid sequence of SEQ ID NO: 140, the LCDR2 comprises the amino acid sequence of SEQ ID NO:141, and the LCDR3 comprises the amino acid sequence of SEQ ID NO: 142;
   (g3) the HCDR1 comprises the amino acid sequence of SEQ ID NO:145, the HCDR2 comprises the amino acid sequence of SEQ ID NO: 146, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO: 142;
   (g4) the HCDR1 comprises the amino acid sequence of SEQ ID NO: 147, the HCDR2 comprises the amino acid sequence of SEQ ID NO: 148, the HCDR3 comprises the amino acid sequence of SEQ ID NO:149, the LCDR1 comprises the amino acid sequence of SEQ ID NO:150, the LCDR2 comprises the amino acid sequence of SEQ ID NO:151, and the LCDR3 comprises the amino acid sequence of SEQ ID NO:152;
   (g5) the HCDR1 comprises the amino acid sequence of SEQ ID NO: 153, the HCDR2 comprises the amino acid sequence of SEQ ID NO:154, the HCDR3 comprises the amino acid sequence of SEQ ID NO: 155, the LCDR1 comprises the amino acid sequence of SEQ ID NO: 156, the LCDR2 comprises the amino acid sequence of AAS, and the LCDR3 comprises the amino acid sequence of SEQ ID NO: 142;
   (h1) the HCDR1 comprises the amino acid sequence of SEQ ID NO: 1, the HCDR2 comprises the amino acid sequence of SEQ ID NO: 160, the HCDR3 comprises the amino acid sequence of SEQ ID NO: 161, the LCDR1 comprises the amino acid sequence of SEQ ID NO:162, the LCDR2 comprises the amino acid sequence of SEQ ID NO:163, and the LCDR3 comprises the amino acid sequence of SEQ ID NO:76;
   (h2) the HCDR1 comprises the amino acid sequence of SEQ ID NO: 164, the HCDR2 comprises the amino acid sequence of SEQ ID NO:165, the HCDR3 comprises the amino acid sequence of SEQ ID NO: 161, the LCDR1 comprises the amino acid sequence of SEQ ID NO: 162 the LCDR2 comprises the amino acid sequence of SEQ ID NO: 163, and the LCDR3 comprises the amino acid sequence of SEQ ID NO: 76;
   (h3) the HCDR1 comprises the amino acid sequence of SEQ ID NO: 166, the HCDR2 comprises the amino acid sequence of SEQ ID NO: 167, the HCDR3 comprises the amino acid sequence of SEQ ID NO: 161, the LCDR1 comprises the amino acid sequence of SEQ ID NO: 162, the LCDR2 comprises the amino acid sequence of SEQ ID NO: 163, and the LCDR3 comprises the amino acid sequence of SEQ ID NO:76;
   (h4) the HCDR1 comprises the amino acid sequence of SEQ ID NO: 11, the HCDR2 comprises the amino acid sequence of SEQ ID NO:168, the HCDR3 comprises the amino acid sequence of SEQ ID NO: 169, the LCDR1 comprises the amino acid sequence of SEQ ID NO: 170, the LCDR2 comprises the amino acid sequence of SEQ ID NO:171, and the LCDR3 comprises the amino acid sequence of SEQ ID NO:86; and
   (h5) the HCDR1 comprises the amino acid sequence of SEQ ID NO: 172, the HCDR2 comprises the amino acid sequence of SEQ ID NO:173, the HCDR3 comprises the amino acid sequence of SEQ ID NO: 174, the LCDR1 comprises the amino acid sequence of SEQ ID NO: 175, the LCDR2 comprises the amino acid sequence of VAS and the LCDR3 comprises the amino acid sequence of SEQ ID NO: 76.
17. The binding agent of embodiment 15, wherein the second antigen binding region that binds to CD3ε comprises CDR sequences selected from the group consisting of:
   (a) the HCDR1 comprises the amino acid sequence of SEQ ID NO:179, the HCDR2 comprises the amino acid sequence of SEQ ID NO: 180, the HCDR3 comprises the amino acid sequence of SEQ ID NO: 181, the LCDR1 comprises the amino acid sequence of SEQ ID NO: 182, the LCDR2 comprises the amino acid sequence of SEQ ID NO: 183, and the LCDR3 comprises the amino acid sequence of SEQ ID NO:184;
   (b) the HCDR1 comprises the amino acid sequence of SEQ ID NO: 185, the HCDR2 comprises the amino acid sequence of SEQ ID NO:186, the HCDR3 comprises the amino acid sequence of SEQ ID NO: 181, the LCDR1 comprises the amino acid sequence of SEQ ID NO: 182 the LCDR2 comprises the amino acid sequence of SEQ ID NO: 183, and the LCDR3 comprises the amino acid sequence of SEQ ID NO: 184;
   (c) the HCDR1 comprises the amino acid sequence of SEQ ID NO: 187, the HCDR2 comprises the amino acid sequence of SEQ ID NO: 188, the HCDR3 comprises the amino acid sequence of SEQ ID NO: 181, the LCDR1 comprises the amino acid sequence of SEQ ID NO: 182, the LCDR2 comprises the amino acid sequence of SEQ ID NO:183, and the LCDR3 comprises the amino acid sequence of SEQ ID NO: 184;
   (d) the HCDR1 comprises the amino acid sequence of SEQ ID NO: 189, the HCDR2 comprises the amino acid sequence of SEQ ID NO: 190, the HCDR3 comprises the amino acid sequence of SEQ ID NO:191, the LCDR1 comprises the amino acid sequence of SEQ ID NO:192, the LCDR2 comprises the amino acid sequence of SEQ ID NO: 193, and the LCDR3 comprises the amino acid sequence of SEQ ID NO: 194; and
   (e) the HCDR1 comprises the amino acid sequence of SEQ ID NO: 195, the HCDR2 comprises the amino acid sequence of SEQ ID NO: 196, the HCDR3 comprises the amino acid sequence of SEQ ID NO: 197, the LCDR1 comprises the amino acid sequence of SEQ ID NO: 198, the LCDR2 comprises the amino acid sequence of DSS and the LCDR3 comprises the amino acid sequence of SEQ ID NO: 184.
18. The binding agent of any one of embodiments 14 to 17, wherein
   (i) the first binding region comprises a VH domain comprising the amino acid sequence of SEQ ID NO:22, and a VL domain comprising the amino acid sequence of SEQ ID NO:23; and
   (ii) the second binding region comprises a VH domain comprising the amino acid sequence of SEQ ID NO:200, and a VL domain comprising the amino acid sequence of SEQ ID NO:201.
19. The binding agent of any one of embodiments 12 to 18, wherein the first antigen binding region comprises a Fab, and the second antigen binding region comprises a stapled scFv fragment (spFv).
20. The binding agent of embodiment 19, wherein the spFv comprises at least one disulfide bond between the VH or VL and the linker.
21. The binding agent of any one of embodiments 12 to 20, wherein the binding agent further comprises an immunoglobulin (Ig) constant region, a fragment of the Ig constant region, wherein optionally the fragment of the Ig constant region is an Fc region or an CH3 domain.
22. A binding agent comprising
   (i) a first polypeptide comprising a spFv that binds CD3ε, a CH2 domain and a CH3 domain;
   (ii) a second polypeptide comprising a VH domain that binds ENPP3, a CH2 domain and a CH3 domain; and
   (iii) a third polypeptide comprising a VL domain that binds ENPP3,
      wherein the spFv that binds CD3ε comprises a VH domain comprising a HCDR1, HCDR2, and HCDR3 of a VH as set forth in SEQ ID NO:200, and a VL domain comprising a LCDR1, LCDR2, and LCDR3 of a VL as set forth in SEQ ID NO:201; and wherein the second and third polypeptide comprising a VH and VL that bind to ENPP3, wherein the VH and VL that bind to ENPP3 are selected from the group consisting of:
   (a) a VH domain comprising a HCDR1, HCDR2, and HCDR3 of a VH as set forth in SEQ ID NO:22, and a VL domain comprising a LCDR1, LCDR2, and LCDR3 of a VL as set forth in SEQ ID NO:23;
   (b) a VH domain comprising a HCDR1, HCDR2, and HCDR3 of a VH as set forth in SEQ ID NO:45, and a VL domain comprising a LCDR1, LCDR2, and LCDR3 of a VL as set forth in SEQ ID NO:46;
   (c) a VH domain comprising a HCDR1, HCDR2, and HCDR3 of a VH as set forth in SEQ ID NO:68, and a VL domain comprising a LCDR1, LCDR2, and LCDR3 of a VL as set forth in SEQ ID NO:69 or SEQ ID NO:70;
   (d) a VH domain comprising a HCDR1, HCDR2, and HCDR3 of a VH as set forth in SEQ ID NO:91, and a VL domain comprising a LCDR1, LCDR2, and LCDR3 of a VL as set forth in SEQ ID NO:92;
   (e) a VH domain comprising a HCDR1, HCDR2, and HCDR3 of a VH as set forth in SEQ ID NO:113, and a VL domain comprising a LCDR1, LCDR2, and LCDR3 of a VL as set forth in SEQ ID NO:114 or SEQ ID NO:115;
   (f) a VH domain comprising a HCDR1, HCDR2, and HCDR3 of a VH as set forth in SEQ ID NO:134, and a VL domain comprising a LCDR1, LCDR2, and LCDR3 of a VL as set forth in SEQ ID NO:135 or SEQ ID NO:136;
   (g) a VH domain comprising a HCDR1, HCDR2, and HCDR3 of a VH as set forth in SEQ ID NO:158, and a VL domain comprising a LCDR1, LCDR2, and LCDR3 of a VL as set forth in SEQ ID NO: 159; and
   (h) a VH domain comprising a HCDR1, HCDR2, and HCDR3 of a VH as set forth in SEQ ID NO:177, and a VL domain comprising a LCDR1, LCDR2, and LCDR3 of a VL as set forth in SEQ ID NO:178.
23. The binding agent of embodiment 22, wherein:
   (i) the spFv that binds CD3ε comprises CDRs selected from the group consisting of:
   (a) the HCDR1 comprises the amino acid sequence of SEQ ID NO:179, the HCDR2 comprises the amino acid sequence of SEQ ID NO: 180, the HCDR3 comprises the amino acid sequence of SEQ ID NO:181, the LCDR1 comprises the amino acid sequence of SEQ ID NO:182, the LCDR2 comprises the amino acid sequence of SEQ ID NO: 183, and the LCDR3 comprises the amino acid sequence of SEQ ID NO:184;
   (b) the HCDR1 comprises the amino acid sequence of SEQ ID NO: 185, the HCDR2 comprises the amino acid sequence of SEQ ID NO: 186, the HCDR3 comprises the amino acid sequence of SEQ ID NO: 181, the LCDR1 comprises the amino acid sequence of SEQ ID NO: 182 the LCDR2 comprises the amino acid sequence of SEQ ID NO:183, and the LCDR3 comprises the amino acid sequence of SEQ ID NO:184;
   (c) the HCDR1 comprises the amino acid sequence of SEQ ID NO: 187, the HCDR2 comprises the amino acid sequence of SEQ ID NO: 188, the HCDR3 comprises the amino acid sequence of SEQ ID NO: 181, the LCDR1 comprises the amino acid sequence of SEQ ID NO:182, the LCDR2 comprises the amino acid sequence of SEQ ID NO: 183, and the LCDR3 comprises the amino acid sequence of SEQ ID NO:184;
   (d) the HCDR1 comprises the amino acid sequence of SEQ ID NO: 189, the HCDR2 comprises the amino acid sequence of SEQ ID NO: 190, the HCDR3 comprises the amino acid sequence of SEQ ID NO:191, the LCDR1 comprises the amino acid sequence of SEQ ID NO: 192, the LCDR2 comprises the amino acid sequence of SEQ ID NO: 193, and the LCDR3 comprises the amino acid sequence of SEQ ID NO:194; and
   (e) the HCDR1 comprises the amino acid sequence of SEQ ID NO:195, the HCDR2 comprises the amino acid sequence of SEQ ID NO: 196, the HCDR3 comprises the amino acid sequence of SEQ ID NO: 197, the LCDR1 comprises the amino acid sequence of SEQ ID NO:198, the LCDR2 comprises the amino acid sequence of DSS and the LCDR3 comprises the amino acid sequence of SEQ ID NO:184; and
   (ii) the Fab that binds ENPP3 comprises:
   (a) in the VH domain that binds ENPP3, HCDR1 comprises the amino acid sequence of SEQ ID NO:1; HCDR2 comprises the amino acid sequence of SEQ ID NO:2; HCDR3 comprises the amino acid sequence of SEQ ID NO: 3; and in the VL domain that binds ENPP3, LCDR1 comprises the amino acid sequence of SEQ ID NO:4; LCDR2 comprises the amino acid sequence of SEQ ID NO:5; and LCDR3 comprises the amino acid sequence of SEQ ID NO:6;
   (b) in the VH domain that binds ENPP3, HCDR1 comprises the amino acid sequence of SEQ ID NO:7; HCDR2 comprises the amino acid sequence of SEQ ID NO:8; HCDR3 comprises the amino acid sequence of SEQ ID NO: 3; and in the VL domain that binds ENPP3, LCDR1 comprises the amino acid sequence of SEQ ID NO:4; LCDR2 comprises the amino acid sequence of SEQ ID NO:5; and LCDR3 comprises the amino acid sequence of SEQ ID NO:6;
   (c) in the VH domain that binds ENPP3, HCDR1 comprises the amino acid sequence of SEQ ID NO:9; HCDR2 comprises the amino acid sequence of SEQ ID NO:10; HCDR3 comprises the amino acid sequence of SEQ ID NO: 3; and in the VL domain that binds ENPP3, LCDR1 comprises the amino acid sequence of SEQ ID NO:4; LCDR2 comprises the amino acid sequence of SEQ ID NO:5; and LCDR3 comprises the amino acid sequence of SEQ ID NO:6;
   (d) in the VH domain that binds ENPP3, HCDR1 comprises the amino acid sequence of SEQ ID NO:11; HCDR2 comprises the amino acid sequence of SEQ ID NO: 12; HCDR3 comprises the amino acid sequence of SEQ ID NO: 13; and in the VL domain that binds ENPP3, LCDR1 comprises the amino acid sequence of SEQ ID NO:14; LCDR2 comprises the amino acid sequence of SEQ ID NO:15; and LCDR3 comprises the amino acid sequence of SEQ ID NO:16; and
   (e) in the VH domain that binds ENPP3, HCDR1 comprises the amino acid sequence of SEQ ID NO:17; HCDR2 comprises the amino acid sequence of SEQ ID NO:18; HCDR3 comprises the amino acid sequence of SEQ ID NO: 19; and in the VL domain that binds ENPP3, LCDR1 comprises the amino acid sequence of SEQ ID NO:20; LCDR2 comprises the amino acid sequence of GAS; and LCDR3 comprises the amino acid sequence of SEQ ID NO: 6.
24. The binding agent of embodiment 22 or 23, wherein the spFv that binds CD3ε comprises a VH domain comprising the amino acid sequence of SEQ ID NO:200, and a VL domain comprising the amino acid sequence of SEQ ID NO:201; the VH domain that binds ENPP3 comprises the amino acid sequence of SEQ ID NO:22, and the VL domain that binds ENPP3 comprises the amino acid sequence of SEQ ID NO:23.
25. The binding agent of any one of embodiments 22 to 24, wherein the spFv that binds CD3ε comprises the amino acid sequence of SEQ ID NO:248; the VH domain that binds ENPP3 comprises the amino acid sequence of SEQ ID NO:22, and the VL domain that binds ENPP3 comprises the amino acid sequence of SEQ ID NO:23.
26. The binding agent of any one of embodiments 22 to 25, wherein:
   (i) the first polypeptide comprising a spFv that binds CD3ε, a CH2 domain and a CH3 domain comprises the amino acid sequence of SEQ ID NO:255;
   (ii) the second polypeptide comprising a VH domain that binds ENPP3, a CH2 domain and a CH3 domain comprises the amino acid sequence of SEQ ID NO: 256; and
   (iii) the third polypeptide comprising a VL domain that binds ENPP3 comprises the amino acid sequence of SEQ ID NO:257.
27. The binding agent of any one of embodiments 22 to 26, wherein the spFv comprises at least one disulfide bond between the VH or VL and the linker.
28. A composition comprising the binding agent of any one of embodiments 1 to 27, and a pharmaceutically acceptable carrier.
29. A polynucleotide comprising nucleotide sequences encoding a VH, a VL, or both a VH and a VL of the binding agent of any one of embodiments 1 to 27.
30. A vector comprising the polynucleotide of embodiment 29.
31. A cell comprising the polynucleotide of embodiment 29.
32. A kit comprising the binding agent of any one of embodiments 1 to 27.
33. A method of making a binding agent which binds to an epitope of ENPP3, comprising culturing the cell of embodiment 31 to express the binding agent.
34. A method of directing a T cell to a target cell expressing ENPP3, comprising contacting the T cell with an effective amount of the binding agent of any one of embodiments 12 to 27 or a composition comprising the binding agent and a pharmaceutically acceptable carrier, wherein the antigen binding region that binds to CD3ε binds the T cell and the antigen binding region that binds to ENPP3 binds to the target cell.
35. A method of treating a cancer or tumor in a subject in need thereof, comprising administering an effective amount the binding agent of any one of embodiments 12 to 27 or a composition comprising the binding agent and a pharmaceutically acceptable carrier to the subject.
36. The method of embodiment 35, wherein the cancer or tumor is selected from the group consisting of a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.

### 7. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); s or sec=second(s); min=minute(s); h or hr=hour(s); aa=amino acid(s); kb=kilobase(s); nt=nucleotide(s); pg-picogram; ng=nanogram; μg=microgram; mg=milligram; g=gram; kg=kilogram; pl or pL=picoliter(s); dl or dL=deciliter; μl or μL=microliter; ml or mL=milliliter; l or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; TIW=three times a week; QM=monthly; HPLC=high performance liquid chromatography; BW=body weight; U=unit; ns=not statistically significant; PBS=phosphate-buffered saline; PCR=polymerase chain reaction; NHS=N-Hydroxysuccinimide; HSA=human serum albumin; BSA=bovine serum albumin; DMEM=Dulbeco's Modification of Eagle's Medium; GC=genome copy; EDTA=ethylenediaminetetraacetic acid.

The following experimental methods were employed throughout the Examples described herein.

### 7.1 Example 1: Generation of Antibodies Binding to ENPP3

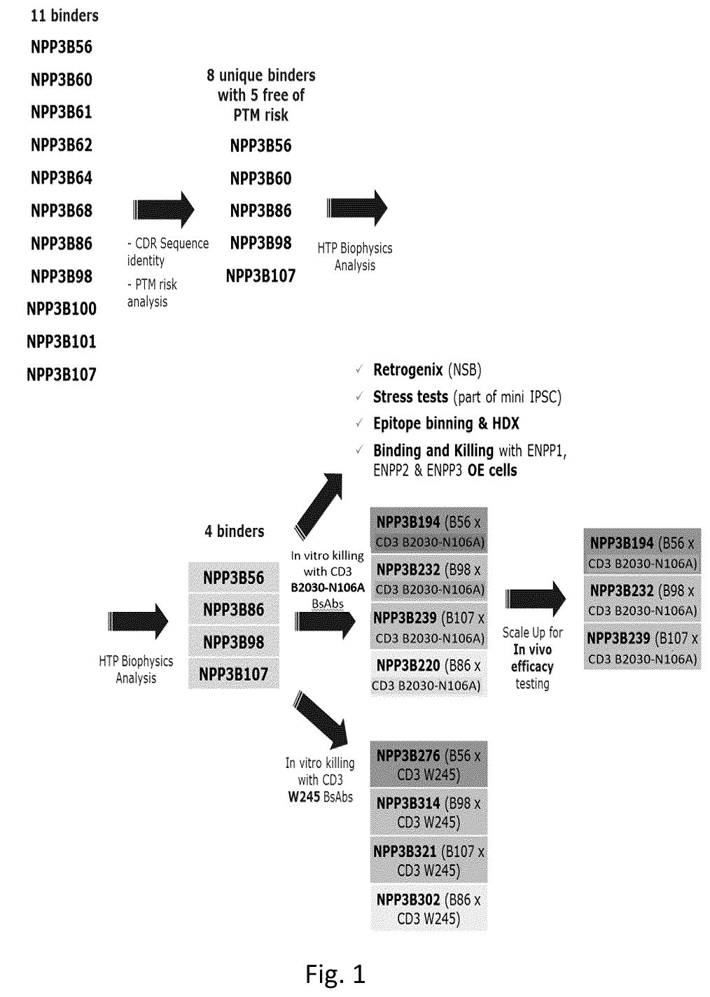

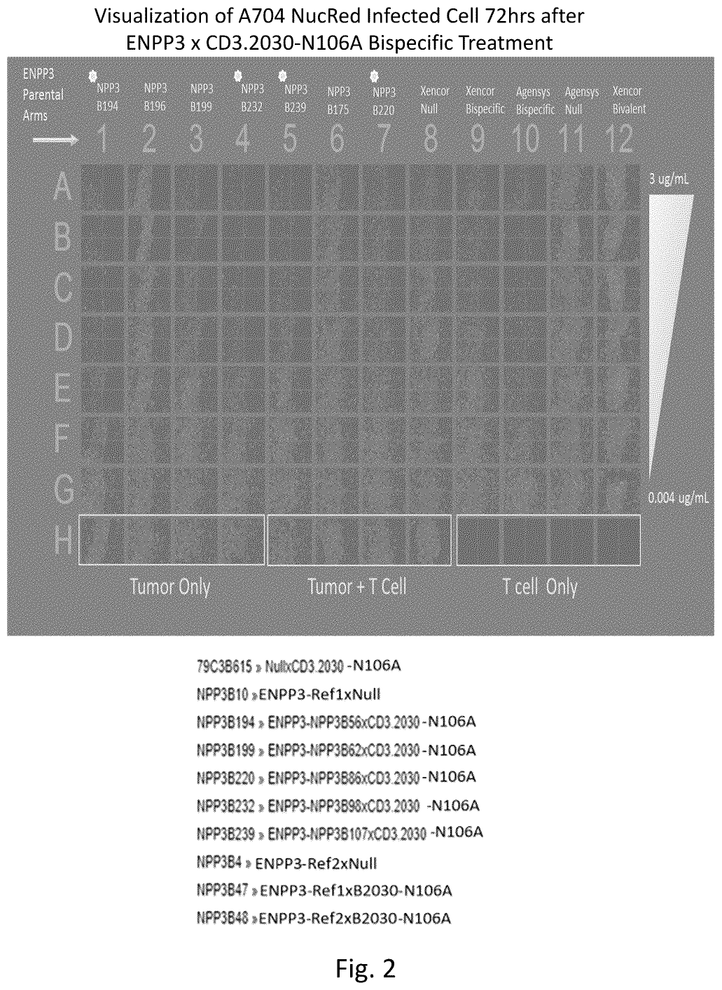

Anti-ENPP3 antibodies were generated using Ablexis transgenic mouse platform, using techniques known in the art. Briefly, Ablexis Kappa mice were immunized using a plasmid comprising full length human ENPP3 DNA sequence. B cells were recovered following standard protocols, and the supernatants from the sorted B cells were screened by protein MSD for binding to human ENPP3 (Uniprot ID: 014638). Fifteen binders were selected based on binding to human ENPP3. Screening cascade is shown in FIG. 1. Eleven binders were selected for further characterization (FIGS. 1-2 and Tables 1-11). The binding affinities for select binders to human and cynomolgus monkey ENPP3 are shown in Table 17.

TABLE 17
Binding affinities of select binders to human
and cyno ENPP3.

| | Binding | |
|---|---|---|
| | to human | |
| | ENPP3 | Binding to cyno ENPP3 |
| Antibody | KD (M) | KD (M) |
| NPP3B100 | 9.77E−11 | No binding up to 300 nM |
| NPP3B101 | 1.93E−10 | 9.58E−09 |
| NPP3B107 | 1.33E−10 | ~20 nM (biphasic) |
| NPP3B56 | 2.15E−10 | ~2 nM (biphasic) |
| NPP3B60 | 4.48E−10 | ~1 nM (biphasic) |
| NPP3B61 | 4.43E−10 | 1.09E−07 |
| NPP3B62 | 2.58E−10 | 1.20E−08 |
| NPP3B64 | 5.36E−10 | ~2 nM (biphasic) |
| NPP3B68 | N/A | N/A |
| NPP3B86 | 2.45E−10 | ~5 nM (biphasic) |
| NPP3B98 | 1.29E−10 | ~1 nM (biphasic) |

Next, the binders were assessed for, among other things, CDR sequence similarity, biophysics characteristics, and developability characteristics, such as binding affinity to human and cyno ENPP3, hydrophobicity, non-specific binding, cross-interaction chromatography (CIC), conformational stability and SPR based binding to other ENPP family members. Based on these results, 5 ENPP3 binders (FIG. 1) were selected for further functional evaluation.

### 7.1.1 Epitope Studies

The epitope on human ENPP3 was determined by hydrogen-deuterium exchange mass spectrometry (HDX-MS).

On-Exchange Experiment for HDX-MS. On-exchange reaction was initiated by mixing 10 μL of 6 μM recombinant human ENPP3 (Sino Biological; SEQ ID NO: 327), which includes residues 46-875 of human ectonucleotide pyrophosphatase/phosphodiesterase (Uniprot ID 014638), with or without 1.2 molar-excess of ligand and 30 μL of H2O or a deuterated buffer (such as 20 mM MES, pH 6.0, 150 mM NaCl in 95% D2O; 20 mM Tris, pH 8.0, 150 mM NaCl in 95% D2O; or DPBS, pH 7.2 in 90% D20). The reaction mixture was incubated for 15, 50, 150, 500, or 1,500 s at 23° C. The on-exchanged solution was quenched by the addition of chilled 40 μL of 8 M urea, 1 M TCEP hydrochloride, pH 4.0 and immediately analyzed.

### ENPP3 (Uniprot ID 014638) (SEQ ID NO: 327):

HHHHHHHHHHGLNDIFEAQKIEWHERKLEKQGSCRKKCFDASFRGLENC
RCDVACKDRGDCCWDFEDTCVESTRIWMCNKFRCGETRLEASLCSCSDD
CLQRKDCCADYKSVCQGETSWLEENCDTAQQSQCPEGFDLPPVILFSMD
GFRAEYLYTWDTLMPNINKLKTCGIHSKYMRAMYPTKTFPNHYTIVTGL
YPESHGIIDNNMYDVNLNKNFSLSSKEQNNPAWWHGQPMWLTAMYQGLK
AATYFWPGSEVAINGSFPSIYMPYNGSVPFEERISTLLKWLDLPKAERP
RFYTMYFEEPDSSGHAGGPVSARVIKALQVVDHAFGMLMEGLKQRNLHN
CVNIILLADHGMDQTYCNKMEYMTDYFPRINFFYMYEGPAPRIRAHNIP
HDFFSFNSEEIVRNLSCRKPDQHFKPYLTPDLPKRLHYAKNVRIDKVHL
FVDQQWLAVRSKSNTNCGGGNHGYNNEFRSMEAIFLAHGPSFKEKTEVE
PFENIEVYNLMCDLLRIQPAPNNGTHGSLNHLLKVPFYEPSHAEEVSKF
SVCGFANPLPTESLDCFCPHLQNSTQLEQVNQMLNLTQEEITATVKVNL
PFGRPRVLQKNVDHCLLYHREYVSGFGKAMRMPMWSSYTVPQLGDTSPL
PPTVPDCLRADVRVPPSESQKCSFYLADKNITHGFLYPPASNRTSDSQY
DALITSNLVPMYEEFRKMWDYFHSVLLIKHATERNGVNVVSGPIFDYNY
DGHFDAPDEITKHLANTDVPIPTHYFVVLTSCKNKSHTPENCPGWLDVL
PFIIPHRPTNVESCPEGKPEALWVEERFTAHIARVRDVELLTGLDFYQD
KVQPVSEILQLKTYLPTFETTI

General Procedure for HDX-MS Data Acquisition. HDX-MS sample preparation was performed with automated HDx system (LEAP Technologies, Morrisville, NC). The columns and pump were; protease, protease type XIII (protease from Aspergillus saitoi, type XIII)/pepsin column (w/w, 1:1; 2.1×30 mm) (NovaBioAssays Inc., Woburn, MA); trap, ACQUITY UPLC BEH C18 VanGuard Pre-column (2.1×5 mm) (Waters, Milford, MA), analytical, Accucore C18 (2.1×100 mm) (Thermo Fisher Scientific, Waltham, MA); and LC pump, VH-P10-A (Thermo Fisher Scientific). The loading pump (from the protease column to the trap column) was set at 600 μL/min with 0.1% aqueous formic acid. The gradient pump (from the trap column to the analytical column) was set from 9% to 33% acetonitrile in 0.1% aqueous formic acid in 20 min at 100 μL/min.

MS Data Acquisition. Mass spectrometric analyses were carried out using an LTQ™ Orbitrap Fusion Lumos mass spectrometer (Thermo Fisher Scientific) with the capillary temperature at 275° C., resolution 120,000, and mass range (m/z) 300-1,500.

HDX-MS Data Extraction. BioPharma Finder 2.0 (Thermo Fisher Scientific) was used for the peptide identification of non-deuterated samples prior to the HDX experiments. HDExaminer version 2.5 (Sierra Analytics, Modesto, CA) was used to extract centroid values from the MS raw data files for the HDX experiments.

HDX-MS Data Analysis. The extracted HDX-MS data were further analyzed in Excel to covert deuterium incorporation to folding free energy (ΔG) described as stretched exponential method in the reference (Y. Hamuro “Quantitative Hydrogen/Deuterium Exchange Mass Spectrometry” J. Am. Soc. Mass Spectrom. 2021, 32, 2711-2727). The segments with the free energy change upon ligand binding smaller than-1 kcal/mol (ΔΔG<−1 kcal/mol) are considered HDX-MS identified epitopes.

Results. The epitope of NPP3W6 against NP4D6 and NPP3B15 were residues 762-780 (PDEITKHLANTDVPIPTHY (SEQ ID NO: 328)).

The epitope of NPP3W6 against NPP3B56 were residues 412-415 (PHDF (SEQ ID NO: 295)), 673-676 (PSES (SEQ ID NO: 296)), 762-768 (PDEITKH (SEQ ID NO: 297)), and 773-775 (DVP).

The epitopes of NPP3W6 against NPP3B62 were residues 572-576 (SLECF (SEQ ID NO: 299)), 582-586 (NSTQL (SEQ ID NO: 300)), and 673-681 (PSESQKCSF (SEQ ID NO: 301)).

The epitopes of NPP3W6 against NPP3B98 were residues 556-562 (VSKFSVC (SEQ ID NO: 302)), 612-616 (RPRVL (SEQ ID NO: 303)), 621-624 (DHCL (SEQ ID NO: 304)), and 856-860 (QPVSE (SEQ ID NO: 305)).

The epitopes of NPP3W6 against NPP3B86 were residues 601-602 (TA), and 695-707 (PPASNRTSDSQYD (SEQ ID NO: 329)).

The sequence numbering of NPP3W6 was converted to that of the native sequence (Uniprot ID: 014638).

### 7.2 Example 2. Generation and Characterization of Bi-Specific ENPP3×CD3 Antibodies

### 7.2.1 Engineering of the ENPP3×CD3 Antibodies

The ENPP3 binding arm of ENPP3×CD3 bispecific antibodies was engineered to comprise an anti-ENPP3 Fab arm derived from ENPP3 mAbs described in Example 1 (sequences shown in Tables 1-11). To prepare the bispecific antibody, the VH1 and VL1 of ENPP3 mAbs were engineered in VH1-CH1-hinge-CH2-CH3 (Heavy Chain 1, HC1) and VL1-CL (Light Chain 1, LC1) formats respectively and expressed as IgG1. Mutations designed to promote selective heterodimerization (“hole” mutation T366S, L368A and Y407V) were also engineered in the Fc domain of HC1. The parental anti-ENPP3 variable regions were formatted as a Fab in the bispecific antibody.

The CD3 binding arms of ENPP3×CD3 antibodies were engineered using VH and VL domains of CD3B2030-N106A antibody, disclosed in WO2022/201053, which is incorporated herein by reference in its entirety. The sequences of CD3B2030-N106A antibody are shown in Table 13B. The CD3B2030-N106A is a low-medium affinity CD3 binding arm. The VH and VL of the CD3B2030-N106A antibody were engineered as either a scFv domain or a stabilized (or stapled) scFv domain herein described as spFv, in VL-linker-VH-Fc orientation.

The generation of the scFv fragment derived from the CD3B2030-N106A antibody is described in WO2022/201053, which is incorporated herein by reference in its entirety. VH of SEQ ID NO: 202 and VL of SEQ ID NO: 203 were used to generate the scFv.

Additional CD3 binding arms of ENPP3×CD3 antibodies were engineered using VH and VL domains of CD3W245 antibody. The generation of the scFv fragment derived from the CD3W245 antibody is described in WO2021240388, which is incorporated herein by reference in its entirety. VH of SEQ ID NO: 220 and VL of SEQ ID NO: 221 were used to generate the scFv.

Table 17A shows the engineered bispecific antibodies.

TABLE 17A
bispecific ENPP3 × CD3 antibodies

| Bispecific | | |
|---|---|---|
| antibody ID | ENPP3 arm | CD3 arm |
| NPP3B194 | NPP3B56 | CD3B2030-N104A |
| NPP3B199 | NPP3B62 | CD3B2030-N104A |
| NPP3B232 | NPP3B98 | CD3B2030-N104A |
| NPP3B239 | NPP3B107 | CD3B2030-N104A |
| NPP3B220 | NPP3B86 | CD3B2030-N104A |
| NPP3B276 | NPP3B56 | CD3W245 |
| NPP3B278 | NPP3B58 | CD3W245 |
| NPP3B281 | NPP3B62 | CD3W245 |
| NPP3B314 | NPP3B98 | CD3W245 |
| NPP3B321 | NPP3B107 | CD3W245 |
| NPP3B302 | NPP3B86 | CD3W245 |

The stabilized scFv (spFv) was generated using the VH and the VL of CD3B2030-N106A binder by engineering disulfide bonds between the VH and the linker and between the VL and the linker. Two structurally conserved surface exposed framework positions (anchor points) that are not involved in antigen binding, were identified, one on the VH at position H105 and one on the VL at position L42 (Chothia numbering), and mutated into cysteine (Cys) residues to generate the VH of SEQ ID NO: 200 and VL of SEQ ID NO: 201. A flexible linker of sequence GGGSGGSGGCPPCGGSGG (SEQ ID NO: 292) comprising two Cys residues was used to conjugate the VL and the VH in the VL-linker-VH (LH) format yielding the spFv of SEQ ID NO: 248. The distance and location of the Cys residues of the linker and the Cys residues of the VH and the VL is critical for the formation of the disulfide bonds between the Cys residues of the Linker and each anchor point of the VH and the VL.

The stapled scFv in the VL-Linker-VH was further engineered into a Heavy Chain 2 (HC2) and expressed as IgG1. Additionally, the Fc heterodimerization (“knob” mutation T366W) were engineered in the Fc domain of HC2.

### 7.2.2 In Vitro Killing Using Bi-Specific ENPP3×CD3 Abs

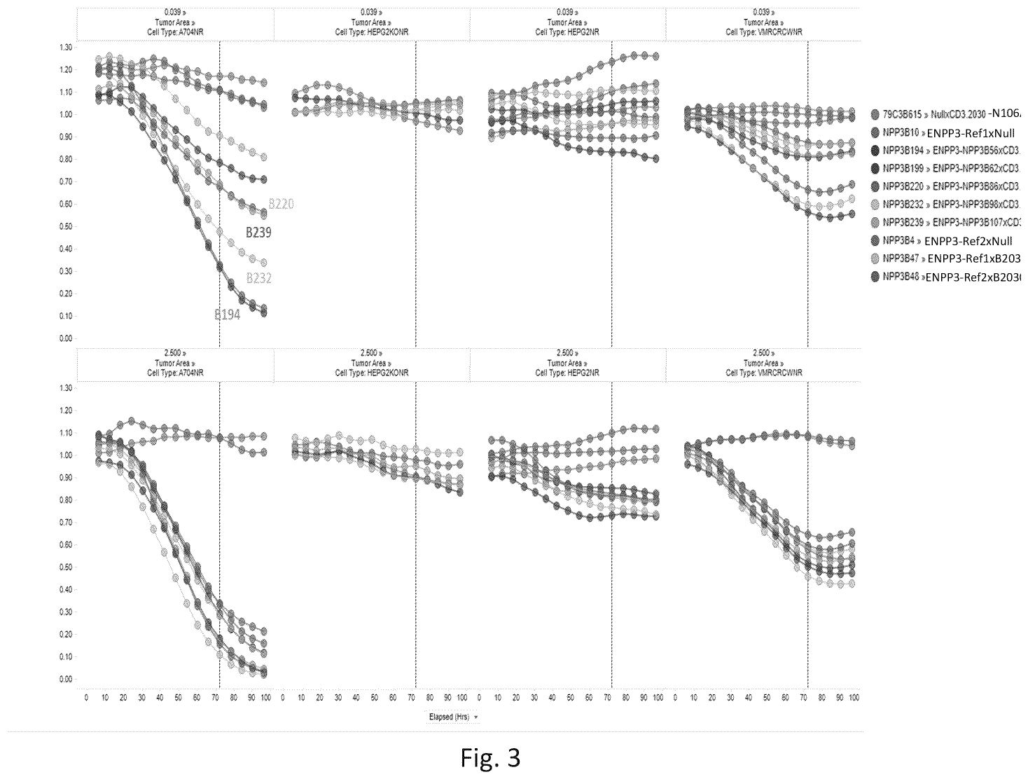

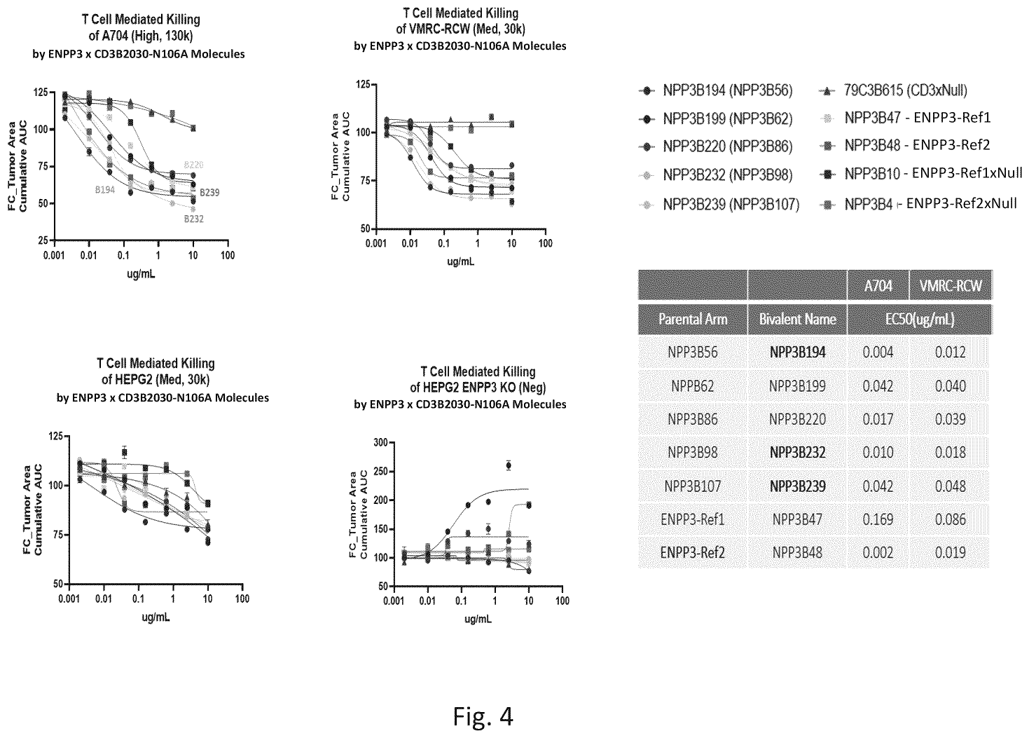

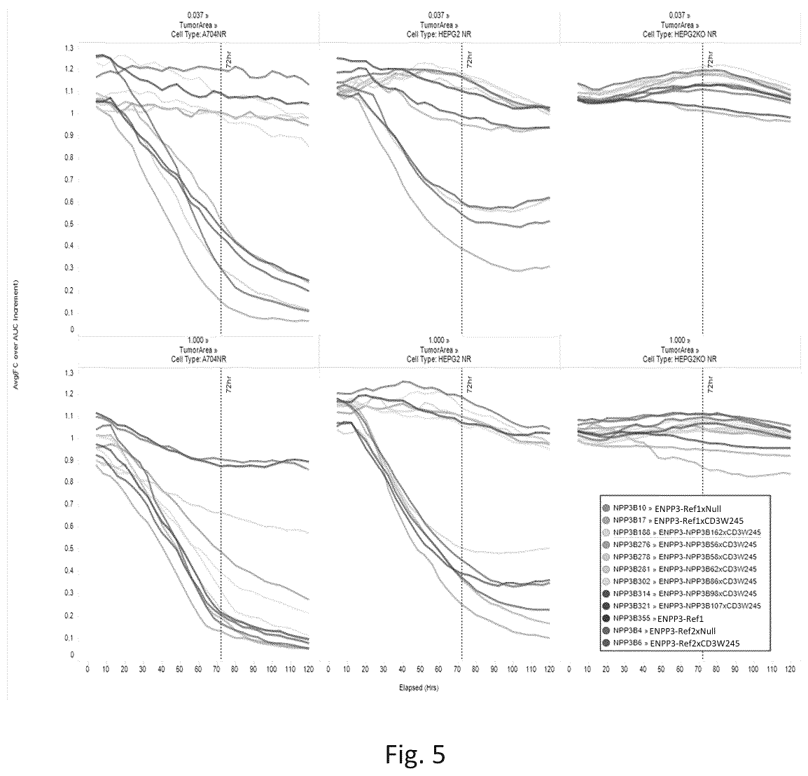

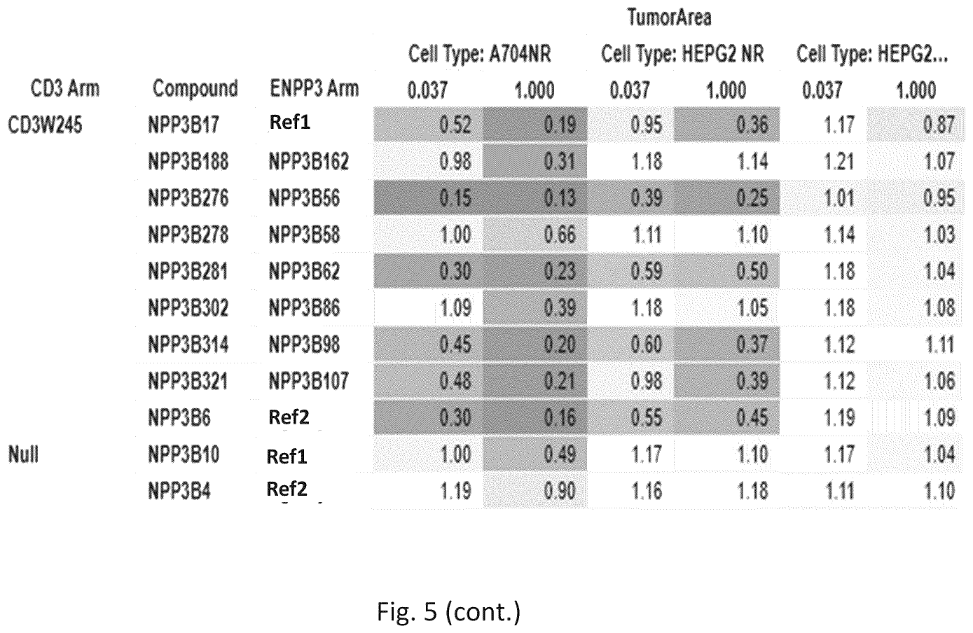

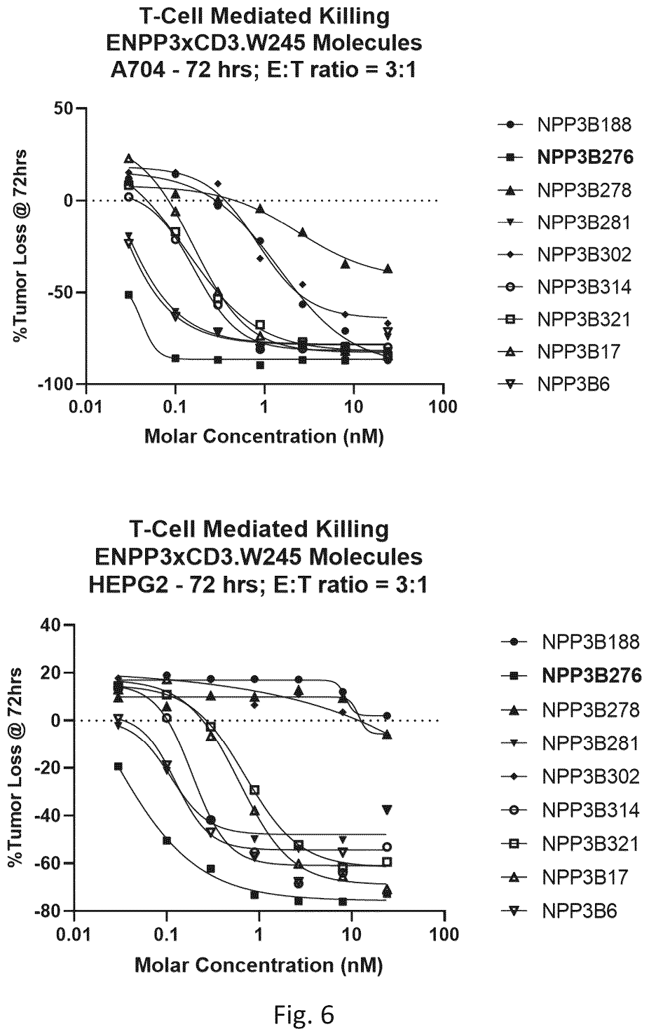

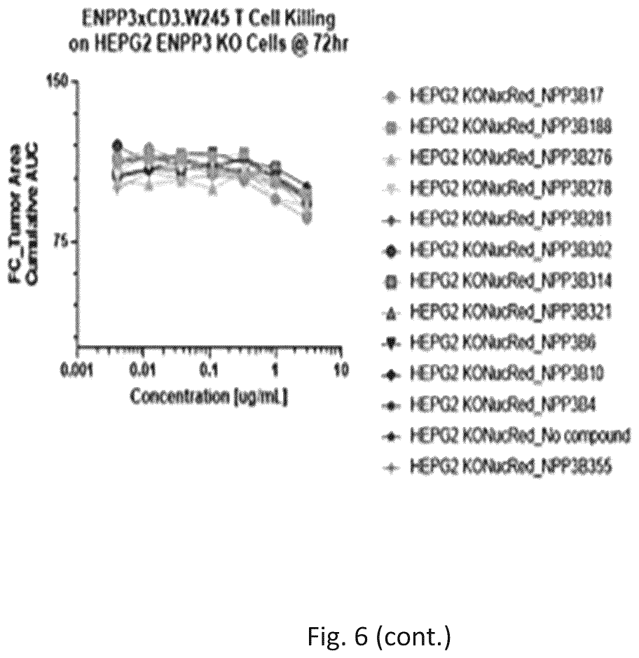

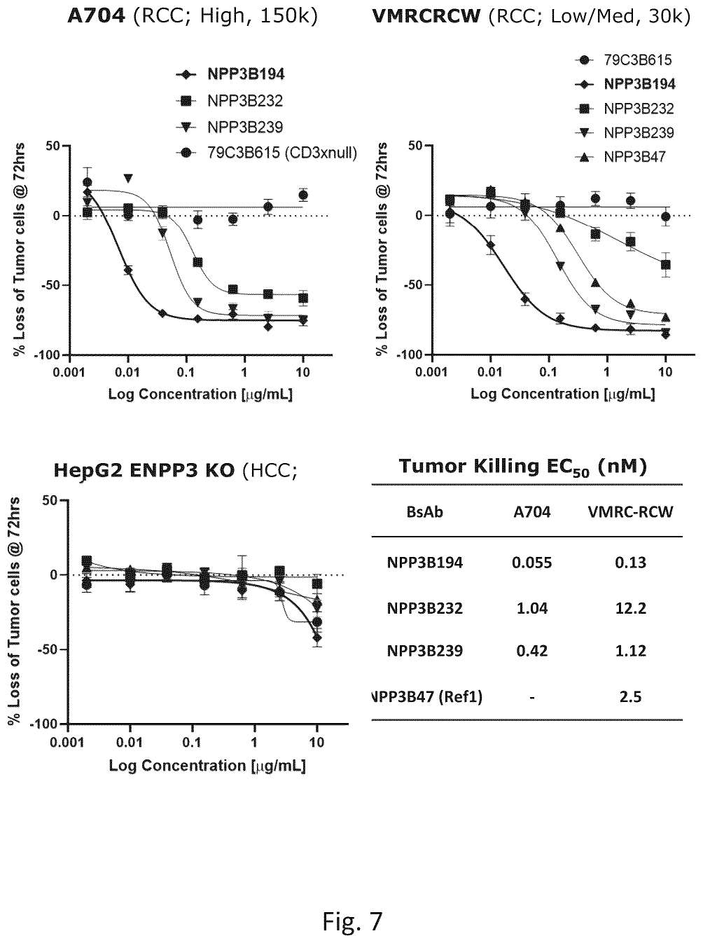

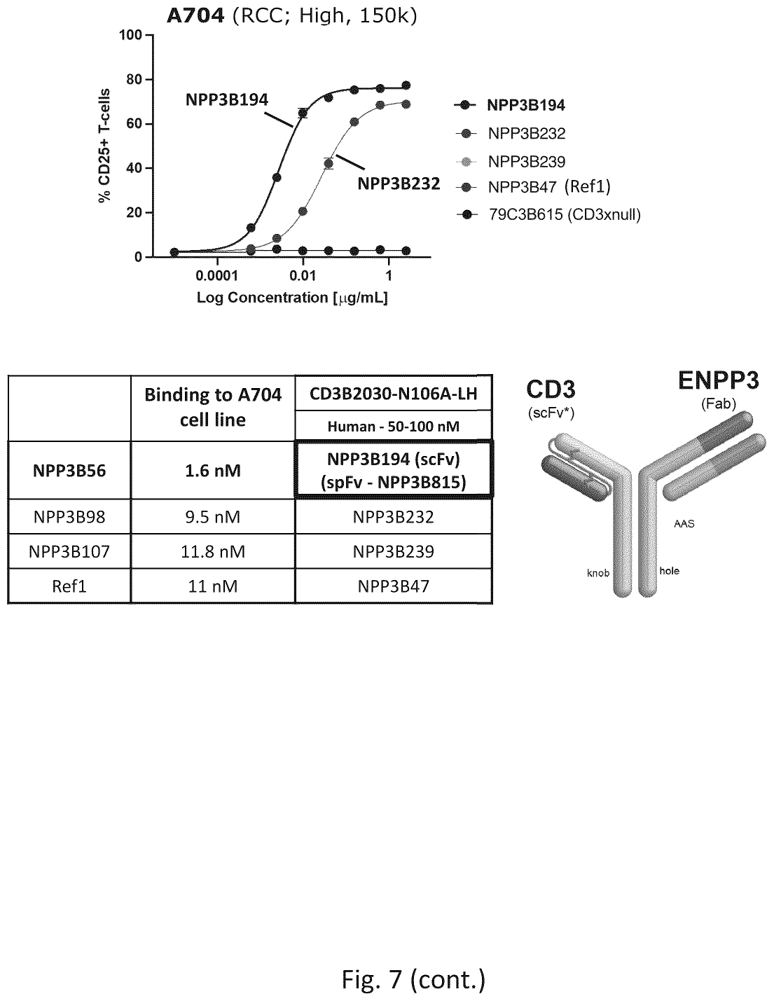

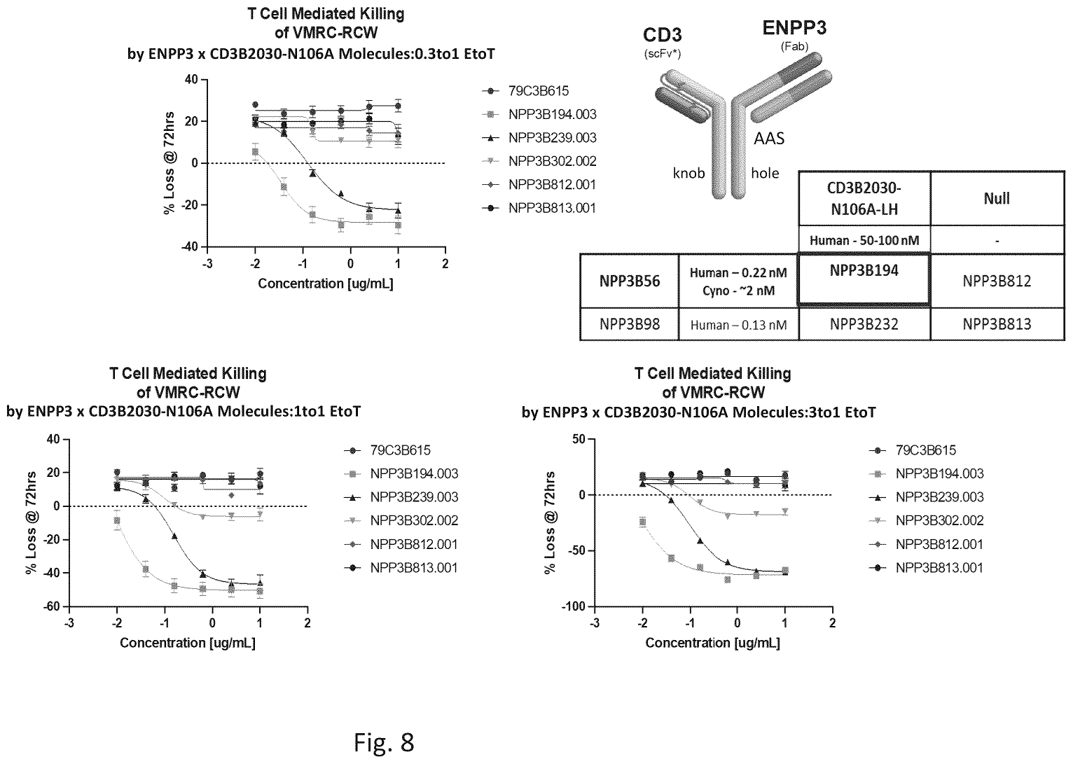

The ability of 5 ENPP3 binders paired with either CD3B2030-N106A or CD3W245 scFv arm in the bi-specific antibody format, to induce T cell-mediated tumor cell killing was assessed using an Incucyte (live cell time-lapse) instrument against a panel of cancer cell lines (FIGS. 2-8) with different ENPP3 expression levels: high (A704), medium (VMRCRCW, HepG2), and negative (HepG2 ENPP3 KO). All five CD3B2030-N106A-based ENPP3×CD3 bispecific Abs, NPP3B194, NPP3B199, NPP3B220, NPP3B232, and NPP3B239 induced dose-dependent T cell-mediated cytotoxicity at an Effector to Target cell (E:T) ratio of 3:1 on all the ENPP3-positive cell lines (A704, VMRCRCW, and HepG2,), while no killing was seen against the negative cell line (HepG2 ENPP3 KO) (FIGS. 2-4). As expected, no T cell-mediated tumor cell killing was seen with the Null×CD3 (79C3B615) control antibody in any of the cell lines tested. All six CD3W245-based ENPP3×CD3 bispecific Abs tested, NPP3B302, NPP3B321, NPP3B276, NPP3B278, NPP3B281, and NPP3B314 induced dose-dependent T cell-mediated cytotoxicity at an Effector to Target cell (E:T) ratio of 3:1 on all the ENPP3-positive cell lines (A704, VMRCRCW, and HepG2,), while no killing was seen against the negative cell line (HepG2 ENPP3 KO) (FIGS. 5 and 6). As expected, no T cell-mediated tumor cell killing was seen with the Null×CD3 (79C3B615) control antibody in any of the cell lines tested. The results showed that the three select bi-specific antibodies NPP4B194, NPP3B232, and NPP3B239 exhibited potent in vitro killing and T-cell activation (FIG. 7). In addition, the select panel of bi-specific antibodies exhibited potent in vitro killing at lower effector-to-target (E:T) ratios (FIG. 8).

7.2.3 In Vitro Functional Data for Select ENPP3 Binders Paired with Select CD3 Arm Binding of NPP3B815 to Tumor Cells and T Cells

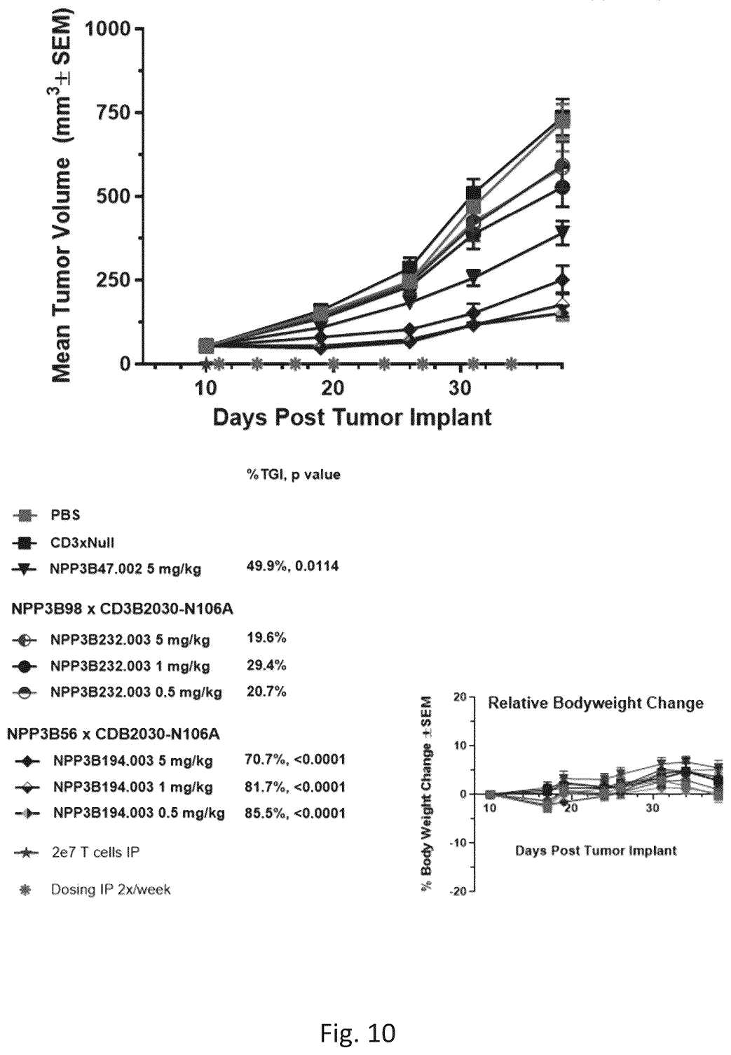

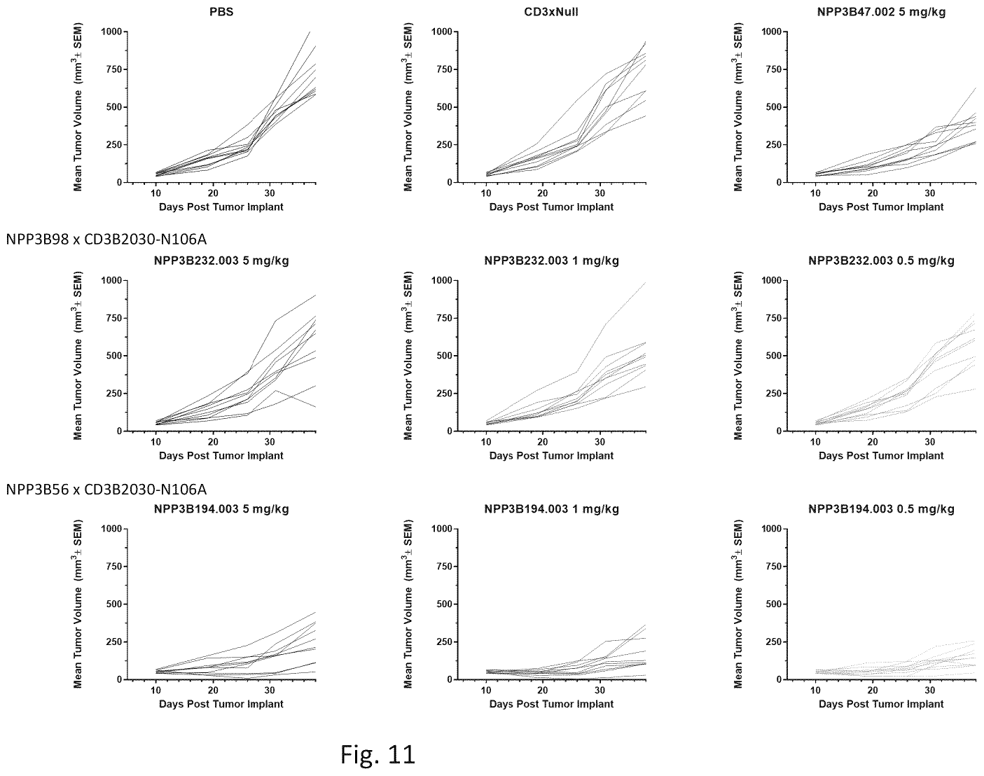

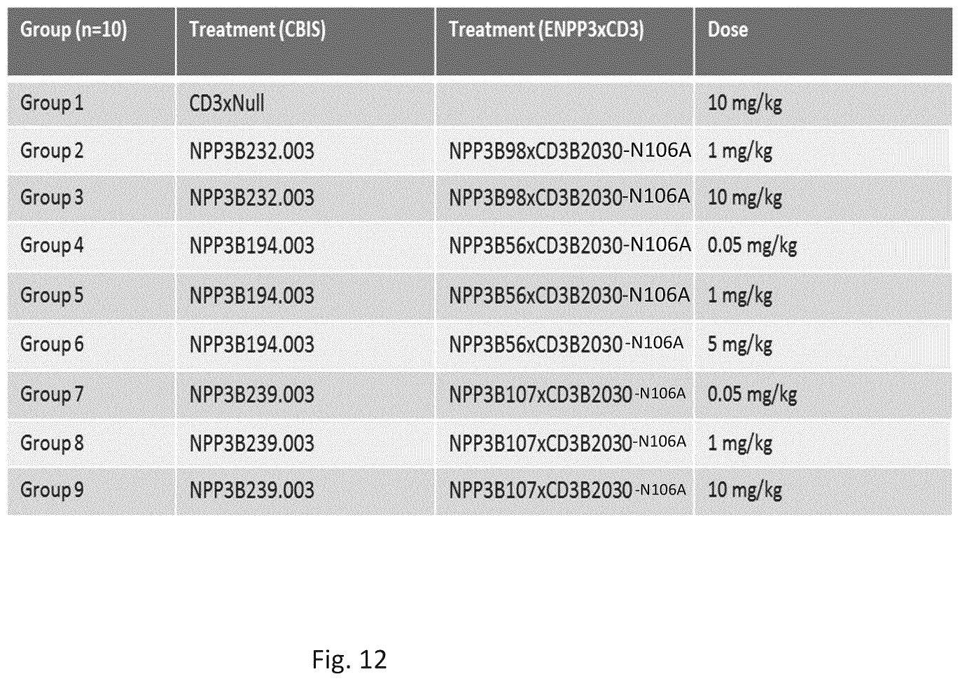

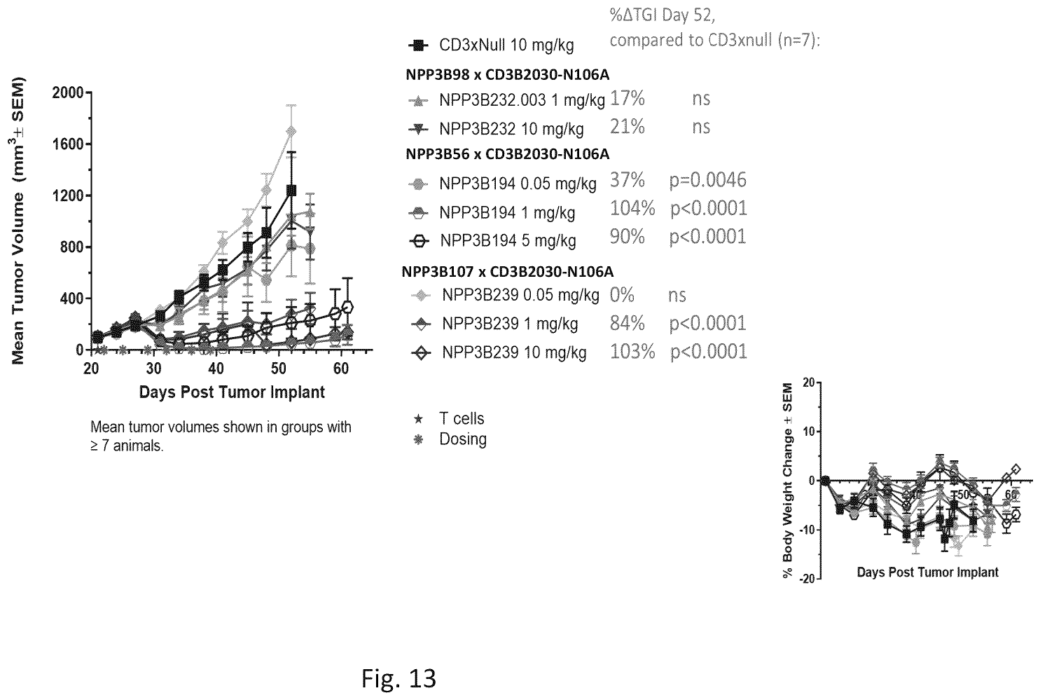

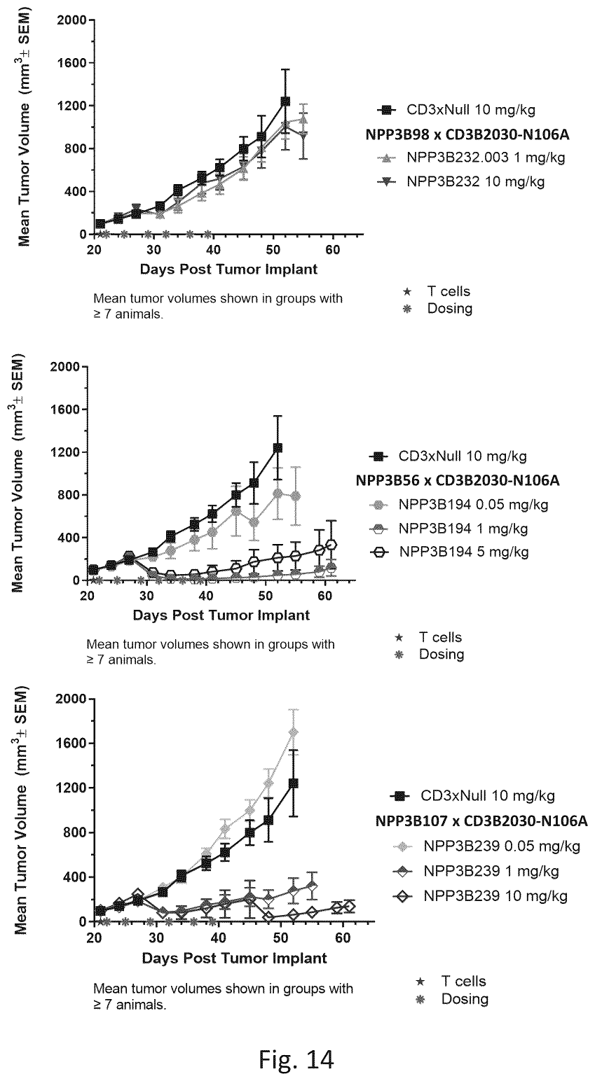

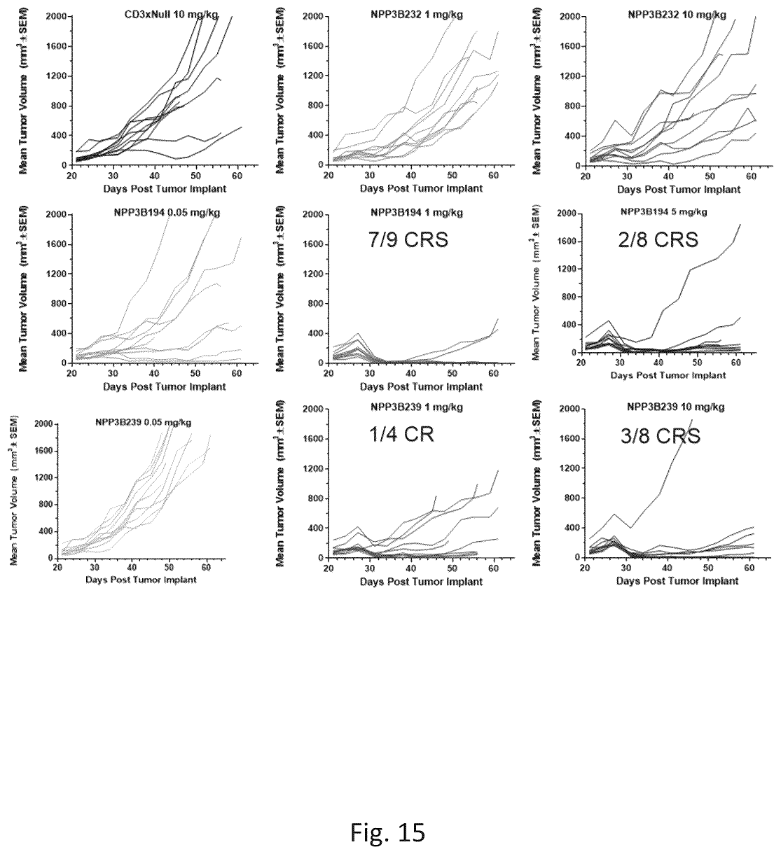

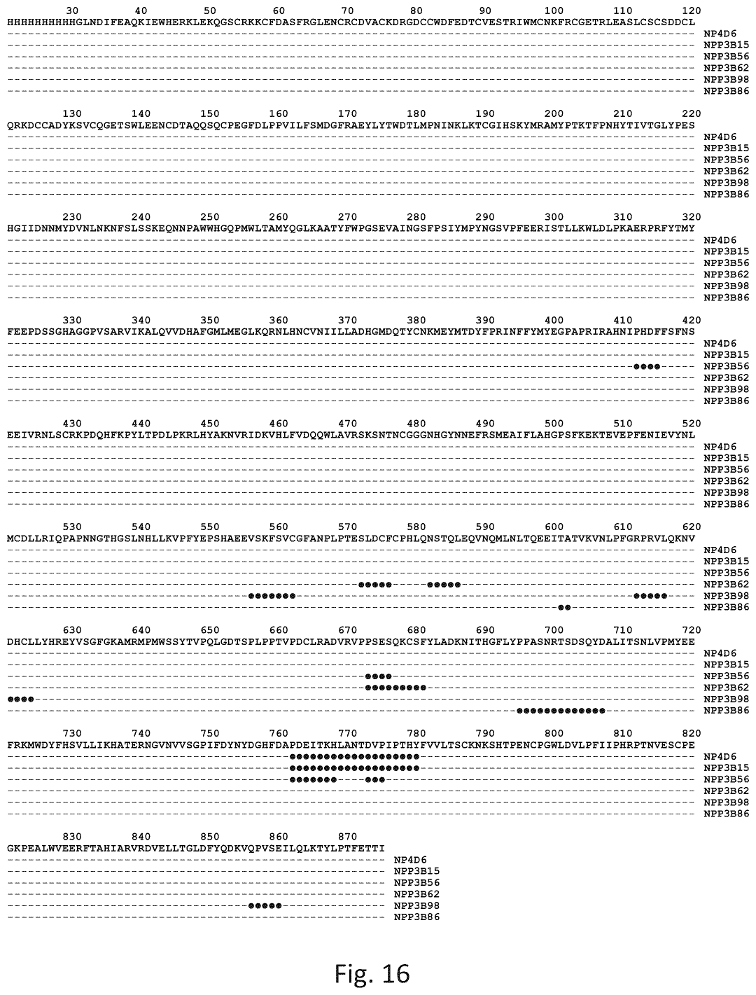

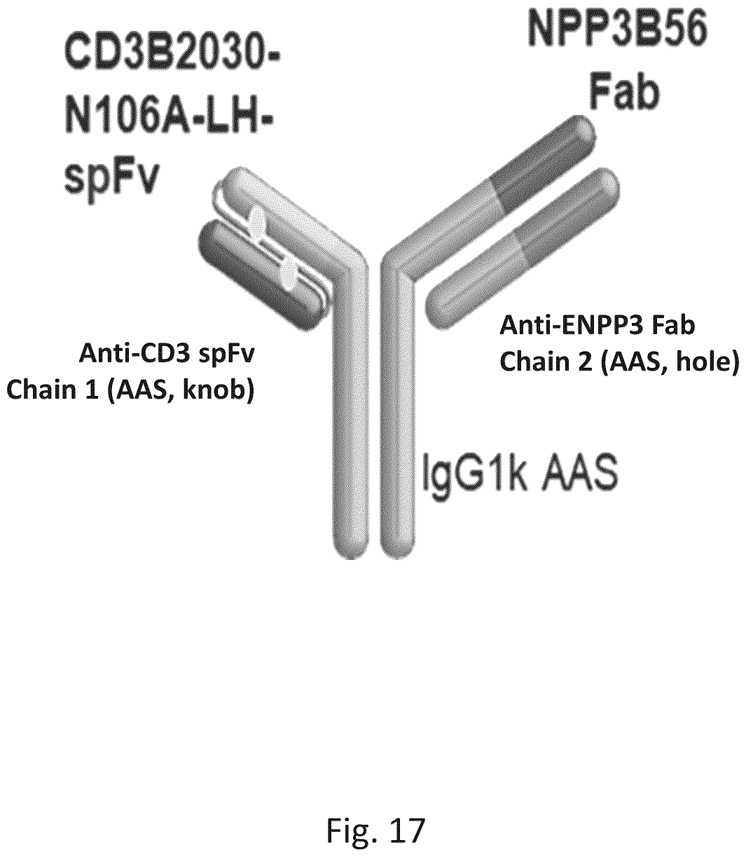

The ability of top 3 ENPP3 binders paired with CD3B2030-N106A to induce T-cell-mediated tumor cell killing was assessed using an Incucyte (live cell time-lapse) instrument against a panel of cancer cell lines (FIG. 10) with different ENPP3 expression levels: high (A704), medium (VMRCRCW), and negative (HepG2 ENPP3-KO). All three CD3B2030-N106A-based ENPP3×CD3 bispecific Abs, NPP3B194, NPP3B232, and NPP3B239 induced dose-dependent T-cell-mediated cytotoxicity at an Effector to Target cell (E:T) ratio of 3:1 on all the ENPP3-positive cell lines (A704 and VMRCRCW), while no killing was seen against the negative cell line (HepG2 ENPP3-KO) (FIG. 10). As expected, no T-cell-mediated tumor cell killing was seen with the Null×CD3 (79C3B615) control antibody in any of the cell lines tested. The T-cell-mediated tumor cell killing was further evaluated for NPP3B194 and NPP3B232 in the VMRCRCW cell line at multiple Effector to Target cell (E:T) ratio of 3:1, 1:1 and 1:3 and dose-dependent T-cell-mediated cytotoxicity was observed with both molecules.

### 7.2.4 Cross-Reactivity to Other ENPP Family Members

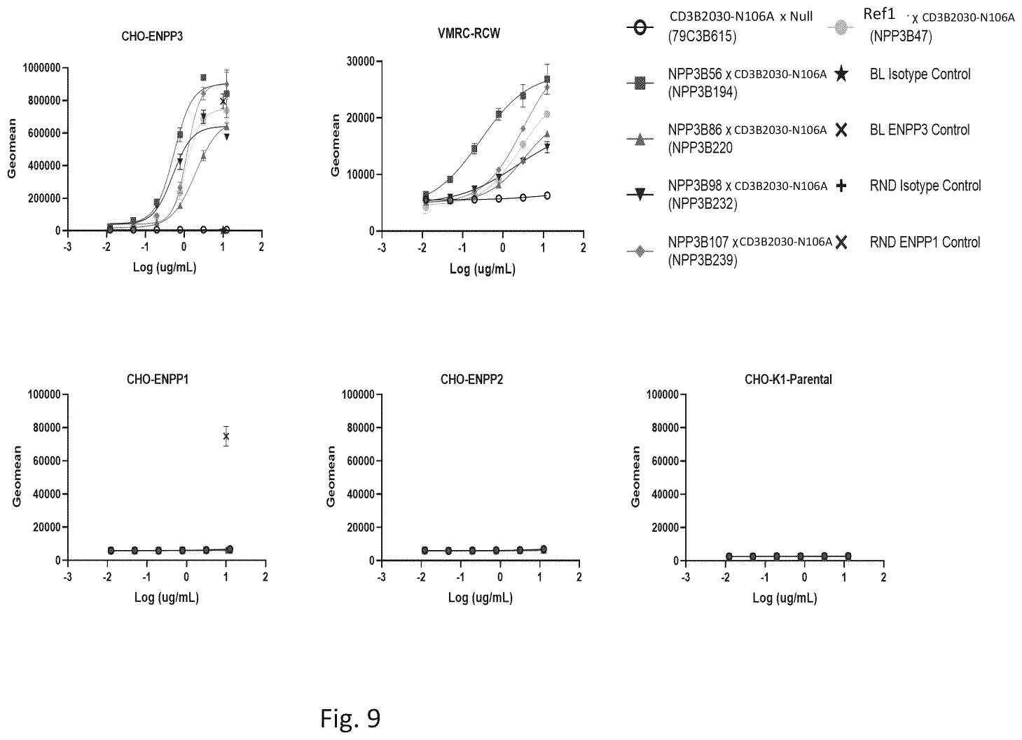

To evaluate the specificity of binders to ENPP3 compared to other ENPP family members, cell binding was assessed on CHOK1 cells overexpressing ENPP1, ENPP2, or ENPP3 (FIG. 9). NPP3B194, NPP3B232 and NPP3B239 exhibited a dose-dependent binding to only the CHOK1 cells overexpressing ENPP3. All Abs tested did not exhibit any binding to the ENPP3-negative cell line CHOK1 Parental or CHOK1 cells overexpressing ENPP1 or ENPP2. No binding was seen to any of the cell lines with the isotype control (79C3B613) antibody.

7.2.5 In Vivo Data for ENPP3 Binders Paired with CD3 Arm in RCC CDX Model, VMRC

Mice bearing established SC VMRCRCW xenografts were IP dosed with NPP3B194, NPP3B232 twice weekly at 5, 1, and 0.5 mg/kg or Null×CD3 control antibody (5 mg/kg) for a total of 8 doses (n=10/group). Significant antitumor efficacy was observed with NPP3B194 treatment at 5.1 and 0.5 mg/kg over time (p<0.001) with 85.5%, 81.7% and 70.7% Δ tumor growth inhibition (TGI), respectively, as compared to Null×CD3-antibody-treated control mice (Fig. Error! Reference source not found.). Limited antitumor efficacy was observed with NPP3B232 treatment at 5.1 and 0.5 mg/kg over time with 21%, 29% and 20% Δ tumor growth inhibition (TGI), respectively, as compared to Null×CD3-antibody-treated control mice (Error! Reference source not found.). No significant change in mouse body weight was observed with any of the treatment conditions.

7.2.6 In Vivo Data for the Select ENPP3 Binders Paired with Select CD3 Arm in HCC CDX Model, HepG2

Mice bearing established SC HepG2 xenografts were IP dosed with NPP3B194 or NPP3B232 twice weekly at 5, 1, and 0.05 mg/kg or with Null×CD3 control antibody for a total of 6 doses (n=10/group).

Treatment with NPP3B194 at 1 and 5 mg/kg resulted in % ΔTGI of 104% and 90%, respectively (p<0.0001) when compared to controls on day 52 and led to 7/9 and 2/8 CRs, respectively on day 61.

Treatment with NPP3B239.003 at 1 and 10 mg/kg resulted in ΔTGI of 84% and 103%, respectively (p<0.0001) when compared to controls on day 52 and led to ¼ and ⅜ CRs, respectively.

Higher body weight loss was observed in groups that had less anti-tumor response, indicating cachexia.

### 7.3 Example 3: Characterization of Bi-Specific ENPP3×CD3 Antibodies

NPP3B815 bispecific antibody (BsAb) was selected for further characterization. The NPP3B815 antibody comprises an anti-CD3 single-chain fragment variable featuring a “stapled” linker (spFv) on heavy chain 1 (HC1), derived from CD3B2030 N106A CD3 binder. The spFv comprises a linker having a central “C₁PPC₂” motif (SEQ ID NO: 323) wherein C₁ forms a disulfide bond with (in the case of the spFv in the “light-heavy” orientation) an engineered cysteine at position L42 (Chothia numbering) in the variable light domain and C₂ forms a disulfide bond with an engineered cysteine at position H105 in the variable heavy domain. Heavy chain 1 comprises the “knob” mutation: T366W. Heavy chain 2 (HC2) comprises the anti-ENPP3 Fab-region, derived from the NPP3B56 antibody and the “hole” mutations: T366S, L368A, Y407V. Table 1 shows CDR and VH/VL amino acid sequences for the ENPP3 binding arm. Table 13A shows CDR and VH/VL amino acid sequences for the CD3 binding arm. Table 16A shows full length sequences of the NPP3B815 antibody. Table 16B shows DNA sequences encoding the NPP3B815 antibody. Table 16C shows codon optimized DNA sequences encoding the NPP3B815 antibody.

NPP3B815 was assessed for, among other things, purity, binding affinity, thermal stability, solubility, serum stability, nonspecific binding, viscosity, and aggregation potential. NPP3B815 maintained high purity, showed no conformational changes after 2 weeks at 37° C. stress test, exhibited high solubility with acceptable viscosity, stability, and high percent monomer (at 150 mg/mL) at 40° C., maintained target binding in human serum, and showed no evidence for nonspecific binding. In summary, NPP3B815 met the ideal or acceptable criteria as demonstrated by the results from different biophysical assays, and the overall intrinsic properties were in favor of manufacturability.

CD3 specific CD3B2030-N106A-LH scFv was generated by a genetic fusion of variable region light chain (VL) and variable region heavy chain (VH) with a flexible linker. The scFv recapitulates the antigen binding specificity and largely the affinity of a parental Fv. However, scFv-containing molecules are prone to aggregation due to low thermal stability and transient separation and intermolecular VL/VH reassociation (‘breathing’).

Both of these liabilities were addressed by scFv ‘stapling’, abbreviated as spFv. To this end, 2 disulfide bonds were engineered between the flexible linker and anchor positions of the VL and VH domains (one on each). This novel strategy is compatible in both VL-VH and VH-VL orientations for almost all Fv domains. Extensive characterization of several molecules demonstrates that spFv molecules not only retain the same binding and function with improved biophysical properties but stapling significantly improved protein quality and aggregation of therapeutics observed in scFv.

The spFv consists of a linker having a central ‘C1PPC2’ motif (SEQ ID NO: 323) wherein C1 forms a disulfide bond with (in the case of the spFv in the ‘light-heavy’ orientation) an engineered cysteine at Position 42 (Chothia numbering) in the VL domain and C2 forms a disulfide bond with an engineered cysteine at Position 105 in the VH domain. HC1 features the ‘knob’ mutation T366W. HC2 features the anti-ENPP3 Fab region, and the ‘hole’ mutations T366S, L368A, and Y407V.

The BsAb was developed to evaluate the therapeutic potential of targeting ENPP3 for T-cell redirection. NPP3B815 was developed for the treatment of advanced solid tumors where ENPP3 is known to be highly expressed on the cell surface.

NPP3B815 was generated by co-expression of the anti-CD3ε spFv ‘knob’ HC with the anti-ENPP3 Fab HC containing the ‘hole’ and paired with the light chain (LC). The anti-CD3ε variable region was derived from Cris7, identified in wild-type mice and humanized in scFv format to identify humanized variants with higher thermal stability than parental Cris7 in scFv format with a range of CD3ε affinities. Briefly, the murine complementarity determining region (CDR) regions were grafted into the IGHV1-69*02-IGHJ1-01 and IGKV3-11*02-IGKJ4-01 human germlines followed by human framework adaption as described previously. The parental HC contained an NG sequence in CDR3 at amino acid Positions 106-107 (Positions 100B-100C in Kabat numbering), representing a potential risk. This risk was eliminated by mutation N106A. Although several mutations could eliminate this risk, N106A was selected based on weaker affinity of the N106A variant compared to the parental v-region, since weaker affinity towards CD3ε may be associated with lower toxicity. The anti-CD3 v-region was then formatted into a spFv in the final molecule NPP3B815. The anti-ENPP3 variable region was discovered by immunizing transgenic mice (Ablexis) with a plasmid expressing full-length ENPP3 (Genedata DNA batch ID VB000066101). The parental anti-ENPP3 variable region, featured in the NPP3B56 mAb was not modified and was formatted as a Fab in the final molecule NPP3B815.

### 7.3.1 Intrinsic Design Properties of Select ENPP3×CD3 Bispecific Antibodies

NPP3B815 exhibited, among other characteristics, a favorable biophysical profile with high affinity (ie, 920 pM) to human ENPP3 and low affinity (ie, 110 nM) to human CD3, high purity, good conformational stability, low hydrophobicity, and no nonspecific binding. Furthermore, NPP3B815 exhibited good stability profiles in serum, under ex vivo physiological conditions, under PTM-inducing forced degradation studies, and under thermal stress at high concentrations. In summary, NPP3B815 exhibited low risk for development with favorable intrinsic properties of manufacturability.

### 7.3.2 Selection of ENPP3-Expressing Cancer Cell Lines for NPP3B815 In Vitro and In Vivo Assessment

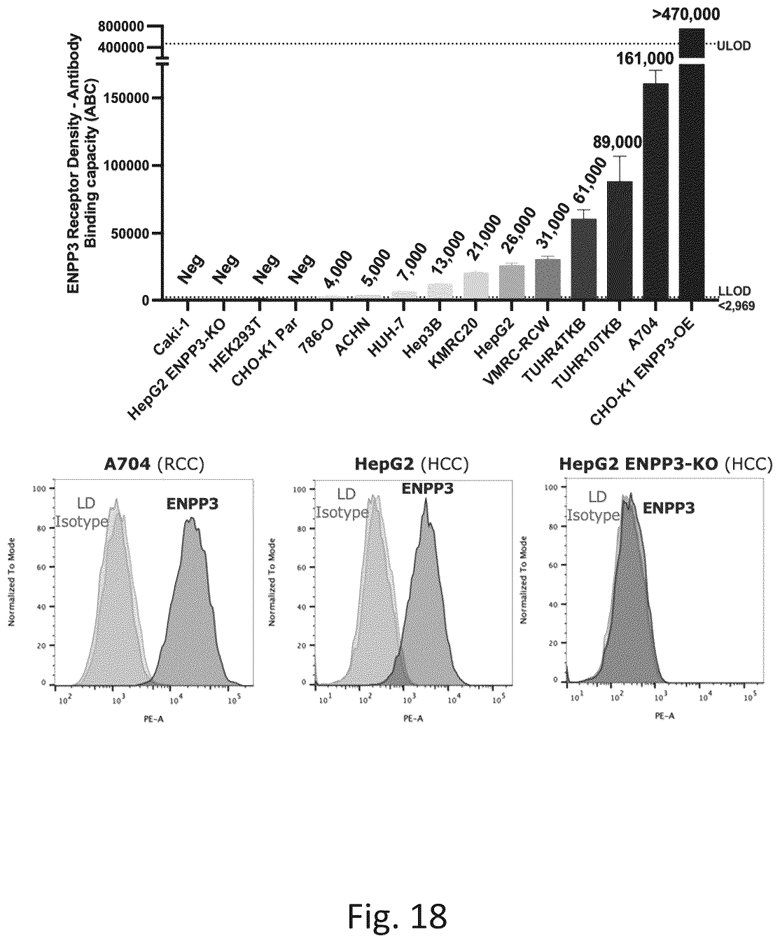

ENPP3 surface expression and receptor density were initially evaluated on a panel of 13 in vitro established RCC and HCC cell lines by flow cytometry using a commercial ENPP3 antibody (clone NP4D6) binding to a similar epitope as the ENPP3 binder arm within NPP3B815. As represented in FIG. 18, ENPP3 endogenous expression ranged from negative (determined as <lower limit of detection [LLOD], 2,969 receptors/cell) in Caki1, HEK293T and RXF393 (PDX) cell lines, low (<15,000 receptors/cell) in 786-O, ACHN, Huh7 and Hep3B, medium (15,000-40,000 receptors/cell) in KMRC20, HepG2 and VMRCRCW, medium-high (50,000-100,000 receptors/cell) in TUHR4TKB and TUHR10TKB and high (>100,000 receptors/cell) in A704 cell lines. Endogenous ENPP3 was knocked out (using clustered regularly interspaced short palindromic repeats [CRISPR]) in HepG2 cell line, thus generating the negative cell line HepG2 ENPP3-KO. Conversely, ENPP3 was overexpressed in the CHO-K1 cell line to generate the overexpression cell line CHO-K1 ENPP30E.

To confirm if similar ENPP3 expression was detectable by NPP3B815 on these cell lines, direct-labeled ENPP3×Null BsAb NPP3B812, which has the same ENPP3 binder NPP3B56 as NPP3B815, was used for flow cytometry-based receptor density studies. The level of ENPP3 expression with NPP3B812 was observed to be comparable to that seen with the commercial antibody in the cell lines tested. While these cell lines represent a range of ENPP3 expression, most of them having lower expression than the ccRCC tumors. Among these cell lines, A704, VMRCRCW, and HepG2 ENPP3-KO cell lines were used for most of the key in vitro functional studies as they represent high, medium, and negative ENPP3 expression levels, respectively.

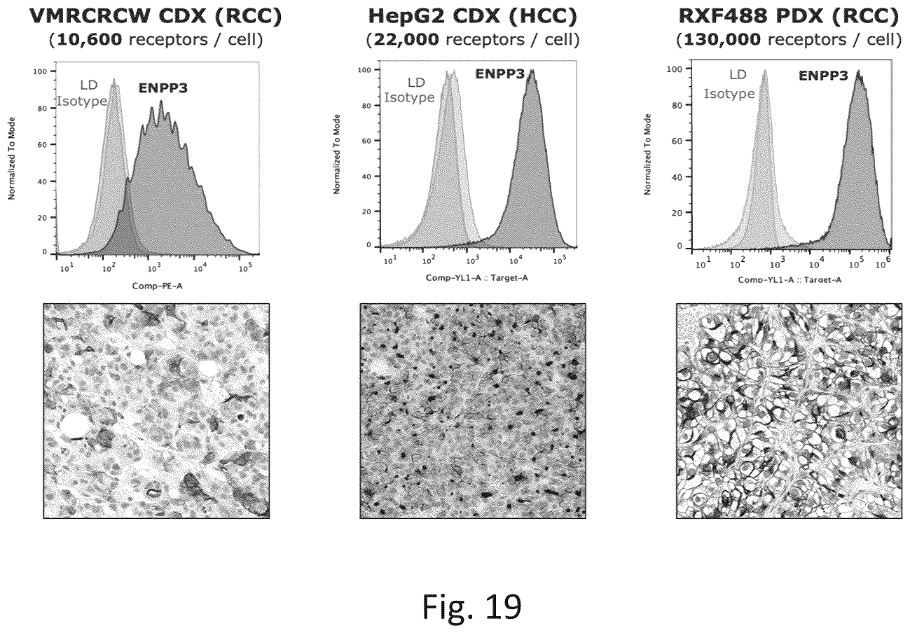

Two of these cell lines (i.e., VMRCRCW and HepG2) have also been established as in vivo CDX models to use for NPP3B815 efficacy studies. Ex vivo ENPP3 expression was measured by IHC and flow cytometry (dissociated tumors) at a tumor volume equivalent to the tumor at randomization in these 2 CDX tumor models. IHC showed ENPP3 positivity in both CDX tumors and receptor density was found to be 10,600 (VMRCRCW) and 22,000 (HepG2) ENPP3 receptors per cell (FIG. 19). To explore a higher ENPP3 expressing tumor model for in vivo studies, several positive ccRCC PDX models were identified by IHC; ENPP3 expression was evaluated ex vivo in the ccRCC PDX model RXF488, which showed high ENPP3 expression by IHC and receptor density measurements of 130,000 receptors/cell.

### 7.3.3 In Vitro Binding of NPP3B815 to Tumor Cells and T Cells

In vitro cell binding of NPP3B815 (NPP3B56×CD3B2030-N106A) was assessed on high (A704), medium (VMRCRCW), and negative endogenous ENPP3-expressing cell lines (Table 18). NPP3B815 exhibited a dose-dependent binding to both positive cell lines, A704 and VMRCRCW, with EC50 values of 1.01 nM and 0.5 nM respectively (Error! Reference source not found. Error! Reference source not found. and Table 19). NPP3B815 did not exhibit binding to the ENPP3-negative cell line HepG2 ENPP3-KO. No binding was seen to any of the cell lines with the Null×CD3 (79C3B615) or isotype control (79C3B613) antibodies.

TABLE 18
ENPP3 expression (receptor density)
in different cancer cell lines

| | Receptor |
|---|---|
| | Density |
| Tumor Cells | (ABC) |
| A704 | 161,000 |
| TUHR10TKB | 89,000 |
| TUHR10TKB | 61,000 |
| VMRCRCW | 31,000 |
| HepG2 | 26,000 |
| HepG2 ENPP3 KO | Negative |

Further, binding of NPP3B815 to human T cells isolated from 6 different healthy donors was assessed. A dose-dependent binding was observed to T cells from all 6 donors, with an average EC50 (±standard error of the mean) of 177±18.5 nM (FIG. 20 and Table 19). As expected, no binding was seen with isotype control antibody 79C3B613 to any of the T-cell donors.

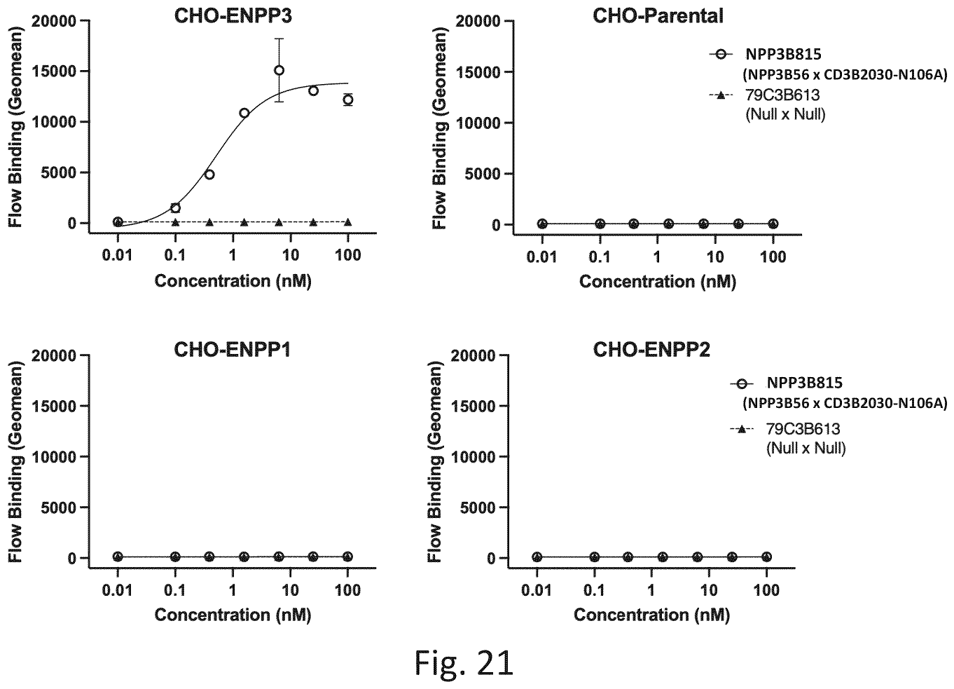

To evaluate the specificity of NPP3B815 to ENPP3 compared to other ENPP family members, cell binding was assessed on CHOK1 cells overexpressing ENPP1, ENPP2, or ENPP3 (FIG. 21). NPP3B815 exhibited a dose-dependent binding to only the CHOK1 cells overexpressing ENPP3, with a binding affinity (EC50) of 0.5 nM. NPP3B815 did not exhibit any binding to the ENPP3-negative cell line CHOK1 Parental or CHOK1 cells overexpressing ENPP1 or ENPP2 (FIG. 21 and Table 19). No binding was seen to any of the cell lines with the isotype control (79C3B613) antibody.

TABLE 19
Cell Binding EC50 values for NPP3B815
across target cells and T-cells.

EC₅₀
(nM)

Target Cells

| | |
|---|---|
| A704 | 1.01 |
| VMRCRCW | 0.5 |
| HepG2 ENPP3 KO | ND |
| CHO Parental | ND |
| CHO ENPP1 OE | ND |
| CHO ENPP2 OE | ND |
| CHO ENPP3 OE | 0.51 |

T-Cells

| | |
|---|---|
| 888668965 | 197.3 |
| 110048457 | 132 |
| 110044049 | 188.5 |
| 110042593 | 145.6 |
| 110042496 | 147.2 |
| 110040278 | 253.3 |

ECx, x % effective concentration; ND, not determined.
EC50 values were not determined if the dose-response curve did not satisfy one or both of the following criteria: R2 has to be >0.9 and the log (95% confidence interval) difference needs to be <1.2.

7.3.4 NPP3B815-Induced Cytotoxicity and T-Cell Activation on a Panel of Cancer Cell Lines with Different Levels of Endogenous ENPP3 Expression

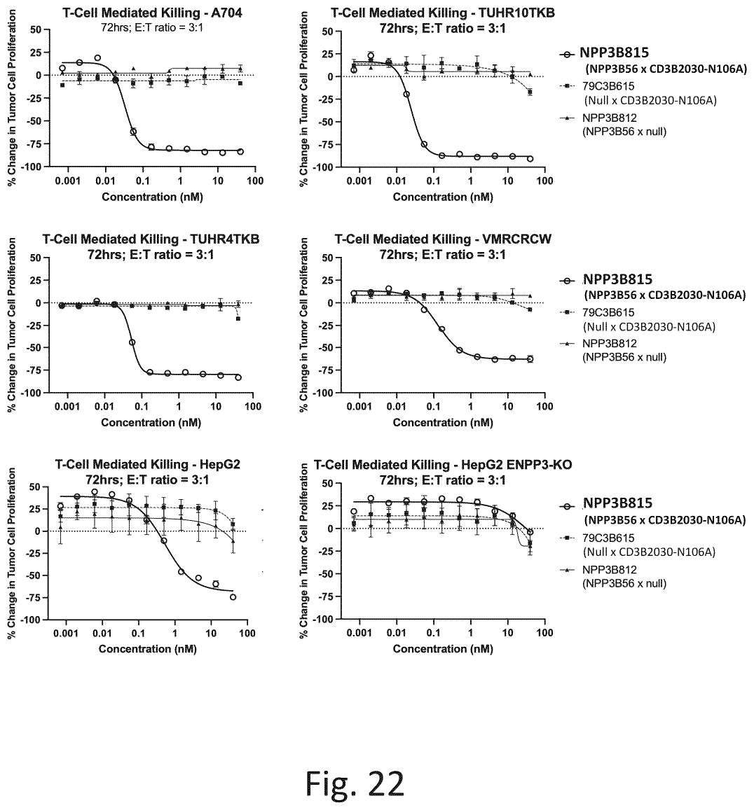

The ability of NPP3B815 (NPP3B56×CD4B2030) to induce T-cell-mediated tumor cell killing was assessed using an Incucyte (live cell time-lapse) instrument against a panel of cancer cell lines (Table 20) with different ENPP3 expression levels: high (A704), medium-high (TUHR10TKB, TUHR4TKB), medium (VMRCRCW, HepG2), and negative (HepG2 ENPP3-KO). NPP3B815-induced dose-dependent T-cell-mediated cytotoxicity at an Effector to Target cell (E:T) ratio of 3:1 on all the ENPP3-positive cell lines (A704, VMRCRCW, HepG2, TUHR4TKB, and TUHR10TKB), while no killing was seen against the negative cell line (HepG2 ENPP3-KO) (FIG. 22). As expected, no T-cell-mediated tumor cell killing was seen with either Null×CD3 (79C3B615) or ENPP3×Null (NPP3B812) control antibodies in any of the cell lines tested. The EC50 and maximum activity values for tumor cell killing are shown in Table 20, as the cell lines with higher ENPP3 expression had lower EC50 values with high max tumor cell killing.

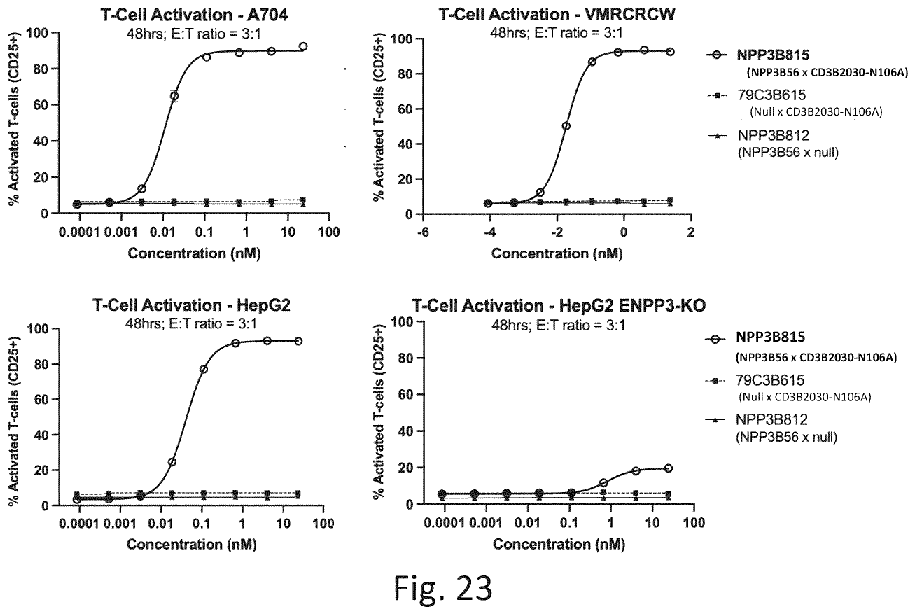

To assess the level of T cell activation induced by the ENPP3×CD3 bispecific antibody, CD25 expression was measured on T cells by flow cytometry at 48 hours post antibody treatment (FIG. 23). A dose-dependent increase in T cell activation (E:T ratio=3:1) was seen with NPP3B815 on all the ENPP3-positive cell lines tested (A704, VMRCRCW, and HepG2, while no T cell activation was seen with NPP3B815 in the negative cell line (HepG2 ENPP3-KO) or with the control antibodies Null×CD3 and ENPP3×Null in any of the cell lines tested. The EC50 and maximum activity values for T-cell activation are shown in Table 20Table.

TABLE 20
EC50 and maximum activity values for tumor cell
killing and T-cell activation in a panel of tumor
cell lines with endogenous ENPP3 expression.

| | Tumor cell killing | | | T-cell activation | | |
|---|---|---|---|---|---|---|
| | (Incucyte) | | | (Flow Cytometry) | | |
| | EC₅₀ | EC₉₀ | Max | EC₅₀ | EC₉₀ | Max |
| Cell line | (nM) | (nM) | killing (%) | (nM) | (nM) | killing (%) |
| A704 | 0.03 | 0.08 | 82 | 0.011 | 0.042 | 90 |
| TUHR10TKB | 0.02 | 0.06 | 88 | — | — | — |
| TUHR4TKB | 0.05 | 0.09 | 80 | — | — | — |
| VMRCRCW | 0.13 | 0.65 | 63 | 0.018 | 0.085 | 93 |
| HepG2 | 0.53 | 3.68 | 68 | 0.041 | 0.175 | 93 |
| HepG2 | ND | ND | ND | 0.842 | 3.834 | 20 |
| ENPP3-KO | | | | | | |

ECx, x % effective concentration; Max, maximal; ND, not determined.
EC50, EC90, and Max killing values were not determined if the dose-response curve did not satisfy one or both of the following criteria: R2 has to be >0.9 and the log (95% confidence interval) difference needs to be <1.2.
[ELN E066020, E069414, E063183, E063179]

### 7.3.5 NPP3B815-Induced Cytotoxicity and T Cell Activation Across Multiple T Cell Donors and E:T Ratios

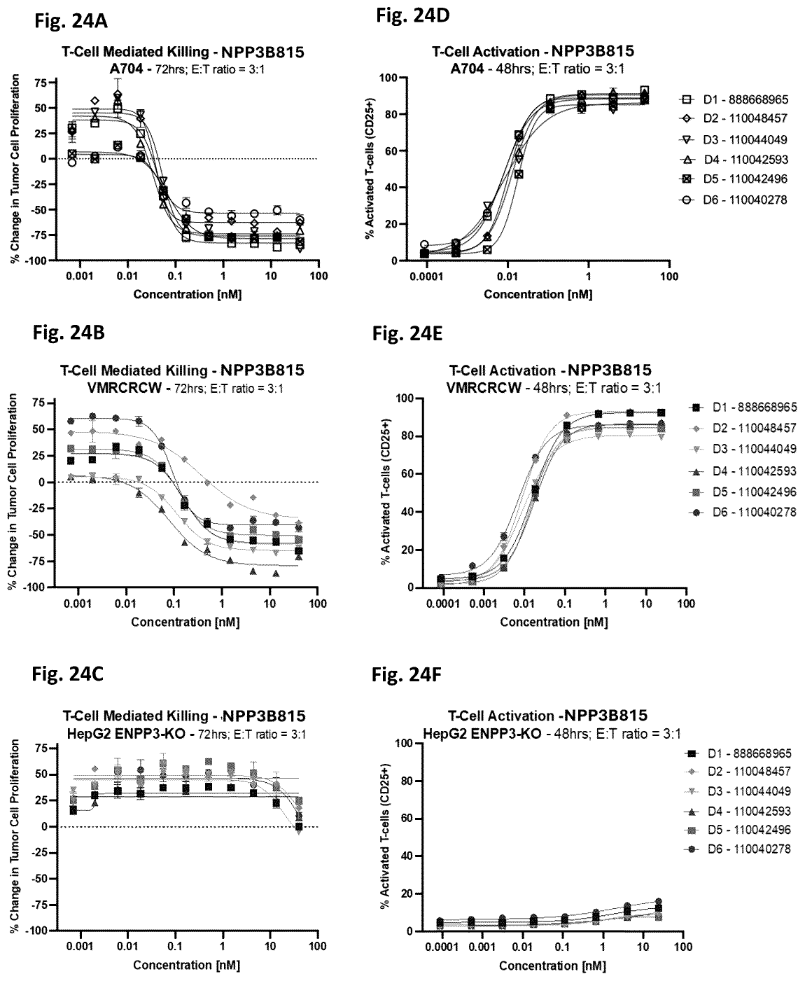

NPP3B815-induced T-cell-mediated tumor cell killing of ENPP3-positive cell lines A704 (high) and VMRCRCW (medium) and of ENPP3-negative cell line HepG2 ENPP3-KO was evaluated in the presence of T cells isolated from 6 different healthy human donors (tested at E:T ratio=3:1) (FIG. 24A). Dose-dependent T-cell-mediated cytotoxicity was seen with NPP3B815 across all 6 donors, with some variability seen in EC50 values and maximum cell killing between the different donors (Table 21). As expected, no T-cell-mediated tumor cell killing was seen with Null×CD3 control antibody 79C3B615 against all cell lines and T-cell donors tested. Like A704, dose-dependent T-cell-mediated cytotoxicity was seen with NPP3B815 across all donors in the ENPP3-medium cell line, VMRCRCW, while no killing was observed against the negative cell line (HepG2 ENPP3-KO). EC50 and maximum killing values are shown in Table 21.

In addition to the cytotoxicity assessment described above, NPP3B815-induced T-cell activation (CD25 expression by flow cytometry at 48 hours) was evaluated with the same 6 T-cell donors at an E:T ratio of 3:1 (FIG. 24B). A dose-dependent increase in T-cell activation was seen with NPP3B815 across all 6 T-cell donors in both ENPP3-positive cell lines (A704 and VMRCRCW). No T-cell activation was seen with NPP3B815 in the negative cell line (HepG2 ENPP3-KO) or with the Null×CD3 control antibody 79C3B615 against all cell lines tested. The EC50 and maximum values for T-cell activation are shown in Table 22.

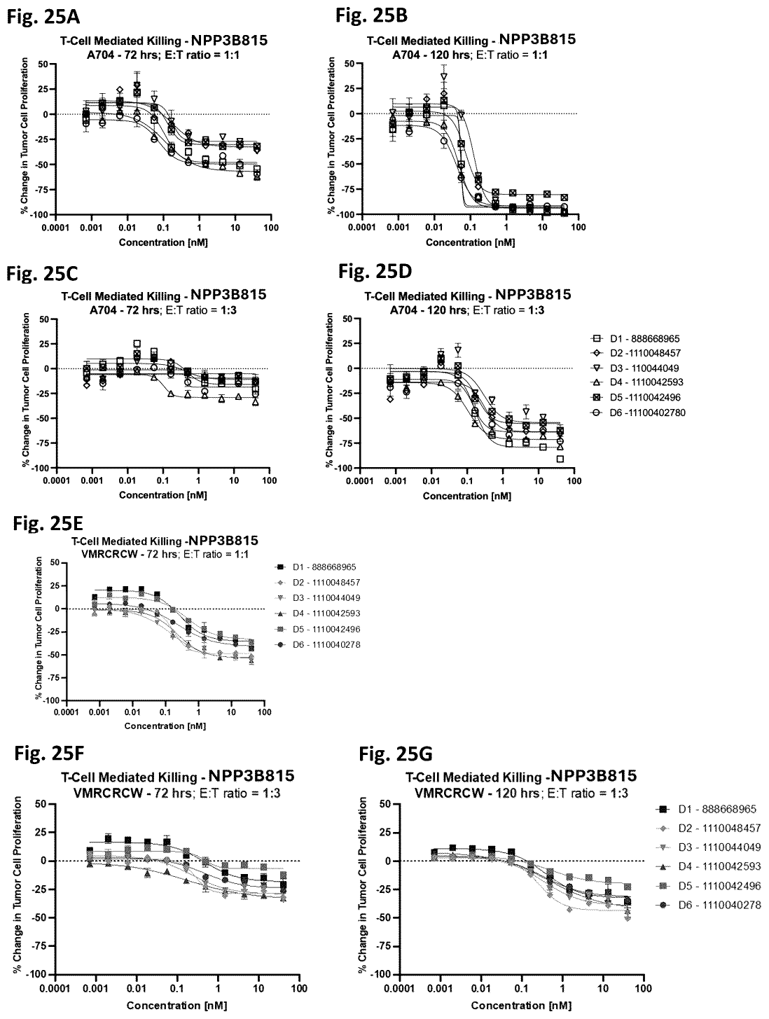

NPP3B815-induced cytotoxicity was also evaluated at lower, more physiologically relevant E:T ratios of 1:1 and 1:3, and a dose-dependent killing was observed in the ENPP3-high cell line, A704 at these E:T ratios (FIG. 25). A modest decrease in the maximum killing activity of NPP3B815 was seen at the lower E:T ratios of 1:1 (41%) and 1:3 (17%), compared to that observed in the ENPP3-high cell line A704 at an ET ratio of 3:1 (71%), when measured at 72 hours (Table 21). NPP3B815Interestingly, with longer incubation time (5 days vs 3 days), the maximum killing in A704 at the lower E:T ratios 1:3 increased from 41% to 91% for E:T=1:1 and from 17% to 65% 35% for E:T=1:3, leading to similar max killing as with higher E:T ratios. Similar cytotoxicity assays at lower E:T ratios were also preformed in the ENPP3-medium cell line VMRCRCW and the negative cell line, HepG2 ENPP3-KO. A dose-dependent killing was observed in VMRCRCW at the lower E:T ratios, while no killing was observed against the negative cell line HepG2 ENPP3-KO (FIG. 25). As expected, no T-cell-mediated tumor cell killing was seen with the Null×CD3 control antibody 79C3B615 at any of the E:T ratios or cell lines tested. The EC50 and maximum activity values for tumor cell killing are shown in Table 21.

TABLE 21
EC50 values for tumor cell cytotoxicity across T-cell donors and E:T ratios.

| | E:T = 3:1 | | E:T = 1:1 | | | | E:T = 1:3 | | | |
|---|---|---|---|---|---|---|---|---|---|---|
| | EC₅₀ | Max | EC₅₀ | Max | EC₅₀ | Max | EC₅₀ | Max | EC₅₀ | Max |
| | (nM) | killing | (nM) | killing | (nM) | killing | (nM) | killing | (nM) | killing |
| | @ | (%) @ | @ | (%) @ | @ | (%) @ | @ | (%) @ | @ | (%) @ |
| T-cell donors | 72 hrs | 72 hrs | 72 hrs | 72 hrs | 120 hrs | 120 hrs | 72 hrs | 72 hrs | 120 hrs | 120 hrs |

A704

| | | | | | | | | | | |
|---|---|---|---|---|---|---|---|---|---|---|
| 888668965 | 0.04545 | 83 | ND | 48 | ND | 93 | ND | 16 | 0.163 | 79 |
| 110048457 | ND | 63 | ND | 33 | ND | 92 | ND | ND | ND | 64 |
| 110044049 | 0.05244 | 76 | ND | 27 | 0.125 | 94 | ND | 10 | ND | 54 |
| 110042593 | 0.03358 | 74 | 0.114 | 57 | 0.048 | 94 | ND | 29 | 0.106 | 71 |
| 110042496 | 0.06961 | 79 | 0.132 | 30 | 0.073 | 80 | ND | 11 | 0.191 | 55 |
| 110040278 | 0.04837 | 53 | 0.075 | 50 | 0.041 | 92 | ND | 19 | 0.149 | 64 |

VMRCRCW

| | | | | | | | | | | |
|---|---|---|---|---|---|---|---|---|---|---|
| 888668965 | 0.1561 | 58 | 0.273 | 35 | 0.345 | 59 | 0.469 | 18 | 0.379 | 32 |
| 110048457 | 0.3716 | 35 | 0.177 | 48 | 0.21 | 66 | 0.276 | 29 | 0.267 | 43 |
| 110044049 | 0.1148 | 65 | 0.151 | 54 | 0.216 | 66 | 0.226 | 29 | 0.337 | 39 |
| 110042593 | 0.07951 | 79 | 0.225 | 53 | 0.218 | 68 | ND | ND | 0.485 | 43 |
| 110042496 | 0.1278 | 50 | 0.465 | 33 | 0.454 | 44 | ND | ND | 0.5 | 20 |
| 110040278 | 0.09229 | 40 | 0.243 | 41 | 0.264 | 53 | 0.456 | 24 | 0.332 | 31 |

HepG2 ENPP3-KO

| | | | | | | | | | | |
|---|---|---|---|---|---|---|---|---|---|---|
| 888668965 | ND | ND | ND | ND | ND | ND | ND | ND | ND | ND |
| 110048457 | ND | ND | ND | ND | ND | ND | ND | ND | ND | ND |
| 110044049 | ND | ND | ND | ND | ND | ND | ND | ND | ND | ND |
| 110042593 | ND | ND | ND | ND | ND | ND | ND | ND | ND | ND |
| 110042496 | ND | ND | ND | ND | ND | ND | ND | ND | ND | ND |
| 110040278 | ND | ND | ND | ND | ND | ND | ND | ND | ND | ND |

ECₓ, x % effective concentration; Max, maximal; ND, not determined.
EC₅₀ and Max killing values were not determined if the dose-response curve did not satisfy one or both of the following criteria: R² has to be >0.9 and the log (95% confidence interval) difference needs to be <1.2.

TABLE 22
EC50 values for T-cell activation across donors (E:T ratio = 3:1).

| | A704 | | VMRCRCW | | HepG2 ENPP3-KO | |
|---|---|---|---|---|---|---|
| | | Max | | Max | | Max |
| T-cell | EC₅₀ | activity | EC₅₀ | activity | EC₅₀ | activity |
| donors | (nM) | (%) | (nM) | (%) | (nM) | (%) |
| 888668965 | 0.008 | 91 | 0.016 | 92 | ND | ND |
| 110048457 | 0.01 | 89 | 0.009 | 93 | 1.142 | 9.414 |
| 110044049 | 0.008 | 86 | 0.009 | 80 | ND | ND |
| 110042593 | 0.013 | 90 | 0.017 | 86 | ND | ND |
| 110042496 | 0.017 | 85 | 0.015 | 85 | ND | ND |
| 110040278 | 0.008 | 88 | 0.007 | 86 | ND | ND |

EC₅₀ and Max activity values were not determined if the dose-response curve did not satisfy one or both of the following criteria: R² has to be >0.9 and the log (95% confidence interval) difference needs to be <1.2.

### 7.3.6 NPP3B815-Induced Cytokine Release in Presence of T Cells and Tumor Cells

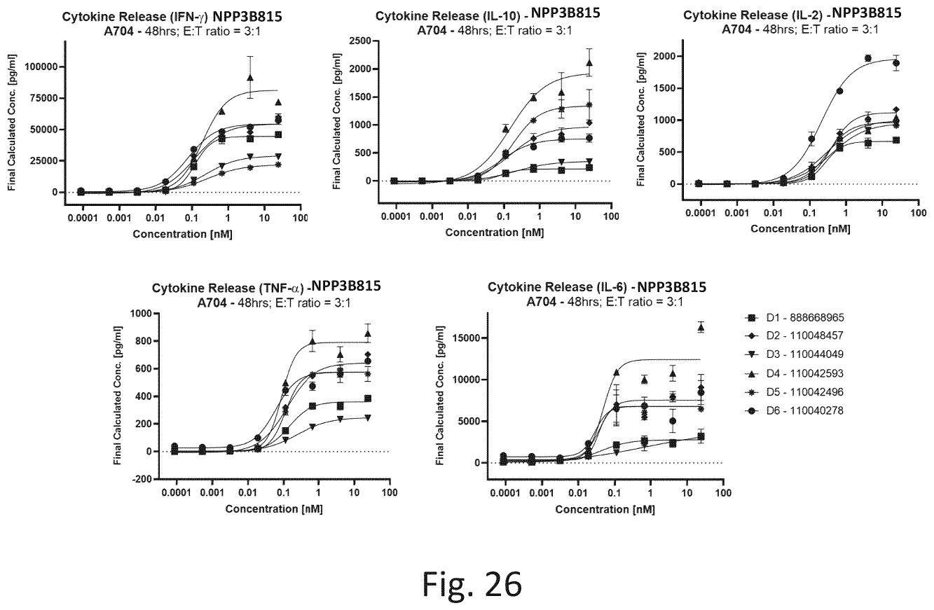

To further characterize T cell activation by NPP3B815, cytokine release profiles of 10 established inflammatory cytokines were measured using the Human Vplex Proinflammatory Panel (Meso Scale Discovery [MSD]) in the ENPP3-high cell line A704 at 48 hours post incubation with T cells from 6 different healthy human donors at an ET ratio of 3:1. A dose-dependent increase in all pro-inflammatory cytokines tested was seen with NPP3B815 across all 6 T cell donors with some variability in response seen between donors (FIG. 26). Some cytokines such as interferon (IFN)-γ, interleukin (IL)-2, IL-10, IL-6, and tumor necrosis factor (TNF)-α exhibited a strong dose-dependent induction with NPP3B815 treatment (FIG. 26 and Table 23), while others (IL-1β, IL-12p70, IL-4, IL-13, IL-8) had a modest to low dose-dependent induction. Concentrations of induced cytokines widely vary upon NPP3B815 treatment from the lowest induced cytokine (IL-1β) in the single-digit pg/mL to the highest (IFN-γ) in the 3-digit ng/ml range. The Null×CD3 control antibody 79C3B615 induced low/no cytokine response as expected, except in the case of IL-8, which showed high background signal.

TABLE 23
EC50 values for cytokine release with T-cell donors.

| T-cell donors | IFN-g | IL-10 | IL-2 | TNF-a | IL-6 |
|---|---|---|---|---|---|
| 888668965 | 0.122 | 0.085 | 0.268 | 0.143 | 0.041 |
| 110048457 | 0.117 | 0.156 | 0.390 | 0.124 | 0.037 |
| 110044049 | 0.240 | 0.210 | 0.395 | 0.241 | 0.782 |
| 110042593 | 0.198 | 0.151 | 0.295 | 0.087 | 0.047 |
| 110042496 | 0.264 | 0.177 | 0.305 | 0.112 | 0.036 |
| 110040278 | 0.078 | 0.084 | 0.211 | 0.059 | 0.029 |

ECx, x % effective concentration; Max, maximal; ND, not determined; PBMC, peripheral blood mononuclear cell.
EC50, EC90, and Max killing values were not determined if the dose-response curve did not satisfy one or both of the following criteria: R2 has to be >0.9 and the log (95% confidence interval) difference needs to be <1.2.

7.3.7 NPP3B815-Induced Cytotoxicity from PBMC Donors

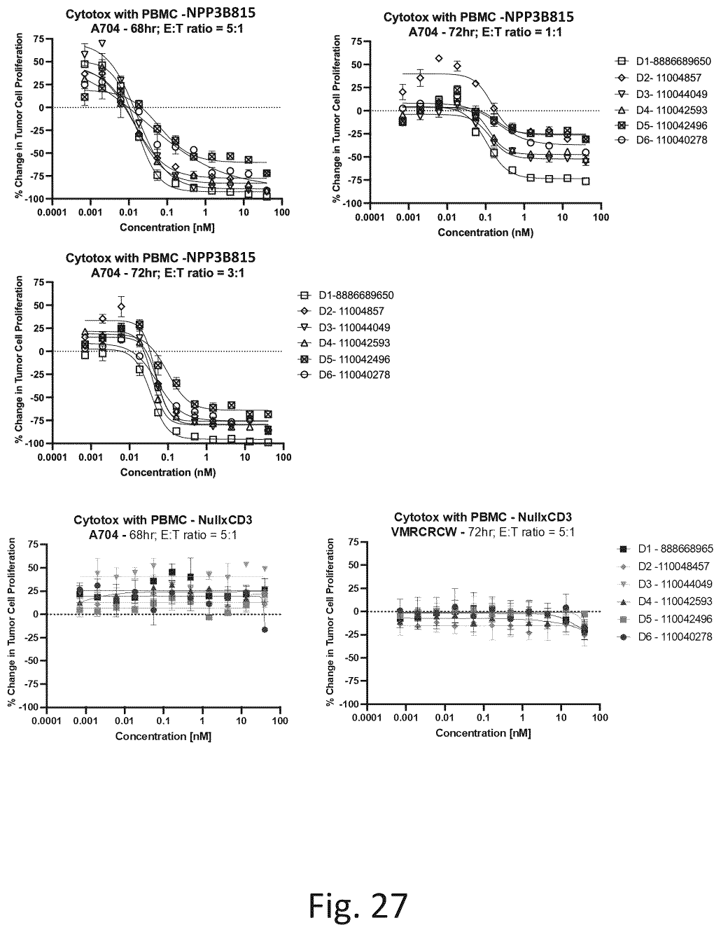

NPP3B815-induced tumor cell killing was evaluated in the presence of PBMCs isolated from corresponding 6 donors from which human T-cell were previously isolated. Dose-dependent cytotoxicity was seen with NPP3B815 treatment in the presence of all 6 PBMC donors in the ENPP3-high cell line A704 at an E:T ratio of 5:1 (FIG. 27). Dose-dependent cytotoxicity was also seen with NPP3B815 treatment in the presence of all 6 PBMC donors in the ENPP3-medium cell line VMRCRCW, while no killing was observed against the negative cell line HepG2 ENPP3-KO.

Further, NPP3B815-induced cytotoxicity in the presence of PBMCs was also evaluated at lower, more physiologically relevant E:T ratios of 3:1 and 1:1. A dose-dependent killing of ENPP3-positive cell line A704 was observed at the lower E:T ratios in the presence of all 6 PBMC donors (FIG. 27). A moderate decrease in the maximum killing activity of NPP3B815 was seen at the lowest E:T ratio of 1:1 (44%), when compared to that observed at an E:T ratio of 5:1 (82%) and 3:1 (78%). As expected, no tumor cell killing was seen with the Null×CD3 control antibody 79C3B615 in the presence of any of the PBMC donors and cell lines tested (FIG. 27). EC50 and maximum killing values are shown in Error! Reference source not found.

TABLE 24
EC50 values for tumor cell cytotoxicity with PBMC donors.

| | E:T = 5:1 | | E:T = 3:1 | | E:T = 1:1 | |
|---|---|---|---|---|---|---|
| | | Max | | Max | | Max |
| | EC₅₀ | killing | EC₅₀ | killing | EC₅₀ | killing |
| T-cell donors | (nM) | (%) | (nM) | (%) | (nM) | (%) |
| | A704 | | | | | |
| 888668965 | 0.015 | 92 | 0.036 | 95 | 0.107 | 73 |
| 110048457 | 0.013 | 78 | 0.046 | 76 | ND | 26 |
| 110044049 | 0.016 | 89 | 0.052 | 79 | 0.131 | 52 |
| 110042593 | 0.015 | 83 | 0.039 | 80 | 0.124 | 47 |
| 110042496 | 0.061 | 60 | 0.097 | 64 | ND | 25 |
| 110040278 | 0.028 | 89 | 0.052 | 75 | ND | 37 |

VMRCRCW

| | | | | | | |
|---|---|---|---|---|---|---|
| 888668965 | 0.062 | 73 | — | — | — | — |
| 110048457 | 0.063 | 69 | — | — | — | — |
| 110044049 | 0.051 | 70 | — | — | — | — |
| 110042593 | 0.033 | 73 | — | — | — | — |
| 110042496 | 0.065 | 52 | — | — | — | — |
| 110040278 | 0.033 | 64 | — | — | — | — |

HepG2 ENPP3 KO

| | | | | | | |
|---|---|---|---|---|---|---|
| 888668965 | ND | ND | — | — | — | — |
| 110048457 | ND | ND | — | — | — | — |
| 110044049 | ND | ND | — | — | — | — |
| 110042593 | ND | ND | — | — | — | — |
| 110042496 | ND | ND | — | — | — | — |
| 110040278 | ND | ND | — | — | — | — |

ECₓ, x % effective concentration; Max, maximal; ND, not determined; PBMC, peripheral blood mononuclear cell.
EC₅₀, EC₉₀, and Max killing values were not determined if the dose-response curve did not satisfy one or both of the following criteria: R² has to be >0.9 and the log (95% confidence interval) difference needs to be <1.2.

7.3.8 Cell Binding and Cytotoxicity with Cyno ENPP3 Expressing Cells.

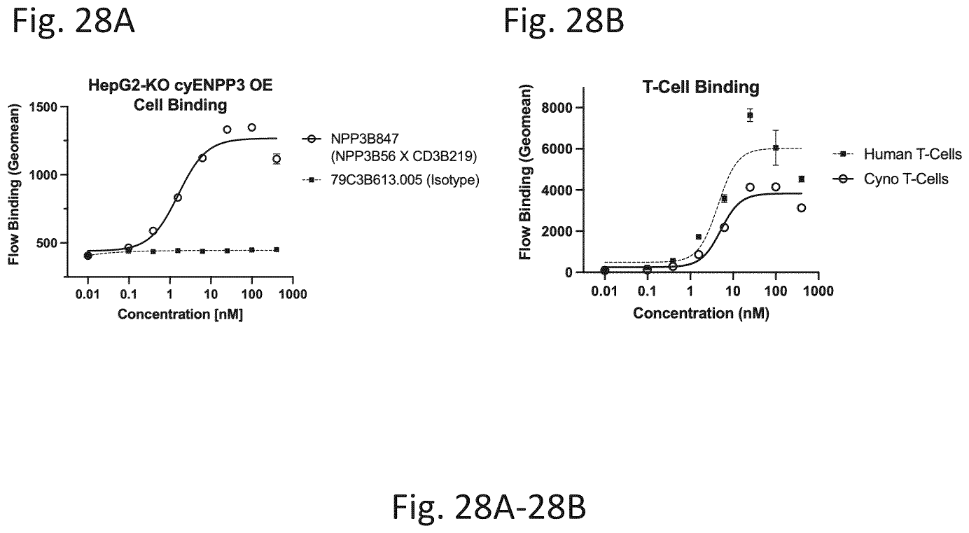

NPP3B815's ENPP3-binding arm is cynomolgus monkey cross-reactive, but its CD3-binding arm is human specific. Therefore, a tool molecule was generated (i.e., NPP3B847) containing the same ENPP3-binding arm as NPP3B815 but including a cynomolgus cross-reactive CD3-binding arm, CD3B219 (unstapled, scFv). In vitro cell binding of the tool molecule NPP3B847 was assessed on human and cyno T-cells as well as a cyno ENPP3 cell line, HepG2-KO cyENPP3 OE. NPP3B847 exhibited a dose-dependent binding to the cyno ENPP3 overexpressing cell line HepG2-KO cyENPP3 OE with an EC50 value of 1.6 nM, with no binding seen with the isotype control (79C3B613) antibody (FIG. 28A and Table 25). Further, comparable dose-dependent binding of NPP3B847 was observed with both human and cynomolgus monkey T cells, with binding affinity (EC50) values being 4.5 nM and 5.2 nM, respectively (FIG. 28B and Table 25).

TABLE 25
Summary of molecular characteristics of
NPP3B815 and NPP3B847

| | | Tool |
|---|---|---|
| | | molecule |
| | Species | NPP3B847 |
| CD3 Binder | | CD3B219 |
| ENPP3 (cell-based) binding | NHP | 1.6 nM |
| CD3 binding (cell-based) | Human | 4.5 nM |
| | NHP | 5.2 nM |

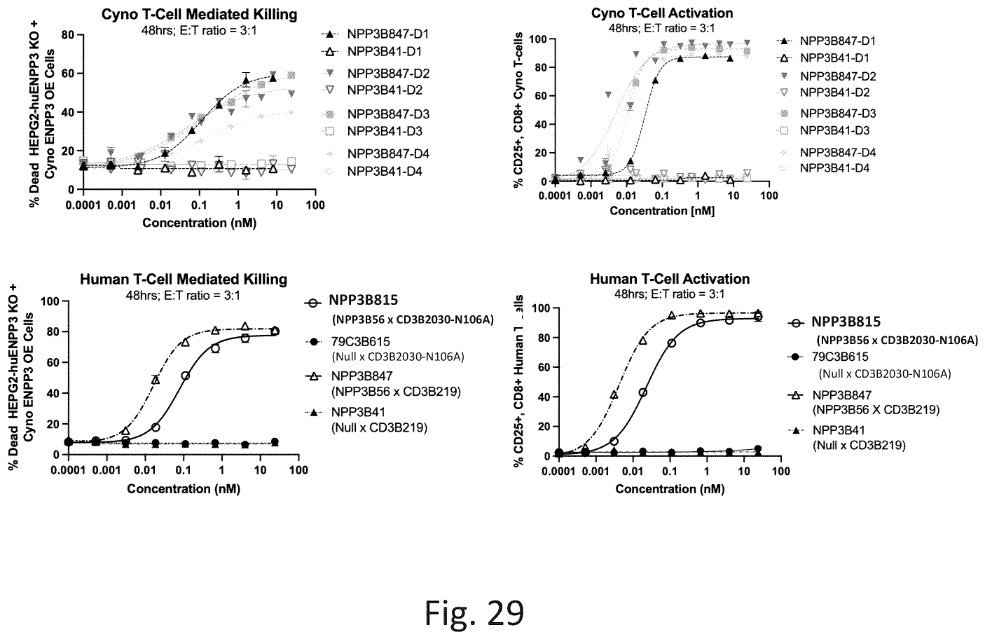

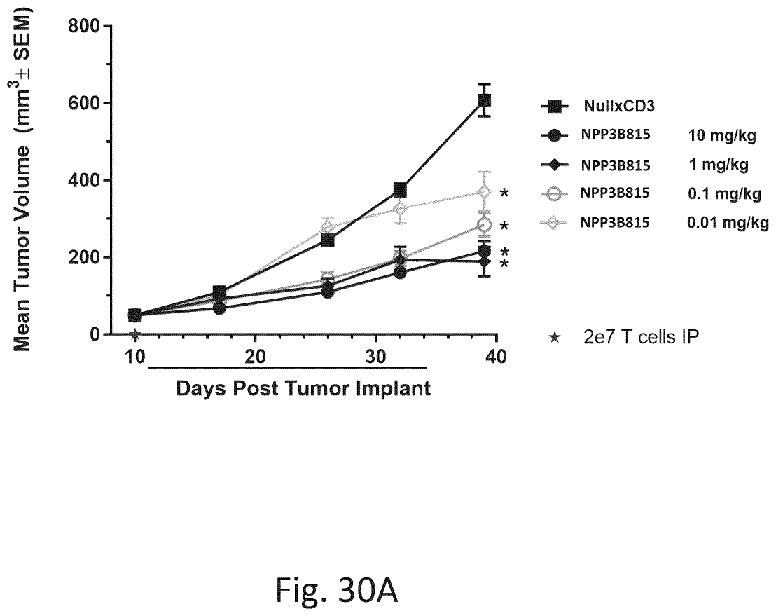

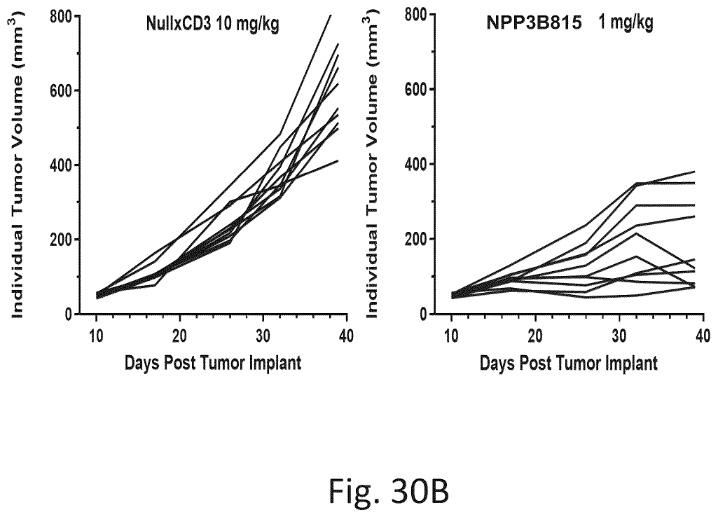

An assessment of the in vitro functional activity of the tool molecule NPP3B847 was conducted by assessing T-cell activation and T-cell-mediated tumor cell cytotoxicity in the presence of human or cynomolgus monkey T cells. These readouts were measured by flow cytometry in HepG2 ENPP3-KO cells overexpressing cyno ENPP3 (FIG. 29 and Table 26) and compared to the activity of the clinical candidate NPP3B815 in the presence of human T-cells.

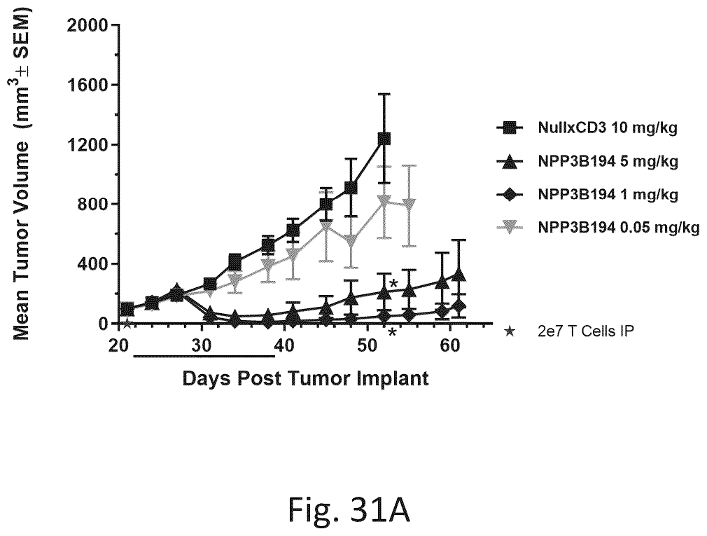

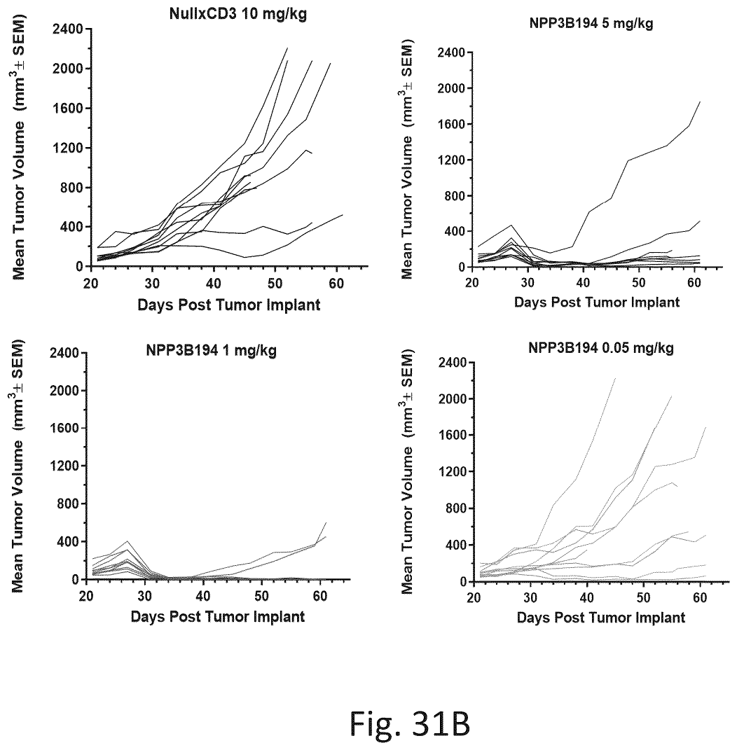

NPP3B847-induced T-cell-mediated cell killing of HepG3-KO cyno ENPP3-OE cell line was evaluated in the presence of cyno T cells isolated from 4 different donors (tested at E:T ratio=3:1) (FIG. 29). Dose-dependent T-cell-mediated cytotoxicity was seen with NPP3B847 across all 4 donors, with some variability seen in EC50 values (0.05 nM-0.14 nM) between the different donors (Table 26). As expected, no T-cell-mediated tumor cell killing was seen with Null×CD3 control antibody NPP3B41 with cyno T-cell donors tested. Further, NPP3B847 induced T-cell-mediated cell killing of HepG2-KO cyno ENPP3-OE cell line was compared to that of NPP3B815 using human T-cells as effector cells at an E:T ratio=3:1 (FIG. 31). Dose-dependent T-cell-mediated cytotoxicity was seen with both NPP3B847 and NPP3B815 at EC50 values of 0.016 nM and 0.079 nM respectively, with no killing seen with Null×CD3 control antibody NPP3B41 with human T-cells (Table 26).

In addition to assessing cytotoxicity, NPP3B847-induced T-cell activation (CD25 expression by flow cytometry at 48 hours) was evaluated with the same 4 cyno T-cell donors at an E:T ratio of 3:1 (FIG. 29). A dose-dependent increase in T-cell activation (EC50=0.005 nM-0.033 nM) was seen with NPP3B847 across all 4 T-cell donors tested in the HepG3-KO cyno ENPP3-OE cell line and no activity was seen with Null×CD3 control antibody NPP3B41. Further, human T-cell activation was evaluated with both NPP3B847 and NPP3B815 using HepG2-KO cyno ENPP3-OE cell line at an E:T ratio=3:1 (FIG. 29). Dose-dependent human T-cell-activation was seen with both NPP3B847 and NPP3B815 at an EC50 value of 0.004 nM and 0.023 nM, respectively, with no activation seen with Null×CD3 control antibody NPP3B41. The EC50 values for cyno and human T-cell activation with NPP3B815 and NPP3B41 are shown in Table 26.

TABLE 26
Summary of EC50 values from cytotoxicity and T-cell activation assays using NPP3B815 and NPP3B847.

| | Cyno T cells | | | | | | | | Human T cells | |
|---|---|---|---|---|---|---|---|---|---|---|
| | Cytotoxicity EC₅₀ (nM) | | | | T-cell Activation EC₅₀ (nM) | | | | Cytoto | T-cell |
| | Donor | Donor | Donor | Donor | Donor | Donor | Donor | Donor | x. EC₅₀ | activation |
| Molecule | 1 | 2 | 3 | 4 | 1 | 2 | 3 | 4 | (nM) | EC₅₀ (nM) |
| NPP3B847 | 0.114 | 0.053 | 0.1115 | 0.137 | 0.033 | 0.005 | 0.011 | 0.008 | 0.016 | 0.004 |

| | | | | |
|---|---|---|---|---|
| NPP3B815 | N/A | N/A | 0.079 | 0.023 |

cyno, cynomolgus monkey; cytotox., cytotoxicity; EC50, 50% effective concentration; N/A, not applicable.

### 7.3.9 In Vivo Efficacy

The antitumor activity of the ENPP3×CD3 stapled (spFv) BsAb NPP3B815 or the unstapled (scFv) version NPP3B194 compared with Null×CD3 control antibody was evaluated in the established ENPP3-low (˜10,600 receptors/cell) human RCC CDX model VMRCRCW or the ENPP3-medium (˜22,000 receptors/cell) human HCC HepG2 model, respectively. Efficacy studies were performed in female immune-compromised NSG mice humanized with donor CD3+ pan T cells. Twice-weekly treatment with NPP3B815 or NPP3B194 administered intraperitoneally (IP) was initiated after SC tumors were established and 1 day post IP T cell engraftment. Engraftment of human T cells can lead to body weight loss due to eventual graft-versus-host disease (GvHD); however, treatment with NPP3B815 did not result in significant body weight loss as compared to the Null×CD3-treated control group. In the HepG2 tumor model, body weight loss due to tumor-induced cachexia was observed across all groups.

In Study ONC2022-035, mice bearing established SC VMRCRCW xenografts were IP dosed with NPP3B815 twice weekly at 10, 1, 0.1, and 0.01 mg/kg or Null×CD3 control antibody (10 mg/kg) for a total of 8 doses (n=10/group). Significant antitumor efficacy was observed with NPP3B815 treatment at 10 and 1 mg/kg over time (p<0.05) with 70% and 75% Δ tumor growth inhibition (TGI), respectively, as compared to Null×CD3-antibody-treated control mice on Day 39 post tumor implantation (Fig. Error! Reference source not found.A). Treatment with NPP3B815 at 0.1 and 0.01 mg/kg resulted in 58% and 42% ΔTGI, respectively, as compared to the Null×CD3-antibody-treated animals on Day 39. While statistically significant (p<0.05) the data at these lower NPP3B815 doses are not biologically significant (Johnson et al., 2001, Br J Cancer, 84 (10): 1424-1431). The lack of biologically significant efficacy (i.e., >60% TGI) observed from treatment with NPP3B815 at 0.1 and 0.01 mg/kg demonstrated that 1 mg/kg is the minimally efficacious dose against low ENPP3 target level model.

In Study P764Y, mice bearing established SC HepG2 xenografts were IP dosed with NPP3B194 (ScFv version of NPP3B815) twice weekly at 5, 1, and 0.05 mg/kg or with Null×CD3 control antibody for a total of 6 doses (n=10/group). Significant antitumor efficacy was observed with NPP3B194 treatment at 5 and 1 mg/kg over time (p<0.05) with 90% and 104% ΔTGI, respectively, as compared to Null×CD3-antibody-treated control mice on Day 52 post tumor implantation (FIG. 31). Treatment with NPP3B194 at 0.05 mg/kg was not statistically significant as compared to the Null×CD3-antibody-treated animals on Day 52. Treatment with NPP3B194 at 5 and 1 mg/kg resulted in 2 of 8 and 7 of 9 complete tumor regressions on the last day of study (Day 61), respectively. All efficacy results are summarized in Table 27.

TABLE 27
Summary of efficacy results for NPP3B815 or NPP3B194.

| | | | Animals | |
|---|---|---|---|---|
| Study type | Species/test | Treatment | per group | |
| [reference] | system | duration | (M/F) | Dose groups and key results |
| VMRCRCW SC | NSG mice/T-cell | 4 weeks | 10 (F) | 42% ΔTGI at 0.01 mg/kg; |
| established [Study | Donor D204071 | (NPP3B815) | | 58% ΔTGI at 0.1 mg/kg; |
| ONC2022-035] | Lot 19055100, | | | 75% ΔTGI at 1 mg/kg; 70% |
| | HemaCare | | | ΔTGI at 10 mg/kg; q3d-q4d |
| | | | | IP for 8 doses |
| HepG2 SC | NSG mice/T-cell | 3 weeks | 10 (F) | 104% ΔTGI at 1 mg/kg, |
| established | Donor D204071 | (NPP3B194) | | 7 of 9 CRs; 90% ΔTGI at |
| [Study P764Y] | Lot 19057273, | | | 5 mg/kg, 2 of 8 CRs; 38% |
| | HemaCare | | | ΔTGI at 0.05 mg/kg (ns); |
| | | | | q3d-q4d IP for 6 doses |

CR, complete response; F, female; IP, intraperitoneal; M, male; NSG, non-obese diabetic (NOD) severe combined immunodeficiency (scid) gamma or NOD.
Cg-Prkdc^{scid} Il2rg^{tm1Wjl}/SzJ; q3d-q4d, twice a week; SC, subcutaneous; TGI, tumor growth inhibition.
p < 0.05 versus control except where noted as not significant (ns).

Next, the effect of NPP3B815 on Growth of HepG2 Established Xenografts in T-Cell-humanized Mice (Study P903S) was evaluated. HepG2 model expresses relatively low ENPP3 target levels. Mice were randomized into groups of 12 animals each, 21 days post human HCC HepG2 tumor implantation according to tumor volume such that all groups had mean values of 114 mm³. Mice enrolled in study were humanized with T cells on the day of randomization and treatment of Null×CD3 at 5 mg/kg or NPP3B815 at 5, 1, 0.5, 0.1, and 0.05 mg/kg was administered IP the following day (Day 22) and twice weekly thereafter for a total of 8 doses.

Mice bearing human HCC HepG2 tumors and treated with NPP3B815 resulted in significant protection against body weight loss in groups treated with NPP3B815 at 5, 1, and 0.5 mg/kg compared to Null×CD3 control animals on Day 45, when at least 9 of 12 mice remained per group. Lower doses of NPP3B815 at 0.1 and 0.05 mg/kg did not have significantly different body weights as compared to Null×CD3 control treatment on Day 45. HepG2 tumors induce cachexia in tumor-bearing animals and severity is proportional to tumor size. The study was terminated on Day 65.

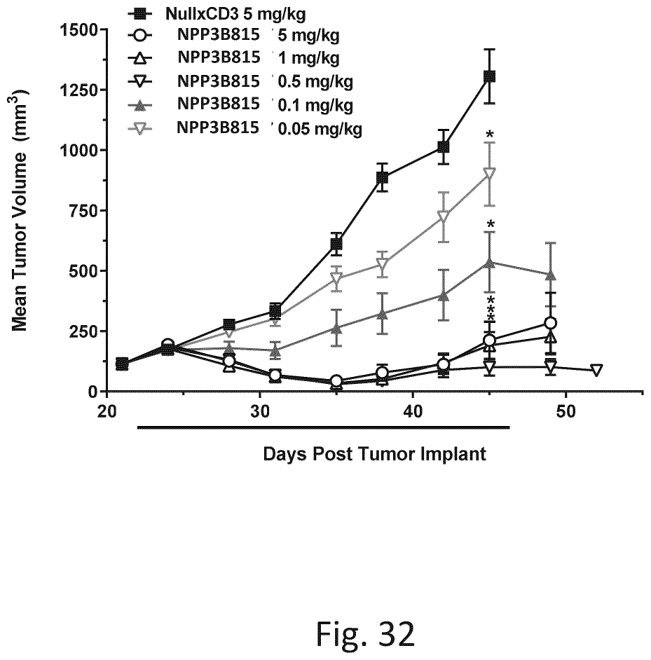

In mice bearing established human HCC HepG2 xenografts, significant anti-tumor efficacy was observed with NPP3B815 treatment at 5, 1, 0.5, 0.1, and 0.05 mg/kg resulting in 92%, 94%, 101%, 65%, and 34% ΔTGI, respectively, as compared to the Null×CD3 antibody treated animals on Day 45 post tumor implantation, when groups had at least 9 of 12 animals remaining (p≤0.05, see FIG. 32). While statistically significant, treatment with NPP3B815 at 0.1 and 0.05 mg/kg did not result in a biologically significant anti-tumor activity (>60% TGI) (59% and 31% TGI, respectively) as compared to the Null×CD3 antibody treated animals on Day 45. The lack of biologically significant efficacy observed from treatment with NPP3B815 at 0.1 and 0.05 mg/kg demonstrated that 0.5 mg/kg is the minimally efficacious dose in the low ENPP3 target level HepG2 model. Treatment with NPP3B815 at 1 and 0.5 mg/kg resulted in 1 CR of 12 mice on Day 49 and 56, respectively. In summary, NPP3B815 at 5, 1, and 0.5 mg/kg significantly inhibited tumor growth resulting in 92%, 94%, and 101% ΔTGI, respectively, at Day 45, compared to Null×CD3 control mice with 1 of 10 complete response (CR) each in the 0.5 and 1 mg/kg dose groups.

Next, the effect of NPP3B815 on growth of RXF 488 established xenografts in T-Cell-humanized Mice (Study P903H2) were evaluated. RCC RXF 488 PDX tumor model expresses relatively high ENPP3 target levels. Mice were randomized into groups of 12 animals each, 18 days post RCC PDX RXF 488 tumor fragment implantation according to tumor volume such that all groups had mean values of 138 mm³. Mice enrolled in study were humanized with T cells on the day of randomization and treatment of Null×CD3 at 5 mg/kg or NPP3B815 at 5, 0.5, 0.05, and 0.005 mg/kg was administered IP the following day (Day 19) and twice weekly thereafter for a total of 8 doses.

Mice bearing RCC RXF 488 tumors and treated with NPP3B815 resulted in significant protection against body weight loss in groups treated with NPP3B815 at 5, 0.5, 0.05, and 0.005 mg/kg compared to Null×CD3 control animals on Day 42, when at least 7 of 12 mice remained per group. RCC RXF 488 tumors induce cachexia in tumor-bearing animals and severity was proportional to tumor size. The study was terminated on Day 60.

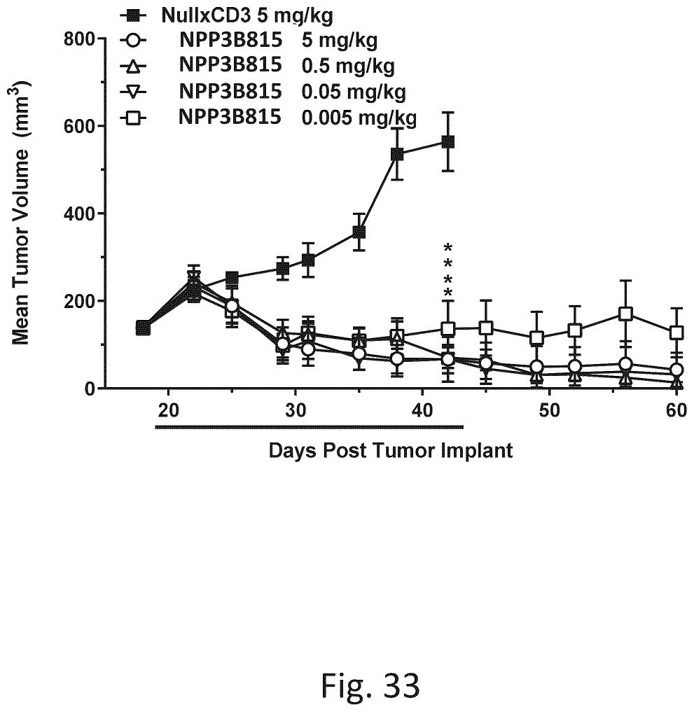

In mice bearing SC RCC RXF 488 PDX tumors, significant anti-tumor efficacy was observed with NPP3B815 treatment at 5, 0.5, 0.05, and 0.005 mg/kg resulting in 117%, 116%, 117% and 100% ΔTGI, respectively, as compared to the Null×CD3 antibody treated animals on Day 42 post tumor implantation, when groups had at least 7 of 12 animals remaining (p≤0.05, see FIG. 33). Treatment with NPP3B815 at 5, 0.5, 0.05, and 0.005 mg/kg resulted in 72%, 89%, 74% (p≤0.05) and 2% (not significant) treatment response (TR) and 7, 6, 8, and 4 of 12 complete response (CR), respectively, on Day 60. Thus, all dose levels down to 0.005 mg/kg were highly efficacious in the higher RXF 488 PDX tumor model.

What is claimed is:

1. A binding agent comprising an antigen binding region that binds to an epitope of ENPP3.

2. The binding agent of claim 1, wherein the epitope of ENPP3 is selected from the group consisting of SEQ ID NO:295-297, DVP, and SEQ ID NO:299-305.

3. The binding agent of claim 1, wherein the antigen binding region comprises a VH and VL domain selected from the group consisting of:
(a) a VH domain comprising a HCDR1, HCDR2, and HCDR3 of a VH as set forth in SEQ ID NO:22, and a VL domain comprising a LCDR1, LCDR2, and LCDR3 of a VL as set forth in SEQ ID NO:23;
(b) a VH domain comprising a HCDR1, HCDR2, and HCDR3 of a VH as set forth in SEQ ID NO:45, and a VL domain comprising a LCDR1, LCDR2, and LCDR3 of a VL as set forth in SEQ ID NO:46;
(c) a VH domain comprising a HCDR1, HCDR2, and HCDR3 of a VH as set forth in SEQ ID NO:68, and a VL domain comprising a LCDR1, LCDR2, and LCDR3 of a VL as set forth in SEQ ID NO:69 or SEQ ID NO:70;
(d) a VH domain comprising a HCDR1, HCDR2, and HCDR3 of a VH as set forth in SEQ ID NO:91, and a VL domain comprising a LCDR1, LCDR2, and LCDR3 of a VL as set forth in SEQ ID NO:92;
(e) a VH domain comprising a HCDR1, HCDR2, and HCDR3 of a VH as set forth in SEQ ID NO:113, and a VL domain comprising a LCDR1, LCDR2, and LCDR3 of a VL as set forth in SEQ ID NO: 114 or SEQ ID NO:115;
(f) a VH domain comprising a HCDR1, HCDR2, and HCDR3 of a VH as set forth in SEQ ID NO: 134, and a VL domain comprising a LCDR1, LCDR2, and LCDR3 of a VL as set forth in SEQ ID NO: 135 or SEQ ID NO: 136;
(g) a VH domain comprising a HCDR1, HCDR2, and HCDR3 of a VH as set forth in SEQ ID NO:158, and a VL domain comprising a LCDR1, LCDR2, and LCDR3 of a VL as set forth in SEQ ID NO: 159; and
(h) a VH domain comprising a HCDR1, HCDR2, and HCDR3 of a VH as set forth in SEQ ID NO:177, and a VL domain comprising a LCDR1, LCDR2, and LCDR3 of a VL as set forth in SEQ ID NO:178.

4. The binding agent of claim 3, wherein the CDR sequences are selected from the group consisting of:
(a1) the HCDR1 comprises the amino acid sequence of SEQ ID NO:1, the HCDR2 comprises the amino acid sequence of SEQ ID NO:2, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
(a2) the HCDR1 comprises the amino acid sequence of SEQ ID NO:7, the HCDR2 comprises the amino acid sequence of SEQ ID NO:8, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
(a3) the HCDR1 comprises the amino acid sequence of SEQ ID NO:9, the HCDR2 comprises the amino acid sequence of SEQ ID NO: 10, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
(a4) the HCDR1 comprises the amino acid sequence of SEQ ID NO:11, the HCDR2 comprises the amino acid sequence of SEQ ID NO: 12, the HCDR3 comprises the amino acid sequence of SEQ ID NO:13, the LCDR1 comprises the amino acid sequence of SEQ ID NO:14, the LCDR2 comprises the amino acid sequence of SEQ ID NO:15, and the LCDR3 comprises the amino acid sequence of SEQ ID NO: 16;
(a5) the HCDR1 comprises the amino acid sequence of SEQ ID NO: 17, the HCDR2 comprises the amino acid sequence of SEQ ID NO: 18, the HCDR3 comprises the amino acid sequence of SEQ ID NO:19, the LCDR1 comprises the amino acid sequence of SEQ ID NO:20, the LCDR2 comprises the amino acid sequence of GAS, and the LCDR3 comprises the amino acid sequence of SEQ ID NO:6;
(b1) the HCDR1 comprises the amino acid sequence of SEQ ID NO:24, the HCDR2 comprises the amino acid sequence of SEQ ID NO:25, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
(b2) the HCDR1 comprises the amino acid sequence of SEQ ID NO:30, the HCDR2 comprises the amino acid sequence of SEQ ID NO:31, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
(b3) the HCDR1 comprises the amino acid sequence of SEQ ID NO:32, the HCDR2 comprises the amino acid sequence of SEQ ID NO:33, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
(b4) the HCDR1 comprises the amino acid sequence of SEQ ID NO:34, the HCDR2 comprises the amino acid sequence of SEQ ID NO:35, the HCDR3 comprises the amino acid sequence of SEQ ID NO:36, the LCDR1 comprises the amino acid sequence of SEQ ID NO:37, the LCDR2 comprises the amino acid sequence of SEQ ID NO:38, and the LCDR3 comprises the amino acid sequence of SEQ ID NO: 39;
(b5) the HCDR1 comprises the amino acid sequence of SEQ ID NO:40, the HCDR2 comprises the amino acid sequence of SEQ ID NO:41, the HCDR3 comprises the amino acid sequence of SEQ ID NO:42, the LCDR1 comprises the amino acid sequence of SEQ ID NO:43, the LCDR2 comprises the amino acid sequence of DAS, and the LCDR3 comprises the amino acid sequence of SEQ ID NO:29;
(c1) the HCDR1 comprises the amino acid sequence of SEQ ID NO:47, the HCDR2 comprises the amino acid sequence of SEQ ID NO:48, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
(c2) the HCDR1 comprises the amino acid sequence of SEQ ID NO:53, the HCDR2 comprises the amino acid sequence of SEQ ID NO:54, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
(c3) the HCDR1 comprises the amino acid sequence of SEQ ID NO:55, the HCDR2 comprises the amino acid sequence of SEQ ID NO:56, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
(c4) the HCDR1 comprises the amino acid sequence of SEQ ID NO:57, the HCDR2 comprises the amino acid sequence of SEQ ID NO:58, the HCDR3 comprises the amino acid sequence of SEQ ID NO:59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO: 62;
(c5) the HCDR1 comprises the amino acid sequence of SEQ ID NO:63, the HCDR2 comprises the amino acid sequence of SEQ ID NO:64, the HCDR3 comprises the amino acid sequence of SEQ ID NO:65, the LCDR1 comprises the amino acid sequence of SEQ ID NO:66, the LCDR2 comprises the amino acid sequence of QIS, and the LCDR3 comprises the amino acid sequence of SEQ ID NO:52;
(d1) the HCDR1 comprises the amino acid sequence of SEQ ID NO:71, the HCDR2 comprises the amino acid sequence of SEQ ID NO:72, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
(d2) the HCDR1 comprises the amino acid sequence of SEQ ID NO:77, the HCDR2 comprises the amino acid sequence of SEQ ID NO:78, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
(d3) the HCDR1 comprises the amino acid sequence of SEQ ID NO:79, the HCDR2 comprises the amino acid sequence of SEQ ID NO:80, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
(d4) the HCDR1 comprises the amino acid sequence of SEQ ID NO:81, the HCDR2 comprises the amino acid sequence of SEQ ID NO:82, the HCDR3 comprises the amino acid sequence of SEQ ID NO:83, the LCDR1 comprises the amino acid sequence of SEQ ID NO:84, the LCDR2 comprises the amino acid sequence of SEQ ID NO:85, and the LCDR3 comprises the amino acid sequence of SEQ ID NO: 86;
(d5) the HCDR1 comprises the amino acid sequence of SEQ ID NO:87, the HCDR2 comprises the amino acid sequence of SEQ ID NO:88, the HCDR3 comprises the amino acid sequence of SEQ ID NO:89, the LCDR1 comprises the amino acid sequence of SEQ ID NO:90, the LCDR2 comprises the amino acid sequence of GAS, and the LCDR3 comprises the amino acid sequence of SEQ ID NO:76;
(e1) the HCDR1 comprises the amino acid sequence of SEQ ID NO:93, the HCDR2 comprises the amino acid sequence of SEQ ID NO:94, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97 and the LCDR3 comprises the amino acid sequence of SEQ ID NO: 98;
(e2) the HCDR1 comprises the amino acid sequence of SEQ ID NO:99, the HCDR2 comprises the amino acid sequence of SEQ ID NO: 100, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO: 98;
(e3) the HCDR1 comprises the amino acid sequence of SEQ ID NO: 101, the HCDR2 comprises the amino acid sequence of SEQ ID NO: 102, the HCDR3 comprises the amino acid sequence of SEQ ID NO:95, the LCDR1 comprises the amino acid sequence of SEQ ID NO:96 the LCDR2 comprises the amino acid sequence of SEQ ID NO:97, and the LCDR3 comprises the amino acid sequence of SEQ ID NO: 98;
(e4) the HCDR1 comprises the amino acid sequence of SEQ ID NO: 103, the HCDR2 comprises the amino acid sequence of SEQ ID NO: 104, the HCDR3 comprises the amino acid sequence of SEQ ID NO: 105, the LCDR1 comprises the amino acid sequence of SEQ ID NO:106, the LCDR2 comprises the amino acid sequence of SEQ ID NO:107, and the LCDR3 comprises the amino acid sequence of SEQ ID NO:108;
(e5) the HCDR1 comprises the amino acid sequence of SEQ ID NO: 109, the HCDR2 comprises the amino acid sequence of SEQ ID NO: 110, the HCDR3 comprises the amino acid sequence of SEQ ID NO:111, the LCDR1 comprises the amino acid sequence of SEQ ID NO:112, the LCDR2 comprises the amino acid sequence of GAS, and the LCDR3 comprises the amino acid sequence of SEQ ID NO: 98;
(f1) the HCDR1 comprises the amino acid sequence of SEQ ID NO: 116, the HCDR2 comprises the amino acid sequence of SEQ ID NO: 117, the HCDR3 comprises the amino acid sequence of SEQ ID NO:118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120;
(f2) the HCDR1 comprises the amino acid sequence of SEQ ID NO: 121, the HCDR2 comprises the amino acid sequence of SEQ ID NO: 122, the HCDR3 comprises the amino acid sequence of SEQ ID NO: 118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO:120;
(f3) the HCDR1 comprises the amino acid sequence of SEQ ID NO: 123, the HCDR2 comprises the amino acid sequence of SEQ ID NO: 124, the HCDR3 comprises the amino acid sequence of SEQ ID NO: 118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120;
(f4) the HCDR1 comprises the amino acid sequence of SEQ ID NO: 125, the HCDR2 comprises the amino acid sequence of SEQ ID NO:126, the HCDR3 comprises the amino acid sequence of SEQ ID NO: 127, the LCDR1 comprises the amino acid sequence of SEQ ID NO:128, the LCDR2 comprises the amino acid sequence of SEQ ID NO:15, and the LCDR3 comprises the amino acid sequence of SEQ ID NO: 129;
(f5) the HCDR1 comprises the amino acid sequence of SEQ ID NO: 130, the HCDR2 comprises the amino acid sequence of SEQ ID NO:131, the HCDR3 comprises the amino acid sequence of SEQ ID NO:132, the LCDR1 comprises the amino acid sequence of SEQ ID NO: 133, the LCDR2 comprises the amino acid sequence of GAS, and the LCDR3 comprises the amino acid sequence of SEQ ID NO: 120;
(g1) the HCDR1 comprises the amino acid sequence of SEQ ID NO: 137, the HCDR2 comprises the amino acid sequence of SEQ ID NO:138, the HCDR3 comprises the amino acid sequence of SEQ ID NO:139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO:142;
(g2) the HCDR1 comprises the amino acid sequence of SEQ ID NO: 143, the HCDR2 comprises the amino acid sequence of SEQ ID NO: 144, the HCDR3 comprises the amino acid sequence of SEQ ID NO: 139, the LCDR1 comprises the amino acid sequence of SEQ ID NO:140, the LCDR2 comprises the amino acid sequence of SEQ ID NO: 141, and the LCDR3 comprises the amino acid sequence of SEQ ID NO:142;
(g3) the HCDR1 comprises the amino acid sequence of SEQ ID NO: 145, the HCDR2 comprises the amino acid sequence of SEQ ID NO: 146, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO:142;
(g4) the HCDR1 comprises the amino acid sequence of SEQ ID NO: 147, the HCDR2 comprises the amino acid sequence of SEQ ID NO: 148, the HCDR3 comprises the amino acid sequence of SEQ ID NO: 149, the LCDR1 comprises the amino acid sequence of SEQ ID NO:150, the LCDR2 comprises the amino acid sequence of SEQ ID NO: 151, and the LCDR3 comprises the amino acid sequence of SEQ ID NO:152;
(g5) the HCDR1 comprises the amino acid sequence of SEQ ID NO: 153, the HCDR2 comprises the amino acid sequence of SEQ ID NO:154, the HCDR3 comprises the amino acid sequence of SEQ ID NO: 155, the LCDR1 comprises the amino acid sequence of SEQ ID NO: 156, the LCDR2 comprises the amino acid sequence of AAS, and the LCDR3 comprises the amino acid sequence of SEQ ID NO: 142;
(h1) the HCDR1 comprises the amino acid sequence of SEQ ID NO:1, the HCDR2 comprises the amino acid sequence of SEQ ID NO:160, the HCDR3 comprises the amino acid sequence of SEQ ID NO:161, the LCDR1 comprises the amino acid sequence of SEQ ID NO: 162, the LCDR2 comprises the amino acid sequence of SEQ ID NO: 163, and the LCDR3 comprises the amino acid sequence of SEQ ID NO:76;
(h2) the HCDR1 comprises the amino acid sequence of SEQ ID NO:164, the HCDR2 comprises the amino acid sequence of SEQ ID NO: 165, the HCDR3 comprises the amino acid sequence of SEQ ID NO: 161, the LCDR1 comprises the amino acid sequence of SEQ ID NO: 162 the LCDR2 comprises the amino acid sequence of SEQ ID NO: 163, and the LCDR3 comprises the amino acid sequence of SEQ ID NO: 76;
(h3) the HCDR1 comprises the amino acid sequence of SEQ ID NO: 166, the HCDR2 comprises the amino acid sequence of SEQ ID NO: 167, the HCDR3 comprises the amino acid sequence of SEQ ID NO: 161, the LCDR1 comprises the amino acid sequence of SEQ ID NO:162, the LCDR2 comprises the amino acid sequence of SEQ ID NO: 163, and the LCDR3 comprises the amino acid sequence of SEQ ID NO:76;
(h4) the HCDR1 comprises the amino acid sequence of SEQ ID NO: 11, the HCDR2 comprises the amino acid sequence of SEQ ID NO: 168, the HCDR3 comprises the amino acid sequence of SEQ ID NO: 169, the LCDR1 comprises the amino acid sequence of SEQ ID NO:170, the LCDR2 comprises the amino acid sequence of SEQ ID NO:171, and the LCDR3 comprises the amino acid sequence of SEQ ID NO:86; and
(h5) the HCDR1 comprises the amino acid sequence of SEQ ID NO:172, the HCDR2 comprises the amino acid sequence of SEQ ID NO:173, the HCDR3 comprises the amino acid sequence of SEQ ID NO: 174, the LCDR1 comprises the amino acid sequence of SEQ ID NO:175, the LCDR2 comprises the amino acid sequence of VAS and the LCDR3 comprises the amino acid sequence of SEQ ID NO: 76.

5. The binding agent of any one of claims 1 to 4, wherein the antigen binding region comprises a VH domain and a VL domain selected from the group consisting of:
(a) a VH domain comprising the amino acid sequence of SEQ ID NO:22, and a VL domain comprising the amino acid sequence of SEQ ID NO:23;
(b) a VH domain comprising the amino acid sequence of SEQ ID NO:45, and a VL domain comprising the amino acid sequence of SEQ ID NO:46;
(c) a VH domain comprising the amino acid sequence of SEQ ID NO:68, and a VL domain comprising the amino acid sequence of SEQ ID NO:69;
(c′) a VH domain comprising the amino acid sequence of SEQ ID NO:68, and a VL domain comprising the amino acid sequence of SEQ ID NO:70;
(d) a VH domain comprising the amino acid sequence of SEQ ID NO:91, and a VL domain comprising the amino acid sequence of SEQ ID NO:92;
(e) a VH domain comprising the amino acid sequence of SEQ ID NO:113, and a VL domain comprising the amino acid sequence of SEQ ID NO:114;
(e′) a VH domain comprising the amino acid sequence of SEQ ID NO:113, and a VL domain comprising the amino acid sequence of SEQ ID NO:115;
(f) a VH domain comprising the amino acid sequence of SEQ ID NO:134, and a VL domain comprising the amino acid sequence of SEQ ID NO:135;
(f′) a VH domain comprising the amino acid sequence of SEQ ID NO:134, and a VL domain comprising the amino acid sequence of SEQ ID NO:136;
(g) a VH domain comprising the amino acid sequence of SEQ ID NO: 158, and a VL domain comprising the amino acid sequence of SEQ ID NO: 159; and
(h) a VH domain comprising the amino acid sequence of SEQ ID NO:177, and a VL domain comprising the amino acid sequence of SEQ ID NO:178.

6. The binding agent of any one of claims 1 to 5, wherein the binding agent is a bispecific protein or a multi-specific protein.

7. The binding agent of any one of claims 1 to 6, further comprising an immunoglobulin (Ig) constant region, or a fragment of the Ig constant region, wherein optionally the fragment of the Ig constant region is an Fc region or an CH3 domain.

8. The binding agent of claim 7, wherein the Ig constant region, the fragment of the Ig constant region, the Fc region, or the CH3 domain comprises at least one mutation.

9. The binding agent of claim 8, wherein the at least one mutation is selected from the group consisting of L234A/L235A/D265S, F234A/L235A, L234A/L235A, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

10. The binding agent of claim 8, wherein the at least one mutation is selected from the group consisting of T366S/L368A/Y407V, T366W, T350V, L351Y, F405A, Y407V, T366Y, T366L, F405W, T394W, K392L, T394S, Y407T, Y407A, L351Y/F405A/Y407V, T366I/K392M/T394W, F405A/Y407V, T366L/K392M/T394W, T366L/K392L/T394W, L351Y/Y407A, L351Y/Y407V, T366A/K409F, T366V/K409F, T366A/K409F, T350V/L351Y/F405A/Y407V and T350V/T366L/K392L/T394W, wherein residue numbering is according to the EU index.

11. The binding agent of claim 8, wherein the binding agent comprises knob-in-hole mutations, wherein the knob mutations comprise T366S/L368A/Y407V, and the hole mutation comprises T366W.

12. The binding agent of any one of claims 1 to 11, wherein the agent comprises a bispecific protein comprising an antigen binding region that binds a second antigen other than ENPP3.

13. The binding agent of claim 12, wherein the second antigen is cluster of differentiation 3ε (CD3ε).

14. A binding agent comprising a first antigen binding region that binds to ENPP3 and a second antigen binding region that binds to CD3ε, wherein the first antigen binding region that binds to ENPP3 comprises a VH and VL selected from the group consisting of:
(a) a VH domain comprising a HCDR1, HCDR2, and HCDR3 of a VH as set forth in SEQ ID NO:22, and a VL domain comprising a LCDR1, LCDR2, and LCDR3 of a VL as set forth in SEQ ID NO:23;
(b) a VH domain comprising a HCDR1, HCDR2, and HCDR3 of a VH as set forth in SEQ ID NO:45, and a VL domain comprising a LCDR1, LCDR2, and LCDR3 of a VL as set forth in SEQ ID NO:46;
(c) a VH domain comprising a HCDR1, HCDR2, and HCDR3 of a VH as set forth in SEQ ID NO:68, and a VL domain comprising a LCDR1, LCDR2, and LCDR3 of a VL as set forth in SEQ ID NO:69 or SEQ ID NO:70;
(d) a VH domain comprising a HCDR1, HCDR2, and HCDR3 of a VH as set forth in SEQ ID NO:91, and a VL domain comprising a LCDR1, LCDR2, and LCDR3 of a VL as set forth in SEQ ID NO:92;
(e) a VH domain comprising a HCDR1, HCDR2, and HCDR3 of a VH as set forth in SEQ ID NO: 113, and a VL domain comprising a LCDR1, LCDR2, and LCDR3 of a VL as set forth in SEQ ID NO: 114 or SEQ ID NO:115;
(f) a VH domain comprising a HCDR1, HCDR2, and HCDR3 of a VH as set forth in SEQ ID NO:134, and a VL domain comprising a LCDR1, LCDR2, and LCDR3 of a VL as set forth in SEQ ID NO:135 or SEQ ID NO:136;
(g) a VH domain comprising a HCDR1, HCDR2, and HCDR3 of a VH as set forth in SEQ ID NO:158, and a VL domain comprising a LCDR1, LCDR2, and LCDR3 of a VL as set forth in SEQ ID NO:159; and
(h) a VH domain comprising a HCDR1, HCDR2, and HCDR3 of a VH as set forth in SEQ ID NO:177, and a VL domain comprising a LCDR1, LCDR2, and LCDR3 of a VL as set forth in SEQ ID NO:178.

15. The binding agent of claim 14, wherein in the second antigen binding region that binds to CD3ε comprises a VH domain comprising a HCDR1, HCDR2, and HCDR3 of a VH as set forth in SEQ ID NO:200, and a VL domain comprising a LCDR1, LCDR2, and LCDR3 of a VL as set forth in SEQ ID NO:201.

17. The binding agent of claim 14, wherein the first antigen binding region that binds to ENPP3 comprises CDR sequences selected from the group consisting of:
(a1) the HCDR1 comprises the amino acid sequence of SEQ ID NO:1, the HCDR2 comprises the amino acid sequence of SEQ ID NO:2, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
(a2) the HCDR1 comprises the amino acid sequence of SEQ ID NO:7, the HCDR2 comprises the amino acid sequence of SEQ ID NO:8, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
(a3) the HCDR1 comprises the amino acid sequence of SEQ ID NO:9, the HCDR2 comprises the amino acid sequence of SEQ ID NO: 10, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO: 6;
(a4) the HCDR1 comprises the amino acid sequence of SEQ ID NO: 11, the HCDR2 comprises the amino acid sequence of SEQ ID NO:12, the HCDR3 comprises the amino acid sequence of SEQ ID NO:13, the LCDR1 comprises the amino acid sequence of SEQ ID NO: 14, the LCDR2 comprises the amino acid sequence of SEQ ID NO:15, and the LCDR3 comprises the amino acid sequence of SEQ ID NO: 16;
(a5) the HCDR1 comprises the amino acid sequence of SEQ ID NO: 17, the HCDR2 comprises the amino acid sequence of SEQ ID NO: 18, the HCDR3 comprises the amino acid sequence of SEQ ID NO:19, the LCDR1 comprises the amino acid sequence of SEQ ID NO:20, the LCDR2 comprises the amino acid sequence of GAS, and the LCDR3 comprises the amino acid sequence of SEQ ID NO:6;
(b1) the HCDR1 comprises the amino acid sequence of SEQ ID NO:24, the HCDR2 comprises the amino acid sequence of SEQ ID NO:25, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
(b2) the HCDR1 comprises the amino acid sequence of SEQ ID NO:30, the HCDR2 comprises the amino acid sequence of SEQ ID NO:31, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
(b3) the HCDR1 comprises the amino acid sequence of SEQ ID NO:32, the HCDR2 comprises the amino acid sequence of SEQ ID NO:33, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO: 29;
(b4) the HCDR1 comprises the amino acid sequence of SEQ ID NO:34, the HCDR2 comprises the amino acid sequence of SEQ ID NO:35, the HCDR3 comprises the amino acid sequence of SEQ ID NO:36, the LCDR1 comprises the amino acid sequence of SEQ ID NO:37, the LCDR2 comprises the amino acid sequence of SEQ ID NO:38, and the LCDR3 comprises the amino acid sequence of SEQ ID NO: 39;
(b5) the HCDR1 comprises the amino acid sequence of SEQ ID NO:40, the HCDR2 comprises the amino acid sequence of SEQ ID NO:41, the HCDR3 comprises the amino acid sequence of SEQ ID NO:42, the LCDR1 comprises the amino acid sequence of SEQ ID NO:43, the LCDR2 comprises the amino acid sequence of DAS, and the LCDR3 comprises the amino acid sequence of SEQ ID NO:29;
(c1) the HCDR1 comprises the amino acid sequence of SEQ ID NO:47, the HCDR2 comprises the amino acid sequence of SEQ ID NO:48, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
(c2) the HCDR1 comprises the amino acid sequence of SEQ ID NO:53, the HCDR2 comprises the amino acid sequence of SEQ ID NO:54, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
(c3) the HCDR1 comprises the amino acid sequence of SEQ ID NO:55, the HCDR2 comprises the amino acid sequence of SEQ ID NO:56, the HCDR3 comprises the amino acid sequence of SEQ ID NO:49, the LCDR1 comprises the amino acid sequence of SEQ ID NO:50, the LCDR2 comprises the amino acid sequence of SEQ ID NO:51, and the LCDR3 comprises the amino acid sequence of SEQ ID NO: 52;
(c4) the HCDR1 comprises the amino acid sequence of SEQ ID NO:57, the HCDR2 comprises the amino acid sequence of SEQ ID NO:58, the HCDR3 comprises the amino acid sequence of SEQ ID NO:59, the LCDR1 comprises the amino acid sequence of SEQ ID NO:60, the LCDR2 comprises the amino acid sequence of SEQ ID NO:61, and the LCDR3 comprises the amino acid sequence of SEQ ID NO: 62;
(c5) the HCDR1 comprises the amino acid sequence of SEQ ID NO:63, the HCDR2 comprises the amino acid sequence of SEQ ID NO:64, the HCDR3 comprises the amino acid sequence of SEQ ID NO:65, the LCDR1 comprises the amino acid sequence of SEQ ID NO:66, the LCDR2 comprises the amino acid sequence of QIS, and the LCDR3 comprises the amino acid sequence of SEQ ID NO:52;
(d1) the HCDR1 comprises the amino acid sequence of SEQ ID NO:71, the HCDR2 comprises the amino acid sequence of SEQ ID NO:72, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
(d2) the HCDR1 comprises the amino acid sequence of SEQ ID NO: 77, the HCDR2 comprises the amino acid sequence of SEQ ID NO:78, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
(d3) the HCDR1 comprises the amino acid sequence of SEQ ID NO:79, the HCDR2 comprises the amino acid sequence of SEQ ID NO:80, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO: 76;
(d4) the HCDR1 comprises the amino acid sequence of SEQ ID NO:81, the HCDR2 comprises the amino acid sequence of SEQ ID NO:82, the HCDR3 comprises the amino acid sequence of SEQ ID NO:83, the LCDR1 comprises the amino acid sequence of SEQ ID NO:84, the LCDR2 comprises the amino acid sequence of SEQ ID NO:85, and the LCDR3 comprises the amino acid sequence of SEQ ID NO: 86;
(d5) the HCDR1 comprises the amino acid sequence of SEQ ID NO:87, the HCDR2 comprises the amino acid sequence of SEQ ID NO:88, the HCDR3 comprises the amino acid sequence of SEQ ID NO:89, the LCDR1 comprises the amino acid sequence of SEQ ID NO:90, the LCDR2 comprises the amino acid sequence of GAS, and the LCDR3 comprises the amino acid sequence of SEQ ID NO:76;
(e1) the HCDR1 comprises the amino acid sequence of SEQ ID NO:93, the HCDR2 comprises the amino acid sequence of SEQ ID NO:94, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97 and the LCDR3 comprises the amino acid sequence of SEQ ID NO: 98;
(e2) the HCDR1 comprises the amino acid sequence of SEQ ID NO:99, the HCDR2 comprises the amino acid sequence of SEQ ID NO: 100, the HCDR3 comprises the amino acid sequence of SEQ ID NO:95, the LCDR1 comprises the amino acid sequence of SEQ ID NO:96, the LCDR2 comprises the amino acid sequence of SEQ ID NO:97, and the LCDR3 comprises the amino acid sequence of SEQ ID NO: 98;
(e3) the HCDR1 comprises the amino acid sequence of SEQ ID NO: 101, the HCDR2 comprises the amino acid sequence of SEQ ID NO: 102, the HCDR3 comprises the amino acid sequence of SEQ ID NO:95, the LCDR1 comprises the amino acid sequence of SEQ ID NO:96 the LCDR2 comprises the amino acid sequence of SEQ ID NO:97, and the LCDR3 comprises the amino acid sequence of SEQ ID NO: 98;
(e4) the HCDR1 comprises the amino acid sequence of SEQ ID NO: 103, the HCDR2 comprises the amino acid sequence of SEQ ID NO: 104, the HCDR3 comprises the amino acid sequence of SEQ ID NO: 105, the LCDR1 comprises the amino acid sequence of SEQ ID NO: 106, the LCDR2 comprises the amino acid sequence of SEQ ID NO: 107, and the LCDR3 comprises the amino acid sequence of SEQ ID NO:108;
(e5) the HCDR1 comprises the amino acid sequence of SEQ ID NO: 109, the HCDR2 comprises the amino acid sequence of SEQ ID NO:110, the HCDR3 comprises the amino acid sequence of SEQ ID NO: 111, the LCDR1 comprises the amino acid sequence of SEQ ID NO:112, the LCDR2 comprises the amino acid sequence of GAS, and the LCDR3 comprises the amino acid sequence of SEQ ID NO: 98;
(f1) the HCDR1 comprises the amino acid sequence of SEQ ID NO:116, the HCDR2 comprises the amino acid sequence of SEQ ID NO:117, the HCDR3 comprises the amino acid sequence of SEQ ID NO: 118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120;
(f2) the HCDR1 comprises the amino acid sequence of SEQ ID NO: 121, the HCDR2 comprises the amino acid sequence of SEQ ID NO: 122, the HCDR3 comprises the amino acid sequence of SEQ ID NO: 118, the LCDR1 comprises the amino acid sequence of SEQ ID NO:119, the LCDR2 comprises the amino acid sequence of SEQ ID NO:5, and the LCDR3 comprises the amino acid sequence of SEQ ID NO:120;
(f3) the HCDR1 comprises the amino acid sequence of SEQ ID NO: 123, the HCDR2 comprises the amino acid sequence of SEQ ID NO: 124, the HCDR3 comprises the amino acid sequence of SEQ ID NO: 118, the LCDR1 comprises the amino acid sequence of SEQ ID NO: 119, the LCDR2 comprises the amino acid sequence of SEQ ID NO:5, and the LCDR3 comprises the amino acid sequence of SEQ ID NO:120;
(f4) the HCDR1 comprises the amino acid sequence of SEQ ID NO: 125, the HCDR2 comprises the amino acid sequence of SEQ ID NO: 126, the HCDR3 comprises the amino acid sequence of SEQ ID NO: 127, the LCDR1 comprises the amino acid sequence of SEQ ID NO: 128, the LCDR2 comprises the amino acid sequence of SEQ ID NO: 15, and the LCDR3 comprises the amino acid sequence of SEQ ID NO:129;
(f5) the HCDR1 comprises the amino acid sequence of SEQ ID NO: 130, the HCDR2 comprises the amino acid sequence of SEQ ID NO:131, the HCDR3 comprises the amino acid sequence of SEQ ID NO: 132, the LCDR1 comprises the amino acid sequence of SEQ ID NO:133, the LCDR2 comprises the amino acid sequence of GAS, and the LCDR3 comprises the amino acid sequence of SEQ ID NO: 120;
(g1) the HCDR1 comprises the amino acid sequence of SEQ ID NO:137, the HCDR2 comprises the amino acid sequence of SEQ ID NO:138, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO:142;
(g2) the HCDR1 comprises the amino acid sequence of SEQ ID NO: 143, the HCDR2 comprises the amino acid sequence of SEQ ID NO: 144, the HCDR3 comprises the amino acid sequence of SEQ ID NO: 139, the LCDR1 comprises the amino acid sequence of SEQ ID NO: 140, the LCDR2 comprises the amino acid sequence of SEQ ID NO:141, and the LCDR3 comprises the amino acid sequence of SEQ ID NO:142;
(g3) the HCDR1 comprises the amino acid sequence of SEQ ID NO: 145, the HCDR2 comprises the amino acid sequence of SEQ ID NO: 146, the HCDR3 comprises the amino acid sequence of SEQ ID NO:139, the LCDR1 comprises the amino acid sequence of SEQ ID NO: 140, the LCDR2 comprises the amino acid sequence of SEQ ID NO: 141, and the LCDR3 comprises the amino acid sequence of SEQ ID NO:142;
(g4) the HCDR1 comprises the amino acid sequence of SEQ ID NO: 147, the HCDR2 comprises the amino acid sequence of SEQ ID NO: 148, the HCDR3 comprises the amino acid sequence of SEQ ID NO: 149, the LCDR1 comprises the amino acid sequence of SEQ ID NO: 150, the LCDR2 comprises the amino acid sequence of SEQ ID NO: 151, and the LCDR3 comprises the amino acid sequence of SEQ ID NO:152;
(g5) the HCDR1 comprises the amino acid sequence of SEQ ID NO: 153, the HCDR2 comprises the amino acid sequence of SEQ ID NO: 154, the HCDR3 comprises the amino acid sequence of SEQ ID NO: 155, the LCDR1 comprises the amino acid sequence of SEQ ID NO: 156, the LCDR2 comprises the amino acid sequence of AAS, and the LCDR3 comprises the amino acid sequence of SEQ ID NO: 142;
(h1) the HCDR1 comprises the amino acid sequence of SEQ ID NO:1, the HCDR2 comprises the amino acid sequence of SEQ ID NO: 160, the HCDR3 comprises the amino acid sequence of SEQ ID NO:161, the LCDR1 comprises the amino acid sequence of SEQ ID NO: 162, the LCDR2 comprises the amino acid sequence of SEQ ID NO: 163, and the LCDR3 comprises the amino acid sequence of SEQ ID NO:76;
(h2) the HCDR1 comprises the amino acid sequence of SEQ ID NO: 164, the HCDR2 comprises the amino acid sequence of SEQ ID NO: 165, the HCDR3 comprises the amino acid sequence of SEQ ID NO: 161, the LCDR1 comprises the amino acid sequence of SEQ ID NO: 162 the LCDR2 comprises the amino acid sequence of SEQ ID NO: 163, and the LCDR3 comprises the amino acid sequence of SEQ ID NO: 76;
(h3) the HCDR1 comprises the amino acid sequence of SEQ ID NO: 166, the HCDR2 comprises the amino acid sequence of SEQ ID NO: 167, the HCDR3 comprises the amino acid sequence of SEQ ID NO: 161, the LCDR1 comprises the amino acid sequence of SEQ ID NO:162, the LCDR2 comprises the amino acid sequence of SEQ ID NO:163, and the LCDR3 comprises the amino acid sequence of SEQ ID NO:76;
(h4) the HCDR1 comprises the amino acid sequence of SEQ ID NO:11, the HCDR2 comprises the amino acid sequence of SEQ ID NO: 168, the HCDR3 comprises the amino acid sequence of SEQ ID NO: 169, the LCDR1 comprises the amino acid sequence of SEQ ID NO: 170, the LCDR2 comprises the amino acid sequence of SEQ ID NO:171, and the LCDR3 comprises the amino acid sequence of SEQ ID NO:86; and
(h5) the HCDR1 comprises the amino acid sequence of SEQ ID NO: 172, the HCDR2 comprises the amino acid sequence of SEQ ID NO: 173, the HCDR3 comprises the amino acid sequence of SEQ ID NO: 174, the LCDR1 comprises the amino acid sequence of SEQ ID NO: 175, the LCDR2 comprises the amino acid sequence of VAS and the LCDR3 comprises the amino acid sequence of SEQ ID NO: 76.

17. The binding agent of claim 15, wherein the second antigen binding region that binds to CD3ε comprises CDR sequences selected from the group consisting of:
(a) the HCDR1 comprises the amino acid sequence of SEQ ID NO: 179, the HCDR2 comprises the amino acid sequence of SEQ ID NO:180, the HCDR3 comprises the amino acid sequence of SEQ ID NO: 181, the LCDR1 comprises the amino acid sequence of SEQ ID NO: 182, the LCDR2 comprises the amino acid sequence of SEQ ID NO:183, and the LCDR3 comprises the amino acid sequence of SEQ ID NO:184;
(b) the HCDR1 comprises the amino acid sequence of SEQ ID NO: 185, the HCDR2 comprises the amino acid sequence of SEQ ID NO: 186, the HCDR3 comprises the amino acid sequence of SEQ ID NO: 181, the LCDR1 comprises the amino acid sequence of SEQ ID NO: 182 the LCDR2 comprises the amino acid sequence of SEQ ID NO: 183, and the LCDR3 comprises the amino acid sequence of SEQ ID NO: 184;
(c) the HCDR1 comprises the amino acid sequence of SEQ ID NO: 187, the HCDR2 comprises the amino acid sequence of SEQ ID NO: 188, the HCDR3 comprises the amino acid sequence of SEQ ID NO: 181, the LCDR1 comprises the amino acid sequence of SEQ ID NO: 182, the LCDR2 comprises the amino acid sequence of SEQ ID NO:183, and the LCDR3 comprises the amino acid sequence of SEQ ID NO:184;
(d) the HCDR1 comprises the amino acid sequence of SEQ ID NO: 189, the HCDR2 comprises the amino acid sequence of SEQ ID NO:190, the HCDR3 comprises the amino acid sequence of SEQ ID NO: 191, the LCDR1 comprises the amino acid sequence of SEQ ID NO:192, the LCDR2 comprises the amino acid sequence of SEQ ID NO: 193, and the LCDR3 comprises the amino acid sequence of SEQ ID NO: 194; and
(e) the HCDR1 comprises the amino acid sequence of SEQ ID NO: 195, the HCDR2 comprises the amino acid sequence of SEQ ID NO: 196, the HCDR3 comprises the amino acid sequence of SEQ ID NO:197, the LCDR1 comprises the amino acid sequence of SEQ ID NO:198, the LCDR2 comprises the amino acid sequence of DSS and the LCDR3 comprises the amino acid sequence of SEQ ID NO: 184.

37. The binding agent of any one of claims 14 to 17, wherein
(i) the first binding region comprises a VH domain comprising the amino acid sequence of SEQ ID NO:22, and a VL domain comprising the amino acid sequence of SEQ ID NO:23; and
(ii) the second binding region comprises a VH domain comprising the amino acid sequence of SEQ ID NO:200, and a VL domain comprising the amino acid sequence of SEQ ID NO:201.

38. The binding agent of any one of claims 12 to 18, wherein the first antigen binding region comprises a Fab, and the second antigen binding region comprises a stapled scFv fragment (spFv).

39. The binding agent of claim 19, wherein the spFv comprises at least one disulfide bond between the VH or VL and the linker.

40. The binding agent of any one of claims 12 to 20, wherein the binding agent further comprises an immunoglobulin (Ig) constant region, a fragment of the Ig constant region, wherein optionally the fragment of the Ig constant region is an Fc region or an CH3 domain.

41. A binding agent comprising
(i) a first polypeptide comprising a spFv that binds CD3ε, a CH2 domain and a CH3 domain;
(ii) a second polypeptide comprising a VH domain that binds ENPP3, a CH2 domain and a CH3 domain; and
(iii) a third polypeptide comprising a VL domain that binds ENPP3,
wherein the spFv that binds CD3ε comprises a VH domain comprising a HCDR1, HCDR2, and HCDR3 of a VH as set forth in SEQ ID NO:200, and a VL domain comprising a LCDR1, LCDR2, and LCDR3 of a VL as set forth in SEQ ID NO:201; and wherein the second and third polypeptide comprising a VH and VL that bind to ENPP3, wherein the VH and VL that bind to ENPP3 are selected from the group consisting of:
(a) a VH domain comprising a HCDR1, HCDR2, and HCDR3 of a VH as set forth in SEQ ID NO:22, and a VL domain comprising a LCDR1, LCDR2, and LCDR3 of a VL as set forth in SEQ ID NO:23;
(b) a VH domain comprising a HCDR1, HCDR2, and HCDR3 of a VH as set forth in SEQ ID NO:45, and a VL domain comprising a LCDR1, LCDR2, and LCDR3 of a VL as set forth in SEQ ID NO:46;
(c) a VH domain comprising a HCDR1, HCDR2, and HCDR3 of a VH as set forth in SEQ ID NO:68, and a VL domain comprising a LCDR1, LCDR2, and LCDR3 of a VL as set forth in SEQ ID NO:69 or SEQ ID NO:70;
(d) a VH domain comprising a HCDR1, HCDR2, and HCDR3 of a VH as set forth in SEQ ID NO:91, and a VL domain comprising a LCDR1, LCDR2, and LCDR3 of a VL as set forth in SEQ ID NO:92;
(e) a VH domain comprising a HCDR1, HCDR2, and HCDR3 of a VH as set forth in SEQ ID NO:113, and a VL domain comprising a LCDR1, LCDR2, and LCDR3 of a VL as set forth in SEQ ID NO:114 or SEQ ID NO:115;
(f) a VH domain comprising a HCDR1, HCDR2, and HCDR3 of a VH as set forth in SEQ ID NO:134, and a VL domain comprising a LCDR1, LCDR2, and LCDR3 of a VL as set forth in SEQ ID NO: 135 or SEQ ID NO:136;
(g) a VH domain comprising a HCDR1, HCDR2, and HCDR3 of a VH as set forth in SEQ ID NO:158, and a VL domain comprising a LCDR1, LCDR2, and LCDR3 of a VL as set forth in SEQ ID NO:159; and
(h) a VH domain comprising a HCDR1, HCDR2, and HCDR3 of a VH as set forth in SEQ ID NO:177, and a VL domain comprising a LCDR1, LCDR2, and LCDR3 of a VL as set forth in SEQ ID NO:178.

42. The binding agent of claim 22, wherein:
(i) the spFv that binds CD3ε comprises CDRs selected from the group consisting of:
(a) the HCDR1 comprises the amino acid sequence of SEQ ID NO: 179, the HCDR2 comprises the amino acid sequence of SEQ ID NO: 180, the HCDR3 comprises the amino acid sequence of SEQ ID NO:181, the LCDR1 comprises the amino acid sequence of SEQ ID NO:182, the LCDR2 comprises the amino acid sequence of SEQ ID NO: 183, and the LCDR3 comprises the amino acid sequence of SEQ ID NO:184;
(b) the HCDR1 comprises the amino acid sequence of SEQ ID NO: 185, the HCDR2 comprises the amino acid sequence of SEQ ID NO:186, the HCDR3 comprises the amino acid sequence of SEQ ID NO: 181, the LCDR1 comprises the amino acid sequence of SEQ ID NO: 182 the LCDR2 comprises the amino acid sequence of SEQ ID NO: 183, and the LCDR3 comprises the amino acid sequence of SEQ ID NO:184;
(c) the HCDR1 comprises the amino acid sequence of SEQ ID NO:187, the HCDR2 comprises the amino acid sequence of SEQ ID NO:188, the HCDR3 comprises the amino acid sequence of SEQ ID NO: 181, the LCDR1 comprises the amino acid sequence of SEQ ID NO:182, the LCDR2 comprises the amino acid sequence of SEQ ID NO:183, and the LCDR3 comprises the amino acid sequence of SEQ ID NO:184;
(d) the HCDR1 comprises the amino acid sequence of SEQ ID NO: 189, the HCDR2 comprises the amino acid sequence of SEQ ID NO: 190, the HCDR3 comprises the amino acid sequence of SEQ ID NO:191, the LCDR1 comprises the amino acid sequence of SEQ ID NO: 192, the LCDR2 comprises the amino acid sequence of SEQ ID NO: 193, and the LCDR3 comprises the amino acid sequence of SEQ ID NO:194; and
(e) the HCDR1 comprises the amino acid sequence of SEQ ID NO: 195, the HCDR2 comprises the amino acid sequence of SEQ ID NO: 196, the HCDR3 comprises the amino acid sequence of SEQ ID NO:197, the LCDR1 comprises the amino acid sequence of SEQ ID NO: 198, the LCDR2 comprises the amino acid sequence of DSS and the LCDR3 comprises the amino acid sequence of SEQ ID NO:184; and
(ii) the Fab that binds ENPP3 comprises:
(a) in the VH domain that binds ENPP3, HCDR1 comprises the amino acid sequence of SEQ ID NO:1; HCDR2 comprises the amino acid sequence of SEQ ID NO:2; HCDR3 comprises the amino acid sequence of SEQ ID NO: 3; and in the VL domain that binds ENPP3, LCDR1 comprises the amino acid sequence of SEQ ID NO:4; LCDR2 comprises the amino acid sequence of SEQ ID NO:5; and LCDR3 comprises the amino acid sequence of SEQ ID NO:6;
(b) in the VH domain that binds ENPP3, HCDR1 comprises the amino acid sequence of SEQ ID NO:7; HCDR2 comprises the amino acid sequence of SEQ ID NO:8; HCDR3 comprises the amino acid sequence of SEQ ID NO: 3; and in the VL domain that binds ENPP3, LCDR1 comprises the amino acid sequence of SEQ ID NO:4; LCDR2 comprises the amino acid sequence of SEQ ID NO:5; and LCDR3 comprises the amino acid sequence of SEQ ID NO:6;
(c) in the VH domain that binds ENPP3, HCDR1 comprises the amino acid sequence of SEQ ID NO:9; HCDR2 comprises the amino acid sequence of SEQ ID NO:10; HCDR3 comprises the amino acid sequence of SEQ ID NO: 3; and in the VL domain that binds ENPP3, LCDR1 comprises the amino acid sequence of SEQ ID NO:4; LCDR2 comprises the amino acid sequence of SEQ ID NO:5; and LCDR3 comprises the amino acid sequence of SEQ ID NO:6;
(d) in the VH domain that binds ENPP3, HCDR1 comprises the amino acid sequence of SEQ ID NO:11; HCDR2 comprises the amino acid sequence of SEQ ID NO:12; HCDR3 comprises the amino acid sequence of SEQ ID NO: 13; and in the VL domain that binds ENPP3, LCDR1 comprises the amino acid sequence of SEQ ID NO:14; LCDR2 comprises the amino acid sequence of SEQ ID NO: 15; and LCDR3 comprises the amino acid sequence of SEQ ID NO:16; and
(e) in the VH domain that binds ENPP3, HCDR1 comprises the amino acid sequence of SEQ ID NO:17; HCDR2 comprises the amino acid sequence of SEQ ID NO:18; HCDR3 comprises the amino acid sequence of SEQ ID NO: 19; and in the VL domain that binds ENPP3, LCDR1 comprises the amino acid sequence of SEQ ID NO:20; LCDR2 comprises the amino acid sequence of GAS; and LCDR3 comprises the amino acid sequence of SEQ ID NO: 6.

43. The binding agent of claim 22 or 23, wherein the spFv that binds CD3ε comprises a VH domain comprising the amino acid sequence of SEQ ID NO:200, and a VL domain comprising the amino acid sequence of SEQ ID NO:201; the VH domain that binds ENPP3 comprises the amino acid sequence of SEQ ID NO:22, and the VL domain that binds ENPP3 comprises the amino acid sequence of SEQ ID NO:23.

44. The binding agent of any one of claims 22 to 24, wherein the spFv that binds CD3ε comprises the amino acid sequence of SEQ ID NO:248; the VH domain that binds ENPP3 comprises the amino acid sequence of SEQ ID NO:22, and the VL domain that binds ENPP3 comprises the amino acid sequence of SEQ ID NO:23.

45. The binding agent of any one of claims 22 to 25, wherein:
(i) the first polypeptide comprising a spFv that binds CD3ε, a CH2 domain and a CH3 domain comprises the amino acid sequence of SEQ ID NO:255;
(ii) the second polypeptide comprising a VH domain that binds ENPP3, a CH2 domain and a CH3 domain comprises the amino acid sequence of SEQ ID NO: 256; and
(iii) the third polypeptide comprising a VL domain that binds ENPP3 comprises the amino acid sequence of SEQ ID NO:257.

46. The binding agent of any one of claims 22 to 26, wherein the spFv comprises at least one disulfide bond between the VH or VL and the linker.

47. A composition comprising the binding agent of any one of claims 1 to 27, and a pharmaceutically acceptable carrier.

48. A polynucleotide comprising nucleotide sequences encoding a VH, a VL, or both a VH and a VL of the binding agent of any one of claims 1 to 27.

49. A vector comprising the polynucleotide of claim 29.

50. A cell comprising the polynucleotide of claim 29.

51. A kit comprising the binding agent of any one of claims 1 to 27.

52. A method of making a binding agent which binds to an epitope of ENPP3, comprising culturing the cell of claim 31 to express the binding agent.

53. A method of directing a T cell to a target cell expressing ENPP3, comprising contacting the T cell with an effective amount of the binding agent of any one of claims 12 to 27 or a composition comprising the binding agent and a pharmaceutically acceptable carrier, wherein the antigen binding region that binds to CD3ε binds the T cell and the antigen binding region that binds to ENPP3 binds to the target cell.

54. A method of treating a cancer or tumor in a subject in need thereof, comprising administering an effective amount the binding agent of any one of claims 12 to 27 or a composition comprising the binding agent and a pharmaceutically acceptable carrier to the subject.

55. The method of claim 35, wherein the cancer or tumor is selected from the group consisting of a clear cell renal cell carcinoma (CCRCC), a papillary renal cancer, an endometrioid uterine cancer, endometrioid ovarian cancer, a colorectal cancer, a lung andenocarcinoma, or a liver hepatocellular carcinoma.


SEQUENCE LISTING (329 sequences)

SEQ ID NO: 1 - protein, 5 aa, synthetic construct
DYSMN

SEQ ID NO: 2 - protein, 17 aa, synthetic construct
SISSISSYVKYADSVKG

SEQ ID NO: 3 - protein, 6 aa, synthetic construct
GHYFDY

SEQ ID NO: 4 - protein, 11 aa, synthetic construct
RASQSVSSNLA

SEQ ID NO: 5 - protein, 7 aa, synthetic construct
GASTRAT

SEQ ID NO: 6 - protein, 9 aa, synthetic construct
QQYNNWPRT

SEQ ID NO: 7 - protein, 7 aa, synthetic construct
GFTFSDY

SEQ ID NO: 8 - protein, 6 aa, synthetic construct
SSISSY

SEQ ID NO: 9 - protein, 10 aa, synthetic construct
GFTFSDYSMN

SEQ ID NO: 10 - protein, 10 aa, synthetic construct
SISSISSYVK

SEQ ID NO: 11 - protein, 6 aa, synthetic construct
SDYSMN

SEQ ID NO: 12 - protein, 13 aa, synthetic construct
WVSSISSISSYVK

SEQ ID NO: 13 - protein, 7 aa, synthetic construct
ARGHYFD

SEQ ID NO: 14 - protein, 7 aa, synthetic construct
SSNLAWY

SEQ ID NO: 15 - protein, 10 aa, synthetic construct
LLIYGASTRA

SEQ ID NO: 16 - protein, 8 aa, synthetic construct
QQYNNWPR

SEQ ID NO: 17 - protein, 8 aa, synthetic construct
GFTFSDYS

SEQ ID NO: 18 - protein, 8 aa, synthetic construct
ISSISSYV

SEQ ID NO: 19 - protein, 8 aa, synthetic construct
ARGHYFDY

SEQ ID NO: 20 - protein, 6 aa, synthetic construct
QSVSSN

SEQ ID NO: 21 - skipped

SEQ ID NO: 22 - protein, 115 aa, synthetic construct
EVQLVESGGGRVKPGGSLRLSCAASGFTFSDYSMNWVRQAPGKGLEWVSSISSISSYVKYADSVKGRFTMSRDNAKNSLFLQMNSLRDEDMAVYYCARGHYFDYWGQGTLVTVSS

SEQ ID NO: 23 - protein, 107 aa, synthetic construct
EIVMTQSPATLSVSPGERATLSCRASQSVSSNLAWYQQKPGLAPRLLIYGASTRATGIPARFSGSGSGTEFTLTISSLQSEDFAVYYCQQYNNWPRTFGQGTKVEIK

SEQ ID NO: 24 - protein, 5 aa, synthetic construct
NYYIF

SEQ ID NO: 25 - protein, 17 aa, synthetic construct
WINPNSGGTNYAQKFQG

SEQ ID NO: 26 - protein, 16 aa, synthetic construct
GGYSGYALSSHYGMDV

SEQ ID NO: 27 - protein, 12 aa, synthetic construct
RASQSVSSSYLA

SEQ ID NO: 28 - protein, 7 aa, synthetic construct
DASSRAT

SEQ ID NO: 29 - protein, 9 aa, synthetic construct
QQYDSSPLT

SEQ ID NO: 30 - protein, 7 aa, synthetic construct
GYTVTNY

SEQ ID NO: 31 - protein, 6 aa, synthetic construct
NPNSGG

SEQ ID NO: 32 - protein, 10 aa, synthetic construct
GYTVTNYYIF

SEQ ID NO: 33 - protein, 10 aa, synthetic construct
WINPNSGGTN

SEQ ID NO: 34 - protein, 6 aa, synthetic construct
TNYYIF

SEQ ID NO: 35 - protein, 13 aa, synthetic construct
WMGWINPNSGGTN

SEQ ID NO: 36 - protein, 17 aa, synthetic construct
ARGGYSGYALSSHYGMD

SEQ ID NO: 37 - protein, 8 aa, synthetic construct
SSSYLAWY

SEQ ID NO: 38 - protein, 10 aa, synthetic construct
LLIYDASSRA

SEQ ID NO: 39 - protein, 8 aa, synthetic construct
QQYDSSPL

SEQ ID NO: 40 - protein, 8 aa, synthetic construct
GYTVTNYY

SEQ ID NO: 41 - protein, 8 aa, synthetic construct
INPNSGGT

SEQ ID NO: 42 - protein, 18 aa, synthetic construct
ARGGYSGYALSSHYGMDV

SEQ ID NO: 43 - protein, 7 aa, synthetic construct
QSVSSSY

SEQ ID NO: 44 - skipped

SEQ ID NO: 45 - protein, 125 aa, synthetic construct
QVQLVQSGAEVKKPGASVKVSCKASGYTVTNYYIFWVRQAPGQGLEWMGWINPNSGGTNYAQKFQGRVTMTRDTSINTAYMKLSRLGSDDTAVYYCARGGYSGYALSSHYGMDVWGQGTMVTVSS

SEQ ID NO: 46 - protein, 108 aa, synthetic construct
EIVLTQSPGTLSLSPGERATLSCRASQSVSSSYLAWYQQKPGQAPRLLIYDASSRATGIPDRFSGSGSGTDFTLIISRLEPEDFAVYYCQQYDSSPLTFGGGTKVEIK

SEQ ID NO: 47 - protein, 5 aa, synthetic construct
TYYIH

SEQ ID NO: 48 - protein, 17 aa, synthetic construct
IINPSGGSTNYAQKFQG

SEQ ID NO: 49 - protein, 11 aa, synthetic construct
DYYYYYYGMDV

SEQ ID NO: 50 - protein, 16 aa, synthetic construct
RSSQSLVRNGGYTYLN

SEQ ID NO: 51 - protein, 7 aa, synthetic construct
QISNRFS

SEQ ID NO: 52 - protein, 9 aa, synthetic construct
MQATQFPHT

SEQ ID NO: 53 - protein, 7 aa, synthetic construct
GYTFTTY

SEQ ID NO: 54 - protein, 6 aa, synthetic construct
NPSGGS

SEQ ID NO: 55 - protein, 10 aa, synthetic construct
GYTFTTYYIH

SEQ ID NO: 56 - protein, 10 aa, synthetic construct
IINPSGGSTN

SEQ ID NO: 57 - protein, 6 aa, synthetic construct
TTYYIH

SEQ ID NO: 58 - protein, 13 aa, synthetic construct
WMGIINPSGGSTN

SEQ ID NO: 59 - protein, 12 aa, synthetic construct
ARDYYYYYYGMD

SEQ ID NO: 60 - protein, 12 aa, synthetic construct
VRNGGYTYLNWL

SEQ ID NO: 61 - protein, 10 aa, synthetic construct
LLIYQISNRF

SEQ ID NO: 62 - protein, 8 aa, synthetic construct
MQATQFPH

SEQ ID NO: 63 - protein, 8 aa, synthetic construct
GYTFTTYY

SEQ ID NO: 64 - protein, 8 aa, synthetic construct
INPSGGST

SEQ ID NO: 65 - protein, 13 aa, synthetic construct
ARDYYYYYYGMDV

SEQ ID NO: 66 - protein, 11 aa, synthetic construct
QSLVRNGGYTY

SEQ ID NO: 67 - skipped

SEQ ID NO: 68 - protein, 120 aa, synthetic construct
QVQLVQSGAEVKKPGASVKVSCKASGYTFTTYYIHWVRQAPGQGLEWMGIINPSGGSTNYAQKFQGRVTMTRDTSTRIVYMELGSLRSEDTAVYYCARDYYYYYYGMDVWGQGTTVTVSS

SEQ ID NO: 69 - protein, 112 aa, synthetic construct
DIVMTQSPLSLPVTLGQPASISCRSSQSLVRNGGYTYLNWLQQRPGQPPRLLIYQISNRFSGVPDRFSGSGAGTDFTLTISRVEAEDVGVYYCMQATQFPHTFGQGTKLEIK

SEQ ID NO: 70 - protein, 112 aa, synthetic construct
DIVMTQTPLSSPVTLGQPASISCRSSQSLVRNGGYTYLNWLQQRPGQPPRLLIYQISNRFSGVPDRFSGSGAGTDFTLTISRVEAEDVGVYYCMQATQFPHTFGQGTKLEIK

SEQ ID NO: 71 - protein, 5 aa, synthetic construct
NYWIG

SEQ ID NO: 72 - protein, 17 aa, synthetic construct
IIYPSDSNTRYSPSFQG

SEQ ID NO: 73 - protein, 10 aa, synthetic construct
QNYYYYTMDV

SEQ ID NO: 74 - protein, 11 aa, synthetic construct
RASQVIRSYLA

SEQ ID NO: 75 - protein, 7 aa, synthetic construct
GASTLQS

SEQ ID NO: 76 - protein, 9 aa, synthetic construct
QQLNSYPRT

SEQ ID NO: 77 - protein, 7 aa, synthetic construct
GYGFTNY

SEQ ID NO: 78 - protein, 6 aa, synthetic construct
YPSDSN

SEQ ID NO: 79 - protein, 10 aa, synthetic construct
GYGFTNYWIG

SEQ ID NO: 80 - protein, 10 aa, synthetic construct
IIYPSDSNTR

SEQ ID NO: 81 - protein, 6 aa, synthetic construct
TNYWIG

SEQ ID NO: 82 - protein, 13 aa, synthetic construct
WMGIIYPSDSNTR

SEQ ID NO: 83 - protein, 11 aa, synthetic construct
TRQNYYYYTMD

SEQ ID NO: 84 - protein, 7 aa, synthetic construct
RSYLAWY

SEQ ID NO: 85 - protein, 10 aa, synthetic construct
LLIYGASTLQ

SEQ ID NO: 86 - protein, 8 aa, synthetic construct
QQLNSYPR

SEQ ID NO: 87 - protein, 8 aa, synthetic construct
GYGFTNYW

SEQ ID NO: 88 - protein, 8 aa, synthetic construct
IYPSDSNT

SEQ ID NO: 89 - protein, 12 aa, synthetic construct
TRQNYYYYTMDV

SEQ ID NO: 90 - protein, 6 aa, synthetic construct
QVIRSY

SEQ ID NO: 91 - protein, 119 aa, synthetic construct
QVQLVQSGAEVKKPGESLKISCKGSGYGFTNYWIGWVRQMPGKGLEWMGIIYPSDSNTRYSPSFQGQVTISADKSINTAYLQWSSLKASDTAMYYCTRQNYYYYTMDVWGQGTTVTVSS

SEQ ID NO: 92 - protein, 107 aa, synthetic construct
DIQMTQSPSFLSASVGDRVTITCRASQVIRSYLAWYQQKPGKAPKLLIYGASTLQSGFPSRFSGSGSGTEFTLTISSLQPEDFATYYCQQLNSYPRTFGQGTKVEIK

SEQ ID NO: 93 - protein, 5 aa, synthetic construct
SYYWS

SEQ ID NO: 94 - protein, 22 aa, synthetic construct
RIYTTGRIYTSGYTNYNPSLKS

SEQ ID NO: 95 - protein, 11 aa, synthetic construct
GGELFTDGFDI

SEQ ID NO: 96 - protein, 12 aa, synthetic construct
RASQSVISTYLA

SEQ ID NO: 97 - protein, 7 aa, synthetic construct
GASSRAT

SEQ ID NO: 98 - protein, 9 aa, synthetic construct
QQYDTSPIT

SEQ ID NO: 99 - protein, 7 aa, synthetic construct
GGSISSY

SEQ ID NO: 100 - protein, 11 aa, synthetic construct
YTTGRIYTSGY

SEQ ID NO: 101 - protein, 10 aa, synthetic construct
GGSISSYYWS

SEQ ID NO: 102 - protein, 15 aa, synthetic construct
RIYTTGRIYTSGYTN

SEQ ID NO: 103 - protein, 6 aa, synthetic construct
SSYYWS

SEQ ID NO: 104 - protein, 18 aa, synthetic construct
WIGRIYTTGRIYTSGYTN

SEQ ID NO: 105 - protein, 12 aa, synthetic construct
ARGGELFTDGFD

SEQ ID NO: 106 - protein, 8 aa, synthetic construct
ISTYLAWY

SEQ ID NO: 107 - protein, 10 aa, synthetic construct
LLIYGASSRA

SEQ ID NO: 108 - protein, 8 aa, synthetic construct
QQYDTSPI

SEQ ID NO: 109 - protein, 8 aa, synthetic construct
GGSISSYY

SEQ ID NO: 110 - protein, 13 aa, synthetic construct
IYTTGRIYTSGYT

SEQ ID NO: 111 - protein, 13 aa, synthetic construct
ARGGELFTDGFDI

SEQ ID NO: 112 - protein, 7 aa, synthetic construct
QSVISTY

SEQ ID NO: 113 - protein, 125 aa, synthetic construct
QVQLQESGPGLVRPSETLSLTCTVSGGSISSYYWSWIRQPAGKGLEWIGRIYTTGRIYTSGYTNYNPSLKSRVTMSIDTSKIQFSLRLNSVTAADTAVYWCARGGELFTDGFDIWGQGTMVTVSS

SEQ ID NO: 114 - protein, 108 aa, synthetic construct
EIVMTQSPGTLSLSPGERATLSCRASQSVISTYLAWYQQNPGQAPRLLIYGASSRATGIPDRFSGSGSGTDFTLTISRLEPEDFAVYYCQQYDTSPITFGQGTRLEIK

SEQ ID NO: 115 - protein, 108 aa, synthetic construct
EIVMTQSPGTLSLSPGERATLSCRASQSVISTYLAWYQQNPGQAPRLLIYGASSRATGIPDRFSGSGSGTDFTLTISRLEPEDFVVYYCQQYDTSPITFGQGTRLEIK

SEQ ID NO: 116 - protein, 5 aa, synthetic construct
SHYWS

SEQ ID NO: 117 - protein, 16 aa, synthetic construct
NIFYSGSTNYNPSLKS

SEQ ID NO: 118 - protein, 8 aa, synthetic construct
HDHVWFDP

SEQ ID NO: 119 - protein, 11 aa, synthetic construct
RASQSVSRNLV

SEQ ID NO: 120 - protein, 10 aa, synthetic construct [sequence omitted]

SEQ ID NO: 121 - protein, 7 aa, synthetic construct
GGSISSH

SEQ ID NO: 122 - protein, 5 aa, synthetic construct
FYSGS

SEQ ID NO: 123 - protein, 10 aa, synthetic construct
GGSISSHYWS

SEQ ID NO: 124 - protein, 9 aa, synthetic construct
NIFYSGSTN

SEQ ID NO: 125 - protein, 6 aa, synthetic construct
SSHYWS

SEQ ID NO: 126 - protein, 12 aa, synthetic construct
WIGNIFYSGSTN

SEQ ID NO: 127 - protein, 9 aa, synthetic construct
ARHDHVWFD

SEQ ID NO: 128 - protein, 7 aa, synthetic construct
SRNLVWY

SEQ ID NO: 129 - protein, 9 aa, synthetic construct
QQYNNWPPW

SEQ ID NO: 130 - protein, 8 aa, synthetic construct
GGSISSHY

SEQ ID NO: 131 - protein, 7 aa, synthetic construct
IFYSGST

SEQ ID NO: 132 - protein, 10 aa, synthetic construct
ARHDHVWFDP

SEQ ID NO: 133 - protein, 6 aa, synthetic construct
QSVSRN

SEQ ID NO: 134 - protein, 116 aa, synthetic construct
QVQLQESGPGLVKTSETLSLTCTVSGGSISSHYWSWIRQPPGKGLEWIGNIFYSGSTNYNPSLKSRVTMSVDTSKNHFSLKLISVTTADTAIYYCARHDHVWFDPWGQGTLVTVSS

SEQ ID NO: 135 - protein, 108 aa, synthetic construct
EIVLTQSPGTLSVFPGERATLSCRASQSVSRNLVWYQQKPGQAPRLLIYGASTRATGIPDRFSGSGSGTEFTLTISSLQSEDFAVYYCQQYNNWPPWTFGQGTKVEIK

SEQ ID NO: 136 - protein, 108 aa, synthetic construct
EIVLTQSPATLSVFPGERATLSCRASQSVSRNLVWYQQKPGQAPRLLIYGASTRATGIPDRFSGSGSGTEFTLTISSLQSEDFAVYYCQQYNNWPPWTFGQGTKVEIK

SEQ ID NO: 137 - protein, 5 aa, synthetic construct
NYYWS

SEQ ID NO: 138 - protein, 16 aa, synthetic construct
RIYASGSTNYNPSLKS

SEQ ID NO: 139 - protein, 14 aa, synthetic construct
EGYSSTLYDNAFDF

SEQ ID NO: 140 - protein, 11 aa, synthetic construct
RASQGISTWLA

SEQ ID NO: 141 - protein, 7 aa, synthetic construct
AASSLQS

SEQ ID NO: 142 - protein, 9 aa, synthetic construct
QQANSFPLT

SEQ ID NO: 143 - protein, 7 aa, synthetic construct
GGSISNY

SEQ ID NO: 144 - protein, 5 aa, synthetic construct
YASGS

SEQ ID NO: 145 - protein, 10 aa, synthetic construct
GGSISNYYWS

SEQ ID NO: 146 - protein, 9 aa, synthetic construct
RIYASGSTN

SEQ ID NO: 147 - protein, 6 aa, synthetic construct
SNYYWS

SEQ ID NO: 148 - protein, 12 aa, synthetic construct
WIGRIYASGSTN

SEQ ID NO: 149 - protein, 15 aa, synthetic construct
AREGYSSTLYDNAFD

SEQ ID NO: 150 - protein, 7 aa, synthetic construct
STWLAWY

SEQ ID NO: 151 - protein, 10 aa, synthetic construct
LLIYAASSLQ

SEQ ID NO: 152 - protein, 8 aa, synthetic construct
QQANSFPL

SEQ ID NO: 153 - protein, 8 aa, synthetic construct
GGSISNYY

SEQ ID NO: 154 - protein, 7 aa, synthetic construct
IYASGST

SEQ ID NO: 155 - protein, 16 aa, synthetic construct
AREGYSSTLYDNAFDF

SEQ ID NO: 156 - protein, 6 aa, synthetic construct
QGISTW

SEQ ID NO: 157 - skipped

SEQ ID NO: 158 - protein, 122 aa, synthetic construct
QVQLQESGPGLVKPSETLSLTCTVSGGSISNYYWSWIRQPAGKGLEWIGRIYASGSTNYNPSLKSRVTMSVDTSKNQFSLRLRSVTATDTAVYYCAREGYSSTLYDNAFDFWGQGTMVTVSS

SEQ ID NO: 159 - protein, 107 aa, synthetic construct
DIQMTQSPSSVSASVGGRVTITCRASQGISTWLAWYQQKPGKAPKLLIYAASSLQSGVPSRFSGSGSGTDFTLTISSLQPEDFATYYCQQANSFPLTFGGGTKVEIK

SEQ ID NO: 160 - protein, 17 aa, synthetic construct
SISSGSSYIYYADSLKG

SEQ ID NO: 161 - protein, 7 aa, synthetic construct
GLDYFDY

SEQ ID NO: 162 - protein, 11 aa, synthetic construct
RASQDISNYLA

SEQ ID NO: 163 - protein, 7 aa, synthetic construct
VASTLQS

SEQ ID NO: 164 - protein, 7 aa, synthetic construct
GFIFSDY

SEQ ID NO: 165 - protein, 6 aa, synthetic construct
SSGSSY

SEQ ID NO: 166 - protein, 10 aa, synthetic construct
GFIFSDYSMN

SEQ ID NO: 167 - protein, 10 aa, synthetic construct
SISSGSSYIY

SEQ ID NO: 168 - protein, 13 aa, synthetic construct
WVSSISSGSSYIY

SEQ ID NO: 169 - protein, 8 aa, synthetic construct
VRGLDYFD

SEQ ID NO: 170 - protein, 7 aa, synthetic construct
SNYLAWY

SEQ ID NO: 171 - protein, 10 aa, synthetic construct
LLIYVASTLQ

SEQ ID NO: 172 - protein, 8 aa, synthetic construct
GFIFSDYS

SEQ ID NO: 173 - protein, 8 aa, synthetic construct
ISSGSSYI

SEQ ID NO: 174 - protein, 9 aa, synthetic construct
VRGLDYFDY

SEQ ID NO: 175 - protein, 6 aa, synthetic construct
QDISNY

SEQ ID NO: 176 - skipped

SEQ ID NO: 177 - protein, 116 aa, synthetic construct
EVQLVESGGGLVKPGGSLRLSCAASGFIFSDYSMNWVRQAPGKGLEWVSSISSGSSYIYYADSLKGRFTISRDNAKNSLYLQMSSLRAEDTAVYYCVRGLDYFDYWGQGALVTVSS

SEQ ID NO: 178 - protein, 107 aa, synthetic construct
DIQMTQSPSFLSASVGDRVTITCRASQDISNYLAWYQQKPGRAPKLLIYVASTLQSGVPSRFSGSGSGTEFTLTISSLQPEDFATYYCQQLNSYPRTFGQGTKVEIK

SEQ ID NO: 179 - protein, 5 aa, synthetic construct
RSTMH

SEQ ID NO: 180 - protein, 17 aa, synthetic construct
YINPSSAYTNYNQKFQG

SEQ ID NO: 181 - protein, 12 aa, synthetic construct
PQVHYDYAGFPY

SEQ ID NO: 182 - protein, 10 aa, synthetic construct
SASSSVSYMN

SEQ ID NO: 183 - protein, 7 aa, synthetic construct
DSSKLAS

SEQ ID NO: 184 - protein, 9 aa, synthetic construct
QQWSRNPPT

SEQ ID NO: 185 - protein, 7 aa, synthetic construct
GYTFTRS

SEQ ID NO: 186 - protein, 6 aa, synthetic construct
NPSSAY

SEQ ID NO: 187 - protein, 10 aa, synthetic construct
GYTFTRSTMH

SEQ ID NO: 188 - protein, 10 aa, synthetic construct
YINPSSAYTN

SEQ ID NO: 189 - protein, 6 aa, synthetic construct
TRSTMH

SEQ ID NO: 190 - protein, 13 aa, synthetic construct
WIGYINPSSAYTN

SEQ ID NO: 191 - protein, 13 aa, synthetic construct
ASPQVHYDYAGFP

SEQ ID NO: 192 - protein, 6 aa, synthetic construct
SYMNWY

SEQ ID NO: 193 - protein, 10 aa, synthetic construct
RWIYDSSKLA

SEQ ID NO: 194 - protein, 8 aa, synthetic construct
QQWSRNPP

SEQ ID NO: 195 - protein, 8 aa, synthetic construct
GYTFTRST

SEQ ID NO: 196 - protein, 8 aa, synthetic construct
INPSSAYT

SEQ ID NO: 197 - protein, 14 aa, synthetic construct
ASPQVHYDYAGFPY

SEQ ID NO: 198 - protein, 5 aa, synthetic construct
SSVSY

SEQ ID NO: 199 - skipped

SEQ ID NO: 200 - protein, 121 aa, synthetic construct
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGCGTLVTVSS

SEQ ID NO: 201 - protein, 106 aa, synthetic construct
EIVLTQSPATLSASPGERVTLSCSASSSVSYMNWYQQKPGCAPRRWIYDSSKLASGVPARFSGSGSGRDYTLTISSLEPEDFAVYYCQQWSRNPPTFGGGTKVEIK

SEQ ID NO: 202 - protein, 121 aa, synthetic construct
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSS

SEQ ID NO: 203 - protein, 106 aa, synthetic construct
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIK

SEQ ID NO: 204 - protein, 5 aa, synthetic construct
RYNMN

SEQ ID NO: 205 - protein, 17 aa, synthetic construct
SISTSSNYIYYADSVKG

SEQ ID NO: 206 - protein, 7 aa, synthetic construct
GWGPFDY

SEQ ID NO: 207 - protein, 11 aa, synthetic construct
RARQSIGTAIH

SEQ ID NO: 208 - protein, 7 aa, synthetic construct
YASESIS

SEQ ID NO: 209 - protein, 9 aa, synthetic construct
QQSGSWPYT

SEQ ID NO: 210 - protein, 7 aa, synthetic construct
GFTFSRY

SEQ ID NO: 211 - protein, 6 aa, synthetic construct
STSSNY

SEQ ID NO: 212 - protein, 6 aa, synthetic construct
GWGPFD

SEQ ID NO: 213 - protein, 7 aa, synthetic construct
RQSIGTA

SEQ ID NO: 214 - skipped

SEQ ID NO: 215 - protein, 6 aa, synthetic construct
SGSWPY

SEQ ID NO: 216 - protein, 8 aa, synthetic construct
GFTFSRYN

SEQ ID NO: 217 - protein, 8 aa, synthetic construct
ISTSSNYI

SEQ ID NO: 218 - protein, 9 aa, synthetic construct
TRGWGPFDY

SEQ ID NO: 219 - protein, 6 aa, synthetic construct
QSIGTA

SEQ ID NO: 220 - protein, 116 aa, synthetic construct
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 221 - protein, 107 aa, synthetic construct
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 222 - protein, 7 aa, synthetic construct
NNNAAWS

SEQ ID NO: 223 - protein, 18 aa, synthetic construct
RTYYRSKWLYDYAVSVKS

SEQ ID NO: 224 - protein, 8 aa, synthetic construct
GYSSSFDY

SEQ ID NO: 225 - protein, 14 aa, synthetic construct
TGTSSNIGTYKFVS

SEQ ID NO: 226 - protein, 7 aa, synthetic construct
EVSKRPS

SEQ ID NO: 227 - protein, 10 aa, synthetic construct
VSYAGSGTLL

SEQ ID NO: 228 - protein, 120 aa, synthetic construct
QVQLQQSGPGLVKPSQTLSLTCAISGDSVFNNNAAWSWIRQSPSRGLEWLGRTYYRSKWLYDYAVSVKSRITINPDTSKNQFSLQLNSVTPEDTAVYYCARGYSSSFDYWGQGTLVTVSS

SEQ ID NO: 229 - protein, 110 aa, synthetic construct
QSALTQPASVSGSPGQSITISCTGTSSNIGTYKFVSWYQQHPGKAPKVMIYEVSKRPSGVSNRFSGSKSGNTASLTISGLQAEDEADYYCVSYAGSGTLLFGGGTKLTVL

SEQ ID NO: 230 - protein, 5 aa, synthetic construct
RSTMH

SEQ ID NO: 231 - protein, 17 aa, synthetic construct
YINPSSAYTNYNQKFQG

SEQ ID NO: 232 - protein, 12 aa, synthetic construct
PQVHYDYAGFPY

SEQ ID NO: 233 - protein, 10 aa, synthetic construct
SASSSVSYMN

SEQ ID NO: 234 - protein, 7 aa, synthetic construct
DSSKLAS

SEQ ID NO: 235 - protein, 9 aa, synthetic construct
QQWSRNPPT

SEQ ID NO: 236 - protein, 7 aa, synthetic construct
GYTFTRS

SEQ ID NO: 237 - protein, 6 aa, synthetic construct
NPSSAY

SEQ ID NO: 238 - protein, 11 aa, synthetic construct
PQVHYDYAGFP

SEQ ID NO: 239 - protein, 6 aa, synthetic construct
SSSVSY

SEQ ID NO: 240 - skipped

SEQ ID NO: 241 - protein, 6 aa, synthetic construct
WSRNPP

SEQ ID NO: 242 - protein, 8 aa, synthetic construct
GYTFTRST

SEQ ID NO: 243 - protein, 8 aa, synthetic construct
INPSSAYT

SEQ ID NO: 244 - protein, 14 aa, synthetic construct
ASPQVHYDYAGFPY

SEQ ID NO: 245 - protein, 5 aa, synthetic construct
SSVSY

SEQ ID NO: 246 - protein, 121 aa, synthetic construct
QVQLVQSGAEVKKPGSSVKVSCKASGYTFTRSTMHWVKQAPGQGLEWMGYINPSSAYTNYNQKFQGRVTLTADKSTSTAYMELSSLRSEDTAVYYCASPQVHYDYAGFPYWGQGTLVTVSS

SEQ ID NO: 247 - protein, 106 aa, synthetic construct
EIVLTQSPATLSASPGERVTLSCSASSSVSYMNWYQQKPGQAPRRLIYDSSKLASGVPARFSGSGSGRDYTLTISSLEPEDFAVYYCQQWSRNPPTFGGGTKVEIK

SEQ ID NO: 248 - protein, 245 aa, synthetic construct
EIVLTQSPATLSASPGERVTLSCSASSSVSYMNWYQQKPGCAPRRWIYDSSKLASGVPARFSGSGSGRDYTLTISSLEPEDFAVYYCQQWSRNPPTFGGGTKVEIKGGGSGGSGGCPPCGGSGGQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGCGTLVTVSS

SEQ ID NO: 249 - protein, 247 aa, synthetic construct
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSGGSEGKSSGSGSESKSTGGSEIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIK

SEQ ID NO: 250 - protein, 247 aa, synthetic construct
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSS

SEQ ID NO: 251 - protein, 243 aa, synthetic construct
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 252 - protein, 243 aa, synthetic construct
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 253 - protein, 247 aa, synthetic construct
QVQLVQSGAEVKKPGSSVKVSCKASGYTFTRSTMHWVKQAPGQGLEWMGYINPSSAYTNYNQKFQGRVTLTADKSTSTAYMELSSLRSEDTAVYYCASPQVHYDYAGFPYWGQGTLVTVSSGGSEGKSSGSGSESKSTGGSEIVLTQSPATLSASPGERVTLSCSASSSVSYMNWYQQKPGQAPRRLIYDSSKLASGVPARFSGSGSGRDYTLTISSLEPEDFAVYYCQQWSRNPPTFGGGTKVEIK

SEQ ID NO: 254 - protein, 247 aa, synthetic construct
EIVLTQSPATLSASPGERVTLSCSASSSVSYMNWYQQKPGQAPRRLIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWMGYINPSSAYTNYNQKFQGRVTLTADKSTSTAYMELSSLRSEDTAVYYCASPQVHYDYAGFPYWGQGTLVTVSS

SEQ ID NO: 255 - protein, 477 aa, synthetic construct
EIVLTQSPATLSASPGERVTLSCSASSSVSYMNWYQQKPGCAPRRWIYDSSKLASGVPARFSGSGSGRDYTLTISSLEPEDFAVYYCQQWSRNPPTFGGGTKVEIKGGGSGGSGGCPPCGGSGGQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 445 aa, synthetic construct
EVQLVESGGGRVKPGGSLRLSCAASGFTFSDYSMNWVRQAPGKGLEWVSSISSISSYVKYADSVKGRFTMSRDNAKNSLFLQMNSLRDEDMAVYYCARGHYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 257 - protein, 214 aa, synthetic construct
EIVMTQSPATLSVSPGERATLSCRASQSVSSNLAWYQQKPGLAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 20 aa, synthetic construct
GGSEGKSSGSGSESKSTGGS

SEQ ID NO: 259 - protein, 8 aa, synthetic construct
GGGSGGGS

SEQ ID NO: 260 - protein, 12 aa, synthetic construct
GGGSGGGSGGGS

SEQ ID NO: 261 - protein, 16 aa, synthetic construct
GGGSGGGSGGGSGGGS

SEQ ID NO: 262 - protein, 20 aa, synthetic construct
GGGSGGGSGGGSGGGSGGGS

SEQ ID NO: 263 - protein, 15 aa, synthetic construct
GGGGSGGGGSGGGGS

SEQ ID NO: 264 - protein, 20 aa, synthetic construct
GGGGSGGGGSGGGGSGGGGS

SEQ ID NO: 265 - protein, 25 aa, synthetic construct
GGGGSGGGGSGGGGSGGGGSGGGGS

SEQ ID NO: 266 - protein, 18 aa, synthetic construct
GSTSGSGKPGSGEGSTKG

SEQ ID NO: 267 - protein, 14 aa, synthetic construct
IRPRAIGGSKPRVA

SEQ ID NO: 268 - protein, 15 aa, synthetic construct
GKGGSGKGGSGKGGS

SEQ ID NO: 269 - protein, 15 aa, synthetic construct
GGKGSGGKGSGGKGS

SEQ ID NO: 270 - protein, 15 aa, synthetic construct
GGGKSGGGKSGGGKS

SEQ ID NO: 271 - protein, 15 aa, synthetic construct
GKGKSGKGKSGKGKS

SEQ ID NO: 272 - protein, 15 aa, synthetic construct
GGGKSGGKGSGKGGS

SEQ ID NO: 273 - protein, 15 aa, synthetic construct
GKPGSGKPGSGKPGS

SEQ ID NO: 274 - protein, 20 aa, synthetic construct
GKPGSGKPGSGKPGSGKPGS

SEQ ID NO: 275 - protein, 20 aa, synthetic construct
GKGKSGKGKSGKGKSGKGKS

SEQ ID NO: 276 - protein, 14 aa, synthetic construct
STAGDTHLGGEDFD

SEQ ID NO: 277 - protein, 15 aa, synthetic construct
GEGGSGEGGSGEGGS

SEQ ID NO: 278 - protein, 15 aa, synthetic construct
GGEGSGGEGSGGEGS

SEQ ID NO: 279 - protein, 15 aa, synthetic construct
GEGESGEGESGEGES

SEQ ID NO: 280 - protein, 15 aa, synthetic construct
GGGESGGEGSGEGGS

SEQ ID NO: 281 - protein, 20 aa, synthetic construct
GEGESGEGESGEGESGEGES

SEQ ID NO: 282 - protein, 18 aa, synthetic construct
GSTSGSGKPGSGEGSTKG

SEQ ID NO: 283 - protein, 19 aa, synthetic construct
PRGASKSGSASQTGSAPGS

SEQ ID NO: 284 - protein, 19 aa, synthetic construct
GTAAAGAGAAGGAAAGAAG

SEQ ID NO: 285 - protein, 19 aa, synthetic construct
GTSGSSGSGSGGSGSGGGG

SEQ ID NO: 286 - protein, 20 aa, synthetic construct
GKPGSGKPGSGKPGSGKPGS

SEQ ID NO: 287 - protein, 4 aa, synthetic construct
GSGS

SEQ ID NO: 288 - protein, 10 aa, synthetic construct
APAPAPAPAP

SEQ ID NO: 289 - protein, 20 aa, synthetic construct
APAPAPAPAPAPAPAPAPAP

SEQ ID NO: 290 - protein, 32 aa, synthetic construct
AEAAAKEAAAKEAAAAKEAAAAKEAAAAKAAA

SEQ ID NO: 291 - protein, 16 aa, synthetic construct
GTEGKSSGSGSESKST

SEQ ID NO: 292 - protein, 18 aa, synthetic construct
GGGSGGSGGCPPCGGSGG

SEQ ID NO: 293 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 294 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 295 - protein, 4 aa, Homo sapiens
PHDF

SEQ ID NO: 296 - protein, 4 aa, Homo sapiens
PSES

SEQ ID NO: 297 - protein, 7 aa, Homo sapiens
PDEITKH

SEQ ID NO: 298 - skipped

SEQ ID NO: 299 - protein, 5 aa, Homo sapiens
SLECF

SEQ ID NO: 300 - protein, 5 aa, Homo sapiens
NSTQL

SEQ ID NO: 301 - protein, 9 aa, Homo sapiens
PSESQKCSF

SEQ ID NO: 302 - protein, 7 aa, Homo sapiens
VSKFSVC

SEQ ID NO: 303 - protein, 5 aa, Homo sapiens
RPRVL

SEQ ID NO: 304 - protein, 4 aa, Homo sapiens
DHCL

SEQ ID NO: 305 - protein, 5 aa, Homo sapiens
QPVSE

SEQ ID NO: 306 - protein, 7 aa, synthetic construct
NNNAAWS

SEQ ID NO: 307 - protein, 18 aa, synthetic construct
RTYYRSKWLYDYAVSVKS

SEQ ID NO: 308 - protein, 8 aa, synthetic construct
GYSSSFDY

SEQ ID NO: 309 - protein, 14 aa, synthetic construct
TGTSSNIGTYKFVS

SEQ ID NO: 310 - protein, 7 aa, synthetic construct
EVSKRPS

SEQ ID NO: 311 - protein, 10 aa, synthetic construct
VSYAGSGTLL

SEQ ID NO: 312 - protein, 120 aa, synthetic construct
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 313 - protein, 110 aa, synthetic construct
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 314 - protein, 447 aa, synthetic construct
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 315 - protein, 216 aa, synthetic construct
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 316 - DNA, 345 bp, synthetic construct
  source 1..345: mol_type = other DNA
gaggtgcagctggttgaatctggcggcggaagagtgaagcctggcggatctctgagactgtcttgtgccgcctctggcttcaccttctccgactactccatgaactgggtccgacaggctcctggcaaaggcctggaatgggtgtcctctatctcctccatctccagctacgtgaagtacgccgactccgtgaagggcagattcaccatgtccagagacaacgccaagaactccctgttcctgcagatgaacagcctgcgcgacgaggacatggccgtgtactattgtgccagaggccactacttcgactactggggacagggcacactggtcacagtctcttct

SEQ ID NO: 317 - DNA, 321 bp, synthetic construct
  source 1..321: mol_type = other DNA
gagatcgtgatgacccagtctcctgccacactgtctgtgtctcccggcgagagagctaccctgtcttgtagagcctctcagtccgtgtcctccaacctggcctggtatcagcagaagcctggactggctccccggctgttgatctatggcgcttctaccagagccacaggcatccccgctagattctccggctctggctctggcacagagtttaccctgaccatctccagcctgcagtccgaggatttcgccgtgtactactgccagcagtacaacaactggccccggacctttggccagggcaccaaggtggaaatcaag

SEQ ID NO: 318 - DNA, 363 bp, synthetic construct
  source 1..363: mol_type = other DNA
caggttcagctggttcagtctggcgccgaagtgaagaaacctggctcctccgtgaaagtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggttcactacgactacgccggctttccttattggggctgtggcaccctggtcaccgtttcttct

SEQ ID NO: 319 - DNA, 318 bp, synthetic construct
  source 1..318: mol_type = other DNA
gagatcgtgctgacccagtctcctgccacactgagtgcttctccaggcgagagagtgaccctgtcctgctccgcttcctcctccgtgtcctacatgaactggtatcagcagaagcccggctgcgcccctagaagatggatctacgactcctccaagctggcctctggcgtgcctgctagattttccggctctggctctggcagagactataccctgacaatctccagcctggaacctgaggacttcgccgtgtactactgccagcagtggtctaggaaccctcctacctttggcggaggcaccaaggtggaaatcaag

SEQ ID NO: 320 - DNA, 1431 bp, synthetic construct
  source 1..1431: mol_type = other DNA
gagatcgtgctgacccagtctcctgccacactgagtgcttctccaggcgagagagtgaccctgtcctgctccgcttcctcctccgtgtcctacatgaactggtatcagcagaagcccggctgcgcccctagaagatggatctacgactcctccaagctggcctctggcgtgcctgctagattttccggctctggctctggcagagactataccctgacaatctccagcctggaacctgaggacttcgccgtgtactactgccagcagtggtctaggaaccctcctacctttggcggaggcaccaaggtggaaatcaagggcggaggttctggtggatctggcggatgtcctccttgtggtggaagcggcggacaggttcagctggttcagtctggcgccgaagtgaagaaacctggctcctccgtgaaagtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggttcactacgactacgccggctttccttattggggctgtggcaccctggtcaccgtttcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 321 - DNA, 1335 bp, synthetic construct
  source 1..1335: mol_type = other DNA
gaggtgcagctggttgaatctggcggcggaagagtgaagcctggcggatctctgagactgtcttgtgccgcctctggcttcaccttctccgactactccatgaactgggtccgacaggctcctggcaaaggcctggaatgggtgtcctctatctcctccatctccagctacgtgaagtacgccgactccgtgaagggcagattcaccatgtccagagacaacgccaagaactccctgttcctgcagatgaacagcctgcgcgacgaggacatggccgtgtactattgtgccagaggccactacttcgactactggggacagggcacactggtcacagtctcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 322 - DNA, 642 bp, synthetic construct
  source 1..642: mol_type = other DNA
gagatcgtgatgacccagtctcctgccacactgtctgtgtctcccggcgagagagctaccctgtcttgtagagcctctcagtccgtgtcctccaacctggcctggtatcagcagaagcctggactggctccccggctgttgatctatggcgcttctaccagagccacaggcatccccgctagattctccggctctggctctggcacagagtttaccctgaccatctccagcctgcagtccgaggatttcgccgtgtactactgccagcagtacaacaactggccccggacctttggccagggcaccaaggtggaaatcaagcgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 323 - protein, 4 aa, synthetic construct
  REGION 1: note = When part of an spFv VL domain, an intrachain disulfide bond will be formed
  REGION 4: note = When part of an spFv VH domain, an intrachain disulfide bond will be formed
CPPC

SEQ ID NO: 324 - DNA, 1434 bp, synthetic construct
  source 1..1434: mol_type = other DNA
gagatcgtgctgacccagtctcctgccacactgagtgcttctccaggcgagagagtgaccctgtcctgctccgcttcctcctccgtgtcctacatgaactggtatcagcagaagcccggctgcgcccctagaagatggatctacgactcctccaagctggcctctggcgtgcctgctagattttccggctctggctctggcagagactataccctgacaatctccagcctggaacctgaggacttcgccgtgtactactgccagcagtggtctaggaaccctcctacctttggcggaggcaccaaggtggaaatcaagggcggaggttctggtggatctggcggatgtcctccttgtggtggaagcggcggacaggttcagctggttcagtctggcgccgaagtgaagaaacctggctcctccgtgaaagtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggttcactacgactacgccggctttccttattggggctgtggcaccctggtcaccgtttcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaatga

SEQ ID NO: 325 - DNA, 1338 bp, synthetic construct
  source 1..1338: mol_type = other DNA
gaggtgcagctggttgaatctggcggcggaagagtgaagcctggcggatctctgagactgtcttgtgccgcctctggcttcaccttctccgactactccatgaactgggtccgacaggctcctggcaaaggcctggaatgggtgtcctctatctcctccatctccagctacgtgaagtacgccgactccgtgaagggcagattcaccatgtccagagacaacgccaagaactccctgttcctgcagatgaacagcctgcgcgacgaggacatggccgtgtactattgtgccagaggccactacttcgactactggggacagggcacactggtcacagtctcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaatga

SEQ ID NO: 326 - DNA, 645 bp, synthetic construct
  source 1..645: mol_type = other DNA
gagatcgtgatgacccagtctcctgccacactgtctgtgtctcccggcgagagagctaccctgtcttgtagagcctctcagtccgtgtcctccaacctggcctggtatcagcagaagcctggactggctccccggctgttgatctatggcgcttctaccagagccacaggcatccccgctagattctccggctctggctctggcacagagtttaccctgaccatctccagcctgcagtccgaggatttcgccgtgtactactgccagcagtacaacaactggccccggacctttggccagggcaccaaggtggaaatcaagcgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 327 - protein, 855 aa, Homo sapiens
HHHHHHHHHHGLNDIFEAQKIEWHERKLEKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 328 - protein, 19 aa, Homo sapiens
PDEITKHLANTDVPIPTHY

SEQ ID NO: 329 - protein, 13 aa, Homo sapiens
PPASNRTSDSQYD